(19) United States
(12) Patent Application Publication
(10) Pub. No.: US 2025/0270635 A1
(43) Pub. Date: Aug. 28, 2025

(54) MULTIVALENT NUCLEOTIDE CONSTRUCTS

(71) Applicant: Singular Genomics Systems, Inc., San Diego, CA (US)

(72) Inventor: Ronald Graham, Carlsbad, CA (US)

(21) Appl. No.: 19/057,416

(22) Filed: Feb. 19, 2025

Related U.S. Application Data
(60) Provisional application No. 63/557,047, filed on Feb. 23, 2024.
(60) Provisional application No. 63/659,284, filed on Jun. 12, 2024.

(51) Int. Cl. C12Q 1/6869; C07H 19/14; C12Q 1/6876; G01N 33/68

(52) U.S. Cl. CPC C12Q 1/6869; C07H 19/14; C12Q 1/6876; G01N 33/6818

(57) ABSTRACT

Disclosed herein, inter alia, are multivalent nucleotide constructs and methods for making and using such constructs.

### CROSS-REFERENCES TO RELATED APPLICATIONS

This application claims the benefit of U.S. Provisional Application No. 63/557,047, filed Feb. 23, 2024; and U.S. Provisional Application No. 63/659,284, filed Jun. 12, 2024, both of which are incorporated herein by reference in their entirety and for all purposes.

### BACKGROUND

DNA sequencing is a fundamental tool in biological and medical research; it is an essential technology for the paradigm of personalized precision medicine. Accordingly, there is a need for efficient sequencing reagents, such as modified nucleotides and nucleotide constructs, that can facilitate the generation of sequencing runs with uniform coverage and improved efficiency. Disclosed herein, inter alia, are solutions to these and other problems in the art.

### BRIEF SUMMARY

In an aspect is provided a compound having the formula:

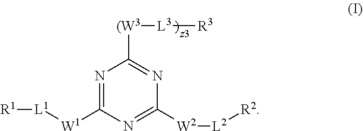

R¹ is a nucleotide moiety. R² is a nucleotide moiety or a fluorescent moiety. R³ is a bioconjugate-reactive moiety. W¹ is —O—, —NR^{1A}—, or —S—. W² is —O—, —NR^{2A}—, or —S—. W³ is —O—, —NR^{3A}—, or —S—. R^{1A}, R^{2A}, and R^{3A} are independently hydrogen or substituted or unsubstituted alkyl. L¹, L², and L³ and are independently covalent linkers. z3 is 0 or 1.

In an aspect is provided a biomolecule attached to a nucleotide construct, wherein said nucleotide construct has the formula:

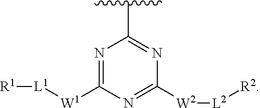

R¹ is a nucleotide moiety. R² is a nucleotide moiety or a fluorescent moiety. W¹ is —O—, —NR^{1A}—, or —S—. W² is —O—, —NR^{2A}—, or —S—. R^{1A} and R^{2A} are independently hydrogen or substituted or unsubstituted alkyl; and L¹ and L² and are independently covalent linkers.

In an aspect is provided a method of imaging a biomolecule, the method including directing an excitation beam onto a biomolecule including a detectable moiety and detecting a light emission from the detectable moiety, wherein the biomolecule is as described herein.

In an aspect is provided a method of making a compound of formula (I), the method including mixing compound A and compound B in a reaction vessel. Compound A has the formula:

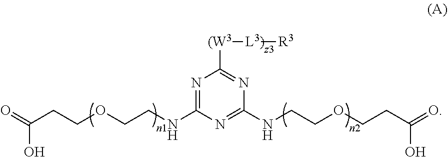

Compound B has the formula:

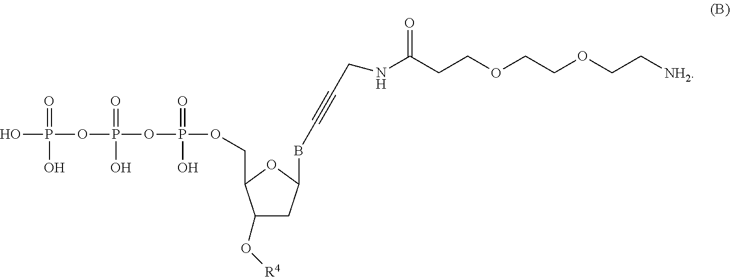

B is a nucleobase. R³ is a bioconjugate reactive moiety. R⁴ is hydrogen or a reversible terminator moiety. W³ is —O—,
“\*MERGEFORMAT\*MERGEFORMAT —NR^{3A}—, or —S—. R^{3A} is hydrogen or substituted or unsubstituted alkyl. L³ is a covalent linker. n1 and n2 are independently an integer from 1 to 10. z3 is 0 or 1.

### BRIEF DESCRIPTION OF THE DRAWINGS

FIGS. 1A and 1B. FIG. 1A provides a structure of the compound described herein. The structure includes a central trifunctional scaffolding moiety (depicted as a triangle shape) that is linked to a bioconjugate reactive moiety (depicted as an arc shape), a fluorescent moiety (depicted as a star shape), and a nucleotide moiety (illustrated with the pentagon bonded to a rectangle and a circle, where the rectangle depicts a nucleobase and the circle depicts a phosphate group). The bioconjugate reactive moiety, a fluorescent moiety, and a nucleotide moiety are all linked to the central trifunctional scaffold moiety via linker moieties (represented by the squiggly line). FIG. 1B illustrates a multivalent nucleotide construct resulting from a chemical reaction of the bioconjugate reactive moiety of the compound of FIG. 1A and a second bioconjugate reactive moiety attached onto a protein (depicted as a cloud shape) via a linker moiety (represented as a squiggly line).

### DETAILED DESCRIPTION

The aspects and embodiments described herein relate to multivalent nucleotide constructs and methods for making and using such constructs.

### I. Definitions

All patents, patent applications, articles and publications mentioned herein, both supra and infra, are hereby expressly incorporated herein by reference in their entireties. The practice of the technology described herein will employ, unless indicated specifically to the contrary, conventional methods of chemistry, biochemistry, organic chemistry, molecular biology, bioinformatics, microbiology, recombinant DNA techniques, genetics, immunology, and cell biology that are within the skill of the art, many of which are described below for the purpose of illustration. Examples of such techniques are available in the literature. See, e.g., Singleton et al., DICTIONARY OF MICROBIOLOGY AND MOLECULAR BIOLOGY 2nd ed., J. Wiley & Sons (New York, NY 1994); and Sambrook and Green, Molecular Cloning: A Laboratory Manual, 4th Edition (2012). Methods, devices and materials similar or equivalent to those described herein can be used in the practice of this invention.

Unless defined otherwise herein, all technical and scientific terms used herein have the same meaning as commonly understood by one of ordinary skill in the art to which this disclosure belongs. Various scientific dictionaries that include the terms included herein are well known and available to those in the art. Although any methods and materials similar or equivalent to those described herein find use in the practice or testing of the disclosure, some preferred methods and materials are described. Accordingly, the terms defined immediately below are more fully described by reference to the specification as a whole. It is to be understood that this disclosure is not limited to the particular methodology, protocols, and reagents described, as these may vary, depending upon the context in which they are used by those of skill in the art. The following definitions are provided to facilitate understanding of certain terms used frequently herein and are not meant to limit the scope of the present disclosure.

As used herein, the singular terms “a”, “an”, and “the” include the plural reference unless the context clearly indicates otherwise. Reference throughout this specification to, for example, “one embodiment”, “an embodiment”, “another embodiment”, “a particular embodiment”, “a related embodiment”, “a certain embodiment”, “an additional embodiment”, or “a further embodiment” or combinations thereof means that a particular feature, structure or characteristic described in connection with the embodiment is included in at least one embodiment of the present disclosure. Thus, the appearances of the foregoing phrases in various places throughout this specification are not necessarily all referring to the same embodiment. Furthermore, the particular features, structures, or characteristics may be combined in any suitable manner in one or more embodiments.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference in their entirety for all purposes.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

The abbreviations used herein have their conventional meaning within the chemical and biological arts. The chemical structures and formulae set forth herein are constructed according to the standard rules of chemical valency known in the chemical arts.

Where substituent groups are specified by their conventional chemical formulae, written from left to right, they equally encompass the chemically identical substituents that would result from writing the structure from right to left, e.g., —CH₂O—is equivalent to —OCH₂—.

The term “alkyl,” by itself or as part of another substituent, means, unless otherwise stated, a straight (i.e., unbranched) or branched carbon chain (or carbon), or combination thereof, which may be fully saturated, mono- or polyunsaturated and can include mono-, di-, and multivalent radicals. The alkyl may include a designated number of carbons (e.g., C₁-C₁₀ means one to ten carbons). In embodiments, the alkyl is fully saturated. In embodiments, the alkyl is monounsaturated. In embodiments, the alkyl is polyunsaturated. Alkyl is an uncyclized chain. Examples of saturated hydrocarbon radicals include, but are not limited to, groups such as methyl, ethyl, n-propyl, isopropyl, n-butyl, t-butyl, isobutyl, sec-butyl, methyl, homologs and isomers of, for example, n-pentyl, n-hexyl, n-heptyl, n-octyl, and the like. An unsaturated alkyl group is one having one or more double bonds or triple bonds. Examples of unsaturated alkyl groups include, but are not limited to, vinyl, 2-propenyl, crotyl, 2-isopentenyl, 2-(butadienyl), 2,4-pentadienyl, 3-(1,4-pentadienyl), ethynyl, 1- and 3-propynyl, 3-butynyl, and the higher homologs and isomers. An alkoxy is an alkyl attached to the remainder of the molecule via an oxygen linker (—O—). An alkyl moiety may be an alkenyl moiety. An alkyl moiety may be an alkynyl moiety. An alkenyl includes one or more double bonds. An alkynyl includes one or more triple bonds.

The term “alkylene,” by itself or as part of another substituent, means, unless otherwise stated, a divalent radical derived from an alkyl, as exemplified, but not limited by,
“\*MERGEFORMAT\*MERGEFORMAT —CH₂CH₂CH₂CH₂—. Typically, an alkyl (or alkylene) group will have from 1 to 24 carbon atoms, with those groups having 10 or fewer carbon atoms being preferred herein. A “lower alkyl” or “lower alkylene” is a shorter chain alkyl or alkylene group, generally having eight or fewer carbon atoms. The term “alkenylene,” by itself or as part of another substituent, means, unless otherwise stated, a divalent radical derived from an alkene. The term “alkynylene” by itself or as part of another substituent, means, unless otherwise stated, a divalent radical derived from an alkyne. The term “alkynylene” by itself or as part of another substituent, means, unless otherwise stated, a divalent radical derived from an alkyne. In embodiments, the alkylene is fully saturated. In embodiments, the alkylene is monounsaturated. In embodiments, the alkylene is polyunsaturated. An alkenylene includes one or more double bonds. An alkynylene includes one or more triple bonds.

The term “heteroalkyl,” by itself or in combination with another term, means, unless otherwise stated, a stable straight or branched chain, or combinations thereof, including at least one carbon atom and at least one heteroatom (e.g., O, N, P, Si, and S), and wherein the nitrogen and sulfur atoms may optionally be oxidized, and the nitrogen heteroatom may optionally be quaternized. The heteroatom(s) (e.g., O, N, S, Si, or P) may be placed at any interior position of the heteroalkyl group or at the position at which the alkyl group is attached to the remainder of the molecule. Heteroalkyl is an uncyclized chain. Examples include, but are not limited to:
“\*MERGEFORMAT\*MERGEFORMAT —CH₂—CH₂—O—CH₃, —CH₂—CH₂—NH—CH₃, —CH₂—CH₂—N(CH₃)—CH₃, —CH₂—S—CH₂—CH₃,
“\*MERGEFORMAT\*MERGEFORMAT —CH₂—S—CH₂, —S(O)—CH₃, —CH₂—CH₂—S(O)₂—CH₃, —CH═CHO—CH₃, —Si(CH₃)₃,
“\*MERGEFORMAT\*MERGEFORMAT —CH₂—CH═N—OCH₃, —CH═CH—N(CH₃)—CH₃, —O—CH₃, —O—CH₂—CH₃, and —CN. Up to two or three heteroatoms may be consecutive, such as, for example, —CH₂—NH—OCH₃ and —CH₂—O—Si(CH₃)₃. A heteroalkyl moiety may include one heteroatom (e.g., O, N, S, Si, or P). A heteroalkyl moiety may include two optionally different heteroatoms (e.g., O, N, S, Si, or P). A heteroalkyl moiety may include three optionally different heteroatoms (e.g., O, N, S, Si, or P). A heteroalkyl moiety may include four optionally different heteroatoms (e.g., O, N, S, Si, or P). A heteroalkyl moiety may include five optionally different heteroatoms (e.g., O, N, S, Si, or P). A heteroalkyl moiety may include up to 8 optionally different heteroatoms (e.g., O, N, S, Si, or P). The term “heteroalkenyl,” by itself or in combination with another term, means, unless otherwise stated, a heteroalkyl including at least one double bond. A heteroalkenyl may optionally include more than one double bond and/or one or more triple bonds in additional to the one or more double bonds. The term “heteroalkynyl,” by itself or in combination with another term, means, unless otherwise stated, a heteroalkyl including at least one triple bond. A heteroalkynyl may optionally include more than one triple bond and/or one or more double bonds in additional to the one or more triple bonds. In embodiments, the heteroalkyl is fully saturated. In embodiments, the heteroalkyl is monounsaturated. In embodiments, the heteroalkyl is polyunsaturated.

Similarly, the term “heteroalkylene,” by itself or as part of another substituent, means, unless otherwise stated, a divalent radical derived from heteroalkyl, as exemplified, but not limited by, —CH₂—CH₂—S—CH₂—CH₂— and —CH₂—S—CH₂—CH₂—NH—CH₂—. For heteroalkylene groups, heteroatoms can also occupy either or both of the chain termini (e.g., alkyleneoxy, alkylenedioxy, alkyleneamino, alkylenediamino, and the like). Still further, for alkylene and heteroalkylene linking groups, no orientation of the linking group is implied by the direction in which the formula of the linking group is written. For example, the formula —C(O)₂R′— represents both —C(O)₂R′—and —R′C(O)₂—. As described above, heteroalkyl groups, as used herein, include those groups that are attached to the remainder of the molecule through a heteroatom, such as
“\*MERGEFORMAT\*MERGEFORMAT —C(O)R′, —C(O)NR′, —NR′R″, —OR′, —SR′, and/or —SO₂R′. Where “heteroalkyl” is recited, followed by recitations of specific heteroalkyl groups, such as —NR′R″ or the like, it will be understood that the terms heteroalkyl and —NR′R″ are not redundant or mutually exclusive. Rather, the specific heteroalkyl groups are recited to add clarity. Thus, the term “heteroalkyl” should not be interpreted herein as excluding specific heteroalkyl groups, such as —NR′R″ or the like. The term “heteroalkenylene,” by itself or as part of another substituent, means, unless otherwise stated, a divalent radical derived from a heteroalkene. The term “heteroalkynylene” by itself or as part of another substituent, means, unless otherwise stated, a divalent radical derived from a heteroalkyne. In embodiments, the heteroalkylene is fully saturated. In embodiments, the heteroalkylene is monounsaturated. In embodiments, the heteroalkylene is polyunsaturated. A heteroalkenylene includes one or more double bonds. A heteroalkynylene includes one or more triple bonds.

The terms “cycloalkyl” and “heterocycloalkyl,” by themselves or in combination with other terms, mean, unless otherwise stated, cyclic versions of “alkyl” and “heteroalkyl,” respectively. Cycloalkyl and heterocycloalkyl are not aromatic. Additionally, for heterocycloalkyl, a heteroatom can occupy the position at which the heterocycle is attached to the remainder of the molecule. Examples of cycloalkyl include, but are not limited to, cyclopropyl, cyclobutyl, cyclopentyl, cyclohexyl, 1-cyclohexenyl, 3-cyclohexenyl, cycloheptyl, and the like. Examples of heterocycloalkyl include, but are not limited to, 1-(1,2,5,6-tetrahydropyridyl), 1-piperidinyl, 2-piperidinyl, 3-piperidinyl, 4-morpholinyl, 3-morpholinyl, tetrahydrofuran-2-yl, tetrahydrofuran-3-yl, tetrahydrothien-2-yl, tetrahydrothien-3-yl, 1-piperazinyl, 2-piperazinyl, and the like. A “cycloalkylene” and a “heterocycloalkylene,” alone or as part of another substituent, means a divalent radical derived from a cycloalkyl and heterocycloalkyl, respectively. In embodiments, the cycloalkyl is fully saturated. In embodiments, the cycloalkyl is monounsaturated. In embodiments, the cycloalkyl is polyunsaturated. In embodiments, the heterocycloalkyl is fully saturated. In embodiments, the heterocycloalkyl is monounsaturated. In embodiments, the heterocycloalkyl is polyunsaturated.

In embodiments, the term “cycloalkyl” means a monocyclic, bicyclic, or a multicyclic cycloalkyl ring system. In embodiments, monocyclic ring systems are cyclic hydrocarbon groups containing from 3 to 8 carbon atoms, where such groups can be saturated or unsaturated, but not aromatic. In embodiments, cycloalkyl groups are fully saturated. A bicyclic or multicyclic cycloalkyl ring system refers to multiple rings fused together wherein at least one of the fused rings is a cycloalkyl ring and wherein the multiple rings are attached to the parent molecular moiety through any carbon atom contained within a cycloalkyl ring of the multiple rings.

In embodiments, a cycloalkyl is a cycloalkenyl. The term “cycloalkenyl” is used in accordance with its plain ordinary meaning. In embodiments, a cycloalkenyl is a monocyclic, bicyclic, or a multicyclic cycloalkenyl ring system. A bicyclic or multicyclic cycloalkenyl ring system refers to multiple rings fused together wherein at least one of the fused rings is a cycloalkenyl ring and wherein the multiple rings are attached to the parent molecular moiety through any carbon atom contained within a cycloalkenyl ring of the multiple rings.

In embodiments, the term “heterocycloalkyl” means a monocyclic, bicyclic, or a multicyclic heterocycloalkyl ring system. In embodiments, heterocycloalkyl groups are fully saturated. A bicyclic or multicyclic heterocycloalkyl ring system refers to multiple rings fused together wherein at least one of the fused rings is a heterocycloalkyl ring and wherein the multiple rings are attached to the parent molecular moiety through any atom contained within a heterocycloalkyl ring of the multiple rings.

The terms “halo” or “halogen,” by themselves or as part of another substituent, mean, unless otherwise stated, a fluorine, chlorine, bromine, or iodine atom. Additionally, terms such as “haloalkyl” are meant to include monohaloalkyl and polyhaloalkyl. For example, the term “halo(C₁-C₄)alkyl” includes, but is not limited to, fluoromethyl, difluoromethyl, trifluoromethyl, 2,2,2-trifluoroethyl, 4-chlorobutyl, 3-bromopropyl, and the like.

The term “acyl” means, unless otherwise stated, —C(O)R where R is a substituted or unsubstituted alkyl, substituted or unsubstituted cycloalkyl, substituted or unsubstituted heteroalkyl, substituted or unsubstituted heterocycloalkyl, substituted or unsubstituted aryl, or substituted or unsubstituted heteroaryl.

The term “aryl” means, unless otherwise stated, a polyunsaturated, aromatic, hydrocarbon substituent, which can be a single ring or multiple rings (preferably from 1 to 3 rings) that are fused together (i.e., a fused ring aryl) or linked covalently. A fused ring aryl refers to multiple rings fused together wherein at least one of the fused rings is an aryl ring and wherein the multiple rings are attached to the parent molecular moiety through any carbon atom contained within an aryl ring of the multiple rings. The term “heteroaryl” refers to aryl groups (or rings) that contain at least one heteroatom such as N, O, or S, wherein the nitrogen and sulfur atoms are optionally oxidized, and the nitrogen atom(s) are optionally quaternized. Thus, the term “heteroaryl” includes fused ring heteroaryl groups (i.e., multiple rings fused together wherein at least one of the fused rings is a heteroaromatic ring and wherein the multiple rings are attached to the parent molecular moiety through any atom contained within a heteroaromatic ring of the multiple rings). A 5,6-fused ring heteroarylene refers to two rings fused together, wherein one ring has 5 members and the other ring has 6 members, and wherein at least one ring is a heteroaryl ring. Likewise, a 6,6-fused ring heteroarylene refers to two rings fused together, wherein one ring has 6 members and the other ring has 6 members, and wherein at least one ring is a heteroaryl ring. And a 6,5-fused ring heteroarylene refers to two rings fused together, wherein one ring has 6 members and the other ring has 5 members, and wherein at least one ring is a heteroaryl ring. A heteroaryl group can be attached to the remainder of the molecule through a carbon or heteroatom. Non-limiting examples of aryl and heteroaryl groups include phenyl, naphthyl, pyrrolyl, pyrazolyl, pyridazinyl, triazinyl, pyrimidinyl, imidazolyl, pyrazinyl, purinyl, oxazolyl, isoxazolyl, thiazolyl, furyl, thienyl, pyridyl, pyrimidyl, benzothiazolyl, benzoxazoyl benzimidazolyl, benzofuran, isobenzofuranyl, indolyl, isoindolyl, benzothiophenyl, isoquinolyl, quinoxalinyl, quinolyl, 1-naphthyl, 2-naphthyl, 4-biphenyl, 1-pyrrolyl, 2-pyrrolyl, 3-pyrrolyl, 3-pyrazolyl, 2-imidazolyl, 4-imidazolyl, pyrazinyl, 2-oxazolyl, 4-oxazolyl, 2-phenyl-4-oxazolyl, 5-oxazolyl, 3-isoxazolyl, 4-isoxazolyl, 5-isoxazolyl, 2-thiazolyl, 4-thiazolyl, 5-thiazolyl, 2-furyl, 3-furyl, 2-thienyl, 3-thienyl, 2-pyridyl, 3-pyridyl, 4-pyridyl, 2-pyrimidyl, 4-pyrimidyl, 5-benzothiazolyl, purinyl, 2-benzimidazolyl, 5-indolyl, 1-isoquinolyl, 5-isoquinolyl, 2-quinoxalinyl, 5-quinoxalinyl, 3-quinolyl, and 6-quinolyl. Substituents for each of the above noted aryl and heteroaryl ring systems are selected from the group of acceptable substituents described below. An “arylene” and a “heteroarylene,” alone or as part of another substituent, mean a divalent radical derived from an aryl and heteroaryl, respectively. A heteroaryl group substituent may be —O— bonded to a ring heteroatom nitrogen.

Spirocyclic rings are two or more rings wherein adjacent rings are attached through a single atom. The individual rings within spirocyclic rings may be identical or different. Individual rings in spirocyclic rings may be substituted or unsubstituted and may have different substituents from other individual rings within a set of spirocyclic rings. Possible substituents for individual rings within spirocyclic rings are the possible substituents for the same ring when not part of spirocyclic rings (e.g., substituents for cycloalkyl or heterocycloalkyl rings). Spirocyclic rings may be substituted or unsubstituted cycloalkyl, substituted or unsubstituted cycloalkylene, substituted or unsubstituted heterocycloalkyl or substituted or unsubstituted heterocycloalkylene and individual rings within a spirocyclic ring group may be any of the immediately previous list, including having all rings of one type (e.g., all rings being substituted heterocycloalkylene wherein each ring may be the same or different substituted heterocycloalkylene). When referring to a spirocyclic ring system, heterocyclic spirocyclic rings means a spirocyclic rings wherein at least one ring is a heterocyclic ring and wherein each ring may be a different ring. When referring to a spirocyclic ring system, substituted spirocyclic rings means that at least one ring is substituted and each substituent may optionally be different.

The symbol “ [symbol] ” denotes the point of attachment of a chemical moiety to the remainder of a molecule or chemical formula.

The term “oxo,” as used herein, means an oxygen that is double bonded to a carbon atom.

Each of the above terms (e.g., “alkyl,” “heteroalkyl,” “cycloalkyl,” “heterocycloalkyl,” “aryl,” and “heteroaryl”) includes both substituted and unsubstituted forms of the indicated radical. Preferred substituents for each type of radical are provided below.

Substituents for the alkyl and heteroalkyl radicals (including those groups often referred to as alkylene, alkenyl, heteroalkylene, heteroalkenyl, alkynyl, cycloalkyl, heterocycloalkyl, cycloalkenyl, and heterocycloalkenyl) can be one or more of a variety of groups selected from, but not limited to, —OR′, ═O, ═NR′, =N—OR′, —NR′R″, —SR′, -halogen, —SiR′R″R′″, —OC(O)R′,
“\*MERGEFORMAT\*MERGEFORMAT —C(O)R′, —CO₂R′, —CONR′R″, —OC(O)NR′R″, —NR″C(O)R′, —NR′—C(O)NR″R′″, —NR″C(O)₂R′,
“\*MERGEFORMAT\*MERGEFORMAT —NR—C(NR′R″R′″)=NR″″, —NR—C(NR′R″)═NR′″, —S(O)R′, —S(O)₂R′, —S(O)₂NR′R″, —NRSO₂R′, —NR′NR″R′″, —ONR′R″, —NR′C(O)NR″NR′″R″″, —CN, —NO₂, —NR′SO₂R″, —NR′C(O)R″,
“\*MERGEFORMAT\*MERGEFORMAT —NR′C(O)—OR″, —NR′OR″, in a number ranging from zero to (2m′+1), where m′ is the total number of carbon atoms in such radical. R, R′, R″, R′″, and R″″ each preferably independently refer to hydrogen, substituted or unsubstituted heteroalkyl, substituted or unsubstituted cycloalkyl, substituted or unsubstituted heterocycloalkyl, substituted or unsubstituted aryl (e.g., aryl substituted with 1-3 halogens), substituted or unsubstituted heteroaryl, substituted or unsubstituted alkyl, alkoxy, or thioalkoxy groups, or arylalkyl groups. When a compound described herein includes more than one R group, for example, each of the R groups is independently selected as are each R′, R″, R′″, and R″″ group when more than one of these groups is present. When R′ and R″ are attached to the same nitrogen atom, they can be combined with the nitrogen atom to form a 4-, 5-, 6-, or 7-membered ring. For example, —NR′R″ includes, but is not limited to, 1-pyrrolidinyl and 4-morpholinyl. From the above discussion of substituents, one of skill in the art will understand that the term “alkyl” is meant to include groups including carbon atoms bound to groups other than hydrogen groups, such as haloalkyl (e.g., —CF₃ and —CH₂CF₃) and acyl (e.g., —C(O)CH₃, —C(O)CF₃, —C(O)CH₂OCH₃, and the like).

Similar to the substituents described for the alkyl radical, substituents for the aryl and heteroaryl groups are varied and are selected from, for example: —OR′, —NR′R″, —SR′, halogen,
“\*MERGEFORMAT\*MERGEFORMAT —SiR′R″R″″, —OC(O)R′, —C(O)R′, —CO₂R′, —CONR′R″, —OC(O)NR′R″, —NR″C(O)R′,
“\*MERGEFORMAT\*MERGEFORMAT —NR′—C(O)NR″R′″, —NR″C(O)₂R′, —NR—C(NR′R″R′″)═NR″″, —NR—C(NR′R″)═NR′″, —S(O)R′,
“\*MERGEFORMAT\*MERGEFORMAT —S(O)₂R′, —S(O)₂NR′R″, —NRSO₂R′, —NR′NR″R′″, —ONR′R″, —NR′C(O)NR″NR′″R″″, —CN, —NO₂, —R′, —N₃, —CH(Ph)₂, fluoro(C₁-C₄)alkoxy, and fluoro(C₁-C₄)alkyl, —NR′SO₂R″, —NR′C(O)R″,
“\*MERGEFORMAT\*MERGEFORMAT —NR′C(O)—OR″, —NR′OR″, in a number ranging from zero to the total number of open valences on the aromatic ring system; and where R′, R″, R′″, and R″″ are preferably independently selected from hydrogen, substituted or unsubstituted alkyl, substituted or unsubstituted heteroalkyl, substituted or unsubstituted cycloalkyl, substituted or unsubstituted heterocycloalkyl, substituted or unsubstituted aryl, and substituted or unsubstituted heteroaryl. When a compound described herein includes more than one R group, for example, each of the R groups is independently selected as are each R′, R″, R′″, and R″″ groups when more than one of these groups is present.

Substituents for rings (e.g., cycloalkyl, heterocycloalkyl, aryl, heteroaryl, cycloalkylene, heterocycloalkylene, arylene, or heteroarylene) may be depicted as substituents on the ring rather than on a specific atom of a ring (commonly referred to as a floating substituent). In such a case, the substituent may be attached to any of the ring atoms (obeying the rules of chemical valency) and in the case of fused rings or spirocyclic rings, a substituent depicted as associated with one member of the fused rings or spirocyclic rings (a floating substituent on a single ring), may be a substituent on any of the fused rings or spirocyclic rings (a floating substituent on multiple rings). When a substituent is attached to a ring, but not a specific atom (a floating substituent), and a subscript for the substituent is an integer greater than one, the multiple substituents may be on the same atom, same ring, different atoms, different fused rings, different spirocyclic rings, and each substituent may optionally be different. Where a point of attachment of a ring to the remainder of a molecule is not limited to a single atom (a floating substituent), the attachment point may be any atom of the ring and in the case of a fused ring or spirocyclic ring, any atom of any of the fused rings or spirocyclic rings while obeying the rules of chemical valency. Where a ring, fused rings, or spirocyclic rings contain one or more ring heteroatoms and the ring, fused rings, or spirocyclic rings are shown with one more floating substituents (including, but not limited to, points of attachment to the remainder of the molecule), the floating substituents may be bonded to the heteroatoms. Where the ring heteroatoms are shown bound to one or more hydrogens (e.g., a ring nitrogen with two bonds to ring atoms and a third bond to a hydrogen) in the structure or formula with the floating substituent, when the heteroatom is bonded to the floating substituent, the substituent will be understood to replace the hydrogen, while obeying the rules of chemical valency.

Two or more substituents may optionally be joined to form aryl, heteroaryl, cycloalkyl, or heterocycloalkyl groups. Such so-called ring-forming substituents are typically, though not necessarily, found attached to a cyclic base structure. In one embodiment, the ring-forming substituents are attached to adjacent members of the base structure. For example, two ring-forming substituents attached to adjacent members of a cyclic base structure create a fused ring structure. In another embodiment, the ring-forming substituents are attached to a single member of the base structure. For example, two ring-forming substituents attached to a single member of a cyclic base structure create a spirocyclic structure. In yet another embodiment, the ring-forming substituents are attached to non-adjacent members of the base structure.

As used herein, the terms “heteroatom” or “ring heteroatom” are meant to include oxygen (O), nitrogen (N), sulfur (S), phosphorus (P), and silicon (Si).

A “substituent group,” as used herein, means a group selected from the following moieties:
(A) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —PO₃H, —PO₄H, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
(B) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
   (i) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —PO₃H, —PO₄H, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
   (ii) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₅ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from:
      (a) oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₂, —OCHBr₂, —OCHI₂, —OCHF₂, —PO₃H, —PO₄H, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl), and
      (b) alkyl (e.g., C₁-C₈ alkyl, C₁-C₆ alkyl, or C₁-C₄ alkyl), heteroalkyl (e.g., 2 to 8 membered heteroalkyl, 2 to 6 membered heteroalkyl, or 2 to 4 membered heteroalkyl), cycloalkyl (e.g., C₃-C₈ cycloalkyl, C₃-C₆ cycloalkyl, or C₅-C₆ cycloalkyl), heterocycloalkyl (e.g., 3 to 8 membered heterocycloalkyl, 3 to 6 membered heterocycloalkyl, or 5 to 6 membered heterocycloalkyl), aryl (e.g., C₆-C₁₀ aryl, C₁₀ aryl, or phenyl), heteroaryl (e.g., 5 to 10 membered heteroaryl, 5 to 9 membered heteroaryl, or 5 to 6 membered heteroaryl), substituted with at least one substituent selected from: oxo, halogen, —CCl₃, —CBr₃, —CF₃, —CI₃, —CH₂Cl, —CH₂Br, —CH₂F, —CH₂I, —CHCl₂, —CHBr₂, —CHF₂, —CHI₂, —CN, —OH, —NH₂, —COOH, —CONH₂, —NO₂, —SH, —SO₃H, —SO₄H, —SO₂NH₂, —NHNH₂, —ONH₂, —NHC(O)NHNH₂, —NHC(O)NH₂, —NHSO₂H, —NHC(O)H, —NHC(O)OH, —NHOH, —OCCl₃, —OCF₃, —OCBr₃, —OCI₃, —OCHCl₁₂, —OCHBr₂, —OCHI₂, —OCHF₂, —PO₃H, —PO₄H, —N₃, unsubstituted alkyl (e.g., C₁-C₈ alkyl, C₁-C₆ alkyl, or C₁-C₄ alkyl), unsubstituted heteroalkyl (e.g., 2 to 8 membered heteroalkyl, 2 to 6 membered heteroalkyl, or 2 to 4 membered heteroalkyl), unsubstituted cycloalkyl (e.g., C₃-C₈ cycloalkyl, C₃-C₆ cycloalkyl, or C₅-C₆ cycloalkyl), unsubstituted heterocycloalkyl (e.g., 3 to 8 membered heterocycloalkyl, 3 to 6 membered heterocycloalkyl, or 5 to 6 membered heterocycloalkyl), unsubstituted aryl (e.g., C₆-C₁₀ aryl, C₁₀ aryl, or phenyl), or unsubstituted heteroaryl (e.g., 5 to 10 membered heteroaryl, 5 to 9 membered heteroaryl, or 5 to 6 membered heteroaryl).

A “size-limited substituent” or “size-limited substituent group,” as used herein, means a group selected from all of the substituents described above for a “substituent group,” wherein each substituted or unsubstituted alkyl is a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and each substituted or unsubstituted heteroaryl is a substituted or unsubstituted 5 to 10 membered heteroaryl.

A “lower substituent” or “lower substituent group,” as used herein, means a group selected from all of the substituents described above for a “substituent group,” wherein each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and each substituted or unsubstituted heteroaryl is a substituted or unsubstituted 5 to 9 membered heteroaryl.

In some embodiments, each substituted group described in the compounds herein is substituted with at least one substituent group. More specifically, in some embodiments, each substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene described in the compounds herein are substituted with at least one substituent group. In other embodiments, at least one or all of these groups are substituted with at least one size-limited substituent group. In other embodiments, at least one or all of these groups are substituted with at least one lower substituent group.

In other embodiments of the compounds herein, each substituted or unsubstituted alkyl may be a substituted or unsubstituted C₁-C₂₀ alkyl, each substituted or unsubstituted heteroalkyl is a substituted or unsubstituted 2 to 20 membered heteroalkyl, each substituted or unsubstituted cycloalkyl is a substituted or unsubstituted C₃-C₈ cycloalkyl, each substituted or unsubstituted heterocycloalkyl is a substituted or unsubstituted 3 to 8 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 10 membered heteroaryl. In some embodiments of the compounds herein, each substituted or unsubstituted alkylene is a substituted or unsubstituted C₁-C₂₀ alkylene, each substituted or unsubstituted heteroalkylene is a substituted or unsubstituted 2 to 20 membered heteroalkylene, each substituted or unsubstituted cycloalkylene is a substituted or unsubstituted C₃-C₈ cycloalkylene, each substituted or unsubstituted heterocycloalkylene is a substituted or unsubstituted 3 to 8 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 10 membered heteroarylene.

In some embodiments, each substituted or unsubstituted alkyl is a substituted or unsubstituted C₁-C₈ alkyl, each substituted or unsubstituted heteroalkyl is a substituted or unsubstituted 2 to 8 membered heteroalkyl, each substituted or unsubstituted cycloalkyl is a substituted or unsubstituted C₃-C₇ cycloalkyl, each substituted or unsubstituted heterocycloalkyl is a substituted or unsubstituted 3 to 7 membered heterocycloalkyl, each substituted or unsubstituted aryl is a substituted or unsubstituted C₆-C₁₀ aryl, and/or each substituted or unsubstituted heteroaryl is a substituted or unsubstituted 5 to 9 membered heteroaryl. In some embodiments, each substituted or unsubstituted alkylene is a substituted or unsubstituted C₁-C₈ alkylene, each substituted or unsubstituted heteroalkylene is a substituted or unsubstituted 2 to 8 membered heteroalkylene, each substituted or unsubstituted cycloalkylene is a substituted or unsubstituted C₃-C₇ cycloalkylene, each substituted or unsubstituted heterocycloalkylene is a substituted or unsubstituted 3 to 7 membered heterocycloalkylene, each substituted or unsubstituted arylene is a substituted or unsubstituted C₆-C₁₀ arylene, and/or each substituted or unsubstituted heteroarylene is a substituted or unsubstituted 5 to 9 membered heteroarylene. In some embodiments, the compound (e.g., nucleotide analogue) is a chemical species set forth in the Examples section, claims, embodiments, figures, or tables below.

In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is unsubstituted (e.g., is an unsubstituted alkyl, unsubstituted heteroalkyl, unsubstituted cycloalkyl, unsubstituted heterocycloalkyl, unsubstituted aryl, unsubstituted heteroaryl, unsubstituted alkylene, unsubstituted heteroalkylene, unsubstituted cycloalkylene, unsubstituted heterocycloalkylene, unsubstituted arylene, and/or unsubstituted heteroarylene, respectively). In embodiments, a substituted or unsubstituted moiety (e.g., substituted or unsubstituted alkyl, substituted or unsubstituted heteroalkyl, substituted or unsubstituted cycloalkyl, substituted or unsubstituted heterocycloalkyl, substituted or unsubstituted aryl, substituted or unsubstituted heteroaryl, substituted or unsubstituted alkylene, substituted or unsubstituted heteroalkylene, substituted or unsubstituted cycloalkylene, substituted or unsubstituted heterocycloalkylene, substituted or unsubstituted arylene, and/or substituted or unsubstituted heteroarylene) is substituted (e.g., is a substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene, respectively).

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, wherein if the substituted moiety is substituted with a plurality of substituent groups, each substituent group may optionally be different. In embodiments, if the substituted moiety is substituted with a plurality of substituent groups, each substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one size-limited substituent group, wherein if the substituted moiety is substituted with a plurality of size-limited substituent groups, each size-limited substituent group may optionally be different. In embodiments, if the substituted moiety is substituted with a plurality of size-limited substituent groups, each size-limited substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one lower substituent group, wherein if the substituted moiety is substituted with a plurality of lower substituent groups, each lower substituent group may optionally be different. In embodiments, if the substituted moiety is substituted with a plurality of lower substituent groups, each lower substituent group is different.

In embodiments, a substituted moiety (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene) is substituted with at least one substituent group, size-limited substituent group, or lower substituent group; wherein if the substituted moiety is substituted with a plurality of groups selected from substituent groups, size-limited substituent groups, and lower substituent groups; each substituent group, size-limited substituent group, and/or lower substituent group may optionally be different. In embodiments, if the substituted moiety is substituted with a plurality of groups selected from substituent groups, size-limited substituent groups, and lower substituent groups; each substituent group, size-limited substituent group, and/or lower substituent group is different.

Where a moiety is substituted (e.g., substituted alkyl, substituted heteroalkyl, substituted cycloalkyl, substituted heterocycloalkyl, substituted aryl, substituted heteroaryl, substituted alkylene, substituted heteroalkylene, substituted cycloalkylene, substituted heterocycloalkylene, substituted arylene, and/or substituted heteroarylene), the moiety is substituted with at least one substituent (e.g., a substituent group, a size-limited substituent group, or lower substituent group) and each substituent is optionally different. Additionally, where multiple substituents are present on a moiety, each substituent may be optionally different.

Certain compounds of the present disclosure possess asymmetric carbon atoms (optical or chiral centers) or double bonds; the enantiomers, racemates, diastereomers, tautomers, geometric isomers, stereoisomeric forms that may be defined, in terms of absolute stereochemistry, as (R)- or (S)- or, as (D)- or (L)- for amino acids, and individual isomers are encompassed within the scope of the present disclosure. The compounds of the present disclosure do not include those that are known in art to be too unstable to synthesize and/or isolate. The present disclosure is meant to include compounds in racemic and optically pure forms. Optically active (R)- and (S)-, or (D)- and (L)-isomers may be prepared using chiral synthons or chiral reagents, or resolved using conventional techniques. When the compounds described herein contain olefinic bonds or other centers of geometric asymmetry, and unless specified otherwise, it is intended that the compounds include both E and Z geometric isomers.

As used herein, the term “isomers” refers to compounds having the same number and kind of atoms, and hence the same molecular weight, but differing in respect to the structural arrangement or configuration of the atoms.

The term “tautomer,” as used herein, refers to one of two or more structural isomers which exist in equilibrium and which are readily converted from one isomeric form to another.

It will be apparent to one skilled in the art that certain compounds of this disclosure may exist in tautomeric forms, all such tautomeric forms of the compounds being within the scope of the disclosure.

Unless otherwise stated, structures depicted herein are also meant to include all stereochemical forms of the structure; i.e., the R and S configurations for each asymmetric center. Therefore, single stereochemical isomers as well as enantiomeric and diastereomeric mixtures of the present compounds are within the scope of the disclosure.

Unless otherwise stated, structures depicted herein are also meant to include compounds which differ only in the presence of one or more isotopically enriched atoms. For example, compounds having the present structures except for the replacement of a hydrogen by a deuterium or tritium, or the replacement of a carbon by ¹³C- or ¹⁴C-enriched carbon are within the scope of this disclosure. The compounds of the present disclosure may also contain unnatural proportions of atomic isotopes at one or more of the atoms that constitute such compounds. For example, the compounds may be radiolabeled with radioactive isotopes, such as for example tritium (3H), iodine-125 (¹²⁵I), or carbon-14 (¹⁴C). All isotopic variations of the compounds of the present disclosure, whether radioactive or not, are encompassed within the scope of the present disclosure.

It should be noted that throughout the application that alternatives are written in Markush groups, for example, each amino acid position that contains more than one possible amino acid. It is specifically contemplated that each member of the Markush group should be considered separately, thereby comprising another embodiment, and the Markush group is not to be read as a single unit.

A “scaffold” is used in accordance with its plain ordinary meaning within Chemistry and Biology and refers to a derivatizable molecule capable of functioning as a platform or carrier of diverse, covalently attached chemical moieties. Chemical scaffolds are attractive molecules for joining together various chemical building blocks for the preparation of, for example, therapeutic molecules and chemical probes. Examples of scaffolds include, but are not limited to, amino acids (e.g., glutamic acid, lysine, and cysteine), triazines, and benzenes (Sato, D. et al. Design, Synthesis, and Utility of Defined Molecular Scaffolds. Organics 2021, 2, 161-273).

“Analog,” “analogue” or “derivative” is used in accordance with its plain ordinary meaning within Chemistry and Biology and refers to a chemical compound that is structurally similar to another compound (i.e., a so-called “reference” compound) but differs in composition, e.g., in the replacement of one atom by an atom of a different element, or in the presence of a particular functional group, or the replacement of one functional group by another functional group, or the absolute stereochemistry of one or more chiral centers of the reference compound. Accordingly, an analog is a compound that is similar or comparable in function and appearance but not in structure or origin to a reference compound. In the context of a nucleotide, a nucleotide analog refers to a compound that, like the nucleotide of which it is an analog, can be incorporated into a nucleic acid molecule (e.g., an extension product) by a suitable polymerase, for example, a DNA polymerase in the context of a nucleotide analogue. The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphorothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, e.g., see Eckstein, OLIGONUCLEOTIDES AND ANALOGUES: A PRACTICAL APPROACH, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g. phosphorodiamidate morpholino oligos or locked nucleic acids (LNA)), including those described in U.S. Pat. Nos. 5,235,033 and 5,034,506, and Chapters 6 and 7, ASC Symposium Series 580, CARBOHYDRATE MODIFICATIONS IN ANTISENSE RESEARCH, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

As used herein, a “native” nucleotide is used in accordance with its plain and ordinary meaning and refers to a naturally occurring nucleotide that does not include an exogenous label (e.g., a fluorescent dye, or other label) or chemical modification such as may characterize a nucleotide analog. Examples of native nucleotides useful for carrying out procedures described herein include: dATP (2′-deoxyadenosine-5′-triphosphate); dGTP (2′-deoxyguanosine-5′-triphosphate); dCTP (2′-deoxycytidine-5′-triphosphate); dTTP (2′-deoxythymidine-5′-triphosphate); and dUTP (2′-deoxyuridine-5′-triphosphate).

The terms “a” or “an,” as used in herein means one or more. In addition, the phrase “substituted with a[n],” as used herein, means the specified group may be substituted with one or more of any or all of the named substituents. For example, where a group, such as an alkyl or heteroaryl group, is “substituted with an unsubstituted C₁-C₂₀ alkyl, or unsubstituted 2 to 20 membered heteroalkyl,” the group may contain one or more unsubstituted C₁-C₂₀ alkyls, and/or one or more unsubstituted 2 to 20 membered heteroalkyls.

Moreover, where a moiety is substituted with an R substituent, the group may be referred to as “R-substituted.” Where a moiety is R-substituted, the moiety is substituted with at least one R substituent and each R substituent is optionally different. Where a particular R group is present in the description of a chemical genus (such as Formula (I)), a Roman alphabetic symbol may be used to distinguish each appearance of that particular R group. For example, where multiple R¹⁰ substituents are present, each R¹⁰ substituent may be distinguished as R^{10.1}, R^{10.2}, R^{10.3}, R^{10.4}, etc., wherein each of R^{10.1}, R^{10.2}, R^{10.3}, R^{10.4}, etc. is defined within the scope of the definition of R¹⁰ and optionally differently. Where an R moiety, group, or substituent as disclosed herein is attached through the representation of a single bond and the R moiety, group, or substituent is oxo, a person having ordinary skill in the art will immediately recognize that the oxo is attached through a double bond in accordance with the normal rules of chemical valency.

Descriptions of the compounds of the present disclosure are limited by principles of chemical bonding known to those skilled in the art. Accordingly, where a group may be substituted by one or more of a number of substituents, such substitutions are selected so as to comply with principles of chemical bonding and to give compounds which are not inherently unstable and/or would be known to one of ordinary skill in the art as likely to be unstable under ambient conditions, such as aqueous, neutral, and several known physiological conditions. For example, a heterocycloalkyl or heteroaryl is attached to the remainder of the molecule via a ring heteroatom in compliance with principles of chemical bonding known to those skilled in the art thereby avoiding inherently unstable compounds.

The compounds of the present invention may exist as salts, such as with pharmaceutically acceptable acids. The present invention includes such salts. Non-limiting examples of such salts include hydrochlorides, hydrobromides, phosphates, sulfates, methanesulfonates, nitrates, maleates, acetates, citrates, fumarates, proprionates, tartrates (e.g., (+)-tartrates, (−)-tartrates, or mixtures thereof including racemic mixtures), succinates, benzoates, and salts with amino acids such as glutamic acid, and quaternary ammonium salts (e.g., methyl iodide, ethyl iodide, and the like). These salts may be prepared by methods known to those skilled in the art. The neutral forms of the compounds are preferably regenerated by contacting the salt with a base or acid and isolating the parent compound in the conventional manner. The parent form of the compound may differ from the various salt forms in certain physical properties, such as solubility in polar solvents.

Certain compounds of the present invention can exist in unsolvated forms as well as solvated forms, including hydrated forms. In general, the solvated forms are equivalent to unsolvated forms and are encompassed within the scope of the present invention. Certain compounds of the present invention may exist in multiple crystalline or amorphous forms. In general, all physical forms are equivalent for the uses contemplated by the present invention and are intended to be within the scope of the present invention.

“Contacting” is used in accordance with its plain ordinary meaning and refers to the process of allowing at least two distinct species (e.g., chemical compounds including biomolecules or cells, or bioconjugate reactive moieties) to become sufficiently proximal to react, interact or physically touch. It should be appreciated, however, that the resulting reaction product can be produced directly from a reaction between the added reagents or from an intermediate from one or more of the added reagents that can be produced in the reaction mixture. The term “contacting” may include allowing two species to react, interact, or physically touch, wherein the two species may be a compound as described herein and a nucleotide, linker, protein, or enzyme.

The term “expression” includes any step involved in the production of the polypeptide including, but not limited to, transcription, post-transcriptional modification, translation, post-translational modification, and secretion. Expression can be detected using conventional techniques for detecting protein (e.g., ELISA, Western blotting, flow cytometry, immunofluorescence, immunohistochemistry, etc.).

“Nucleic acid,” “oligonucleotide,” “nucleic acid molecule,” “nucleic acid sequence,” “nucleic acid fragment,” or “polynucleotide” are used interchangeably and are intended to include, but are not limited to, a polymeric form of nucleotides covalently linked together that may have various lengths, either deoxyribonucleotides or ribonucleotides, or analogs, derivatives or modifications thereof. The term “nucleic acid” includes single- or double-stranded DNA, RNA and analogs (derivatives) thereof. Different polynucleotides may have different three-dimensional structures, and may perform various functions, known or unknown. Non-limiting examples of polynucleotides include a gene, a gene fragment, an exon, an intron, intergenic DNA (including, without limitation, heterochromatic DNA), messenger RNA (mRNA), transfer RNA, ribosomal RNA, a ribozyme, cDNA, a recombinant polynucleotide, a branched polynucleotide, a plasmid, a vector, isolated DNA of a sequence, isolated RNA of a sequence, a nucleic acid probe, and a primer. Polynucleotides useful in the methods of the disclosure may include natural nucleic acid sequences and variants thereof, artificial nucleic acid sequences, or a combination of such sequences. As may be used herein, the terms “nucleic acid oligomer” and “oligonucleotide” are used interchangeably and are intended to include, but are not limited to, nucleic acids having a length of 200 nucleotides or less. In some embodiments, an oligonucleotide is a nucleic acid having a length of 2 to 200 nucleotides, 2 to 150 nucleotides, 5 to 150 nucleotides or 5 to 100 nucleotides. Oligonucleotides are typically from about 5, 6, 7, 8, 9, 10, 12, 15, 25, 30, 40, 50 or more nucleotides in length, up to about 100 nucleotides in length. Nucleic acids and polynucleotides are polymers of any length, including longer lengths, e.g., 200, 300, 500, 1000, 2000, 3000, 5000, 7000, 10,000, etc. In some embodiments, an oligonucleotide is a primer configured for extension by a polymerase when the primer is annealed completely or partially to a complementary nucleic acid template. A primer is often a single stranded nucleic acid. In certain embodiments, a primer, or portion thereof, is substantially complementary to a portion of an adapter. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In some embodiments, an oligonucleotide may be immobilized to a solid support. In certain embodiments the nucleic acids herein contain phosphodiester bonds. In other embodiments, nucleic acid analogs are included that may have alternate backbones, comprising, e.g., phosphoramidate, phosphorothioate, phosphorodithioate, or O-methylphosphoroamidite linkages (see, Eckstein, Oligonucleotides and Analogues: A Practical Approach, Oxford University Press); and peptide nucleic acid backbones and linkages. Other analog nucleic acids include those with positive backbones; non-ionic backbones, and non-ribose backbones, including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. A residue of a nucleic acid, as referred to herein, is a monomer of the nucleic acid (e.g., a nucleotide).

“Nucleotide,” as used herein, refers to a nucleoside-5′-polyphosphate compound, or a structural analog thereof, which can be incorporated (e.g., partially incorporated as a nucleoside-5′-monophosphate or derivative thereof) by a nucleic acid polymerase to extend a growing nucleic acid chain (such as a primer). Nucleotides may include bases such as guanine (G), adenine (A), thymine, (T), uracil (U), cytosine (C), or analogues thereof, and may comprise 2, 3, 4, 5, 6, 7, 8, or more phosphates in the phosphate group. Nucleotides can be ribonucleotides, deoxyribonucleotides, or modified versions thereof. Examples of polynucleotides contemplated herein include single and double stranded DNA, single and double stranded RNA, and hybrid molecules having mixtures of single and double stranded DNA and RNA. Examples of nucleic acid, e.g., polynucleotides contemplated herein include any types of RNA, e.g., mRNA, siRNA, miRNA, and guide RNA and any types of DNA, genomic DNA, plasmid DNA, and minicircle DNA, and any fragments thereof. The term “duplex” in the context of polynucleotides refers, in the usual and customary sense, to double strandedness. Nucleotides may be modified at one or more of the base, sugar, or phosphate group. A nucleotide may have a label or tag attached (a “labeled nucleotide” or “tagged nucleotide”). In embodiments, the nucleotide is a modified nucleotide which terminates primer extension reversibly. In embodiments, nucleotides may further include a polymerase-compatible cleavable moiety covalently bound to the 3′ oxygen.

As used herein, a “nucleotide construct” refers to a compound including a scaffold core that links a nucleotide analogue moiety and a biomolecule described herein to a second nucleotide moiety or fluorescent moiety. In embodiments, the scaffold core of the nucleotide construct is a triazine moiety having the formula,

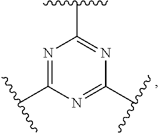 [SMILES: *c1nc(*)nc(*)n1]

which links a nucleotide moiety and a biomolecule described herein to a second nucleotide moiety or fluorescent moiety. In embodiments, the biomolecule attached to the scaffold core of the nucleotide construct is avidin or streptavidin. In embodiments, the biomolecule attached to the scaffold core of the nucleotide construct includes a biotin moiety. In embodiments, the biomolecule attached to the scaffold core of the nucleotide construct includes a biotin moiety and streptavidin. In embodiments, the biomolecule attached to the scaffold core of the nucleotide construct includes a biotin moiety and avidin.

A “nucleoside” is structurally similar to a nucleotide but lacks the phosphate moieties. The term “nucleoside” refers, in the usual and customary sense, to a glycosylamine including a nucleobase and a five-carbon sugar (ribose or deoxyribose). Non-limiting examples of nucleosides include cytidine, uridine, adenosine, guanosine, thymidine and inosine. Nucleosides may be modified at the base and/or the sugar. An example of a nucleoside analog would be one in which the label is linked to the base and there is no phosphate group attached to the sugar molecule.

The terms “identical” or percent “identity,” in the context of two or more nucleic acids or polypeptide sequences, refer to two or more sequences or subsequences that are the same or have a specified percentage of amino acid residues or nucleotides that are the same (i.e., about 60% identity, preferably 65%, 70%, 75%, 80%, 85%, 90%, 91%, 92%, 93%, 94%, 95%, 96%, 97%, 98%, 99%, or higher identity over a specified region, when compared and aligned for maximum correspondence over a comparison window or designated region) as measured using a BLAST or BLAST 2.0 sequence comparison algorithms with default parameters described below, or by manual alignment and visual inspection (see, e.g., NCBI web site www.ncbi.nlm.nih.gov/BLAST/or the like). Such sequences are then said to be “substantially identical.” This definition also refers to, or may be applied to, the complement of a test sequence. The definition also includes sequences that have deletions and/or additions, as well as those that have substitutions. As described below, the preferred algorithms can account for gaps and the like. Preferably, identity exists over a region that is at least about 25 amino acids or nucleotides in length, or more preferably over a region that is 50-100 amino acids or nucleotides in length.

The terms also encompass nucleic acids containing known nucleotide analogs or modified backbone residues or linkages, which are synthetic, naturally occurring, or non-naturally occurring, which have similar binding properties as the reference nucleic acid, and which are metabolized in a manner similar to the reference nucleotides. Examples of such analogs include, without limitation, phosphodiester derivatives including, e.g., phosphoramidate, phosphorodiamidate, phosphorothioate (also known as phosphothioate having double bonded sulfur replacing oxygen in the phosphate), phosphorodithioate, phosphonocarboxylic acids, phosphonocarboxylates, phosphonoacetic acid, phosphonoformic acid, methyl phosphonate, boron phosphonate, or O-methylphosphoroamidite linkages (see, Eckstein, Oligonucleotides and Analogues: A Practical Approach, Oxford University Press) as well as modifications to the nucleotide bases such as in 5-methyl cytidine or pseudouridine; and peptide nucleic acid backbones and linkages. Other analog nucleic acids include those with positive backbones; non-ionic backbones, modified sugars, and non-ribose backbones (e.g., phosphorodiamidate morpholino oligos or locked nucleic acids (LNA) as known in the art), including those described in U.S. Pat. Nos. 5,235,033 and 5,034,506, and Chapters 6 and 7, ASC Symposium Series 580, Carbohydrate Modifications in Antisense Research, Sanghui & Cook, eds. Nucleic acids containing one or more carbocyclic sugars are also included within one definition of nucleic acids. Modifications of the ribose-phosphate backbone may be done for a variety of reasons, e.g., to increase the stability and half-life of such molecules in physiological environments or as probes on a biochip. Mixtures of naturally occurring nucleic acids and analogs can be made; alternatively, mixtures of different nucleic acid analogs, and mixtures of naturally occurring nucleic acids and analogs may be made. In embodiments, the internucleotide linkages in DNA are phosphodiester, phosphodiester derivatives, or a combination of both.

In embodiments, “nucleotide analogue,” “nucleotide analog,” or “nucleotide derivative” shall mean an analogue of A, G, C, T or U (that is, an analogue or derivative of a nucleotide comprising the base A, G, C, T or U), including a phosphate group, which may be recognized by DNA or RNA polymerase (whichever is applicable) and may be incorporated into a strand of DNA or RNA (whichever is appropriate). Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the —OH group at the 3′-position of deoxyribose. Nucleotide analogues and DNA polymerase-based DNA sequencing are also described in U.S. Pat. No. 6,664,079, which is incorporated herein by reference in its entirety for all purposes.

As used herein, the term “modified nucleotide” refers to nucleotide modified in some manner. Typically, a nucleotide contains a single 5-carbon sugar moiety, a single nitrogenous base moiety and 1 to three phosphate moieties. In embodiments, a nucleotide can include a blocking moiety and/or a label moiety. A blocking moiety on a nucleotide prevents formation of a covalent bond between the 3′ hydroxyl moiety of the nucleotide and the 5′ phosphate of another nucleotide. A blocking moiety on a nucleotide can be reversible, whereby the blocking moiety can be removed or modified to allow the 3′ hydroxyl to form a covalent bond with the 5′ phosphate of another nucleotide. A blocking moiety can be effectively irreversible under particular conditions used in a method set forth herein. In embodiments, the blocking moiety is attached to the 3′ oxygen of the nucleotide and is independently —NH₂, —CN, —CH₃, C₂-C₆ allyl (e.g., —CH₂—CH═CH₂), methoxyalkyl (e.g., —CH₂—O—CH₃), or —CH₂N₃. In embodiments, the blocking moiety is attached to the 3′ oxygen of the nucleotide and is independently

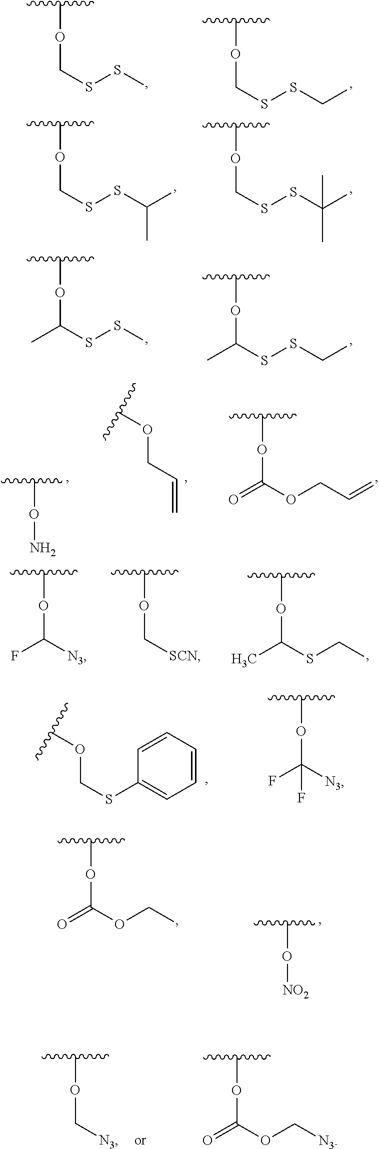

A label moiety of a modified nucleotide can be any moiety that allows the nucleotide to be detected, for example, using a spectroscopic method. Exemplary label moieties are fluorescent labels, mass labels, chemiluminescent labels, electrochemical labels, detectable labels and the like. One or more of the above moieties can be absent from a nucleotide used in the methods and compositions set forth herein. For example, a nucleotide can lack a label moiety or a blocking moiety or both. Examples of nucleotide analogues include, without limitation, 7-deaza-adenine, 7-deaza-guanine, the analogues of deoxynucleotides shown herein, analogues in which a label is attached through a cleavable linker to the 5-position of cytosine or thymine or to the 7-position of deaza-adenine or deaza-guanine, and analogues in which a small chemical moiety is used to cap the OH group at the 3′-position of deoxyribose. Nucleotide analogues and DNA polymerase-based DNA sequencing are also described in U.S. Pat. No. 6,664,079, which is incorporated herein by reference in its entirety for all purposes. Non-limiting examples of detectable labels include labels including fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the dye is a fluorescent dye. Non-limiting examples of dyes, some of which are commercially available, include CF® dyes (Biotium, Inc.), Atto™ dyes (ATTO-TEC GmbH), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, the label is a fluorophore.

In some embodiments, a nucleic acid includes a label. As used herein, the term “label” or “labels” is used in accordance with their plain and ordinary meanings and refer to molecules that can directly or indirectly produce or result in a detectable signal either by themselves or upon interaction with another molecule. Non-limiting examples of detectable labels include fluorescent dyes, biotin, digoxin, haptens, and epitopes. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Non-limiting examples of dyes, some of which are commercially available, include CF® dyes (Biotium, Inc.), ATTO™ dyes (ATTO-TEC GmbH), Alexa Fluor® dyes (Thermo Fisher), DyLight® dyes (Thermo Fisher), Cy® dyes (GE Healthscience), IRDye® dyes (Li-Cor Biosciences, Inc.), and HiLyte™ dyes (Anaspec, Inc.). In embodiments, a particular nucleotide type is associated with a particular label, such that identifying the label identifies the nucleotide with which it is associated. In embodiments, the label is luciferin that reacts with luciferase to produce a detectable signal in response to one or more bases being incorporated into an elongated complementary strand, such as in pyrosequencing. In embodiment, a nucleotide includes a label (such as a dye). In embodiments, the label is not associated with any particular nucleotide, but detection of the label identifies whether one or more nucleotides having a known identity were added during an extension step (such as in the case of pyrosequencing). Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” Examples include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e., cyanine 3 or Cy3®). In embodiments, the cyanine moiety has 5 methine structures (i.e., cyanine 5 or Cy5®). In embodiments, the cyanine moiety has 7 methine structures (i.e., cyanine 7 or Cy7®).

As used herein, the term “removable” group, e.g., a label or a blocking group or protecting group, is used in accordance with its plain and ordinary meaning and refers to a chemical group that can be removed from a nucleotide analogue such that a DNA polymerase can extend the nucleic acid (e.g., a primer or extension product) by the incorporation of at least one additional nucleotide. Removal may be by any suitable method, including enzymatic, chemical, or photolytic cleavage. Removal of a removable group, e.g., a blocking group, does not require that the entire removable group be removed, only that a sufficient portion of it be removed such that a DNA polymerase can extend a nucleic acid by incorporation of at least one additional nucleotide using a nucleotide or nucleotide analogue. In general, the conditions under which a removable group is removed are compatible with a process employing the removable group (e.g., an amplification process or sequencing process).

As used herein, the terms “reversible blocking groups” and “reversible terminators” are used in accordance with their plain and ordinary meanings and refer to a blocking moiety located, for example, at the 3′ position of a modified nucleotide and may be a chemically cleavable moiety such as an allyl group, an azidomethyl group or a methoxymethyl group, or may be an enzymatically cleavable group such as a phosphate ester. Non-limiting examples of nucleotide blocking moieties are described in applications WO 2004/018497, WO 96/07669, U.S. Pat. Nos. 7,057,026, 7,541,444, 5,763,594, 5,808,045, 5,872,244 and 6,232,465 the contents of which are incorporated herein by reference in their entirety. The nucleotides may be labelled or unlabeled. They may be modified with reversible terminators useful in methods provided herein and may be 3′-O-blocked reversible or 3′-unblocked reversible terminators. In nucleotides with 3′-O-blocked reversible terminators, the blocking group —OR [reversible terminating (capping) group] is linked to the oxygen atom of the 3′-OH of the pentose, while the label is linked to the base, which acts as a reporter and can be cleaved. The 3′-O-blocked reversible terminators are known in the art, and may be, for instance, a 3′-ONH₂ reversible terminator, a 3′-O-allyl reversible terminator, or a 3′-O-azidomethyl reversible terminator. In embodiments, the reversible terminator moiety is attached to the 3′-oxygen of the nucleotide, having the formula:

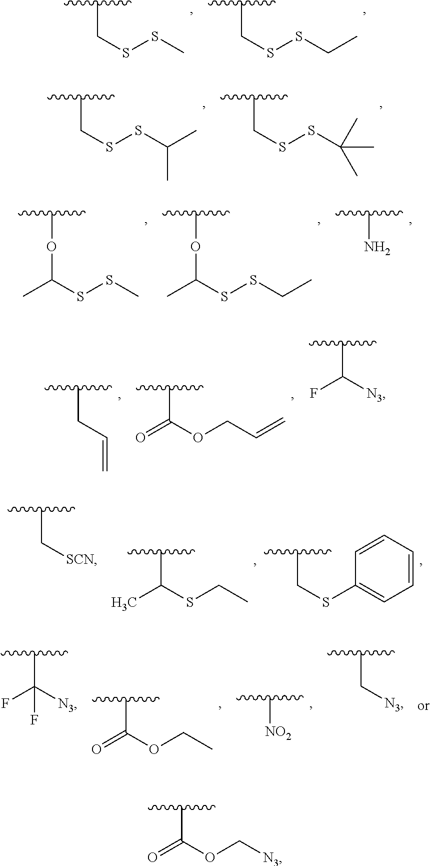

wherein the 3′ oxygen of the nucleotide is not shown in the formulae above. The term “allyl” as described herein refers to an unsubstituted methylene attached to a vinyl group (i.e., —CH═CH₂) having the formula

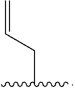 [SMILES: *CC=C]

An “allyl linker” refers to a divalent unsubstituted methylene attached to a vinyl group, having the formula

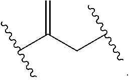 [SMILES: *CC(*)=C]

In embodiments, the reversible terminator moiety is

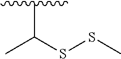 [SMILES: *C(C)SSC]

as described in U.S. Pat. No. 10,738,072, which is incorporated herein by reference for all purposes. For example, a nucleotide including a reversible terminator moiety may be represented by the formula:

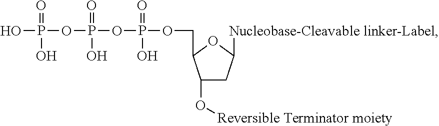

where the nucleobase is adenine or adenine analogue, thymine or thymine analogue, guanine or guanine analogue, or cytosine or cytosine analogue.

In some embodiments, a nucleic acid (e.g., a probe or a primer) includes a molecular identifier or a molecular barcode. As used herein, the term “molecular barcode” (which may be referred to as a “tag”, a “barcode”, a “molecular identifier”, an “identifier sequence” or a “unique molecular identifier” (UMI)) refers to any material (e.g., a nucleotide sequence, a nucleic acid molecule feature) that is capable of distinguishing an individual molecule in a large heterogeneous population of molecules. In embodiments, a barcode is unique in a pool of barcodes that differ from one another in sequence, or is uniquely associated with a particular sample polynucleotide in a pool of sample polynucleotides. In embodiments, every barcode in a pool of adapters is unique, such that sequencing reads including the barcode can be identified as originating from a single sample polynucleotide molecule on the basis of the barcode alone. In other embodiments, individual barcode sequences may be used more than once, but adapters including the duplicate barcodes are associated with different sequences and/or in different combinations of barcoded adaptors, such that sequence reads may still be uniquely distinguished as originating from a single sample polynucleotide molecule on the basis of a barcode and adjacent sequence information (e.g., sample polynucleotide sequence, and/or one or more adjacent barcodes). In embodiments, barcodes are about or at least about 5, 6, 7, 8, 9, 10, 15, 20, 25, 30, 40, 50, 75 or more nucleotides in length. In embodiments, barcodes are shorter than 20, 15, 10, 9, 8, 7, 6, or 5 nucleotides in length. In embodiments, barcodes are about 10 to about 50 nucleotides in length, such as about 15 to about 40 or about 20 to about 30 nucleotides in length. In a pool of different barcodes, barcodes may have the same or different lengths. In general, barcodes are of sufficient length and include sequences that are sufficiently different to allow the identification of sequencing reads that originate from the same sample polynucleotide molecule. In embodiments, each barcode in a plurality of barcodes differs from every other barcode in the plurality by at least three nucleotide positions, such as at least 3, 4, 5, 6, 7, 8, 9, 10, or more nucleotide positions. In some embodiments, substantially degenerate barcodes may be known as random. In some embodiments, a barcode may include a nucleic acid sequence from within a pool of known sequences. In some embodiments, the barcodes may be pre-defined. In embodiments, the barcodes are selected to form a known set of barcodes, e.g., the set of barcodes may be distinguished by a particular Hamming distance. In embodiments, each barcode sequence is unique within the known set of barcodes. In embodiments, each barcode sequence is associated with a particular oligonucleotide.

In embodiments, a nucleic acid (e.g., an adapter or primer) includes a sample barcode. In general, a “sample barcode” is a nucleotide sequence that is sufficiently different from other sample barcode to allow the identification of the sample source based on sample barcode sequence(s) with which they are associated. In embodiments, a plurality of nucleotides (e.g., all nucleotides from a particular sample source, or sub-sample thereof) are joined to a first sample barcode, while a different plurality of nucleotides (e.g., all nucleotides from a different sample source, or different subsample) are joined to a second sample barcode, thereby associating each plurality of polynucleotides with a different sample barcode indicative of sample source. In embodiments, each sample barcode in a plurality of sample barcodes differs from every other sample barcode in the plurality by at least three nucleotide positions, such as at least 3, 4, 5, 6, 7, 8, 9, 10, or more nucleotide positions. In some embodiments, substantially degenerate sample barcodes may be known as random. In some embodiments, a sample barcode may include a nucleic acid sequence from within a pool of known sequences. In some embodiments, the sample barcodes may be pre-defined. In embodiments, the sample barcode includes about 1 to about 10 nucleotides. In embodiments, the sample barcode includes about 3, 4, 5, 6, 7, 8, 9, or about 10 nucleotides. In embodiments, the sample barcode includes about 3 nucleotides. In embodiments, the sample barcode includes about 5 nucleotides. In embodiments, the sample barcode includes about 7 nucleotides. In embodiments, the sample barcode includes about 10 nucleotides. In embodiments, the sample barcode includes about 6 to about 10 nucleotides.

The term “complement” is used in accordance with its plain and ordinary meaning and refers to a nucleotide (e.g., RNA nucleotide or DNA nucleotide) or a sequence of nucleotides capable of base pairing with a complementary nucleotide or sequence of nucleotides (e.g., Watson-Crick base pairing). As described herein and commonly known in the art the complementary (matching) nucleotide of adenosine is thymidine and the complementary (matching) nucleotide of guanosine is cytosine. Thus, a complement may include a sequence of nucleotides that base paired with corresponding complementary nucleotides of a second nucleic acid sequence. The nucleotides of a complement may partially or completely match the nucleotides of the second nucleic acid sequence. Where the nucleotides of the complement completely match each nucleotide of the second nucleic acid sequence, the complement forms base pairs with each nucleotide of the second nucleic acid sequence. Where the nucleotides of the complement partially match the nucleotides of the second nucleic acid sequence only some of the nucleotides of the complement form base pairs with nucleotides of the second nucleic acid sequence. Examples of complementary sequences include coding and non-coding sequences, wherein the non-coding sequence contains complementary nucleotides to the coding sequence and thus forms the complement of the coding sequence. A further example of complementary sequences are sense and antisense sequences, wherein the sense sequence contains complementary nucleotides to the antisense sequence and thus forms the complement of the antisense sequence. Another example of complementary sequences are a template sequence and an amplicon sequence polymerized by a polymerase along the template sequence. “Duplex” means at least two oligonucleotides and/or polynucleotides that are fully or partially complementary undergo Watson-Crick type base pairing among all or most of their nucleotides so that a stable complex is formed. Complementary single stranded nucleic acids and/or substantially complementary single stranded nucleic acids can hybridize to each other under hybridization conditions, thereby forming a nucleic acid that is partially or fully double stranded. When referring to a double-stranded polynucleotide including a first strand hybridized to a second strand, it is understood that each of the first strand and the second strand are independently single-stranded polynucleotides. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

As described herein, the complementarity of sequences may be partial, in which only some of the nucleic acids match according to base pairing, or complete, where all the nucleic acids match according to base pairing. Thus, two sequences that are complementary to each other, may have a specified percentage of nucleotides that complement one another (e.g., about 60%, preferably 65%, 70%, 75%, 80%, 85%, 90%, 91%, 92%, 93%, 94%, 95%, 96%, 97%, 98%, 99%, or higher complementarity over a specified region). In embodiments, two sequences are complementary when they are completely complementary, having 100% complementarity. In embodiments, sequences in a pair of complementary sequences form portions of a single polynucleotide with non-base-pairing nucleotides (e.g., as in a hairpin or loop structure, with or without an overhang) or portions of separate polynucleotides. In embodiments, one or both sequences in a pair of complementary sequences form portions of longer polynucleotides, which may or may not include additional regions of complementarity.

As used herein, an oligonucleotide is understood to be a molecule that has a sequence of bases on a backbone comprised mainly of identical monomer units at defined intervals. The bases are arranged on the backbone in such a way that they can enter into a bond with a nucleic acid having a sequence of bases that are complementary to the bases of the oligonucleotide. The most common oligonucleotides have a backbone of sugar phosphate units. A distinction may be made between oligodeoxyribonucleotides, made up of “dNTPs,” which do not have a hydroxyl group at the 2′ position, and oligoribonucleotides, made up of “NTPs,” which have a hydroxyl group in the 2′ position. Oligonucleotides also may include derivatives, in which the hydrogen of the hydroxyl group is replaced with an organic group, e.g., an allyl group.

Oligonucleotides, as described herein, typically are capable of forming hydrogen bonds with oligonucleotides having a complementary base sequence. These bases may include the natural bases, such as A, G, C, T, and U, as well as artificial, non-standard or non-natural nucleotides such as iso-cytosine and iso-guanine. As described herein, a first sequence of an oligonucleotide is described as being 100% complementary with a second sequence of an oligonucleotide when the consecutive bases of the first sequence (read 5′-to-3′) follow the Watson-Crick rule of base pairing as compared to the consecutive bases of the second sequence (read 3′-to-5′). An oligonucleotide may include nucleotide substitutions. For example, an artificial base may be used in place of a natural base such that the artificial base exhibits a specific interaction that is similar to the natural base.

As used herein, the terms “polynucleotide primer” and “primer” refers to any polynucleotide molecule that may hybridize to a polynucleotide template, be bound by a polymerase, and be extended in a template-directed process for nucleic acid synthesis (e.g., amplification and/or sequencing). The primer may be a separate polynucleotide from the polynucleotide template, or both may be portions of the same polynucleotide (e.g., as in a hairpin structure having a 3′ end that is extended along another portion of the polynucleotide to extend a double-stranded portion of the hairpin). Primers (e.g., forward or reverse primers) may be attached to a solid support. A primer can be of any length depending on the particular technique it will be used for. For example, PCR primers are generally between 10 and 40 nucleotides in length. The length and complexity of the nucleic acid fixed onto the nucleic acid template may vary. In some embodiments, a primer has a length of 200 nucleotides or less. In certain embodiments, a primer has a length of 10 to 150 nucleotides, 15 to 150 nucleotides, 5 to 100 nucleotides, 5 to 50 nucleotides or 10 to 50 nucleotides. In certain embodiments, a primer has a length of 10 to 150 nucleotides, 15 to 150 nucleotides, 5 to 100 nucleotides, 5 to 50 nucleotides or 10 to 50 nucleotides. A primer typically has a length of 10 to 50 nucleotides. For example, a primer may have a length of 10 to 40, 10 to 30, 10 to 20, 25 to 50, 15 to 40, 15 to 30, 20 to 50, 20 to 40, or 20 to 30 nucleotides. In some embodiments, a primer has a length of 18 to 24 nucleotides. One of skill can adjust these factors to provide optimum hybridization and signal production for a given hybridization procedure. The primer permits the addition of a nucleotide residue thereto, or oligonucleotide or polynucleotide synthesis therefrom, under suitable conditions. In an embodiment the primer is a DNA primer, i.e., a primer consisting of, or largely consisting of, deoxyribonucleotide residues. The primers are designed to have a sequence that is the complement of a region of template/target DNA to which the primer hybridizes. The addition of a nucleotide residue to the 3′ end of a primer by formation of a phosphodiester bond results in a DNA extension product. The addition of a nucleotide residue to the 3′ end of the DNA extension product by formation of a phosphodiester bond results in a further DNA extension product. In another embodiment the primer is an RNA primer. In embodiments, a primer is hybridized to a target polynucleotide. A “primer” is complementary to a polynucleotide template, and complexes by hydrogen bonding or hybridization with the template to give a primer/template complex for initiation of synthesis by a polymerase, which is extended by the addition of covalently bonded bases linked at its 3′ end complementary to the template in the process of DNA synthesis.

As used herein, the term “primer binding sequence” refers to a polynucleotide sequence that is complementary to at least a portion of a primer (e.g., a sequencing primer or an amplification primer). Primer binding sequences can be of any suitable length. In embodiments, a primer binding sequence is about or at least about 10, 15, 20, 25, 30, or more nucleotides in length. In embodiments, a primer binding sequence is 10-50, 15-30, or 20-25 nucleotides in length. The primer binding sequence may be selected such that the primer (e.g., sequencing primer) has the preferred characteristics to minimize secondary structure formation or minimize non-specific amplification, for example having a length of about 20-30 nucleotides; approximately 50% GC content, and a Tm of about 55° C. to about 65° C.

Nucleic acids, including e.g., nucleic acids with a phosphorothioate backbone, can include one or more reactive moieties. As used herein, the term reactive moiety includes any group capable of reacting with another molecule, e.g., a nucleic acid or polypeptide through covalent, non-covalent or other interactions. By way of example, the nucleic acid can include an amino acid reactive moiety that reacts with an amino acid on a protein or polypeptide through a covalent, non-covalent or other interaction.

As used herein, a “platform primer” is a primer oligonucleotide immobilized or otherwise bound to a solid support (i.e. an immobilized oligonucleotide). Examples of platform primers include P7 and P5 primers (i.e., Illumina® platform sequences), or S1 and S2 primers (i.e., Singular Genomics® platform sequences), or the reverse complements thereof. A “platform primer binding sequence” refers to a sequence or portion of an oligonucleotide that is capable of binding to a platform primer (e.g., the platform primer binding sequence is complementary to the platform primer). In embodiments, a platform primer binding sequence may form part of an adapter. In embodiments, a platform primer binding sequence is complementary to a platform primer sequence. In embodiments, a platform primer binding sequence is complementary to a primer. Illumina is a registered trademark of Illumina, Inc. Singular Genomics is a registered trademark of Singular Genomics Systems, Inc.

The order of elements within a nucleic acid molecule is typically described herein from 5′ to 3′. In the case of a double-stranded molecule, the “top” strand is typically shown from 5′ to 3′, according to convention, and the order of elements is described herein with reference to the top strand.

As used herein, the term “DNA polymerase” and “nucleic acid polymerase” are used in accordance with their plain ordinary meanings and refer to enzymes capable of synthesizing nucleic acid molecules from nucleotides (e.g., deoxyribonucleotides). Exemplary types of polymerases that may be used in the compositions and methods of the present disclosure include the nucleic acid polymerases such as DNA polymerase, DNA- or RNA-dependent RNA polymerase, and reverse transcriptase. In some cases, the DNA polymerase is 9° N polymerase or a variant thereof, E. coli DNA polymerase I, Bacteriophage T4 DNA polymerase, Sequenase™ (GE Healthcare), Taq DNA polymerase, DNA polymerase from Bacillus stearothermophilus, Bst 2.0 DNA polymerase, 9° N polymerase (exo-)A485L/Y409V, Phi29 DNA Polymerase (φ29 DNA Polymerase), T7 DNA polymerase, DNA polymerase II, DNA polymerase III holoenzyme, DNA polymerase IV, DNA polymerase V, VentR DNA polymerase, Therminator™ II DNA Polymerase, Therminator™ III DNA Polymerase, or Therminator™ IX DNA Polymerase. In embodiments, the polymerase is a protein polymerase. Typically, a DNA polymerase adds nucleotides to the 3′-end of a DNA strand, one nucleotide at a time. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g. Therminator™ γ, 9° N polymerase (exo-), Therminator™ II, Therminator™ III, or Therminator™ IX). In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044). In embodiments, the polymerase is an enzyme described in US 2021/0139884. For example, a polymerase catalyzes the addition of a next correct nucleotide to the 3′—OH group of the primer via a phosphodiester bond, thereby chemically incorporating the nucleotide into the primer. Optionally, the polymerase used in the provided methods is a processive polymerase. Optionally, the polymerase used in the provided methods is a distributive polymerase. Therminator™ is a trademark of New England Biolabs.

As used herein, the term “thermophilic nucleic acid polymerase” refers to a family of DNA polymerases (e.g., 9° N™) and mutants thereof derived from the DNA polymerase originally isolated from the hyperthermophilic archaea, Thermococcus sp. 9 degrees N-7, found in hydrothermal vents at that latitude (East Pacific Rise) (Southworth M W, et al. PNAS. 1996; 93(11):5281-5285). A thermophilic nucleic acid polymerase is a member of the family B DNA polymerases. Site-directed mutagenesis of the 3′-5′ exo motif I (Asp-Ile-Glu or DIE) to AIA, AIE, EIE, EID or DIA yielded polymerase with no detectable 3′ exonuclease activity. Mutation to Asp-Ile-Asp (DID) resulted in reduction of 3′-5′ exonuclease specific activity to <1% of wild type, while maintaining other properties of the polymerase including its high strand displacement activity. The sequence AIA (D141A, E143A) was chosen for reducing exonuclease. Subsequent mutagenesis of key amino acids results in an increased ability of the enzyme to incorporate dideoxynucleotides, ribonucleotides and acyclonucleotides (e.g., Therminator™ II enzyme from New England Biolabs with D141A/E143A/Y409V/A485L mutations); 3′-amino-dNTPs, 3′-azido-dNTPs and other 3′-modified nucleotides (e.g., NEB Therminator™ III DNA Polymerase with D141A/E143A/L408S/Y409A/P410V mutations, NEB Therminator™ IX DNA polymerase), or γ-phosphate labeled nucleotides (e.g., Therminator™ 7: D141A/E143A/W355A/L408 W/R460A/Q461S/K464E/D480V/R484 W/A485L). Typically, these enzymes do not have 5′-3′ exonuclease activity. Additional information about thermophilic nucleic acid polymerases may be found in (Southworth M W, et al. PNAS. 1996; 93(11):5281-5285; Bergen K, et al. ChemBioChem. 2013; 14(9):1058-1062; Kumar S, et al. Scientific Reports. 2012; 2:684; Fuller C W, et al. 2016; 113(19):5233-5238; Guo J, et al. Proceedings of the National Academy of Sciences of the United States of America. 2008; 105(27):9145-9150), which are incorporated herein in their entirety for all purposes.

As used herein, the term “exonuclease activity” is used in accordance with its ordinary meaning in the art, and refers to the removal of a nucleotide from a nucleic acid by an enzyme (e.g. DNA polymerase, a lambda exonuclease, Exo I, Exo III, T5, Exo V, Exo VII or the like). For example, during polymerization, nucleotides are added to the 3′ end of the primer strand. Occasionally a DNA polymerase incorporates an incorrect nucleotide to the 3′-OH terminus of the primer strand, wherein the incorrect nucleotide cannot form a hydrogen bond to the corresponding base in the template strand. Such a nucleotide, added in error, is removed from the primer as a result of the 3′ to 5′ exonuclease activity of the DNA polymerase. In embodiments, exonuclease activity may be referred to as “proofreading.” When referring to 3′-5′ exonuclease activity, it is understood that the DNA polymerase facilitates a hydrolyzing reaction that breaks phosphodiester bonds at the 3′ end of a polynucleotide chain to excise the nucleotide. In embodiments, 3′-5′ exonuclease activity refers to the successive removal of nucleotides in single-stranded DNA in a 3′→5′ direction, releasing deoxyribonucleoside 5′-monophosphates one after another. Methods for quantifying exonuclease activity are known in the art, see for example Southworth et al, PNAS Vol 93, 8281-8285 (1996). In embodiments, 5′-3′ exonuclease activity refers to the successive removal of nucleotides in double-stranded DNA in a 5′→3′ direction. In embodiments, the 5′-3′ exonuclease is lambda exonuclease. For example, lambda exonuclease catalyzes the removal of 5′ mononucleotides from duplex DNA, with a preference for 5′ phosphorylated double-stranded DNA. In other embodiments, the 5′-3′ exonuclease is E. coli DNA Polymerase I.

As used herein, the term “ligase” refers to an enzyme that catalyzes the formation of a new phosphodiester bond as a result of joining the 5′-phosphoryl terminus of DNA or RNA to single-stranded 3′-hydroxyl terminus of DNA or RNA. Ligase enzymes can form circular DNA or RNA templates in a non-template driven reaction, and examples of ligase enzymes include, but are not limited to, as CircLigase™ (Epicentre Biotechnologies), Taq DNA Ligase, HiFi Taq DNA Ligase, T4 ligase, or Ampligase® DNA Ligase (Epicentre Biotechnologies).

As used herein, the term “incorporating” or “chemically incorporating,” when used in reference to a primer and cognate nucleotide, refers to the process of joining the cognate nucleotide to the primer or extension product thereof by formation of a phosphodiester bond.

As used herein, the term “selective” or “selectivity” or the like of a compound refers to the compound's ability to discriminate between molecular targets. For example, a chemical reagent may selectively modify one nucleotide type in that it reacts with one nucleotide type (e.g., cytosines) and not other nucleotide types (e.g., adenine, thymine, or guanine). When used in the context of sequencing, such as in “selectively sequencing,” this term refers to sequencing one or more target polynucleotides from an original starting population of polynucleotides, and not sequencing non-target polynucleotides from the starting population. Typically, selectively sequencing one or more target polynucleotides involves differentially manipulating the target polynucleotides based on known sequence. For example, target polynucleotides may be hybridized to a probe oligonucleotide that may be labeled (such as with a member of a binding pair) or bound to a surface. In embodiments, hybridizing a target polynucleotide to a probe oligonucleotide includes the step of displacing one strand of a double-stranded nucleic acid. Probe-hybridized target polynucleotides may then be separated from non-hybridized polynucleotides, such as by removing probe-bound polynucleotides from the starting population or by washing away polynucleotides that are not bound to a probe. The result is a selected subset of the starting population of polynucleotides, which is then subjected to sequencing, thereby selectively sequencing the one or more target polynucleotides.

As used herein, the term “template polynucleotide” refers to any polynucleotide molecule that may be bound by a polymerase and utilized as a template for nucleic acid synthesis. A template polynucleotide may be a target polynucleotide. In general, the term “target polynucleotide” refers to a nucleic acid molecule or polynucleotide in a starting population of nucleic acid molecules having a target sequence whose presence, amount, and/or nucleotide sequence, or changes in one or more of these, are desired to be determined. The target sequence may be a portion of a gene, a regulatory sequence, genomic DNA, cDNA, RNA including mRNA, miRNA, rRNA, or others. The target sequence may be a target sequence from a sample or a secondary target such as a product of an amplification reaction. A target polynucleotide is not necessarily any single molecule or sequence. For example, a target polynucleotide may be any one of a plurality of target polynucleotides in a reaction, or all polynucleotides in a given reaction, depending on the reaction conditions. For example, in a nucleic acid amplification reaction with random primers, all polynucleotides in a reaction may be amplified. As a further example, a collection of targets may be simultaneously assayed using polynucleotide primers directed to a plurality of targets in a single reaction. As yet another example, all or a subset of polynucleotides in a sample may be modified by the addition of a primer-binding sequence (such as by the ligation of adapters containing the primer binding sequence), rendering each modified polynucleotide a target polynucleotide in a reaction with the corresponding primer polynucleotide(s). In embodiments, the template polynucleotide includes a target nucleic acid sequence and one or more barcode sequences. In embodiments, the template polynucleotide is a barcode sequence.

In embodiments, a target polynucleotide is a cell-free polynucleotide. In general, the terms “cell-free,” “circulating,” and “extracellular” as applied to polynucleotides (e.g. “cell-free DNA” (cfDNA) and “cell-free RNA” (cfRNA)) are used interchangeably to refer to polynucleotides present in a sample from a subject or portion thereof that can be isolated or otherwise manipulated without applying a lysis step to the sample as originally collected (e.g., as in extraction from cells or viruses). Cell-free polynucleotides are thus unencapsulated or “free” from the cells or viruses from which they originate, even before a sample of the subject is collected. Cell-free polynucleotides may be produced as a byproduct of cell death (e.g., apoptosis or necrosis) or cell shedding, releasing polynucleotides into surrounding body fluids or into circulation. Accordingly, cell-free polynucleotides may be isolated from a non-cellular fraction of blood (e.g., serum or plasma), from other bodily fluids (e.g., urine), or from non-cellular fractions of other types of samples.

The term “messenger RNA” or “mRNA” refers to an RNA that is without introns and is capable of being translated into a polypeptide. The term “RNA” refers to any ribonucleic acid, including but not limited to mRNA, tRNA (transfer RNA), rRNA (ribosomal RNA), and/or noncoding RNA (such as lncRNA (long noncoding RNA)). The term “cDNA” refers to a DNA that is complementary or identical to an RNA, in either single stranded or double stranded form.

As used herein, the term “associated” or “associated with” can mean that two or more species are identifiable as being co-located at a point in time. An association can mean that two or more species are or were within a similar container. An association can be an informatics association, where for example digital information regarding two or more species is stored and can be used to determine that one or more of the species were co-located at a point in time. An association can also be a physical association. In some instances, two or more associated species are “tethered”, “coated”, “attached”, or “immobilized” to one another or to a common solid or semisolid support (e.g. a receiving substrate). An association may refer to a relationship, or connection, between two entities. For example, a barcode sequence may be associated with a particular target by binding a probe including the barcode sequence to the target. In embodiments, detecting the associated barcode provides detection of the target. Associated may refer to the relationship between a sample and the DNA molecules, RNA molecules, or polynucleotides originating from or derived from that sample. These relationships may be encoded in oligonucleotide barcodes, as described herein. A polynucleotide is associated with a sample if it is an endogenous polynucleotide, i.e., it occurs in the sample at the time the sample is obtained, or is derived from an endogenous polynucleotide. For example, the RNAs endogenous to a cell are associated with that cell. cDNAs resulting from reverse transcription of these RNAs, and DNA amplicons resulting from PCR amplification of the cDNAs, contain the sequences of the RNAs and are also associated with the cell. The polynucleotides associated with a sample need not be located or synthesized in the sample, and are considered associated with the sample even after the sample has been destroyed (for example, after a cell has been lysed). Barcoding can be used to determine which polynucleotides in a mixture are associated with a particular sample. In embodiments, a proximity probe is associated with a particular barcode, such that identifying the barcode identifies the probe with which it is associated. Because the proximity probe specifically binds to a target, identifying the barcode thus identifies the target.

The term “adapter” as used herein refers to any oligonucleotide that can be ligated to a nucleic acid molecule, thereby generating nucleic acid products that can be sequenced on a sequencing platform (e.g., an Illumina® or Singular Genomics G4® sequencing platform). In embodiments, adapters include two reverse complementary oligonucleotides forming a double-stranded structure. In embodiments, an adapter includes two oligonucleotides that are complementary at one portion and mismatched at another portion, forming a Y-shaped or fork-shaped adapter that is double stranded at the complementary portion and has two overhangs at the mismatched portion. Since Y-shaped adapters have a complementary, double-stranded region, they can be considered a special form of double-stranded adapters. When this disclosure contrasts Y-shaped adapters and double stranded adapters, the term “double-stranded adapter” or “blunt-ended” is used to refer to an adapter having two strands that are fully complementary, substantially (e.g., more than 90% or 95%) complementary, or partially complementary. In embodiments, adapters include sequences that bind to sequencing primers. In embodiments, adapters include sequences that bind to immobilized oligonucleotides (e.g., P7 and P5 sequences) or reverse complements thereof. In embodiments, the adapter is substantially non-complementary to the 3′ end or the 5′ end of any target polynucleotide present in the sample. In embodiments, the adapter can include a sequence that is substantially identical, or substantially complementary, to at least a portion of a primer, for example a universal primer. In embodiments, the adapter can include an index sequence (also referred to as barcode or tag) to assist with downstream error correction, identification or sequencing. In some embodiments, an adapter is hairpin adapter (also referred to herein as a hairpin). In some embodiments, a hairpin adapter includes a single nucleic acid strand including a stem-loop structure. In some embodiments, a hairpin adapter includes a nucleic acid having a 5′-end, a 5′-portion, a loop, a 3′-portion and a 3′-end (e.g., arranged in a 5′ to 3′ orientation). In some embodiments, the 5′ portion of a hairpin adapter is annealed and/or hybridized to the 3′ portion of the hairpin adapter, thereby forming a stem portion of the hairpin adapter. In some embodiments, the 5′ portion of a hairpin adapter is substantially complementary to the 3′ portion of the hairpin adapter. In certain embodiments, a hairpin adapter includes a stem portion (i.e., stem) and a loop, wherein the stem portion is substantially double stranded thereby forming a duplex. In some embodiments, the loop of a hairpin adapter includes a nucleic acid strand that is not complementary (e.g., not substantially complementary) to itself or to any other portion of the hairpin adapter. In some embodiments, a method herein includes ligating a first adapter to a first end of a double stranded nucleic acid, and ligating a second adapter to a second end of a double stranded nucleic acid. In some embodiments, the first adapter and the second adapter are different. For example, in certain embodiments, the first adapter and the second adapter may include different nucleic acid sequences or different structures. In some embodiments, the first adapter is a Y-adapter and the second adapter is a hairpin adapter. In some embodiments, the first adapter is a hairpin adapter and a second adapter is a hairpin adapter. In certain embodiments, the first adapter and the second adapter may include different primer binding sites, different structures, and/or different capture sequences (e.g., a sequence complementary to a capture nucleic acid). In some embodiments, some, all or substantially all of the nucleic acid sequence of a first adapter and a second adapter are the same. In some embodiments, some, all or substantially all of the nucleic acid sequence of a first adapter and a second adapter are substantially different.

As used herein, the term “control” or “control experiment” is used in accordance with its plain and ordinary meaning and refers to an experiment in which the subjects or reagents of the experiment are treated as in a parallel experiment except for omission of a procedure, reagent, or variable of the experiment. In some instances, the control is used as a standard of comparison in evaluating experimental effects.

The term “bioconjugate group” or “bioconjugate reactive moiety” or “bioconjugate reactive group” refers to a chemical moiety which participates in a reaction to form bioconjugate linker (e.g., covalent linker). Non-limiting examples of bioconjugate groups include —NH₂,
“\*MERGEFORMAT\*MERGEFORMAT —COOH, —COOCH₃, —N-hydroxysuccinimide, -maleimide,

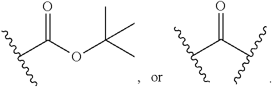

In embodiments, the bioconjugate reactive group may be protected (e.g., with a protecting group). In embodiments, the bioconjugate reactive moiety is

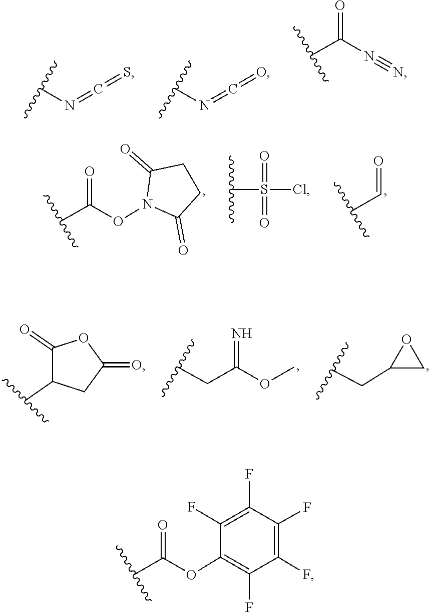

or —NH₂. Additional examples of bioconjugate reactive groups and the resulting bioconjugate reactive linkers may be found in the Bioconjugate Table below:

| Bioconjugate | Bioconjugate | |
|---|---|---|
| reactive group 1 | reactive group 2 | |
| (e.g., electrophilic | (e.g., nucleophilic | Resulting |
| bioconjugate | bioconjugate | Bioconjugate |
| reactive | reactive | reactive |
| moiety) | moiety) | linker |
| activated esters | amines/anilines | carboxamides |
| acrylamides | thiols | thioethers |
| acyl azides | amines/anilines | carboxamides |
| acyl halides | amines/anilines | carboxamides |
| acyl halides | alcohols/phenols | esters |
| acyl nitriles | alcohols/phenols | esters |
| acyl nitriles | amines/anilines | carboxamides |
| aldehydes | amines/anilines | imines |
| aldehydes or ketones | hydrazines | hydrazones |
| aldehydes or ketones | hydroxylamines | oximes |
| alkyl halides | amines/anilines | alkyl amines |
| alkyl halides | carboxylic acids | esters |
| alkyl halides | thiols | thioethers |
| alkyl halides | alcohols/phenols | ethers |
| alkyl sulfonates | thiols | thioethers |
| alkyl sulfonates | carboxylic acids | esters |
| alkyl sulfonates | alcohols/phenols | ethers |
| anhydrides | alcohols/phenols | esters |
| anhydrides | amines/anilines | carboxamides |
| aryl halides | thiols | thiophenols |
| aryl halides | amines | aryl amines |
| aziridines | thiols | thioethers |
| boronates | glycols | boronate esters |
| carbodiimides | carboxylic acids | N-acylureas or anhydrides |
| diazoalkanes | carboxylic acids | esters |
| epoxides | thiols | thioethers |
| haloacetamides | thiols | thioethers |
| haloplatinate | amino | platinum complex |
| haloplatinate | heterocycle | platinum complex |
| haloplatinate | thiol | platinum complex |
| halotriazines | amines/anilines | aminotriazines |
| halotriazines | alcohols/phenols | triazinyl ethers |
| halotriazines | thiols | triazinyl thioethers |
| imido esters | amines/anilines | amidines |
| isocyanates | amines/anilines | ureas |
| isocyanates | alcohols/phenols | urethanes |
| isothiocyanates | amines/anilines | thioureas |
| maleimides | thiols | thioethers |
| phosphoramidites | alcohols | phosphite esters |
| silyl halides | alcohols | silyl ethers |
| sulfonate esters | amines/anilines | alkyl amines |
| sulfonate esters | thiols | thioethers |
| sulfonate esters | carboxylic acids | esters |
| sulfonate esters | alcohols | ethers |
| sulfonyl halides | amines/anilines | sulfonamides |
| sulfonyl halides | phenols/alcohols | sulfonate esters |

As used herein, the term “bioconjugate” or “bioconjugate linker” refers to the resulting association between atoms or molecules of bioconjugate reactive groups. The association can be direct or indirect. For example, a conjugate between a first bioconjugate reactive group (e.g.,
“\*MERGEFORMAT\*MERGEFORMAT —NH₂, —COOH, —N-hydroxysuccinimide, or -maleimide) and a second bioconjugate reactive group (e.g., sulfhydryl, sulfur-containing amino acid, amine, amine sidechain containing amino acid, or carboxylate) provided herein can be direct, e.g., by covalent bond or linker (e.g., a first linker of second linker), or indirect, e.g., by non-covalent bond (e.g., electrostatic interactions (e.g., ionic bond, hydrogen bond, halogen bond), van der Waals interactions (e.g., dipole-dipole, dipole-induced dipole, London dispersion), ring stacking (pi effects), hydrophobic interactions and the like). In embodiments, bioconjugates or bioconjugate linkers are formed using bioconjugate chemistry (i.e., the association of two bioconjugate reactive groups) including, but are not limited to nucleophilic substitutions (e.g., reactions of amines and alcohols with acyl halides, active esters), electrophilic substitutions (e.g., enamine reactions) and additions to carbon-carbon and carbon-heteroatom multiple bonds (e.g., Michael reaction, Diels-Alder addition). These and other useful reactions are discussed in, for example, March, ADVANCED ORGANIC CHEMISTRY, 3rd Ed., John Wiley & Sons, New York, 1985; Hermanson, BIOCONJUGATE TECHNIQUES, Academic Press, San Diego, 1996; and Feeney et al., MODIFICATION OF PROTEINS; Advances in Chemistry Series, Vol. 198, American Chemical Society, Washington, D.C., 1982. In embodiments, the first bioconjugate reactive group (e.g., maleimide moiety) is covalently attached to the second bioconjugate reactive group (e.g., a sulfhydryl). In embodiments, the first bioconjugate reactive group (e.g., haloacetyl moiety) is covalently attached to the second bioconjugate reactive group (e.g., a sulfhydryl). In embodiments, the first bioconjugate reactive group (e.g., pyridyl moiety) is covalently attached to the second bioconjugate reactive group (e.g., a sulfhydryl). In embodiments, the first bioconjugate reactive group (e.g., —N-hydroxysuccinimide moiety) is covalently attached to the second bioconjugate reactive group (e.g., an amine). In embodiments, the first bioconjugate reactive group (e.g., maleimide moiety) is covalently attached to the second bioconjugate reactive group (e.g., a sulfhydryl). In embodiments, the first bioconjugate reactive group (e.g.,
“\*MERGEFORMAT\*MERGEFORMAT -sulfo-N-hydroxysuccinimide moiety) is covalently attached to the second bioconjugate reactive group (e.g., an amine). In embodiments, the first bioconjugate reactive group (e.g., —COOH) is covalently attached to the second bioconjugate reactive group

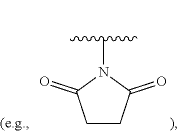

thereby forming a bioconjugate

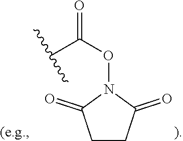 [SMILES: *C(=O)ON1C(=O)CCC1=O]

In embodiments, the first bioconjugate reactive group (e.g., —NH₂) is covalently attached to the second bioconjugate reactive group

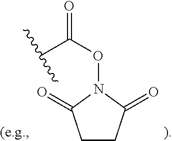 [SMILES: *C(=O)ON1C(=O)CCC1=O]

thereby forming a bioconjugate

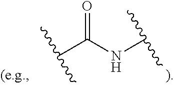 [SMILES: *NC(*)=O]

In embodiments, the first bioconjugate reactive group (e.g., a coupling reagent) is covalently attached to the second bioconjugate reactive group

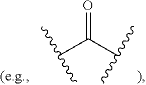 [SMILES: *C(*)=O]

thereby forming a bioconjugate

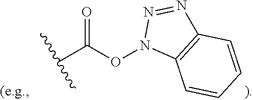 [SMILES: *C(=O)On1nnc2ccccc21]

Useful bioconjugate reactive moieties used for bioconjugate chemistries herein include, for example: (a) carboxyl groups and various derivatives thereof including, but not limited to, N-hydroxysuccinimide esters, N-hydroxybenztriazole esters, acid halides, acyl imidazoles, thioesters, p-nitrophenyl esters, alkyl, alkenyl, alkynyl and aromatic esters; (b) hydroxyl groups which can be converted to esters, ethers, aldehydes, etc. (c) haloalkyl groups wherein the halide can be later displaced with a nucleophilic group such as, for example, an amine, a carboxylate anion, thiol anion, carbanion, or an alkoxide ion, thereby resulting in the covalent attachment of a new group at the site of the halogen atom; (d) dienophile groups which are capable of participating in Diels-Alder reactions such as, for example, maleimido or maleimide groups; (e) aldehyde or ketone groups such that subsequent derivatization is possible via formation of carbonyl derivatives such as, for example, imines, hydrazones, semicarbazones or oximes, or via such mechanisms as Grignard addition or alkyllithium addition; (f) sulfonyl halide groups for subsequent reaction with amines, for example, to form sulfonamides; (g) thiol groups, which can be converted to disulfides, reacted with acyl halides, or bonded to metals such as gold, or react with maleimides; (h) amine or sulfhydryl groups (e.g., present in cysteine), which can be, for example, acylated, alkylated or oxidized; (i) alkenes, which can undergo, for example, cycloadditions, acylation, Michael addition, etc.; (j) epoxides, which can react with, for example, amines and hydroxyl compounds; (k) phosphoramidites and other standard functional groups useful in nucleic acid synthesis; (1) metal silicon oxide bonding; (m) metal bonding to reactive phosphorus groups (e.g., phosphines) to form, for example, phosphate diester bonds; (n) azides coupled to alkynes using copper catalyzed cycloaddition click chemistry; and (o) biotin conjugate can react with avidin or streptavidin to form a avidin-biotin complex or streptavidin-biotin complex.

The term “streptavidin” refers to a tetrameric protein (including homologs, isoforms, and functional fragments thereof) derived from bacterium Streptomyces avidinii. This protein is capable of binding biotin with high affinity (with a dissociation constant, K_{D} of about 1015 M). The term includes any recombinant or naturally-occurring form of streptavidin variants thereof that maintain streptavidin activity (e.g., within at least 30%, 40%, 50%, 60%, 70%, 80%, 90%, 95%, or 100% activity compared to wildtype streptavidin).

The term “avidin” refers to a tetrameric protein (including homologs, isoforms, and functional fragments thereof) derived from birds, reptiles, and amphibians. This protein is capable of binding biotin with high affinity (with a dissociation constant, K_{D} of about 1015 M). The term includes any recombinant or naturally-occurring form of avidin variants thereof that maintain avidin activity (e.g., within at least 30%, 40%, 50%, 60%, 70%, 80%, 90%, 95%, or 100% activity compared to wildtype avidin).

The bioconjugate reactive groups can be chosen such that they do not participate in, or interfere with, the chemical stability of the conjugate described herein. Alternatively, a reactive functional group can be protected from participating in the crosslinking reaction by the presence of a protecting group. In embodiments, the bioconjugate includes a molecular entity derived from the reaction of an unsaturated bond, such as a maleimide, and a sulfhydryl group.

In embodiments, the compounds of the present disclosure use a cleavable linker to attach a label to the molecule. The use of the term “cleavable linker” is not meant to imply that the whole linker is required to be removed. The cleavage site can be located at a position on the linker that ensures that part of the linker remains attached to the molecule after cleavage.

The term “cleavable linker” or “cleavable moiety” refers to a divalent or monovalent, respectively, moiety which is capable of being separated (e.g., detached, split, disconnected, hydrolyzed, a stable bond within the moiety is broken) into distinct entities. A cleavable linker is cleavable (e.g., specifically cleavable) in response to external stimuli (e.g., enzymes, nucleophilic/basic reagents, reducing agents, photo-irradiation, electrophilic/acidic reagents, organometallic and metal reagents, or oxidizing reagents). A chemically cleavable linker refers to a linker which is capable of being split in response to the presence of a chemical (e.g., acid, base, oxidizing agent, reducing agent, Pd(0), tris-(2-carboxyethyl)phosphine, dilute nitrous acid, fluoride, tris(3-hydroxypropyl)phosphine), sodium dithionite (Na₂S₂O₄), or hydrazine (N₂H₄)). In embodiments, a chemically cleavable linker is non-enzymatically cleavable. In embodiments, the cleavable linker is cleaved by contacting the cleavable linker with a cleaving agent. In embodiments, the cleaving agent is a phosphine containing reagent (e.g., TCEP or THPP), sodium dithionite (Na₂S₂O₄), weak acid, hydrazine (N₂H₄), Pd(O), or light-irradiation (e.g., ultraviolet radiation). In embodiments, cleaving includes removing. A “cleavable site” or “scissile linkage” in the context of a polynucleotide is a site which allows controlled cleavage of the polynucleotide strand (e.g., the linker, the primer, or the polynucleotide) by chemical, enzymatic, or photochemical means known in the art and described herein. A scissile site may refer to the linkage of a nucleotide between two other nucleotides in a nucleotide strand (i.e., an internucleosidic linkage). In embodiments, the scissile linkage can be located at any position within the one or more nucleic acid molecules, including at or near a terminal end (e.g., the 3′ end of an oligonucleotide) or in an interior portion of the one or more nucleic acid molecules. In embodiments, conditions suitable for separating a scissile linkage include a modulating the pH and/or the temperature. In embodiments, a scissile site can include at least one acid-labile linkage. For example, an acid-labile linkage may include a phosphoramidate linkage. In embodiments, a phosphoramidate linkage can be hydrolysable under acidic conditions, including mild acidic conditions such as trifluoroacetic acid and a suitable temperature (e.g., 30° C.), or other conditions known in the art, for example Matthias Mag, et al Tetrahedron Letters, Volume 33, Issue 48, 1992, 7319-7322. In embodiments, the scissile site can include at least one photolabile internucleosidic linkage (e.g., o-nitrobenzyl linkages, as described in Walker et al, J. Am. Chem. Soc. 1988, 110, 21, 7170-7177), such as o-nitrobenzyloxymethyl or p-nitrobenzyloxymethyl group(s). In embodiments, the scissile site includes at least one uracil nucleobase. In embodiments, a uracil nucleobase can be cleaved with a uracil DNA glycosylase (UDG) or Formamidopyrimidine DNA Glycosylase Fpg. In embodiments, the scissile linkage site includes a sequence-specific nicking site having a nucleotide sequence that is recognized and nicked by a nicking endonuclease enzyme or a uracil DNA glycosylase.

A photocleavable linker (e.g., including or consisting of an o-nitrobenzyl group) refers to a linker which is capable of being split in response to photo-irradiation (e.g., ultraviolet radiation). An acid-cleavable linker refers to a linker which is capable of being split in response to a change in the pH (e.g., increased acidity). A base-cleavable linker refers to a linker which is capable of being split in response to a change in the pH (e.g., decreased acidity). An oxidant-cleavable linker refers to a linker which is capable of being split in response to the presence of an oxidizing agent. A reductant-cleavable linker refers to a linker which is capable of being split in response to the presence of an reducing agent (e.g., Tris(3-hydroxypropyl)phosphine). In embodiments, the cleavable linker is a dialkylketal linker, an azo linker, an allyl linker, a cyanoethyl linker, a 1-(4,4-dimethyl-2,6-dioxocyclohex-1-ylidene)ethyl linker, or a nitrobenzyl linker.

The term “orthogonally cleavable linker” or “orthogonal cleavable linker” refer to a cleavable linker that is cleaved by a first cleaving agent (e.g., enzyme, nucleophilic/basic reagent, reducing agent, photo-irradiation, electrophilic/acidic reagent, organometallic and metal reagent, oxidizing reagent) in a mixture of two or more different cleaving agents and is not cleaved by any other different cleaving agent in the mixture of two or more cleaving agents. For example, two different cleavable linkers are both orthogonal cleavable linkers when a mixture of the two different cleavable linkers are reacted with two different cleaving agents and each cleavable linker is cleaved by only one of the cleaving agents and not the other cleaving agent. In embodiments, an orthogonally cleavable linker is a cleavable linker that, following cleavage (e.g., following exposure to a cleaving agent), the two separated entities (e.g., fluorescent dye, bioconjugate reactive group) do not further react and form a new orthogonally cleavable linker.

The term “orthogonal detectable label” or “orthogonal detectable moiety” as used herein refer to a detectable label (e.g., fluorescent dye or detectable dye) that is capable of being detected and identified (e.g., by use of a detection means (e.g., emission wavelength, physical characteristic measurement)) in a mixture or a panel (collection of separate samples) of two or more different detectable labels. For example, two different detectable labels that are fluorescent dyes are both orthogonal detectable labels when a panel of the two different fluorescent dyes is subjected to a wavelength of light that is absorbed by one fluorescent dye but not the other and results in emission of light from the fluorescent dye that absorbed the light but not the other fluorescent dye. Orthogonal detectable labels may be separately identified by different absorbance or emission intensities of the orthogonal detectable labels compared to each other and not only be the absolute presence of absence of a signal. An example of a set of four orthogonal detectable labels is the set of Rox™-Labeled Tetrazine, Alexa Fluor® 488-Labeled SHA, Cy5®-Labeled Streptavidin, and R6G-Labeled Dibenzocyclooctyne.

The term “solution” is used in accordance with its plain ordinary meaning in the arts and refers to a liquid mixture in which the minor component (e.g., a solute or compound) is distributed (e.g., uniformly distributed) within the major component (e.g., a solvent).

The term “organic solvent” as used herein is used in accordance with its ordinary meaning in chemistry and refers to a solvent which includes carbon. Non-limiting examples of organic solvents include acetic acid, acetone, acetonitrile, benzene, 1-butanol, 2-butanol, 2-butanone, t-butyl alcohol, carbon tetrachloride, chlorobenzene, chloroform, cyclohexane, 1,2-dichloroethane, diethylene glycol, diethyl ether, diglyme (diethylene glycol, dimethyl ether), 1,2-dimethoxyethane (glyme, DME), dimethylformamide (DMF), dimethyl sulfoxide (DMSO), 1,4-dioxane, ethanol, ethyl acetate, ethylene glycol, glycerin, heptane, hexamethylphosphoramide (HMPA), hexamethylphosphorous, triamide (HMPT), hexane, methanol, methyl t-butyl ether (MTBE), methylene chloride, N-methyl-2-pyrrolidinone (NMP), nitromethane, pentane, petroleum ether (ligroine), 1-propanol, 2-propanol, pyridine, tetrahydrofuran (THF), toluene, triethyl amine, o-xylene, m-xylene, or p-xylene. In embodiments, the organic solvent is or includes chloroform, dichloromethane, methanol, ethanol, tetrahydrofuran, or dioxane.

The term “salt” refers to acid or base salts of the compounds described herein. Illustrative examples of acceptable salts are mineral acid (hydrochloric acid, hydrobromic acid, phosphoric acid, and the like) salts, organic acid (acetic acid, propionic acid, glutamic acid, citric acid and the like) salts, quaternary ammonium (methyl iodide, ethyl iodide, and the like) salts. In embodiments, compounds may be presented with a positive charge, for example

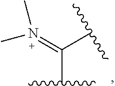

and it is understood an appropriate counter-ion (e.g., chloride ion, fluoride ion, or acetate ion) may also be present, though not explicitly shown. Likewise, for compounds having a negative charge

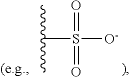 [SMILES: *S(=O)(=O)[O-]]

it is understood an appropriate counter-ion (e.g., a proton, sodium ion, potassium ion, or ammonium ion) may also be present, though not explicitly shown. The protonation state of the compound (e.g., a compound described herein) depends on the local environment (i.e., the pH of the environment), therefore, in embodiments, the compound may be described as having a moiety in a protonated state

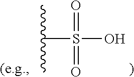 [SMILES: *S(=O)(=O)O]

or an ionic state

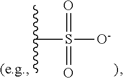 [SMILES: *S(=O)(=O)[O-]]

and it is understood these are interchangeable. In embodiments, the counter-ion is represented by the symbol M (e.g., M⁺ or M⁻).

The term “about” means a range of values including the specified value, which a person of ordinary skill in the art would consider reasonably similar to the specified value. In embodiments, about means within a standard deviation using measurements generally acceptable in the art. In embodiments, about means a range extending to +/−10% of the specified value. In embodiments, about includes the specified value.

The term “leaving group” is used in accordance with its ordinary meaning in chemistry and refers to a moiety (e.g., atom, functional group, or molecule) that separates from the molecule following a chemical reaction (e.g., bond formation, reductive elimination, condensation, or cross-coupling reaction) involving an atom or chemical moiety to which the leaving group is attached, also referred to herein as the “leaving group reactive moiety”, and a complementary reactive moiety (i.e., a chemical moiety that reacts with the leaving group reactive moiety) to form a new bond between the remnants of the leaving groups reactive moiety and the complementary reactive moiety. Thus, the leaving group reactive moiety and the complementary reactive moiety form a complementary reactive group pair. Non limiting examples of leaving groups include hydrogen, hydroxide, halogen (e.g., Br), perfluoroalkylsulfonates (e.g., triflate), tosylates, mesylates, water, alcohols, nitrate, phosphate, thioether, amines, ammonia, fluoride, carboxylate, phenoxides, boronic acid, boronate esters, substituted or unsubstituted piperazinyl, and alkoxides. In embodiments, two molecules are allowed to contact, wherein at least one of the molecules has a leaving group, and upon a reaction and/or bond formation (e.g., acyloin condensation, aldol condensation, Claisen condensation, or Stille reaction) the leaving group(s) separate from the respective molecule. In embodiments, a leaving group is a bioconjugate reactive moiety. In embodiments, the leaving groups is designed to facilitate the reaction. In embodiments, the leaving group is a substituent group.

The term “protecting group” is used in accordance with its ordinary meaning in organic chemistry and refers to a moiety covalently bound to a heteroatom, heterocycloalkyl, or heteroaryl to prevent reactivity of the heteroatom, heterocycloalkyl, or heteroaryl during one or more chemical reactions performed prior to removal of the protecting group. Typically a protecting group is bound to a heteroatom (e.g., O) during a part of a multipart synthesis wherein it is not desired to have the heteroatom react (e.g., a chemical reduction) with the reagent. Following protection the protecting group may be removed (e.g., by modulating the pH). In embodiments the protecting group is an alcohol protecting group. Non-limiting examples of alcohol protecting groups include acetyl, benzoyl, benzyl, methoxymethyl ether (MOM), tetrahydropyranyl (THP), and silyl ether (e.g., trimethylsilyl (TMS)). In embodiments the protecting group is an amine protecting group. Non-limiting examples of amine protecting groups include carbobenzyloxy (Cbz), tert-butyloxycarbonyl (BOC), 9-Fluorenylmethyloxycarbonyl (FMOC), acetyl, benzoyl, benzyl, carbamate, p-methoxybenzyl ether (PMB), and tosyl (Ts).

The term “polymerase-compatible cleavable moiety” or a “reversible terminator moiety” as used herein refers to a cleavable moiety which does not interfere with the function of a polymerase (e.g., DNA polymerase, modified DNA polymerase) in incorporating the nucleotide to which the polymerase-compatible moiety is attached to the 3′ end of the newly formed nucleotide strand. The polymerase-compatible moiety does, however, interfere with the polymerase function by preventing the addition of another nucleotide to the 3′ oxygen of the nucleotide to which the polymerase-compatible moiety is attached. Methods for determining the function of a polymerase contemplated herein are described in B. Rosenblum et al. (Nucleic Acids Res. 1997 Nov. 15; 25(22): 4500-4504); and Z. Zhu et al. (Nucleic Acids Res. 1994 Aug. 25; 22(16): 3418-3422), which are incorporated by reference herein in their entirety for all purposes. In embodiments, the polymerase-compatible cleavable moiety does not decrease the function of a polymerase relative to the absence of the polymerase-compatible cleavable moiety. In embodiments, the polymerase-compatible cleavable moiety does not negatively affect DNA polymerase recognition. In embodiments, the polymerase-compatible cleavable moiety does not negatively affect (e.g., limit) the read length of the DNA polymerase. Additional examples of a polymerase-compatible cleavable moiety may be found in U.S. Pat. No. 6,664,079, Ju J. et al. (2006) Proc Natl Acad Sci USA 103(52):19635-19640; Ruparel H. et al. (2005) Proc Natl Acad Sci USA 102(17):5932-5937; Wu J. et al. (2007) Proc Natl Acad Sci USA 104(104):16462-16467; Guo J. et al. (2008) Proc Natl Acad Sci USA 105(27): 9145-9150 Bentley D. R. et al. (2008) Nature 456(7218):53-59; or Hutter D. et al. (2010) Nucleosides Nucleotides & Nucleic Acids 29:879-895, which are incorporated herein by reference in their entirety for all purposes. In embodiments, a polymerase-compatible moiety includes hydrogen, —N₃, —CN, or halogen. In embodiments, a polymerase-compatible cleavable moiety includes an azido moiety or a dithiol linking moiety. In embodiments, the polymerase-compatible cleavable moiety is independently —NH₂, —CN, —CH₃, C₂-C₆ allyl (e.g., —CH₂—CH═CH₂), methoxyalkyl (e.g., —CH₂—O—CH₃), or
“\*MERGEFORMAT\*MERGEFORMAT —CH₂N₃. In embodiments, the polymerase-compatible cleavable moiety comprises a disulfide moiety. In embodiments, a polymerase-compatible cleavable moiety is a cleavable moiety on a nucleotide, nucleobase, nucleoside, or nucleic acid that does not interfere with the function of a polymerase (e.g., DNA polymerase, modified DNA polymerase). In embodiments, the reversible terminator moiety is

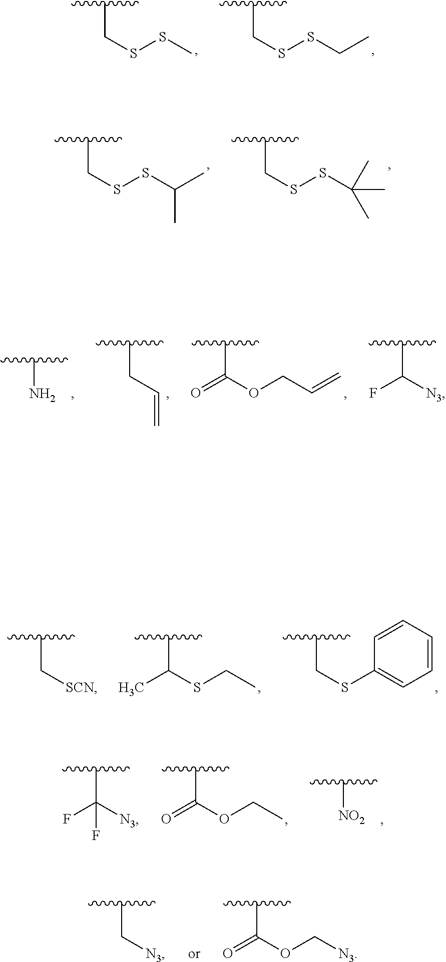

In embodiments, the reversible terminator moiety is

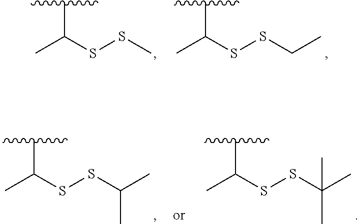

A person of ordinary skill in the art will understand when a variable (e.g., moiety or linker) of a compound or of a compound genus (e.g., a genus described herein) is described by a name or formula of a standalone compound with all valencies filled, the unfilled valence(s) of the variable will be dictated by the context in which the variable is used. For example, when a variable of a compound as described herein is connected (e.g., bonded) to the remainder of the compound through a single bond, that variable is understood to represent a monovalent form (i.e., capable of forming a single bond due to an unfilled valence) of a standalone compound (e.g., if the variable is named “methane” in an embodiment but the variable is known to be attached by a single bond to the remainder of the compound, a person of ordinary skill in the art would understand that the variable is actually a monovalent form of methane, i.e., methyl or
“\*MERGEFORMAT\*MERGEFORMAT —CH₃). Likewise, for a linker variable (e.g., L¹, L², L³, or L⁴ as described herein), a person of ordinary skill in the art will understand that the variable is the divalent form of a standalone compound (e.g., if the variable is assigned to “PEG” or “polyethylene glycol” in an embodiment but the variable is connected by two separate bonds to the remainder of the compound, a person of ordinary skill in the art would understand that the variable is a divalent (i.e., capable of forming two bonds through two unfilled valences) form of PEG instead of the standalone compound PEG).

As used herein, the terms “specific”, “specifically”, “specificity”, or the like of a compound refers to the compound's ability to cause a particular action, such as binding, to a particular molecular target with minimal or no action to other proteins in the cell.

The terms “attached,” “bind,” and “bound” as used herein are used in accordance with their plain and ordinary meanings and refer to an association between atoms or molecules. The association can be direct or indirect. For example, attached molecules may be directly bound to one another, e.g., by a covalent bond or non-covalent bond (e.g. electrostatic interactions (e.g. ionic bond, hydrogen bond, halogen bond), van der Waals interactions (e.g. dipole-dipole, dipole-induced dipole, London dispersion), ring stacking (pi effects), hydrophobic interactions and the like). As a further example, two molecules may be bound indirectly to one another by way of direct binding to one or more intermediate molecules, thereby forming a complex.

“Specific binding” is where the binding is selective between two molecules. A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. In embodiments, the KD (equilibrium dissociation constant) between two specific binding molecules is less than 10⁻⁶ M, less than 10⁻⁷ M, less than 10⁻⁸ M, less than 10⁻⁹ M, less than 10⁻¹¹ M, less than 10⁻¹¹ M, or less than about 10⁻¹² M or less.

As used herein, the terms “sequencing”, “sequence determination”, “determining a nucleotide sequence”, and the like include determination of a partial or complete sequence information (e.g., a sequence) of a polynucleotide being sequenced, and particularly physical processes for generating such sequence information. That is, the term includes sequence comparisons, consensus sequence determination, contig assembly, fingerprinting, and like levels of information about a target polynucleotide, as well as the express identification and ordering of nucleotides in a target polynucleotide. The term also includes the determination of the identification, ordering, and locations of one, two, or three of the four types of nucleotides within a target polynucleotide. In some embodiments, a sequencing process described herein includes contacting a template and an annealed primer with a suitable polymerase under conditions suitable for polymerase extension and/or sequencing.

The term “particle” means a small body made of a rigid or semi-rigid material. The body can have a shape characterized, for example, as a sphere, oval, microsphere, or other recognized particle shape whether having regular or irregular dimensions. The particles may in one way or another rest upon a two dimensional surface by magnetic, gravitational, or ionic forces, or by chemical bonding, or by any other means known to those skilled in the art. In further embodiments, the bead may have magnetic properties. Further the beads may have a density that allows them to rest upon a two dimensional surface in solution. Particles may consist of glass, polystyrene, latex, metal, quantum dot, polymers, silica, metal oxides, ceramics, or any other substance suitable for binding to nucleic acids, or chemicals or proteins which can then attach to nucleic acids. The particles may be rod shaped or spherical or disc shaped, or comprise any other shape. The particles may also be distinguishable by their shape or size or physical location. The particles may be distinguished through spectroscopy by having a composition containing dyes or fluorochromes in various ratios or concentrations. The particles may also be distinguishable by barcode or holographic images or other imprinted forms of particle coding. Where the particles are magnetic particles, they may be attracted to the surface of the chamber by application of a magnetic field and the magnetic particles may be dispersed from the surface of the chamber by removal of the magnetic field. The magnetic particles are preferably paramagnetic or superparamagnetic. In embodiments, the particle comprises a polymer having a plurality of branches, wherein the polymer has a star, comb, cross-linked, bottle brush, or dendrimer configuration. In embodiments, the particle comprises a polymer having a plurality of branches, wherein the compound described herein is attached to the plurality of the branches. In embodiments, a plurality of the compound described herein is attached to the plurality of the branches attached to the particle. In embodiments, the particle includes a plurality of the same compound attached to the plurality of the branches.

The term “gel” in this context refers to a semi-rigid solid that is permeable to liquids and gases. As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers. Hydrogels can contain over 99% water and may comprise natural or synthetic polymers, or a combination thereof. Hydrogels also possess a degree of flexibility very similar to natural tissue, due to their significant water content. A detailed description of suitable hydrogels may be found in published U.S. patent application 2010/0055733, herein specifically incorporated by reference. By “hydrogel subunits” or “hydrogel precursors” is meant hydrophilic monomers, prepolymers, or polymers that can be crosslinked, or “polymerized”, to form a three-dimensional (3D) hydrogel network. Hydrogels can be derived from a single species of monomer or from two or more different monomer species with at least one hydrophilic component. Hydrogels may be prepared by cross-linking hydrophilic biopolymers or synthetic polymers. Thus, in some embodiments, the hydrogel may include a crosslinker. As used herein, the term “crosslinker” refers to a molecule that can form a three-dimensional network when reacted with the appropriate base monomers. Examples of the hydrogel polymers, which may include one or more crosslinkers, include but are not limited to, hyaluronans, chitosans, agar, heparin, sulfate, cellulose, alginates (including alginate sulfate), collagen, dextrans (including dextran sulfate), pectin, carrageenan, polylysine, gelatins (including gelatin type A), agarose, (meth)acrylate-oligolactide-PEO-oligolactide-(meth)acrylate, PEO—PPO-PEO copolymers (Pluronics), poly(phosphazene), poly(methacrylates), poly(N-vinylpyrrolidone), PL(G)A-PEO-PL(G)A copolymers, poly(ethylene imine), polyethylene glycol (PEG)-thiol, PEG-acrylate, acrylamide, N,N′-bis(acryloyl)cystamine, PEG, polypropylene oxide (PPO), polyacrylic acid, poly(hydroxyethyl methacrylate) (PHEMA), poly(methyl methacrylate) (PMMA), poly(N-isopropylacrylamide) (PNIPAAm), poly(lactic acid) (PLA), poly(lactic-co-glycolic acid) (PLGA), polycaprolactone (PCL), poly(vinylsulfonic acid) (PVSA), poly(L-aspartic acid), poly(L-glutamic acid), bisacrylamide, diacrylate, diallylamine, triallylamine, divinyl sulfone, diethyleneglycol diallyl ether, ethyleneglycol diacrylate, polymethyleneglycol diacrylate, polyethyleneglycol diacrylate, trimethylopropoane trimethacrylate, ethoxylated trimethylol triacrylate, or ethoxylated pentaerythritol tetracrylate, or combinations thereof. Thus, for example, a combination may include a polymer and a crosslinker, for example polyethylene glycol (PEG)-thiol/PEG-acrylate, acrylamide/N,N′-bis(acryloyl)cystamine (BACy), or PEG/polypropylene oxide (PPO). In embodiments, the hydrogel includes chemical crosslinks (e.g., intermolecular or intramolecular joining of two or more molecules by a covalent bond) and may be referred to as a chemical hydrogel. In embodiments, the hydrogel includes physical crosslinks (e.g., intermolecular or intramolecular joining of two or more molecules by a non-covalent bond) and may be referred to as a physical hydrogel. In embodiments, the physical hydrogel include one or more crosslinks including hydrogen bonds, hydrophobic interactions, and/or polymer chain entanglements.

As used herein, the term “polymer” refers to macromolecules having one or more structurally unique repeating units. The repeating units are referred to as “monomers,” which are polymerized for the polymer. Typically, a polymer is formed by monomers linked in a chain-like structure. A polymer formed entirely from a single type of monomer is referred to as a “homopolymer.” A polymer formed from two or more unique repeating structural units may be referred to as a “copolymer.” A polymer may be linear or branched, and may be random, block, polymer brush, hyperbranched polymer, bottlebrush polymer, dendritic polymer, or polymer micelles. The term “polymer” includes homopolymers, copolymers, tripolymers, tetra polymers and other polymeric molecules made from monomeric subunits. Copolymers include alternating copolymers, periodic copolymers, statistical copolymers, random copolymers, block copolymers, linear copolymers and branched copolymers. The term “polymerizable monomer” is used in accordance with its meaning in the art of polymer chemistry and refers to a compound that may covalently bind chemically to other monomer molecules (such as other polymerizable monomers that are the same or different) to form a polymer.

The term “polymer” refers to a molecule including repeating subunits (e.g., polymerized monomers). For example, polymeric molecules may be based upon polyethylene glycol (PEG), tetraethylene glycol (TEG), polyvinylpyrrolidone (PVP), poly(xylene), or poly(p-xylylene). The term “polymerizable monomer” is used in accordance with its meaning in the art of polymer chemistry and refers to a compound that may covalently bind chemically to other monomer molecules (such as other polymerizable monomers that are the same or different) to form a polymer. In embodiments, polymer refers to PEG, having the formula:

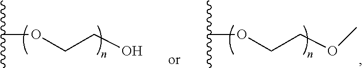

wherein n is an integer from 1 to 30.

Polymers can be hydrophilic, hydrophobic or amphiphilic, as known in the art. Thus, “hydrophilic polymers” are substantially miscible with water and include, but are not limited to, polyethylene glycol and the like. “Hydrophobic polymers” are substantially immiscible with water and include, but are not limited to, polyethylene, polypropylene, polybutadiene, polystyrene, polymers disclosed herein, and the like. “Amphiphilic polymers” have both hydrophilic and hydrophobic properties and are typically copolymers having hydrophilic segment(s) and hydrophobic segment(s). Polymers include homopolymers, random copolymers, and block copolymers, as known in the art. The term “homopolymer” refers, in the usual and customary sense, to a polymer having a single monomeric unit. The term “copolymer” refers to a polymer derived from two or more monomeric species. The term “random copolymer” refers to a polymer derived from two or more monomeric species with no preferred ordering of the monomeric species. The term “block copolymer” refers to polymers having two or homopolymer subunits linked by covalent bond. Thus, the term “hydrophobic homopolymer” refers to a homopolymer which is hydrophobic. The term “hydrophobic block copolymer” refers to two or more homopolymer subunits linked by covalent bonds and which is hydrophobic.

As used herein, the term “hydrogel” refers to a three-dimensional polymeric structure that is substantially insoluble in water, but which is capable of absorbing and retaining large quantities of water to form a substantially stable, often soft and pliable, structure. In embodiments, water can penetrate in between polymer chains of a polymer network, subsequently causing swelling and the formation of a hydrogel. In embodiments, hydrogels are super-absorbent (e.g., containing more than about 90% water) and can be comprised of natural or synthetic polymers.

As used herein, the term “substrate” refers to a solid support material. The substrate can be non-porous or porous. The substrate can be rigid or flexible. As used herein, the terms “solid support” and “solid surface” refers to discrete solid or semi-solid surface. A solid support may encompass any type of solid, porous, or hollow sphere, ball, cylinder, or other similar configuration composed of plastic, ceramic, metal, or polymeric material (e.g., hydrogel) onto which a nucleic acid may be immobilized (e.g., covalently or non-covalently). A nonporous substrate generally provides a seal against bulk flow of liquids or gases. Exemplary solid supports include, but are not limited to, glass and modified or functionalized glass, plastics (including acrylics, polystyrene and copolymers of styrene and other materials, polypropylene, polyethylene, polybutylene, polyurethanes, Teflon™, cyclic olefin copolymers, polyimides etc.), nylon, ceramics, resins, Zeonor® (Zeon Corporation), silica or silica-based materials including silicon and modified silicon, carbon, metals, inorganic glasses, optical fiber bundles, photopatternable dry film resists, UV-cured adhesives and polymers. Particularly useful solid supports for some embodiments have at least one surface located within a flow cell. Solid surfaces can also be varied in their shape depending on the application in a method described herein. For example, a solid surface useful herein can be planar, or contain regions which are concave or convex. In embodiments, the geometry of the concave or convex regions (e.g., wells) of the solid surface conform to the size and shape of the particle to maximize the contact between as substantially circular particle. In embodiments, the wells of an array are randomly located such that nearest neighbor features have random spacing between each other. Alternatively, in embodiments the spacing between the wells can be ordered, for example, forming a regular pattern. The term solid substrate is encompassing of a substrate (e.g., a flow cell) having a surface including a polymer coating covalently attached thereto. In embodiments, the solid substrate is a flow cell.

Broadly speaking, for nucleic acid sequencing applications, a flow cell may be considered a reaction chamber that contains one or more nucleic acid templates tethered to a solid support, to which nucleotides and ancillary reagents are iteratively applied and washed away. The flow cell allows for imaging of the sites at which the nucleic acids are bound, and resulting image data is used for the desired analysis. The latest commercial sequencing instruments use flow cells and massive parallelization to increase sequencing capacity.

The term “flow cell” as used herein refers to a chamber including a solid surface across which one or more fluid reagents can be flowed. Examples of flow cells and related fluidic systems and detection platforms that can be readily used in the methods of the present disclosure are described, for example, in Bentley et al., Nature 456:53-59 (2008). In certain embodiments a substrate includes a surface (e.g., a surface of a flow cell, a surface of a tube, a surface of a chip), for example a metal surface (e.g., steel, gold, silver, aluminum, silicon and copper). In embodiments a substrate (e.g., a substrate surface) is coated and/or includes functional groups and/or inert materials. In certain embodiments a substrate includes a bead, a chip, a capillary, a plate, a membrane, a wafer (e.g., silicon wafers), a comb, or a pin for example. In some embodiments a substrate includes a bead and/or a nanoparticle. A substrate can be made of a suitable material, non-limiting examples of which include a plastic or a suitable polymer (e.g., polycarbonate, poly(vinyl alcohol), poly(divinylbenzene), polystyrene, polyamide, polyester, polyvinylidene difluoride (PVDF), polyethylene, polyurethane, polypropylene, and the like), borosilicate, glass, nylon, Wang resin, Merrifield resin, metal (e.g., iron, a metal alloy, sepharose, agarose, polyacrylamide, dextran, cellulose and the like or combinations thereof. In embodiments a substrate includes a magnetic material (e.g., iron, nickel, cobalt, platinum, aluminum, and the like). In embodiments a substrate includes a magnetic bead (e.g., DYNABEADS™ (Invitrogen), hematite, AMPure XP). Magnets can be used to purify and/or capture nucleic acids bound to certain substrates (e.g., substrates including a metal or magnetic material). The flow cell is typically a glass slide containing small fluidic channels (e.g., a glass slide 75 mm×25 mm×1 mm having one or more channels), through which sequencing solutions (e.g., polymerases, nucleotides, and buffers) may traverse. Though typically glass, suitable flow cell materials may include polymeric materials, plastics, silicon, quartz (fused silica), Borofloat® (SCHOTT) glass, silica, silica-based materials, carbon, metals, an optical fiber or optical fiber bundles, sapphire, or plastic materials such as COCs and epoxies. The particular material can be selected based on properties desired for a particular use. For example, materials that are transparent to a desired wavelength of radiation are useful for analytical techniques that will utilize radiation of the desired wavelength. Conversely, it may be desirable to select a material that does not pass radiation of a certain wavelength (e.g., being opaque, absorptive, or reflective). In embodiments, the material of the flow cell is selected due to the ability to conduct thermal energy. In embodiments, a flow cell includes inlet and outlet ports and a flow channel extending there between.

As used herein, the term “channel” refers to a passage in or on a substrate material that directs the flow of a fluid. A channel may run along the surface of a substrate, or may run through the substrate between openings in the substrate. A channel can have a cross section that is partially or fully surrounded by substrate material (e.g., a fluid impermeable substrate material). For example, a partially surrounded cross section can be a groove, trough, furrow or gutter that inhibits lateral flow of a fluid. The transverse cross section of an open channel can be, for example, U-shaped, V-shaped, curved, angular, polygonal, or hyperbolic. A channel can have a fully surrounded cross section such as a tunnel, tube, or pipe. A fully surrounded channel can have a rounded, circular, elliptical, square, rectangular, or polygonal cross section. In particular embodiments, a channel can be located in a flow cell, for example, being embedded within the flow cell. A channel in a flow cell can include one or more windows that are transparent to light in a particular region of the wavelength spectrum. In embodiments, the channel contains one or more polymers of the disclosure. In embodiments, the channel is filled by the one or more polymers, and flow through the channel (e.g., as in a sample fluid) is directed through the polymer in the channel. In embodiments, the tissue is in a channel of a flow cell.

As used herein, the term “inlet” or “inlet port” refers to the location on a flow cell assembly where the reagents and fluids used for methods described herein enters the flow cell. As used herein, the term “outlet” or “outlet port” refers to the location on a flow cell assembly where the reagents and fluids used for methods described herein exits the flow cell after contacting the reaction chamber containing the cell or tissue to be analyzed.

As used herein, the term “reaction chamber” refers to a contained space or vessel designed for conducting chemical, biological, or physical reactions. A reaction chamber may include features such as inlets and outlets for introducing and removing substances, sensors for monitoring reaction conditions, and mechanisms for agitation or mixing. In embodiments, the reaction chamber is a part of the flow cell where the cell or tissue is in contact with the fluids (e.g., buffers), polymerases, nucleotides, and reagents used for the methods described herein. In embodiments, the reaction chamber is formed when a first solid support and a second solid support configured to provide a channel are attached together. In embodiments, the reaction chamber is an enclosed (i.e., closed) container containing one or two openings for introducing and removing fluids and reagents.

The term “surface” is intended to mean an external part or external layer of a substrate. The surface can be in contact with another material such as a gas, liquid, gel, polymer, organic polymer, second surface of a similar or different material, metal, or coat. The surface, or regions thereof, can be substantially flat. The substrate and/or the surface can have surface features such as wells, pits, channels, ridges, raised regions, pegs, posts or the like.

As used herein, the term “feature” refers a point or area in a pattern that can be distinguished from other points or areas according to its relative location. An individual feature can include one or more polynucleotides. For example, a feature can include a single target nucleic acid molecule having a particular sequence or a feature can include several nucleic acid molecules having the same sequence (and/or complementary sequence, thereof). Different molecules that are at different features of a pattern can be differentiated from each other according to the locations of the features in the pattern. Non-limiting examples of features include wells in a substrate, particles (e.g., beads) in or on a substrate, polymers in or on a substrate, projections from a substrate, ridges on a substrate, or channels in a substrate. In embodiments, the one or more features include a reaction chamber and its contents. In embodiments, the one or more features includes a target (e.g., a nucleic acid, protein, or biomarker), a cell, or a tissue sample. In embodiments, the feature is a nucleotide (e.g., a fluorescently labeled nucleotide). In embodiments, the feature is a nucleic acid. In embodiments, the feature is a protein. In embodiments, the feature is a biomolecule.

The term “microplate”, or “multiwell container” as used herein, refers to a substrate including a surface, the surface including a plurality of reaction chambers separated from each other by interstitial regions on the surface. In embodiments, the microplate has dimensions as provided and described by American National Standards Institute (ANSI) and Society for Laboratory Automation And Screening (SLAS); for example the tolerances and dimensions set forth in ANSI SLAS 1-2004 (R2012); ANSI SLAS 2-2004 (R2012); ANSI SLAS 3-2004 (R2012); ANSI SLAS 4-2004 (R2012); and ANSI SLAS 6-2012, which are incorporated herein by reference. The dimensions of the microplate as described herein and the arrangement of the reaction chambers may be compatible with an established format for automated laboratory equipment. In embodiments, the device described herein provides methods for high-throughput screening. High-throughput screening (HTS) refers to a process that uses a combination of modern robotics, data processing and control software, liquid handling devices, and/or sensitive detectors, to efficiently process a large amount of (e.g., thousands, hundreds of thousands, or millions) samples in biochemical, genetic, or pharmacological experiments, either in parallel or in sequence, within a reasonably short period of time (e.g., days). Preferably, the process is amenable to automation, such as robotic simultaneous handling of 96 samples, 384 samples, 1536 samples or more. A typical HTS robot tests up to 100,000 to a few hundred thousand compounds per day. The samples are often in small volumes, such as no more than 1 mL, 500 l, 200 l, 100 l, 50 l or less. Through this process, one can rapidly identify active compounds, small molecules, antibodies, proteins or polynucleotides in a cell.

The reaction chambers may be provided as wells of a multiwell container (alternatively referred to as reaction chambers), for example a microplate may contain 2, 4, 6, 12, 24, 48, 96, 384, or 1536 sample wells. In embodiments, the 96 and 384 wells are arranged in a 2:3 rectangular matrix. In embodiments, the 24 wells are arranged in a 3:8 rectangular matrix. In embodiments, the 48 wells are arranged in a 3:4 rectangular matrix. In embodiments, the reaction chamber is a microscope slide (e.g., a glass slide about 75 mm by about 25 mm). In embodiments the slide is a concavity slide (e.g., the slide includes a depression). In embodiments, the slide includes a coating for enhanced cell adhesion (e.g., poly-L-lysine, silanes, carbon nanotubes, polymers, epoxy resins, or gold). In embodiments, the microplate is about 5 inches by about 3.33 inches, and includes a plurality of 5 mm diameter wells. In embodiments, the microplate is about 5 inches by about 3.33 inches and includes a plurality of 6 mm diameter wells. In embodiments, the microplate is about 5 inches by about 3.33 inches and includes a plurality of 7 mm diameter wells. In embodiments, the microplate is about 5 inches by about 3.33 inches and includes a plurality of 7.5 mm diameter wells. In embodiments, the microplate is 5 inches by 3.33 inches, and includes a plurality of 7.5 mm diameter wells. In embodiments, the microplate is about 5 inches by about 3.33 inches and includes a plurality of 8 mm diameter wells. In embodiments, the microplate is a flat glass or plastic tray in which an array of wells are formed, wherein each well can hold between from a few microliters to hundreds of microliters of fluid reagents and samples. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 5-7 mm. In embodiments, the microplate has a rectangular shape that measures 127.7 mm±0.5 mm in length by 85.4 mm±0.5 mm in width, and includes 6, 12, 24, 48, or 96 wells, wherein each well has an average diameter of about 6 mm.

The term “well” refers to a discrete concave feature in a substrate having a surface opening that is completely surrounded by interstitial region(s) of the surface. Wells can have any of a variety of shapes at their opening in a surface including but not limited to round, elliptical, square, polygonal, or star shaped (i.e., star shaped with any number of vertices). The cross section of a well taken orthogonally with the surface may be curved, square, polygonal, hyperbolic, conical, or angular. The wells of a microplate are available in different shapes, for example F-Bottom: flat bottom; C-Bottom: bottom with minimal rounded edges; V-Bottom: V-shaped bottom; or U-Bottom: U-shaped bottom. In embodiments, the well is substantially square. In embodiments, the well is square. In embodiments, the well is F-bottom. In embodiments, the microplate includes 24 substantially round flat bottom wells. In embodiments, the microplate includes 48 substantially round flat bottom wells. In embodiments, the microplate includes 96 substantially round flat bottom wells. In embodiments, the microplate includes 384 substantially square flat bottom wells.

The discrete regions (i.e., features, wells) of the microplate may have defined locations in a regular array, which may correspond to a rectilinear pattern, circular pattern, hexagonal pattern, or the like. In embodiments, the pattern of wells includes concentric circles of regions, spiral patterns, rectilinear patterns, hexagonal patterns, and the like. In embodiments, the pattern of wells is arranged in a rectilinear or hexagonal pattern A regular array of such regions is advantageous for detection and data analysis of signals collected from the arrays during an analysis. These discrete regions are separated by interstitial regions. As used herein, the term “interstitial region” refers to an area in a substrate or on a surface that separates other areas of the substrate or surface. For example, an interstitial region can separate one concave feature of an array from another concave feature of the array. The two regions that are separated from each other can be discrete, lacking contact with each other. In another example, an interstitial region can separate a first portion of a feature from a second portion of a feature. In embodiments the interstitial region is continuous whereas the features are discrete, for example, as is the case for an array of wells in an otherwise continuous surface. The separation provided by an interstitial region can be partial or full separation. In embodiments, interstitial regions have a surface material that differs from the surface material of the wells (e.g., the interstitial region contains a photoresist and the surface of the well is glass). In embodiments, interstitial regions have a surface material that is the same as the surface material of the wells (e.g., both the surface of the interstitial region and the surface of well contain a polymer or copolymer).

As used herein, the terms “sequencing”, “sequence determination”, and “determining a nucleotide sequence”, are used in accordance with their ordinary meaning in the art, and refer to determination of partial as well as full sequence information of the nucleic acid being sequenced, and particular physical processes for generating such sequence information. That is, the term includes sequence comparisons, fingerprinting, and like levels of information about a target nucleic acid, as well as the express identification and ordering of nucleotides in a target nucleic acid. The term also includes the determination of the identification, ordering, and locations of one, two, or three of the four types of nucleotides within a target nucleic acid. As used herein, the term “sequencing cycle” is used in accordance with its plain and ordinary meaning and refers to incorporating one or more nucleotides (e.g., nucleotide analogues) to the 3′ end of a polynucleotide with a polymerase, and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “sequencing reaction mixture” is used in accordance with its plain and ordinary meaning and refers to an aqueous mixture that contains the reagents necessary to allow dNTP or dNTP analogue (e.g., a modified nucleotide) to add a nucleotide to a DNA strand by a DNA polymerase. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

As used herein, the term “sequencing cycle” is used in accordance with its plain and ordinary meaning and refers to incorporating one or more nucleotides (e.g., nucleotide analogues) to the 3′ end of a polynucleotide with a polymerase and detecting one or more labels that identify the one or more nucleotides incorporated. In embodiments, one nucleotide (e.g., a modified nucleotide) is incorporated per sequencing cycle. The sequencing may be accomplished by, for example, sequencing by synthesis, pyrosequencing, and the like. In embodiments, a sequencing cycle includes extending a complementary polynucleotide by incorporating a first nucleotide using a polymerase, wherein the polynucleotide is hybridized to a template nucleic acid, detecting the first nucleotide, and identifying the first nucleotide. In embodiments, to begin a sequencing cycle, one or more differently labeled nucleotides and a DNA polymerase can be introduced. Following nucleotide addition, signals produced (e.g., via excitation and emission of a detectable label) can be detected to determine the identity of the incorporated nucleotide (based on the labels on the nucleotides). Reagents can then be added to remove the 3′ reversible terminator and to remove labels from each incorporated base. Reagents, enzymes, and other substances can be removed between steps by washing. Cycles may include repeating these steps, and the sequence of each cluster is read over the multiple repetitions.

As used herein, the term “extension” or “elongation” is used in accordance with their plain and ordinary meanings and refer to synthesis by a polymerase of a new polynucleotide strand complementary to a template strand by adding free nucleotides (e.g., dNTPs) from a reaction mixture that are complementary to the template in the 5′-to-3′ direction. Extension includes condensing the 5′-phosphate group of the dNTPs with the 3′-hydroxy group at the end of the nascent (elongating) DNA strand.

As used herein, the term “sequencing read” is used in accordance with its plain and ordinary meaning and refers to an inferred sequence of nucleotide bases (or nucleotide base probabilities) corresponding to all or part of a single polynucleotide fragment. A sequencing read may include 10, 20, 30, 40, 50, 60, 70, 80, 90, 100, 150, 200, 250, or more nucleotide bases. In embodiments, a sequencing read includes reading a barcode sequence and a template nucleotide sequence. In embodiments, a sequencing read includes reading a template nucleotide sequence. In embodiments, a sequencing read includes reading a barcode and not a template nucleotide sequence. Reads of length 20-40 base pairs (bp) are referred to as ultra-short. Typical sequencers produce read lengths in the range of 100-500 bp. Read length is a factor which can affect the results of biological studies. For example, longer read lengths improve the resolution of de novo genome assembly and detection of structural variants. In embodiments, a sequencing read includes reading a barcode and a template nucleotide sequence. In embodiments, a sequencing read includes reading a template nucleotide sequence. In embodiments, a sequencing read includes reading a barcode and not a template nucleotide sequence. In embodiments, a sequencing read includes a computationally derived string corresponding to the detected label. In some embodiments, a sequencing read may include 300, 400, 500, 600, 700, 800, 900, 1,000, 1,100, 1,200, 1,300, 1,400, 1,500, or more nucleotide bases.

The term “multiplexing” as used herein refers to an analytical method in which the presence and/or amount of multiple targets, e.g., multiple nucleic acid target sequences, can be assayed simultaneously by using the methods and devices as described herein, each of which has at least one different detection characteristic, e.g., fluorescence characteristic (for example excitation wavelength, emission wavelength, emission intensity, FWHM (full width at half maximum peak height), or fluorescence lifetime) or a unique nucleic acid or protein sequence characteristic. As used herein, the term “multiplex” is used to refer to an assay in which multiple (i.e. at least two) different biomolecules are assayed at the same time, and more particularly in the same aliquot of the sample, or in the same reaction mixture. In embodiments, more than two different biomolecules are assayed at the same time. In embodiments, at least 2, 4, 6, 8, 10, 20, 50, 100, 200, 300, 400, 500, 600, 700, 800, 900, 1000, 1100, 1200, 1300, 1400 or 1500 or more biomolecules are detected according to the present method.

As used herein, the term “detection agent” refers to an agent with a label that is capable of specifically binding to a biomolecule of interest to facilitate the detection of the biomolecule of interest. Binding the detection agent to the biomolecule of interest facilitates detecting the label and thus, detection of the biomolecule of interest. An example of a detection agent with a label (e.g., a detectable label) include fluorescently labeled antibodies used for flow cytometry applications. An additional example of a detection agent with a label is a padlock probe capable of hybridizing to a nucleic acid of interest, where the padlock probe harbors an oligonucleotide label that is sequenced to facilitate the detection of the nucleic acid of interest.

Complementary single stranded nucleic acids and/or substantially complementary single stranded nucleic acids can hybridize to each other under hybridization conditions, thereby forming a nucleic acid that is partially or fully double stranded. All or a portion of a nucleic acid sequence may be substantially complementary to another nucleic acid sequence, in some embodiments. As referred to herein, “substantially complementary” refers to nucleotide sequences that can hybridize with each other under suitable hybridization conditions. Hybridization conditions can be altered to tolerate varying amounts of sequence mismatch within complementary nucleic acids that are substantially complementary. Substantially complementary portions of nucleic acids that can hybridize to each other can be 75% or more, 76% or more, 77% or more, 78% or more, 79% or more, 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more or 99% or more complementary to each other. In some embodiments substantially complementary portions of nucleic acids that can hybridize to each other are 100% complementary. Nucleic acids, or portions thereof, that are configured to hybridize to each other often include nucleic acid sequences that are substantially complementary to each other.

“Hybridize” shall mean the annealing of a nucleic acid sequence to another nucleic acid sequence (e.g., one single-stranded nucleic acid (such as a primer) to another nucleic acid) based on the well-understood principle of sequence complementarity. In an embodiment the other nucleic acid is a single-stranded nucleic acid. In some embodiments, one portion of a nucleic acid hybridizes to itself, such as in the formation of a hairpin structure. The propensity for hybridization between nucleic acids depends on the temperature and ionic strength of their milieu, the length of the nucleic acids and the degree of complementarity. The effect of these parameters on hybridization is described in, for example, Sambrook J., Fritsch E. F., Maniatis T., Molecular cloning: a laboratory manual, Cold Spring Harbor Laboratory Press, New York (1989). As used herein, hybridization of a primer, or of a DNA extension product, respectively, is extendable by creation of a phosphodiester bond with an available nucleotide or nucleotide analogue capable of forming a phosphodiester bond, therewith. For example, hybridization can be performed at a temperature ranging from 15° C. to 95° C. In some embodiments, the hybridization is performed at a temperature of about 20° C., about 25° C., about 30° C., about 35° C., about 40° C., about 45° C., about 50° C., about 55° C., about 60° C., about 65° C., about 70° C., about 75° C., about 80° C., about 85° C., about 90° C., or about 95° C. In other embodiments, the stringency of the hybridization can be further altered by the addition or removal of components of the buffered solution.

As used herein, “specifically hybridizes” refers to preferential hybridization under hybridization conditions where two nucleic acids, or portions thereof, that are substantially complementary, hybridize to each other and not to other nucleic acids that are not substantially complementary to either of the two nucleic acids. For example, specific hybridization includes the hybridization of a primer or capture nucleic acid to a portion of a target nucleic acid (e.g., a template, or adapter portion of a template) that is substantially complementary to the primer or capture nucleic acid. In some embodiments nucleic acids, or portions thereof, that are configured to specifically hybridize are often about 80% or more, 81% or more, 82% or more, 83% or more, 84% or more, 85% or more, 86% or more, 87% or more, 88% or more, 89% or more, 90% or more, 91% or more, 92% or more, 93% or more, 94% or more, 95% or more, 96% or more, 97% or more, 98% or more, 99% or more or 100% complementary to each other over a contiguous portion of nucleic acid sequence. A specific hybridization discriminates over non-specific hybridization interactions (e.g., two nucleic acids that a not configured to specifically hybridize, e.g., two nucleic acids that are 80% or less, 70% or less, 60% or less or 50% or less complementary) by about 2-fold or more, often about 10-fold or more, and sometimes about 100-fold or more, 1000-fold or more, 10,000-fold or more, 100,000-fold or more, or 1,000,000-fold or more. Two nucleic acid strands that are hybridized to each other can form a duplex which includes a double stranded portion of nucleic acid.

As used herein, the term “adjacent,” refers to two nucleotide sequences in a nucleic acid, can refer to nucleotide sequences separated by 0 to about 20 nucleotides, more specifically, in a range of about 1 to about 10 nucleotides, or to sequences that directly abut one another. As those of skill in the art appreciate, two nucleotide sequences that that are to ligated together will generally directly abut one another.

A nucleic acid can be amplified by a suitable method. The term “amplification,” “amplified” or “amplifying” as used herein refers to subjecting a target nucleic acid in a sample to a process that linearly or exponentially generates amplicon nucleic acids having the same or substantially the same (e.g., substantially identical) nucleotide sequence as the target nucleic acid, or segment thereof, and/or a complement thereof (which may be referred to herein as an “amplification product” or “amplification products”). In some embodiments an amplification reaction comprises a suitable thermal stable polymerase. Thermal stable polymerases are known and are stable for prolonged periods of time, at temperature greater than 80° C. when compared to common polymerases found in most mammals. In certain embodiments the term “amplification,” “amplified” or “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In certain embodiments an amplified product (e.g., an amplicon) can contain one or more additional and/or different nucleotides than the template sequence, or portion thereof, from which the amplicon was generated (e.g., a primer can contain “extra” nucleotides (such as a 5′ portion that does not hybridize to the template), or one or more mismatched bases within a hybridizing portion of the primer).

As used herein, bridge-PCR (bPCR) amplification is a method for solid-phase amplification as exemplified by the disclosures of U.S. Pat. Nos. 5,641,658; 7,115,400; and U.S. Patent Publ. No. 2008/0009420, each of which is incorporated herein by reference in its entirety. Bridge-PCR involves repeated polymerase chain reaction cycles, cycling between denaturation, annealing, and extension conditions and enables controlled, spatially-localized, amplification, to generate amplification products (e.g., amplicons) immobilized on a solid support in order to form arrays comprised of colonies (or “clusters”) of immobilized nucleic acid molecule.

Amplification according to the present teachings encompasses any means by which at least a part of at least one target nucleic acid is reproduced, typically in a template-dependent manner, including without limitation, a broad range of techniques for amplifying nucleic acid sequences, either linearly or exponentially. Illustrative means for performing an amplifying step include ligase chain reaction (LCR), ligase detection reaction (LDR), ligation followed by Q-replicase amplification, PCR, primer extension, strand displacement amplification (SDA), hyperbranched strand displacement amplification, multiple displacement amplification (MDA), nucleic acid strand-based amplification (NASBA), two-step multiplexed amplifications, rolling circle amplification (RCA), and the like, including multiplex versions and combinations thereof, for example but not limited to, OLA (oligonucleotide ligation assay)/PCR, PCR/OLA, LDR/PCR, PCR/PCR/LDR, PCR/LDR, LCR/PCR, PCR/LCR (also known as combined chain reaction-CCR), and the like. Descriptions of such techniques can be found in, among other sources, Ausbel et al.; PCR Primer: A Laboratory Manual, Diffenbach, Ed., Cold Spring Harbor Press (1995); The Electronic Protocol Book, Chang Bioscience (2002); Msuih et al., J. Clin. Micro. 34:501-07 (1996); The Nucleic Acid Protocols Handbook, R. Rapley, ed., Humana Press, Totowa, N.J. (2002); Abramson et al., Curr Opin Biotechnol. 1993 February; 4(1):41-7, U.S. Pat. Nos. 6,027,998; 6,605,451, Barany et al., PCT Publication No. WO 97/31256; Wenz et al., PCT Publication No. WO 01/92579; Day et al., Genomics, 29(1): 152-162 (1995), Ehrlich et al., Science 252:1643-50 (1991); Innis et al., PCR Protocols: A Guide to Methods and Applications, Academic Press (1990); Favis et al., Nature Biotechnology 18:561-64 (2000); and Rabenau et al., Infection 28:97-102 (2000); Belgrader, Barany, and Lubin, Development of a Multiplex Ligation Detection Reaction DNA Typing Assay, Sixth International Symposium on Human Identification, 1995 (available on the world wide web at: promega.com/geneticidproc/ussymp6proc/blegrad.html-); LCR Kit Instruction Manual, Cat. #200520, Rev. #050002, Stratagene, 2002; Barany, Proc. Natl. Acad. Sci. USA 88:188-93 (1991); Bi and Sambrook, Nucl. Acids Res. 25:2924-2951 (1997); Zirvi et al., Nucl. Acid Res. 27:e40i-viii (1999); Dean et al., Proc Natl Acad Sci USA 99:5261-66 (2002); Barany and Gelfand, Gene 109:1-11 (1991); Walker et al., Nucl. Acid Res. 20:1691-96 (1992); Polstra et al., BMC Inf. Dis. 2:18-(2002); Lage et al., Genome Res. 2003 February; 13(2):294-307, and Landegren et al., Science 241:1077-80 (1988), Demidov, V., Expert Rev Mol Diagn. 2002 November; 2(6):542-8., Cook et al., J Microbiol Methods. 2003 May; 53(2):165-74, Schweitzer et al., Curr Opin Biotechnol. 2001 February; 12(1):21-7, U.S. Pat. Nos. 5,830,711, 6,027,889, 5,686,243, PCT Publication No. WO0056927A3, and PCT Publication No. WO9803673A1.

In some embodiments, amplification includes at least one cycle of the sequential procedures of: annealing at least one primer with complementary or substantially complementary sequences in at least one target nucleic acid; synthesizing at least one strand of nucleotides in a template-dependent manner using a polymerase; and denaturing the newly-formed nucleic acid duplex to separate the strands. The cycle may or may not be repeated. Amplification can include thermocycling or can be performed isothermally.

As used herein, the term “rolling circle amplification (RCA)” refers to a nucleic acid amplification reaction that amplifies a circular nucleic acid template (e.g., single-stranded DNA circles) via a rolling circle mechanism. Rolling circle amplification reaction is initiated by the hybridization of a primer to a circular, often single-stranded, nucleic acid template. The nucleic acid polymerase then extends the primer that is hybridized to the circular nucleic acid template by continuously progressing around the circular nucleic acid template to replicate the sequence of the nucleic acid template over and over again (rolling circle mechanism). The rolling circle amplification typically produces concatemers including tandem repeat units of the circular nucleic acid template sequence. The rolling circle amplification may be a linear RCA (LRCA), exhibiting linear amplification kinetics (e.g., RCA using a single specific primer), or may be an exponential RCA (ERCA) exhibiting exponential amplification kinetics. Rolling circle amplification may also be performed using multiple primers (multiply primed rolling circle amplification or MPRCA) leading to hyper-branched concatemers. For example, in a double-primed RCA, one primer may be complementary, as in the linear RCA, to the circular nucleic acid template, whereas the other may be complementary to the tandem repeat unit nucleic acid sequences of the RCA product. Consequently, the double-primed RCA may proceed as a chain reaction with exponential (geometric) amplification kinetics featuring a ramifying cascade of multiple-hybridization, primer-extension, and strand-displacement events involving both the primers. This often generates a discrete set of concatemeric, double-stranded nucleic acid amplification products. The rolling circle amplification may be performed in vitro under isothermal conditions using a suitable nucleic acid polymerase such as Phi29 DNA polymerase. RCA may be performed by using any of the DNA polymerases that are known in the art (e.g., a Phi29 DNA polymerase, a Bst DNA polymerase, or SD polymerase).

A nucleic acid can be amplified by a thermocycling method or by an isothermal amplification method. In some embodiments a rolling circle amplification method is used. In some embodiments amplification takes place on a solid support (e.g., within a flow cell) where a nucleic acid, nucleic acid library or portion thereof is immobilized. In certain sequencing methods, a nucleic acid library is added to a flow cell and immobilized by hybridization to anchors under suitable conditions. This type of nucleic acid amplification is often referred to as solid phase amplification. In some embodiments of solid phase amplification, all or a portion of the amplified products are synthesized by an extension initiating from an immobilized primer. Solid phase amplification reactions are analogous to standard solution phase amplifications except that at least one of the amplification oligonucleotides (e.g., primers) is immobilized on a solid support.

In some embodiments, solid phase amplification includes a nucleic acid amplification reaction including only one species of oligonucleotide primer immobilized to a surface or substrate. In certain embodiments solid phase amplification includes a plurality of different immobilized oligonucleotide primer species. In some embodiments solid phase amplification may include a nucleic acid amplification reaction including one species of oligonucleotide primer immobilized on a solid surface and a second different oligonucleotide primer species in solution. Multiple different species of immobilized or solution-based primers can be used. Non-limiting examples of solid phase nucleic acid amplification reactions include interfacial amplification, bridge PCR amplification, emulsion PCR, WildFire amplification (e.g., US patent publication US20130012399), the like or combinations thereof.

As used herein, the terms “cluster” and “colony” are used interchangeably to refer to a discrete site on a solid support that includes a plurality of immobilized polynucleotides and a plurality of immobilized complementary polynucleotides. The term “clustered array” refers to an array formed from such clusters or colonies. In this context the term “array” is not to be understood as requiring an ordered arrangement of clusters. The term “array” is used in accordance with its ordinary meaning in the art and refers to a population of different molecules that are attached to one or more solid-phase substrates such that the different molecules can be differentiated from each other according to their relative location. An array can include different molecules that are each located at different addressable features on a solid-phase substrate. The molecules of the array can be nucleic acid primers, nucleic acid probes, nucleic acid templates or nucleic acid enzymes such as polymerases or ligases. Arrays useful in the invention can have densities that ranges from about 2 different features to many millions, billions or higher. The density of an array can be from 2 to as many as a billion or more different features per square cm. For example, an array can have at least about 100 features/cm², at least about 1,000 features/cm², at least about 10,000 features/cm², at least about 100,000 features /cm², at least about 10,000,000 features/cm², at least about 100,000,000 features/cm², at least about 1,000,000,000 features/cm², at least about 2,000,000,000 features/cm² or higher. In embodiments, the arrays have features at any of a variety of densities including, for example, at least about 10 features/cm², 100 features/cm², 500 features/cm², 1,000 features/cm², 5,000 features/cm², 10,000 features/cm², 50,000 features/cm², 100,000 features/cm², 1,000,000 features/cm², 5,000,000 features/cm², or higher.

Provided herein are methods, systems, and compositions for analyzing a sample (e.g., sequencing nucleic acids within a sample) in situ. The term “in situ” is used in accordance with its ordinary meaning in the art and refers to a sample surrounded by at least a portion of its native environment, such as may preserve the relative position of two or more elements. For example, an extracted human cell obtained is considered in situ when the cell is retained in its local microenvironment so as to avoid extracting the target (e.g., nucleic acid molecules or proteins) away from their native environment. An in situ sample (e.g., a cell) can be obtained from a suitable subject. An in situ cell sample may refer to a cell and its surrounding milieu, or a tissue. A sample can be isolated or obtained directly from a subject or part thereof. In embodiments, the methods described herein (e.g., sequencing a plurality of target nucleic acids of a cell in situ) are applied to an isolated cell (i.e., a cell not surrounded by least a portion of its native environment). For the avoidance of any doubt, when the method is performed within a cell (e.g., an isolated cell) the method may be considered in situ. In some embodiments, a sample is obtained indirectly from an individual or medical professional. A sample can be any specimen that is isolated or obtained from a subject or part thereof. A sample can be any specimen that is isolated or obtained from multiple subjects. Non-limiting examples of specimens include fluid or tissue from a subject, including, without limitation, blood or a blood product (e.g., serum, plasma, platelets, buffy coats, or the like), umbilical cord blood, chorionic villi, amniotic fluid, cerebrospinal fluid, spinal fluid, lavage fluid (e.g., lung, gastric, peritoneal, ductal, ear, arthroscopic), a biopsy sample, celocentesis sample, cells (blood cells, lymphocytes, placental cells, stem cells, bone marrow derived cells, embryo or fetal cells) or parts thereof (e.g., mitochondrial, nucleus, extracts, or the like), urine, feces, sputum, saliva, nasal mucous, prostate fluid, lavage, semen, lymphatic fluid, bile, tears, sweat, breast milk, breast fluid, the like or combinations thereof. Non-limiting examples of tissues include organ tissues (e.g., liver, kidney, lung, thymus, adrenals, skin, bladder, reproductive organs, intestine, colon, spleen, brain, the like or parts thereof), epithelial tissue, hair, hair follicles, ducts, canals, bone, eye, nose, mouth, throat, ear, nails, the like, parts thereof or combinations thereof. A sample may include cells or tissues that are normal, healthy, diseased (e.g., infected), and/or cancerous (e.g., cancer cells). A sample obtained from a subject may include cells or cellular material (e.g., nucleic acids) of multiple organisms (e.g., virus nucleic acid, fetal nucleic acid, bacterial nucleic acid, parasite nucleic acid). A sample may include a cell and RNA transcripts. A sample may include a cell and DNA. A sample may include a cell and target proteins. A sample may include DNA, RNA, organelles, carbohydrates, lipids, proteins, or any combination thereof. These components may be extracellular. A sample may include a “target” of the method described herein or any embodiments of the method described herein. A sample may include any compound that may be desired to be detected, for example a peptide or protein, or nucleic acid molecule or a small molecule, including organic and inorganic molecules. A sample may include proteinaceous molecules such as peptides, polypeptides, proteins or prions or any molecule which includes a protein or polypeptide component, etc., or fragments thereof. A sample may include a single molecule or a complex that contains two or more molecular subunits, which may or may not be covalently bound to one another, and which may be the same or different. Thus, in addition to cells or microorganisms, a sample may also include a protein complex. Such a complex may thus be a homo- or hetero-multimer. Aggregates of molecules e.g., proteins may also be target analytes, for example aggregates of the same protein or different proteins. A sample may include a complex between proteins or peptides and nucleic acid molecules such as DNA or RNA. Of particular interest may be the interactions between proteins and nucleic acids, e.g., regulatory factors, such as transcription factors, and interactions between DNA or RNA molecules. A sample can include nucleic acids obtained from one or more subjects. In some embodiments a sample includes nucleic acid obtained from a single subject. A subject can be any living or non-living organism, including but not limited to a human, non-human animal, plant, bacterium, fungus, virus, or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a plant. In some embodiments, a subject is a human subject. A subject can be a patient (e.g., a human patient). In some embodiments a subject is suspected of having a genetic variation or a disease or condition associated with a genetic variation. A “tissue section” as used herein refers to a portion of a biological tissue derived from a biological sample, typically from an organism (e.g., a human or animal subject or patient).

As used herein, the term “tissue” is used in accordance with its plain and ordinary meaning and refers to an organization of cells in a structure, where the structure generally functions as a unit in an organism (e.g., mammals) and may carry out specific functions. In some examples, cells in a tissue are configured in a mass and may not be free from one another. This disclosure describes methods of obtaining single biological samples (e.g., cells or nuclei) from tissues that can be used in various single biological samples (e.g., single-cell/nucleus) workflows. In some examples, blood cells (e.g., lymphocytes) can be considered a tissue. However, blood cells, like lymphocytes, generally are free from one another in the blood. The methods disclosed herein can be used to process those cells to obtain cells and/or nuclei, although dissociation steps may not be necessary when using those types of tissues. Generally, any type of tissue can be used in the methods described herein. Examples of tissues that may be used in the disclosed methods include, but are not limited to connective, epithelial, muscle and nervous tissue. In some examples, the tissues are from mammals. Tissues that contain any type of cells may be used. For example, tissues from abdomen, bladder, brain, esophagus, heart, intestine, kidney, liver, lung, lymph node, olfactory bulb, ovary, pancreas, skin, spleen, stomach, testicle, and the like. The tissue may be normal or tumor tissue (e.g., malignant). This example is not meant to be limiting. Although the conditions used in the disclosed may not be identical for different types of tissue, the methods may be applied to any tissue. The tissues used in the disclosed methods may be in various states. In some examples, the tissues used in the disclosed methods may be fresh, frozen, or fixed.

As used herein, the term “disease state” is used in accordance with its plain and ordinary meaning and refers to any abnormal biological or aberrant state of a cell. The presence of a disease state may be identified by the same collection of biological constituents used to determine the cell's biological state. In general, a disease state will be detrimental to a biological system. A disease state may be a consequence of, inter alia, an environmental pathogen, for example a viral infection (e.g., HIV/AIDS, hepatitis B, hepatitis C, influenza, measles, etc.), a bacterial infection, a parasitic infection, a fungal infection, or infection by some other organism. A disease state may also be the consequence of some other environmental agent, such as a chemical toxin or a chemical carcinogen. As used herein, a disease state further includes genetic disorders wherein one or more copies of a gene is altered or disrupted, thereby affecting its biological function. Exemplary genetic diseases include, but are not limited to polycystic kidney disease, familial multiple endocrine neoplasia type I, neurofibromatoses, Tay-Sachs disease, Huntington's disease, sickle cell anemia, thalassemia, and Down's syndrome, as well as others (see, e.g., The Metabolic and Molecular Bases of Inherited Diseases, 7th ed., McGraw-Hill Inc., New York). Other exemplary diseases include, but are not limited to, cancer, hypertension, Alzheimer's disease, neurodegenerative diseases, and neuropsychiatric disorders such as bipolar affective disorders or paranoid schizophrenic disorders. Disease states are monitored to determine the level or severity (e.g., the stage or progression) of one or more disease states of a subject and, more specifically, detect changes in the biological state of a subject which are correlated to one or more disease states (see, e.g., U.S. Pat. No. 6,218,122, which is incorporated by reference herein in its entirety). In embodiments, methods provided herein are also applicable to monitoring the disease state or states of a subject undergoing one or more therapies. Thus, the present disclosure also provides, in some embodiments, methods for determining or monitoring efficacy of a therapy or therapies (i.e., determining a level of therapeutic effect) upon a subject. In embodiments, methods of the present disclosure can be used to assess therapeutic efficacy in a clinical trial, e.g., as an early surrogate marker for success or failure in such a clinical trial. Within eukaryotic cells, there are hundreds to thousands of signaling pathways that are interconnected. For this reason, perturbations in the function of proteins within a cell have numerous effects on other proteins and the transcription of other genes that are connected by primary, secondary, and sometimes tertiary pathways. This extensive interconnection between the function of various proteins means that the alteration of any one protein is likely to result in compensatory changes in a wide number of other proteins. In particular, the partial disruption of even a single protein within a cell, such as by exposure to a drug or by a disease state which modulates the gene copy number (e.g., a genetic mutation), results in characteristic compensatory changes in the transcription of enough other genes that these changes in transcripts can be used to define a “signature” of particular transcript alterations which are related to the disruption of function, e.g., a particular disease state or therapy, even at a stage where changes in protein activity are undetectable.

The terms “polypeptide,” “peptide” and “protein” are used interchangeably herein to refer to a polymer of amino acid residues, wherein the polymer may optionally be conjugated to a moiety that does not consist of amino acids. The terms apply to amino acid polymers in which one or more amino acid residue is an artificial chemical mimetic of a corresponding naturally occurring amino acid, as well as to naturally occurring amino acid polymers and non-naturally occurring amino acid polymer. A protein may refer to a protein expressed in a cell.

A polypeptide, or a cell is “recombinant” when it is artificial or engineered, or derived from or contains an artificial or engineered protein or nucleic acid (e.g., non-natural or not wild type). For example, a polynucleotide that is inserted into a vector or any other heterologous location, e.g., in a genome of a recombinant organism, such that it is not associated with nucleotide sequences that normally flank the polynucleotide as it is found in nature is a recombinant polynucleotide. A protein expressed in vitro or in vivo from a recombinant polynucleotide is an example of a recombinant polypeptide. Likewise, a polynucleotide sequence that does not appear in nature, for example a variant of a naturally occurring gene, is recombinant.

As used herein, a “single cell” refers to one cell. Single cells useful in the methods described herein can be obtained from a tissue of interest, or from a biopsy, blood sample, or cell culture. Additionally, cells from specific organs, tissues, tumors, neoplasms, or the like can be obtained and used in the methods described herein. In general, cells from any population can be used in the methods, such as a population of prokaryotic or eukaryotic organisms, including bacteria or yeast.

The term “cellular component” is used in accordance with its ordinary meaning in the art and refers to any organelle, nucleic acid, protein, or analyte that is found in a prokaryotic, eukaryotic, archaeal, or other organismic cell type. Examples of cellular components (e.g., a component of a cell) include RNA transcripts, proteins, membranes, lipids, and other analytes.

A “gene” refers to a polynucleotide that is capable of conferring biological function after being transcribed and/or translated. Functionally, a genome is subdivided into genes. Each gene is a nucleic acid sequence that encodes an RNA or polypeptide. A gene is transcribed from DNA into RNA, which can either be non-coding (ncRNA) with a direct function, or an intermediate messenger (mRNA) that is then translated into protein. Typically a gene includes multiple sequence elements, such as for example, a coding element (i.e., a sequence that encodes a functional protein), non-coding element, and regulatory element. Each element may be as short as a few bp to 5 kb. In embodiments, the gene is the protein coding sequence of RNA. Non-limiting examples of genes include developmental genes (e.g., adhesion molecules, cyclin kinase inhibitors, Wnt family members, Pax family members, Winged helix family members, Hox family members, cytokines/lymphokines and their receptors, growth/differentiation factors and their receptors, neurotransmitters and their receptors); oncogenes (e.g., ABL1, BCL1, BCL2, BCL6, CBFA2, CBL, CSF1R, ERBA, ERBB, ERBB2, ETS1, ETS1, ETV6, FGR, FOS, FYN, HCR, HRAS, JUN, KRAS, LCK, LYN, MDM2, MLL, MYB, MYC, MYCL1, MYCN, NRAS, PIM1, PML, RET, SRC, TALl, TCL3, and YES); tumor suppressor genes (e.g., APC, BRCA1, BRCA2, MADH4, MCC, NF1, NF2, RB1, TP53, and WTI1); and enzymes (e.g., ACC synthases and oxidases, ACP desaturases and hydroxylases, ADP-glucose pyrophorylases, ATPases, alcohol dehydrogenases, amylases, amyloglucosidases, catalases, cellulases, chalcone synthases, chitinases, cyclooxygenases, decarboxylases, dextrinases, DNA and RNA polymerases, galactosidases, glucanases, glucose oxidases, granule-bound starch synthases, GTPases, helicases, hemicellulases, integrases, inulinases, invertases, isomerases, kinases, lactases, lipases, lipoxygenases, lysozymes, nopaline synthases, octopine synthases, pectinesterases, peroxidases, phosphatases, phospholipases, phosphorylases, phytases, plant growth regulator synthases, polygalacturonases, proteinases and peptidases, pullanases, recombinases, reverse transcriptases, RUBISCOs, topoisomerases, and xylanases). In embodiments, a gene includes at least one mutation associated with a disease or condition mediated by a mutant form of the gene.

As used herein, “biomaterial” refers to any biological material produced by an organism. In some embodiments, biomaterial includes secretions, extracellular matrix, proteins, lipids, organelles, membranes, cells, portions thereof, and combinations thereof. In some embodiments, cellular material includes secretions, extracellular matrix, proteins, lipids, organelles, membranes, cells, portions thereof, and combinations thereof. In some embodiments, biomaterial includes viruses. In some embodiments, the biomaterial is a replicating virus and thus includes virus infected cells. In embodiments, a biological sample includes biomaterials.

In some embodiments, a sample includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

A subject can be any living or non-living organism, including but not limited to a human, non-human animal, plant, bacterium, fungus, virus or protist. A subject may be any age (e.g., an embryo, a fetus, infant, child, adult). A subject can be of any sex (e.g., male, female, or combination thereof). A subject may be pregnant. In some embodiments, a subject is a mammal. In some embodiments, a subject is a human subject. A subject can be a patient (e.g., a human patient). In some embodiments a subject is suspected of having a genetic variation or a disease or condition associated with a genetic variation.

The methods and kits of the present disclosure may be applied, mutatis mutandis, to the sequencing of RNA, or to determining the identity of a ribonucleotide.

As used herein, the term “kit” refers to any delivery system for delivering materials. In the context of reaction assays, such delivery systems include systems that allow for the storage, transport, or delivery of reaction reagents (e.g., oligonucleotides, enzymes, etc. in the appropriate containers) and/or supporting materials (e.g., packaging, buffers, written instructions for performing a method, etc.) from one location to another. For example, kits include one or more enclosures (e.g., boxes) containing the relevant reaction reagents and/or supporting materials. As used herein, the term “fragmented kit” refers to a delivery system including two or more separate containers that each contain a subportion of the total kit components. The containers may be delivered to the intended recipient together or separately. For example, a first container may contain an enzyme for use in an assay, while a second container contains oligonucleotides. In contrast, a “combined kit” refers to a delivery system containing all of the components of a reaction assay in a single container (e.g., in a single box housing each of the desired components). The term “kit” includes both fragmented and combined kits. Vessels may include any structure capable of supporting or containing a liquid or solid material and may include, tubes, vials, jars, containers, tips, etc. In embodiments, a wall of a vessel may permit the transmission of light through the wall. In embodiments, the vessel may be optically clear. The kit may include the enzyme and/or nucleotides in a buffer.

As used herein the term “determine” can be used to refer to the act of ascertaining, establishing or estimating. A determination can be probabilistic. For example, a determination can have an apparent likelihood of at least 50%, 75%, 90%, 95%, 98%, 99%, 99.9% or higher. In some cases, a determination can have an apparent likelihood of 100%. An exemplary determination is a maximum likelihood analysis or report. As used herein, the term “identify,” when used in reference to a thing, can be used to refer to recognition of the thing, distinction of the thing from at least one other thing or categorization of the thing with at least one other thing. The recognition, distinction or categorization can be probabilistic. For example, a thing can be identified with an apparent likelihood of at least 50%, 75%, 90%, 95%, 98%, 99%, 99.9% or higher. A thing can be identified based on a result of a maximum likelihood analysis. In some cases, a thing can be identified with an apparent likelihood of 100%.

An “antibody” (Ab) is a protein that binds specifically to a particular substance, known as an “antigen” (Ag). An “antibody” or “antigen-binding fragment” is an immunoglobulin that binds a specific “epitope.” The term encompasses polyclonal, monoclonal, and chimeric antibodies. In nature, antibodies are generally produced by lymphocytes in response to immune challenge, such as by infection or immunization. An “antigen” (Ag) is any substance that reacts specifically with antibodies or T lymphocytes (T cells). An antibody may include the entire antibody as well as any antibody fragments capable of binding the antigen or antigenic fragment of interest. Examples include complete antibody molecules, antibody fragments, such as Fab, F(ab′)2, CDRs, VL, VH, and any other portion of an antibody which is capable of specifically binding to an antigen. Antibodies used herein are immunospecific for, and therefore specifically and selectively bind to, for example, proteins either detected (e.g., biological targets of interest) or used for detection (e.g., probes containing oligonucleotide barcodes) in the methods and devices as described herein.

A “monoclonal antibody” includes a collection of identical molecules produced by a single B cell lymphocyte clone which are directed against a single antigenic determinant. Monoclonal antibodies can be distinguished from polyclonal antibodies in that monoclonal antibodies must be individually selected whereas polyclonal antibodies are selected in groups of more than one or, in other words, in bulk. Large amounts of monoclonal antibodies can be produced by immortalization of a polyclonal B cell population using hybridoma technology. Each immortalized B cell can divide, presumably indefinitely, and gives rise to a clonal population of cells that each expresses an identical antibody molecule. The individual immortalized B cell clones, the hybridomas, are segregated and cultured separately.

The term “polyclonal antibody” refers to an antibody that is produced from a different B cell lineages within the body. A polyclonal antibody is directed to many different antigenic determinants on the target cell surface and would bind with sufficient density to allow the effector mechanisms of the immune system to work efficiently.

An immunoglobulin (antibody) structural unit are typically tetrameric glycosylated proteins. Each tetramer is composed of two identical pairs of polypeptide chains, each pair having one “light” chain (about 25 kDa) and one “heavy” chain (about 50-70 kDa). The N-terminus of each chain defines a variable region of about 100 to 110 or more amino acids primarily responsible for antigen recognition. The terms “variable heavy chain,” “VH,” or “VII” refer to the variable region of an immunoglobulin heavy chain, including an Fv, scFv, dsFv or Fab; while the terms “variable light chain,” “VL” or “VL” refer to the variable region of an immunoglobulin light chain, including of an Fv, scFv, dsFv or Fab.

The term “aptamer” refers to oligonucleotide or peptide molecules that bind to a specific target molecule. An aptamer can include any suitable number of nucleotides. “Aptamers” refer to more than one such set of molecules. Different aptamers can have either the same or different numbers of nucleotides. Aptamers may be DNA or RNA and may be single stranded, double stranded, or contain double stranded or triple stranded regions. In embodiments, peptide aptamers consist of one (or more) short variable peptide domains, attached at both ends to a protein scaffold. Aptamers may be designed with any combination of the base modified nucleotides desired. Aptamers to a given target include nucleic acids that are identified from a candidate mixture of nucleic acids, where the aptamer is a ligand of the target, by a method including: (a) contacting the candidate mixture with the target, wherein nucleic acids having an increased affinity to the target relative to other nucleic acids in the candidate mixture can be partitioned from the remainder of the candidate mixture; (b) partitioning the increased affinity nucleic acids from the remainder of the candidate mixture; and (c) amplifying the increased affinity nucleic acids to yield a ligand-enriched mixture of nucleic acids, whereby aptamers of the target molecule are identified. It is recognized that affinity interactions are a matter of degree; however, in this context, the “specific binding affinity” of an aptamer for its target means that the aptamer binds to its target with a much higher degree of affinity than it binds to other, non-target, components in a mixture or sample. An aptamer can be identified using any known method, including the SELEX process. See, e.g., U.S. Pat. No. 5,475,096 entitled “Nucleic Acid Ligands”. Once identified, an aptamer can be prepared or synthesized in accordance with any known method, including chemical synthetic methods and enzymatic synthetic methods.

Nucleic acid aptamers are nucleic acid species that are typically the product of engineering through repeated rounds of in vitro selection, such as SELEX (systematic evolution of ligands by exponential enrichment), to bind to various molecular targets such as small molecules, proteins, nucleic acids, and even cells, tissues and organisms. At the molecular level, aptamers bind to its target site through non-covalent interactions. Aptamers bind to these specific targets because of electrostatic interactions, hydrophobic interactions, and their complementary shapes. In embodiments, peptide aptamers are artificial proteins selected or engineered to bind specific target molecules. These proteins may include or consist of one or more peptide loops of variable sequence displayed by a protein scaffold. They are typically isolated from combinatorial libraries and often subsequently improved by directed mutation or rounds of variable region mutagenesis and selection.

An “antigen” (Ag) is any substance that reacts specifically with antibodies or T lymphocytes (T cells). In general, antigens include molecules or portions thereof that trigger an immune response in a host (e.g., in a subject), and may be recognized by an antibody. Antigens may be foreign to a subject (e.g., as in viral or bacterial proteins, polysaccharides, or other molecules), or native to the subject (e.g., as in an autoimmune response to self-proteins, which optionally may be mutant forms of a native protein). Examples of antigens include, without limitation, viral antigens, bacterial antigens, fungal antigens, cancer or tumor antigens, and allergens. Examples of viral antigens include, but are not limited to, env, gag, rev, tar, tat, nucleocapsid proteins and reverse transcriptase from immunodeficiency viruses (e.g., HIV, FIV), such as HIV-1 gag, HIV-1 env, HIV-1 pol, HIV-1 tat, HIV-1 nef; HBV surface antigen and core antigen, HbsAG, HbcAg; HCV antigens such as hepatitis C core antigen; influenza nucleocapsid proteins; parainfluenza nucleocapsid proteins; HPV E6 and E7 such as human papilloma type 16 E6 and E7 proteins; Epstein-Barr virus LMP-1, LMP-2 and EBNA-2; herpes LAA and glycoprotein D such as HSV glycoprotein D; as well as similar proteins from other viruses. In embodiments, the biomolecule-specific binding moiety is an antibody that is reactive to a plurality of viral antigens within the same viral group. For example, a flavivirus group-reactive antibody such as the monoclonal antibody MAb 6B6C-1, dengue 4G2, or Murray Valley 4A1B-9 is reactive with arbovirus antigens within the flavivirus genus, which includes the West Nile virus, Saint Louis encephalitis virus, Japanese encephalitis virus, and dengue virus. Similarly, for example, an alphavirus group-reactive antibody such as EEE 1A4B-6 or WEE 2A2C-3 is reactive with alphavirus antigens within the alphavirus genus, which includes eastern equine encephalitis virus, western equine encephalitis virus, and Venezuelan equine encephalitis virus. Similarly, for example, a bunyavirus group-reactive antibody such as LAC 10G5.4 is reactive with bunyavirus antigens within the bunyavirus genus, which includes the California serogroup of bunyaviruses, which includes La Crosse virus. Examples of bacterial antigens include, but are not limited, to capsule antigens (e.g., protein or polysaccharide antigens such as CP5 or CP8 from the S. aureus capsule); cell wall (including outer membrane) antigens such as peptidoglycan (e.g., mucopeptides, glycopeptides, mureins, muramic acid residues, and glucose amine residues) polysaccharides, teichoic acids (e.g., ribitol teichoic acids and glycerol teichoic acids), phospholipids, hopanoids, and lipopolysaccharides (e.g., the lipid A or O-polysaccharide moieties of bacteria such as Pseudomonas aeruginosa serotype 011); plasma membrane components including phospholipids, hopanoids, and proteins; proteins and peptidoglycan found within the periplasm; fimbrae antigens, pili antigens, flagellar antigens, and S-layer antigens. S. aureus antigens can be a serotype 5 capsular antigen, a serotype 8 capsular antigen, and antigen shared by serotypes 5 and 8 capsular antigens, a serotype 336 capsular antigen, protein A, coagulase, clumping factor A, clumping factor B, a fibronectin binding protein, a fibrinogen binding protein, a collagen binding protein, an elastin binding protein, a MHC analogous protein, a polysaccharide intracellular adhesion, alpha hemolysin, beta hemolysin, delta hemolysin, gamma hemolysin, Panton-Valentine leukocidin, exfoliative toxin A, exfoliative toxin B, V8 protease, hyaluronate lyase, lipase, staphylokinase, LukED leukocidin, an enterotoxin, toxic shock syndrome toxin-1, poly-N-succinyl beta-1→6 glucosamine, catalase, beta-lactamase, teichoic acid, peptidoglycan, a penicillin binding protein, chemotaxis inhibiting protein, complement inhibitor, Sbi, and von Willebrand factor binding protein. Non-limiting examples of fungal antigens include, but are not limited to, Candida fungal antigen components; Histoplasma fungal antigens such as heat shock protein 60 (HSP60) and other Histoplasma fungal antigen components; cryptococcal fungal antigens such as capsular polysaccharides and other cryptococcal fungal antigen components; coccidiodes fungal antigens such as spherule antigens and other coccidiodes fungal antigen components; and tinea fungal antigens such as trichophytin and other coccidiodes fungal antigen components. Examples of cancer antigens include, but are not limited to, MAGE, MART-1/Melan-A, gplOO, dipeptidyl peptidase IV (DPPIV), adenosine deaminase-binding protein (ADAbp), cyclophilin b, colorectal associated antigen (CRC)—COI 7-1 A/GA733, carcinoembryonic antigen (CEA) and its immunogenic epitopes CAP-1 and CAP-2, etv, aml1, prostate specific antigen (PSA) and its immunogenic epitopes PSA-1, PSA-2, and PSA-3, prostate-specific membrane antigen (PSMA), T-cell receptor/CD3-zeta chain, MAGE-family of tumor antigens (e.g., MAGE-A1, MAGE-A2, MAGE-A3, MAGE-A4, MAGE-A5, MAGE-A6, MAGE-A7, MAGE-A8, MAGE-A9, MAGE-A10, MAGE-A11, MAGE-A12, MAGE-Xp2 (MAGE-B2), MAGE-Xp3 (MAGE-B3), MAGE-Xp4 (MAGE-B4), MAGE-C₁, MAGE-C₂, MAGE-C₃, MAGE-C₄, MAGE-C₅), GAGE-family of tumor antigens (e.g., GAGE-1, GAGE-2, GAGE-3, GAGE-4, GAGE-5, GAGE-6, GAGE-7, GAGE-8, GAGE-9), BAGE, RAGE, LAGE-1, NAG, GnT-V, MUM-1, CDK4, tyrosinase, p53, MUC family, HER2/neu, p21 ras, RCAS1, α-fetoprotein, E-cadherin, α-catenin, β-catenin, γ-catenin, p120ctn, gp100^{P}mel⁷, PRAME, NY-ESO-1, cdc27, adenomatous polyposis coli protein (APC), fodrin, Connexin 37, Ig-idiotype, p15, gp75, GM2 and GD2 gangliosides, viral products such as human papillomavirus proteins, Smad family of tumor antigens, lmp-1, P1 A, EBV-encoded nuclear antigen (EBNA)-I, brain glycogen phosphorylase, SSX-1, SSX-2 (HOM-MEL-40), SSX-1, SSX-4, SSX-5, SCP-1, CT-7, and c-erbB-2. Examples of allergens include, but are not limited to, dust, pollen, pet dander, food such as peanuts, nuts, shellfish, fish, wheat milk, eggs, soy and their derivatives, and sulfites. These lists are not meant to be limiting.

An “affimer” is a non-antibody protein that binds to target proteins with affinity in the nanomolar range. It behaves similarly to an antibody by binding tightly to its target molecule. Affimers are recombinant proteins that are typically engineered to mimic molecular recognition characteristics of monoclonal antibodies.

As used herein, the term “immunoassay” refers to a biochemical test that measures the presence or concentration of a macromolecule or a small molecule in a solution involving a reaction between an antibody and an antigen. The molecule detected by the immunoassay is often referred to as an “analyte” and is in many cases a protein, although it may be other kinds of molecules, of different sizes and types. Immunoassays come in many different formats and variations. Immunoassays may be run in multiple steps with reagents being added and washed away or separated at different points in the assay. Multi-step assays are often called separation immunoassays or heterogeneous immunoassays. Some immunoassays can be carried out simply by mixing the reagents and sample and making a physical measurement. Such assays are called homogenous immunoassays or less frequently non-separation immunoassays. Immunoassays include assays in which the analyte is an antigen, as well as assays in which the analyte is an antibody (e.g., when detecting the presence, absence, or degree of an immune response). In embodiments, an immunoassay includes detecting multiple different analytes from a single sample simultaneously in a common reaction volume.

An “analyte-specific binding agent”, “biomolecule-specific binding agent,” or “probe” is a substance that allows for selective binding to another substance (e.g., an analyte or biomolecule). A particular example of specific binding is that which occurs between an antibody and an antigen. Typically, specific binding can be distinguished from non-specific when the dissociation constant (KD) is less than about 1×10⁻⁵ M or less than about 1×10⁻⁶ M or 1×10⁻⁷ M. Specific binding can be detected, for example, by ELISA, immunoprecipitation, coprecipitation, with or without chemical crosslinking, two-hybrid assays and the like. A binding agent is typically a biological or synthetic molecule that has high affinity for another molecule or macromolecule, through covalent or non-covalent bonding. Examples of a binding agent can include streptavidin, antibody, antigen, enzyme, enzyme cofactor or inhibitor, hormone, or hormone receptor. This binding agent can bind to an analyte (e.g., a protein), often in a heterogeneous population of proteins and other biologics. Thus, under designated immunoassay conditions, the specified binding agents bind to a particular analyte at least two times the background and more typically more than 10 to 100 times background. In embodiments, a probe includes a biomolecule-specific binding agent, which is indirectly linked to a detectable moiety or agent (e.g., a fluorochrome) via a covalent bond to central ring moiety (e.g., Ring A or Ring B). In embodiments, the probe forms a complex with a target biomolecule, wherein the probe includes a biomolecule-specific binding agent and a fluorescent moiety, which facilitates detection of the target biomolecule.

As used herein, the term “analyte” or “target biomolecule” refers to a component, substance, or constituent of interest in an analytical procedure whose presence, absence, or amount is desired to be determined or measured. In an immunoassay, for example, the analyte may be a protein, protein fragment, polypeptide, an antibody, antigen expressing antibody or a molecule detectable with an antibody, an antigen, or a ligand. The term “analyte” also refers to detectable components of structured elements such as cells, including all animal and plant cells, and microorganisms, such as fungi, viruses, bacteria including, but not limited to, all gram positive and gram-negative bacteria, and protozoa.

In some embodiments, a “sample” includes one or more nucleic acids, or fragments thereof. A sample can include nucleic acids obtained from one or more subjects. In some embodiments a sample includes nucleic acid obtained from a single subject. In some embodiments, a sample includes a mixture of nucleic acids. A mixture of nucleic acids can include two or more nucleic acid species having different nucleotide sequences, different fragment lengths, different origins (e.g., genomic origins, cell or tissue origins, subject origins, the like or combinations thereof), or combinations thereof. A sample may include synthetic nucleic acid.

The term “covalent linker” is used in accordance with its ordinary meaning and refers to a divalent moiety which connects at least two moieties to form a molecule.

The term “non-covalent linker” is used in accordance with its ordinary meaning and refers to a divalent moiety which includes at least two molecules that are not covalently linked to each other but are capable of interacting with each other via a non-covalent bond (e.g., electrostatic interactions (e.g., ionic bond, hydrogen bond, halogen bond) or van der Waals interactions (e.g., dipole-dipole, dipole-induced dipole, London dispersion). In embodiments, the non-covalent linker is the result of two molecules that are not covalently linked to each other that interact with each other via a non-covalent bond.

Where a range of values is provided, it is understood that each intervening value, to the tenth of the unit of the lower limit unless the context clearly indicates otherwise, between the upper and lower limit of that range, and any other stated or unstated intervening value in, or smaller range of values within, that stated range is encompassed within the invention. The upper and lower limits of any such smaller range (within a more broadly recited range) may independently be included in the smaller ranges, or as particular values themselves, and are also encompassed within the invention, subject to any specifically excluded limit in the stated range. Where the stated range includes one or both of the limits, ranges excluding either or both of those included limits are also included in the invention.

As used herein, the term “upstream” refers to a region in the nucleic acid sequence that is towards the 5′ end of a particular reference point, and the term “downstream” refers to a region in the nucleic acid sequence that is toward the 3′ end of the reference point.

As used herein, the terms “incubate,” and “incubation refer collectively to altering the temperature of an object in a controlled manner such that conditions are sufficient for conducting the desired reaction. Thus, it is envisioned that the terms encompass heating a receptacle (e.g., a microplate) to a desired temperature and maintaining such temperature for a fixed time interval. Also included in the terms is the act of subjecting a receptacle to one or more heating and cooling cycles (i.e., “temperature cycling” or “thermal cycling”). While temperature cycling typically occurs at relatively high rates of change in temperature, the term is not limited thereto, and may encompass any rate of change in temperature.

As used herein, “biological activity” may include the in vivo activities of a compound or physiological responses that result upon in vivo administration of a compound, composition or other mixture. Biological activity, thus, may encompass therapeutic effects and pharmaceutical activity of such compounds, compositions and mixtures. Biological activities may be observed in vitro systems designed to test or use such activities.

The term “isolated” means altered or removed from the natural state. For example, a nucleic acid or a polypeptide naturally present in a living animal is not isolated, but the same nucleic acid or polypeptide partially or completely separated from the coexisting materials of its natural state is isolated. An isolated nucleic acid or protein can exist in substantially purified form, or can exist in a non-native environment such as, for example, a host cell. In embodiments, “isolated” refers to a nucleic acid, polynucleotide, polypeptide, protein, or other component that is partially or completely separated from components with which it is normally associated (other proteins, nucleic acids, cells, etc.).

The term “synthetic target” as used herein refers to a modified protein or nucleic acid such as those constructed by synthetic methods. In embodiments, a synthetic target is artificial or engineered, or derived from or contains an artificial or engineered protein or nucleic acid (e.g., non-natural or not wild type). For example, a polynucleotide that is inserted or removed such that it is not associated with nucleotide sequences that normally flank the polynucleotide as it is found in nature is a synthetic target polynucleotide.

The term “nucleic acid sequencing device” and the like means an integrated system of one or more chambers, ports, and channels that are interconnected and in fluid communication and designed for carrying out an analytical reaction or process, either alone or in cooperation with an appliance or instrument that provides support functions, such as sample introduction, fluid and/or reagent driving means, temperature control, detection systems, data collection and/or integration systems, for the purpose of determining the nucleic acid sequence of a template polynucleotide. Nucleic acid sequencing devices may further include valves, pumps, and specialized functional coatings on interior walls. Nucleic acid sequencing devices may include a receiving unit, or platen, that orients the flow cell such that a maximal surface area of the flow cell is available to be exposed to an optical lens. Other nucleic acid sequencing devices include those provided by Singular Genomics® (e.g., the G4® system), Illumina™ (e.g., HiSeq™, MiSeq™, NextSeq™, or NovaSeq™ systems), Life Technologies™ (e.g., ABI PRISM™, or SOLiD™ systems), Pacific Biosciences (e.g., systems using SMRT™ Technology such as the Sequel™ or RS II™ systems), or Qiagen (e.g., Genereader™ system). Nucleic acid sequencing devices may further include fluidic reservoirs (e.g., bottles), valves, pressure sources, pumps, sensors, control systems, valves, pumps, and specialized functional coatings on interior walls. In embodiments, the device includes a plurality of a sequencing reagent reservoirs and a plurality of clustering reagent reservoirs. In embodiments, the clustering reagent reservoir includes amplification reagents (e.g., an aqueous buffer containing enzymes, salts, and nucleotides, denaturants, crowding agents, etc.) In embodiments, the reservoirs include sequencing reagents (such as an aqueous buffer containing enzymes, salts, and nucleotides); a wash solution (an aqueous buffer); a cleave solution (an aqueous buffer containing a cleaving agent, such as a reducing agent); or a cleaning solution (a dilute bleach solution, dilute NaOH solution, dilute HCl solution, dilute antibacterial solution, or water). The fluid of each of the reservoirs can vary. The fluid can be, for example, an aqueous solution which may contain buffers (e.g., saline-sodium citrate (SSC), ascorbic acid, tris(hydroxymethyl)aminomethane or “Tris”), aqueous salts (e.g., KCl or (NH4)2SO4)), nucleotides, polymerases, cleaving agent (e.g., tri-n-butyl-phosphine, triphenyl phosphine and its sulfonated versions (i.e., tris(3-sulfophenyl)-phosphine, TPPTS), and tri(carboxyethyl)phosphine (TCEP) and its salts, cleaving agent scavenger compounds (e.g., 2′-Dithiobisethanamine or 11-Azido-3,6,9-trioxaundecane-1-amine), chelating agents (e.g., EDTA), detergents, surfactants, crowding agents, or stabilizers (e.g., PEG, Tween, BSA). Non-limited examples of reservoirs include cartridges, pouches, vials, containers, and eppendorf tubes. In embodiments, the device is configured to perform fluorescent imaging. In embodiments, the device includes one or more light sources (e.g., one or more lasers). In embodiments, the illuminator or light source is a radiation source (i.e., an origin or generator of propagated electromagnetic energy) providing incident light to the sample. A radiation source can include an illumination source producing electromagnetic radiation in the ultraviolet (UV) range (about 200 to 390 nm), visible (VIS) range (about 390 to 770 nm), or infrared (IR) range (about 0.77 to 25 microns), or other range of the electromagnetic spectrum. In embodiments, the illuminator or light source is a lamp such as an arc lamp or quartz halogen lamp. In embodiments, the illuminator or light source is a coherent light source. In embodiments, the light source is a laser, LED (light emitting diode), a mercury or tungsten lamp, or a super-continuous diode. In embodiments, the light source provides excitation beams having a wavelength between 200 nm to 1500 nm. In embodiments, the laser provides excitation beams having a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the illuminator or light source is a light-emitting diode (LED). The LED can be, for example, an Organic Light Emitting Diode (OLED), a Thin Film Electroluminescent Device (TFELD), or a Quantum dot based inorganic organic LED. The LED can include a phosphorescent OLED (PHOLED). In embodiments, the nucleic acid sequencing device includes an imaging system (e.g., an imaging system as described herein). The imaging system capable of exciting one or more of the identifiable labels (e.g., a fluorescent label) linked to a nucleotide and thereafter obtain image data for the identifiable labels. The image data (e.g., detection data) may be analyzed by another component within the device. The imaging system may include a system described herein and may include a fluorescence spectrophotometer including an objective lens and/or a solid-state imaging device. The solid-state imaging device may include a charge coupled device (CCD) and/or a complementary metal oxide semiconductor (CMOS). The system may also include circuitry and processors, including systems using microcontrollers, reduced instruction set computers (RISC), application specific integrated circuits (ASICs), field programmable gate array (FPGAs), logic circuits, and any other circuit or processor capable of executing functions described herein. The set of instructions may be in the form of a software program. As used herein, the terms “software” and “firmware” are interchangeable, and include any computer program stored in memory for execution by a computer, including RAM memory, ROM memory, EPROM memory, EEPROM memory, and non-volatile RAM (NVRAM) memory. In embodiments, the device includes a thermal control assembly useful to control the temperature of the reagents.

The term “image” is used according to its ordinary meaning and refers to a representation of all or part of an object. The representation may be an optically detected reproduction. For example, an image can be obtained from fluorescent, luminescent, scatter, or absorption signals. The part of the object that is present in an image can be the surface or other xy plane of the object. Typically, an image is a 2 dimensional representation of a 3 dimensional object. An image may include signals at differing intensities (i.e., signal levels). An image can be provided in a computer readable format or medium. An image is derived from the collection of focus points of light rays coming from an object (e.g., the sample), which may be detected by any image sensor.

As used herein, the term “signal” is intended to include, for example, fluorescent, luminescent, scatter, or absorption impulse or electromagnetic wave transmitted or received. Signals can be detected in the ultraviolet (UV) range (about 200 to 390 nm), visible (VIS) range (about 391 to 770 nm), infrared (IR) range (about 0.771 to 25 microns), or other range of the electromagnetic spectrum. The term “signal level” refers to an amount or quantity of detected energy or coded information. For example, a signal may be quantified by its intensity, wavelength, energy, frequency, power, luminance, or a combination thereof. Other signals can be quantified according to characteristics such as voltage, current, electric field strength, magnetic field strength, frequency, power, temperature, etc. Absence of signal is understood to be a signal level of zero or a signal level that is not meaningfully distinguished from noise.

The term “xy coordinates” refers to information that specifies location, size, shape, and/or orientation in an xy plane. The information can be, for example, numerical coordinates in a Cartesian system. The coordinates can be provided relative to one or both of the x and y axes or can be provided relative to another location in the xy plane (e.g., a fiducial). The term “xy plane” refers to a 2 dimensional area defined by straight line axes x and y. When used in reference to a detecting apparatus and an object observed by the detector, the xy plane may be specified as being orthogonal to the direction of observation between the detector and object being detected.

As used herein, the term “tissue section” refers to a piece of tissue that has been obtained from a subject, optionally fixed and attached to a surface, e.g., a microscope slide.

The term “clonotype” is used in accordance with its ordinary meaning in the art and refers to a recombined nucleic acid which encodes an immune receptor or a portion thereof. For example, a clonotype refers to a recombined nucleic acid, usually extracted from a T cell or B cell, but which may also be from a cell-free source, which encodes a T cell receptor (TCR) or B cell receptor (BCR), or a portion thereof. In embodiments, clonotypes may encode all or a portion of a VDJ rearrangement of IgH, a DJ rearrangement of IgH, a VJ rearrangement of IgK, a VJ rearrangement of IgL, a VDJ rearrangement of TCR 3, a DJ rearrangement of TCR 3, a VJ rearrangement of TCR a, a VJ rearrangement of TCRy, a VDJ rearrangement of TCR 6, a VD rearrangement of TCR 6, a Kde-V rearrangement, or the like. Clonotypes may also encode translocation breakpoint regions involving immune receptor genes, such as Bcl1-JH or Bcl2-JH. In one aspect, clonotypes have sequences that are sufficiently long to represent or reflect the diversity of the immune molecules that they are derived from consequently, clonotypes may vary widely in length. In some embodiments, clonotypes have lengths in the range of from 25 to 400 nucleotides; in other embodiments, clonotypes have lengths in the range of from 25 to 200 nucleotides.

A “immune repertoire” refers to the collection of T cell receptors and B cell receptors (e.g., immunoglobulin) that constitutes an organism's adaptive immune system.

A “locus” is used in accordance with its ordinary meaning and refers to a location of a gene or other DNA sequence on a chromosome. The Immunoglobulin Heavy (IGH) locus refers to a collection of located on chromosome 14 and is responsible for the production of heavy chain immunoglobulins, composed of several sub-loci, including V, D, J, C and S regions, which are involved in the process of antibody diversity. The IGH locus is responsible for the production of IgM, IgD, IgG, IgE, and IgA. The Immunoglobulin Kappa (IGK) locus refers to a collection of genes located on chromosome 2 and is responsible for the production of kappa light chain immunoglobulins, composed of V, J, and C regions, which are involved in the process of antibody diversity. The IGK locus is responsible for the production of IgM, IgD, IgG, IgE, and IgA. The Immunoglobulin Lambda (IGL) locus refers to a collection of genes located on chromosome 22 and is responsible for the production of lambda light chain immunoglobulins, composed of V, J, and C regions, which are involved in the process of antibody diversity.

By aqueous solution herein is meant a liquid including at least 20 vol % water. In embodiments, aqueous solution includes at least 50%, for example at least 75 vol %, at least 95 vol %, above 98 vol %, or 100 vol % of water as the continuous phase.

As used herein, the term “code,” means a system of rules to convert information, such as signals obtained from a detection apparatus, into another form or representation, such as a base call or nucleic acid sequence. For example, signals that are produced by one or more incorporated nucleotides can be encoded by a digit. The digit can have several potential values, each value encoding a different signal state. For example, a binary digit will have a first value for a first signal state and a second value for a second signal state. A digit can have a higher radix including, for example, a ternary digit having three potential values, a quaternary digit having four potential values, etc. A series of digits can form a codeword. The length of the codeword is the same as the number of sequencing steps performed. Exemplary codes include, but are not limited to, a Hamming code. A Hamming code is used in accordance with its ordinary meaning in computer science, mathematics, telecommunication sciences and refers to a code that can be used to detect and correct the errors that can occur when the data is moved or stored. The Hamming distance refers to the difference in integer number between two codewords of equal length, and may be determined using known techniques in the art such as the Hamming distance test or the Hamming distance algorithm. For example, for two codewords (i.e., two sequenced barcodes that have been converted to a string of integers), a difference of 0 indicates that the codewords (i.e., the sequences) are identical. A difference of 1 in integer value indicates a Hamming distance of 1, thus 1 base difference between the oligos. Hamming distance is the number of positions for which the corresponding bit values in the two strings are different. In other words, the test measures the minimum number of substitutions that would be necessary to change one bit string into the other.

As used herein, the term “identification oligonucleotide” can also refer to a “barcode” or “index” or “unique molecular identifier (UMI)” and refers to a known nucleic acid sequence which has feature(s) that can be identified. Typically, an identification oligonucleotide is unique to a particular feature in a pool of identification oligonucleotide that differ from one another in sequence, and each of which is associated with a different feature. In embodiments, identification oligonucleotides are about or at least about 5, 6, 7, 8, 9, 10, 15, 20, 25, 30, 40, 50, 75 or more nucleotides in length. In embodiments, identification oligonucleotides are shorter than 20, 15, 10, 9, 8, 7, 6, or 5 nucleotides in length. In embodiments, identification oligonucleotides are 10-50 nucleotides in length, such as 15-40 or 20-30 nucleotides in length. In a pool of different identification oligonucleotides, identification oligonucleotides may have the same or different lengths. In general, identification oligonucleotides are of sufficient length and include sequences that are sufficiently different to allow the identification of associated features (e.g., a binding agent or analyte) based on identification oligonucleotides with which they are associated. In embodiments, an identification oligonucleotide can be identified accurately after the mutation, insertion, or deletion of one or more nucleotides in the identification oligonucleotide sequence, such as the mutation, insertion, or deletion of 1, 2, 3, 4, 5, or more nucleotides. In embodiments, each identification oligonucleotide in a plurality of identification oligonucleotides differs from every other identification oligonucleotide in the plurality by at least three nucleotide positions, such as at least 3, 4, 5, 6, 7, 8, 9, 10, or more nucleotide positions.

As used herein, the terms “biomolecule” refer to an agent (e.g., a compound, macromolecule, or small molecule), and the like derived from a biological system (e.g., an organism, a cell, or a tissue). The biomolecule may contain multiple individual components that collectively construct the biomolecule, for example, in embodiments, the biomolecule is a polynucleotide wherein the polynucleotide is composed of nucleotide monomers. The biomolecule may be or may include DNA, RNA, organelles, carbohydrates, lipids, proteins, as well as analogs, fragments, or any combination thereof. These components may be extracellular. In some examples, the biomolecule may be referred to as a clump or aggregate of combinations of components. In some instances, the biomolecule may include one or more constituents of a cell but may not include other constituents of the cell. In embodiments, a biomolecule is a molecule produced by a biological system (e.g., an organism). The biomolecule may be any substance (e.g., molecule) or entity that is desired to be detected by the method of the invention. The biomolecule is the “target” of the assay method of the invention. The biomolecule may accordingly be any compound that may be desired to be detected, for example a peptide or protein, or nucleic acid molecule or a small molecule, including organic and inorganic molecules. The biomolecule may be a cell or a microorganism, including a virus, or a fragment or product thereof. Biomolecules of particular interest may thus include proteinaceous molecules such as peptides, polypeptides, proteins or prions or any molecule which includes a protein or polypeptide component, etc., or fragments thereof. The biomolecule may be a single molecule or a complex that contains two or more molecular subunits, which may or may not be covalently bound to one another, and which may be the same or different. Thus, in addition to cells or microorganisms, such a complex biomolecule may also be a protein complex. Such a complex may thus be a homo- or hetero-multimer. Aggregates of molecules e.g., proteins may also be target analytes, for example aggregates of the same protein or different proteins. The biomolecule may also be a complex between proteins or peptides and nucleic acid molecules such as DNA or RNA. Of particular interest may be the interactions between proteins and nucleic acids, e.g., regulatory factors, such as transcription factors, and interactions between DNA or RNA molecules. The term “biomolecule” also refers to a conjugate formed as a result of covalently linking a compound as described herein and a biomolecule (e.g., a nucleic acid, a protein, or an antibody). In embodiments, a target biomolecule is detected by contacting a sample including the target biomolecules with a fluorescently labeled probe including a biomolecule-specific agent and fluorophore. In embodiments, a plurality of biomolecules are detected by contacting a sample including the target biomolecules with a plurality of fluorescently labeled probe including a biomolecule-specific agent and fluorophore.

The terms “detect” and “detecting” as used herein refer to the act of viewing (e.g., imaging, indicating the presence of, quantifying, or measuring (e.g., spectroscopic measurement), an agent based on an identifiable characteristic of the agent, for example, the light emitted from the present compounds. For example, the compound described herein can be bound to an agent, and, upon being exposed to an absorption light, will emit an emission light. The presence of an emission light can indicate the presence of the agent. Likewise, the quantification of the emitted light intensity can be used to measure the concentration of the agent.

The term “detectable moiety” or “detectable agent” or “detectable label” can also refer to a “label” or “labels” and generally refer to molecules that can directly or indirectly produce or result in a detectable signal either by themselves or upon interaction with another molecule. Examples of detectable agents (i.e., labels) include imaging agents, including fluorescent and luminescent substances, molecules, or compositions, including, but not limited to, a variety of organic or inorganic small molecules commonly referred to as “dyes,” “labels,” or “indicators.” In embodiments, “detectable moiety” or “detectable agent” or “detectable label” refers to a compound containing a fluorescent dye moiety or derivatives thereof, which can be used to detect a target analyte or biomolecule of interest. Detection of a detectable label is typically accomplished by measuring an emission wavelength emitted by the fluorescent dye moiety following its absorption of an excitation light at a specific wavelength. In embodiments, a detectable label is conjugated to a biomolecule through a covalent linker. In embodiments, a detectable label is conjugated to a biomolecule through a cleavable linker. Examples of detectable moieties include fluorescein, rhodamine, acridine dyes, Alexa Fluor® dyes, and cyanine dyes. In embodiments, the detectable moiety is a fluorescent molecule (e.g., acridine dye, cyanine, dye, fluorine dye, oxazine dye, phenanthridine dye, or rhodamine dye). In embodiments, the detectable moiety is a fluorescent molecule (e.g., acridine dye, cyanine, dye, fluorine dye, oxazine dye, phenanthridine dye, or rhodamine dye). The term “cyanine” or “cyanine moiety” as described herein refers to a detectable moiety containing two nitrogen groups separated by a polymethine chain. In embodiments, the cyanine moiety has 3 methine structures (i.e. cyanine 3 or Cy3®). In embodiments, the cyanine moiety has 5 methine structures (i.e. cyanine 5 or Cy5®). In embodiments, the cyanine moiety has 7 methine structures (i.e., cyanine 7 or Cy7®). In embodiments, a detectable moiety is a moiety (e.g., monovalent form) of a detectable agent.

The term “rhodamine” as is used in accordance with its ordinary meaning in the art and refers to a detectable moiety including a xanthene backbone. Structurally, rhodamine is a family of related polycyclic dyes with a xanthene core, i.e.,

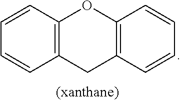 [SMILES: c1ccc2c(c1)Cc1ccccc1O2]

Generally speaking, functional groups on the conjugated moiety of the xanthene core have the ability to fine tune the fluorescent colors. Non-limiting examples of rhodamine dyes include Rhodamine B, Rhodamine 6G, Rhodamine 123, and Rhodamine WH. Rhodamine derivatives have also been disclosed, such as in PCT Int. Appl, WO 2009108905; U.S. Pat. Nos. 5,728,529; 5,686,261; and by Kim et al. (Journal of Physical Chemistry A (2006), 110(1), 20-27)).

The term “sulforhodamine 101 moiety” is used in accordance with its ordinary meaning in the art and refers to detectable moiety containing xanthene backbone with the following structure:

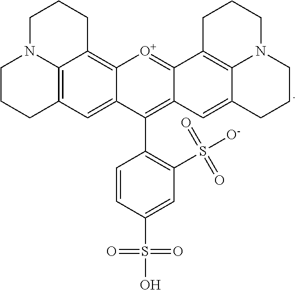 [SMILES: O=S(=O)([O-])c1cc(S(=O)(=O)O)ccc1-c1c2cc3c4c(c2[o+]c2c5c6c(cc12)CCCN6CCC5)CCCN4CCC3]

The sulforhodamine 101 moiety is a red fluorescent dye and commonly used for astrocyte identification. An example of a commercially available dye with a sulforhodamine 101 moiety is Texas Red.

The term “fluorescein moiety” is used in accordance with its ordinary meaning in the art and refers to a detectable moiety containing xanthene backbone having the following structural formula:

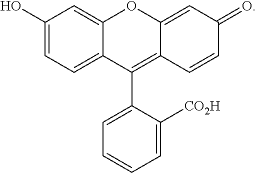 [SMILES: O=C(O)c1ccccc1-c1c2ccc(=O)cc-2oc2cc(O)ccc12]

Dyes with fluorescein moieties are commonly used as fluorescent probes in life sciences and medical applications due to their hydrophilicity, high absorptivity, and high quantum yield. Examples of detectable agents containing fluorescein moieties include fluorescein reactive dyes, which are fluorescein dyes derivatized with different bioconjugation moieties (e.g., maleimide, NHS, or isothiocyanate moieties).

The term “fluorescein isothiocyanate moiety” is used in accordance with its ordinary meaning in the art and refers to a detectable moiety containing xanthene backbone derived from a fluorescein moiety. Detectable agents harboring a fluorescein isothiocyanate moiety has the following structural formula:

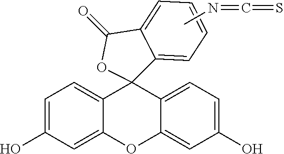

and are primarily used to label primary amines of a biomolecule. Commercially available forms of detectable agents with a fluorescein isothiocyanate moiety include fluorescein 5-isothiocyanate (5-FITC), fluorescein 6-isothiocyanate (6-FITC), or a mixture of the two isomers.

The term “cyanine” or “cyanine moiety” is used in accordance with its ordinary meaning in the art and refers to a detectable moiety containing two nitrogen groups separated by a polymethine chain. In embodiments, the cyanine moiety has 3 methine structures (i.e., cyanine 3 or Cy3). In embodiments, the cyanine moiety has 5 methine structures (i.e., cyanine 5 or Cy5). In embodiments, the cyanine moiety has 7 methine structures (i.e., cyanine 7 or Cy7). Cyanine dyes refer to a family of dyes in which the chromophoric system includes conjugated double bonds connecting two end groups consisting of an electron acceptor and an electron donor. There are three types of cyanine dyes: (1) closed chain cyanines of the general structure:

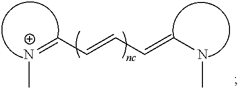

(2) hemicyanines of the general structure:

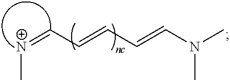

and (3) open chain cyanines of the general structure:

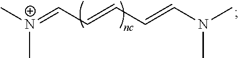

where nc is an integer from 1 to 9.

The term “indocyanine green moiety” is used in accordance with its ordinary meaning in the art and refers to a detectable moiety from the cyanine family of dyes. Specifically, an indocyanine green moiety consists of a cyanine 7 dye moiety of the following structure:

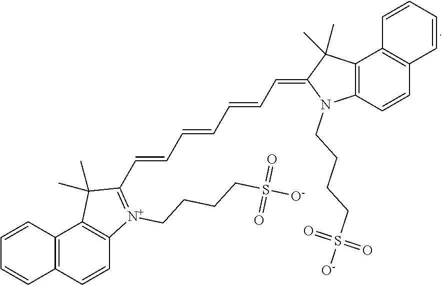 [SMILES: CC1(C)C(/C=C/C=C/C=C/C=C2\N(CCCCS(=O)(=O)[O-])c3ccc4ccccc4c3C2(C)C)=[N+](CCCCS(=O)(=O)[O-])c2ccc3ccccc3c21]

The term “triarylmethane” is used in accordance with its ordinary meaning in the art and refers to a detectable moiety containing a triarylmethane backbone. A triarylmethane dye is derived from a triaryl methane compound and is used for colorimetric assays, analytical chemistry, and are used to color fabrics and plastics, as well as in inks and paints. Examples of triaryl methane dyes include Malachite Green, Crystal Violet, Methyl Violet, Methylene Blue, and Phenol Red.

The term “coumarin moiety” is used in accordance with its ordinary meaning in the art and refers to a detectable moiety containing a benzene and α-pyrone rings of the general structure:

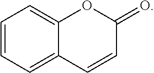 [SMILES: O=c1ccc2ccccc2o1]

Dyes with a coumarin moiety are typically excited with electromagnetic radiation from the UV range and emit between 400-470 nm. Examples of commercially available dyes derived containing a coumarin moiety include DiFMUP (6,8-difluoro-4-methylumbelliferyl phosphate) and AMC (7-amino-4-methylcoumarin).

The term “detecting a fluorescent moiety” is used in accordance with its ordinary meaning in the art and refers to the process of measuring light emitted from a fluorescent compound using a detector (e.g., charge-coupled device (CCD), avalanche photodiodes, or photomultiplier tubes (PMTs)). In embodiments, detecting a fluorescent moiety includes detecting a complex including a biomolecule and probe including a biomolecule-specific agent and a fluorescent moiety. In embodiments, detecting a light emission includes detecting light with a wavelength of 400-800 nm. In embodiments, detecting a light emission includes detecting light with a wavelength of 443 nm, 506 nm, 512 nm, 514 nm, 517 nm, 518 nm, 519 nm, 520 nm, 521 nm, 523 nm, 526 nm, 527 nm, 533 nm, 537 nm, 540 nm, 548 nm, 550 nm, 554 nm, 555 nm, 556 nm, 565 nm, 568 nm, 572 nm, 573 nm, 574 nm, 575 nm, 578 nm, 580 nm, 590 nm, 591 nm, 595 nm, 596 nm, 603 nm, 605 nm, 615 nm, 617 nm, 618 nm, 619 nm, 630 nm, 647 nm, 650 nm, 665 nm, 670 nm, 690 nm, 694 nm, 702 nm, 723 nm, or 775 nm.

The terms “fluorophore,” “fluorescent agent,” “fluorescent dye,” or “fluorescent dye moiety” are used interchangeably and refer to a substance, compound, agent, or composition (e.g., compound) that can absorb light at one or more wavelengths and re-emit light at one or more longer wavelengths, relative to the one or more wavelengths of absorbed light. In general, a dye is a molecule, compound, or substance that can provide an optically detectable signal, such as a colorimetric, luminescent, bioluminescent, chemiluminescent, phosphorescent, or fluorescent signal. In embodiments, the label is a dye. In embodiments, the dye is a fluorescent dye. Examples of fluorophores that may be included in the compounds and compositions described herein include fluorescent proteins, xanthene derivatives (e.g., fluorescein, rhodamine, Oregon green, eosin, or Texas red), cyanine and derivatives (e.g., cyanine, indocarbocyanine, oxacarbocyanine, thiacarbocyanine, or merocyanine), napththalene derivatives (e.g., dansyl or prodan derivatives), coumarin and derivatives, oxadiazole derivatives (e.g., pyridyloxazole, nitrobenzoxadiazole or benzoxadiazole), anthracene derivatives (e.g., anthraquinones, DRAQ5™, DRAQ7™, or CyTrak Orange™), pyrene derivatives (e.g., Cascade Blue® and derivatives), oxazine derivatives (e.g., Nile red, Nile blue, cresyl violet, or oxazine 170), acridine derivatives (e.g., proflavin, acridine orange, acridine yellow), arylmethine derivatives (e.g., auramine, crystal violet, or malachite green), tetrapyrrole derivatives (e.g., porphin, phthalocyanine, bilirubin), CF Dye™, DRAQ™, CyTRAK™, BODIPY®, ATTO™ dyes (ATTO-TEC GmbH), Alexa Fluor™, DyLight™ Fluor™, Atto™, Tracy™, FluoProbes™ Abberior Dyes™, DY™ dyes, MegaStokes Dyes™, Sulfo Cy™, Seta™ dyes, SeTau™ dyes, Square Dyes™, Quasar™ dyes, Cal Fluor™ dyes, SureLight Dyes™, PerCP™ Phycobilisomes™, APC™, APCXL™, RPE™, and/or BPE™. A fluorescent moiety is a radical of a fluorescent agent. The emission from the fluorophores can be detected by any number of methods, including but not limited to, fluorescence spectroscopy, fluorescence microscopy, fluorimeters, fluorescent plate readers, infrared scanner analysis, laser scanning confocal microscopy, automated confocal nanoscanning, laser spectrophotometers, fluorescent-activated cell sorters (FACS), image-based analyzers and fluorescent scanners (e.g., gel/membrane scanners).

### II. Compositions & Kits

In an aspect is provided a compound having the formula:

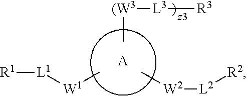

wherein Ring A is a cycloalkyl, heterocycloalkyl, aryl, or heteroaryl. R¹ is a nucleotide moiety. R² is a nucleotide moiety or a fluorescent moiety. R³ is hydrogen, halogen, or a bioconjugate reactive moiety. W¹ is —O—, —NR^{1A}—, or —S—. W² is —O—, —NR^{2A}—, or —S—. W³ is —O—, —NR^{3A}—, or —S—. R^{1A}, R^{2A}, and R^{3A} are independently hydrogen or substituted or unsubstituted alkyl. L¹, L², and L³ and are independently covalent linkers. z3 is 0 or 1.

In embodiments, Ring A is a cycloalkyl (e.g., C₃-C₈, C₃-C₆, or C₅-C₆), heterocycloalkyl (e.g., 3 to 8, 3 to 6, or 5 to 6 membered), aryl (e.g., C₆-C₁₀, C₁₀, or phenyl), or heteroaryl (e.g., 5 to 10, 5 to 9, or 5 to 6 membered). In embodiments, Ring A is an aryl or heteroaryl. In embodiments, Ring A is a C₆-C₁₀ or 5 to 10 membered heteroaryl. In embodiments, Ring A is

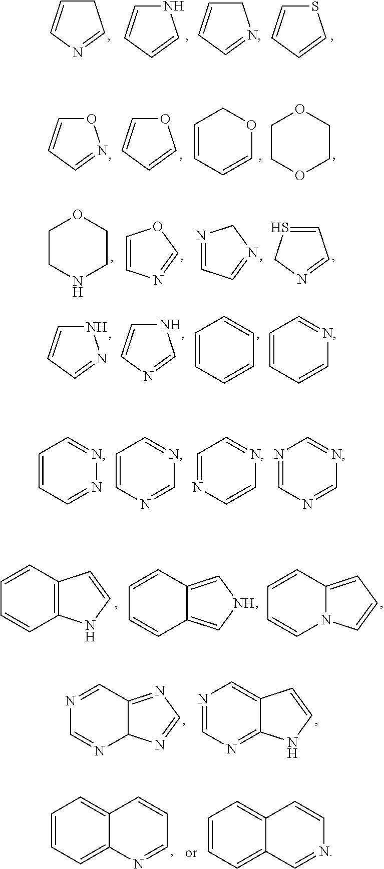

In embodiments, Ring A is optionally further substituted with a substituent group (e.g., oxo) in addition to being substituted with R³, R⁴, and R⁵, are as described herein, including in embodiments. In embodiments, Ring A is a benzene-based heterotrifunctional cross-linker as described by Viault et al (Viault et al. Org. Biomol. Chem., 2013, 11, 2693-2705). For example, Ring A may include three different and orthogonal bioconjugate reactive moieties, such as aminooxy, azido, and thiol moieties. In embodiments, the compound has the formula:

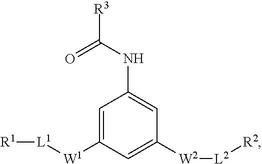

wherein R¹, R², R³, L, and L² are as described herein.

In an aspect is provided a compound having the formula:

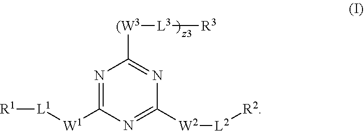

R¹ is a nucleotide moiety. R² is a nucleotide moiety or a fluorescent moiety. R³ is a bioconjugate reactive moiety. W¹ is —O—, —NR^{1A}—, or —S—. W² is —O—, —NR^{2A}—, or —S—. W³ is —O—, —NR^{3A}—, or —S—. R^{1A}, R^{2A}, and R^{3A} are independently hydrogen or substituted or unsubstituted alkyl. L¹, L², and L³ and are independently covalent linkers. z3 is 0 or 1.

In embodiments, the compound has the formula:

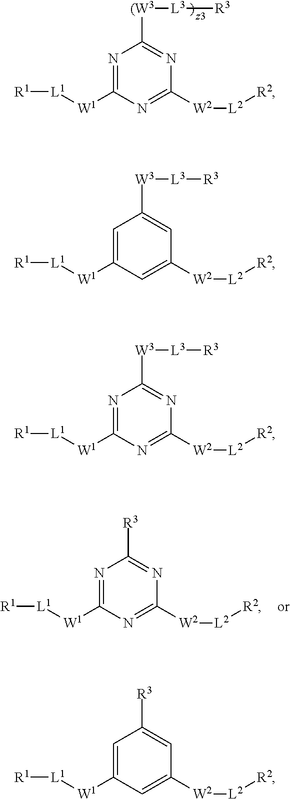

wherein W³, L³, z3, R³, R¹, L¹, W¹, W², L², R² are as described herein.

In embodiments, R¹ is a nucleotide moiety. In embodiments, the nucleotide moiety includes the formula:

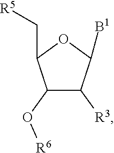

wherein R⁴ is hydrogen or a reversible terminator; R⁵ is a polyphosphate moiety, monophosphate moiety; R⁶ is a hydrogen or —OH, and B is a nucleobase. In embodiments, the nucleotide moiety is connected to the remainder of the compound via a divalent linker, for example the R⁵ moiety or a divalent linker attached to B. In embodiments, the nucleobase is cytosine or a derivative thereof, guanine or a derivative thereof, adenine or a derivative thereof, thymine or a derivative thereof, uracil or a derivative thereof, hypoxanthine or a derivative thereof, xanthine or a derivative thereof, 7-methylguanine or a derivative thereof, 5,6-dihydrouracil or a derivative thereof, 5-methylcytosine or a derivative thereof, or 5-hydroxymethylcytosine or a derivative thereof. In embodiments, the nucleobase is a substituted cytosine or a derivative thereof, substituted guanine or a derivative thereof, substituted adenine or a derivative thereof, substituted thymine or a derivative thereof, substituted uracil or a derivative thereof, substituted hypoxanthine or a derivative thereof, substituted xanthine or a derivative thereof, substituted 7-methylguanine or a derivative thereof, substituted 5,6-dihydrouracil or a derivative thereof, substituted 5-methylcytosine or a derivative thereof, or substituted 5-hydroxymethylcytosine or a derivative thereof. In embodiments, the nucleobase is a substituted cytosine, substituted guanine, substituted adenine, substituted thymine, substituted uracil, substituted hypoxanthine, substituted xanthine, substituted 7-methylguanine, substituted 5,6-dihydrouracil, substituted 5-methylcytosine, or a substituted 5-hydroxymethylcytosine. In embodiments, the nucleobase is substituted with at least one substituent group, size-limited substituent group, or lower substituent group. In embodiments, when the nucleobase is substituted, it is substituted with at least one substituent group. In embodiments, when the nucleobase is substituted, it is substituted with at least one size-limited substituent group. In embodiments, when the nucleobase is substituted, it is substituted with at least one lower substituent group.

In embodiments, B is

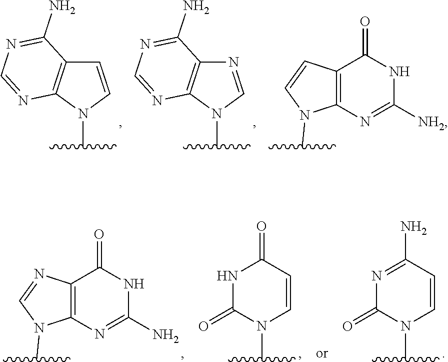

In embodiments, B is

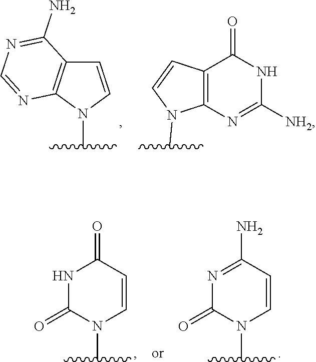

In embodiments, B is

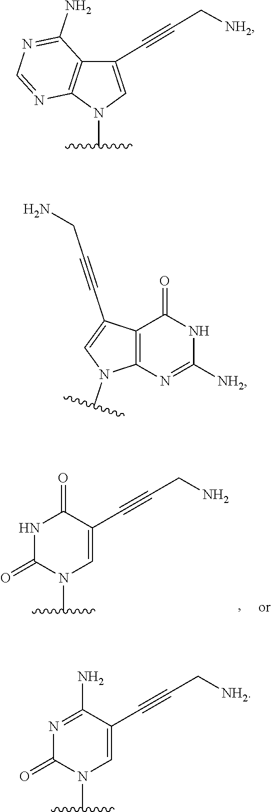 [SMILES: *n1cc(C#CCN)c2c(N)ncnc21 ; *n1cc(C#CCN)c2c(=O)[nH]c(N)nc21 ; *n1cc(C#CCN)c(=O)[nH]c1=O ; *n1cc(C#CCN)c(N)nc1=O]

In embodiments, B is

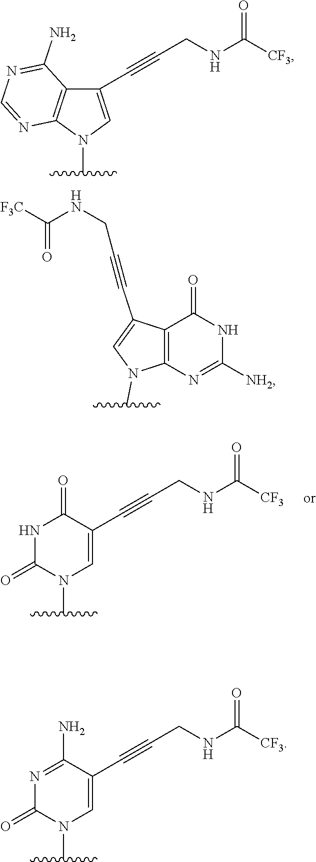

In embodiments, R⁴ is a reversible terminator moiety (e.g., a reversible terminator moiety known in the art). In embodiments, R⁴ is hydrogen. In embodiments, R⁴ is

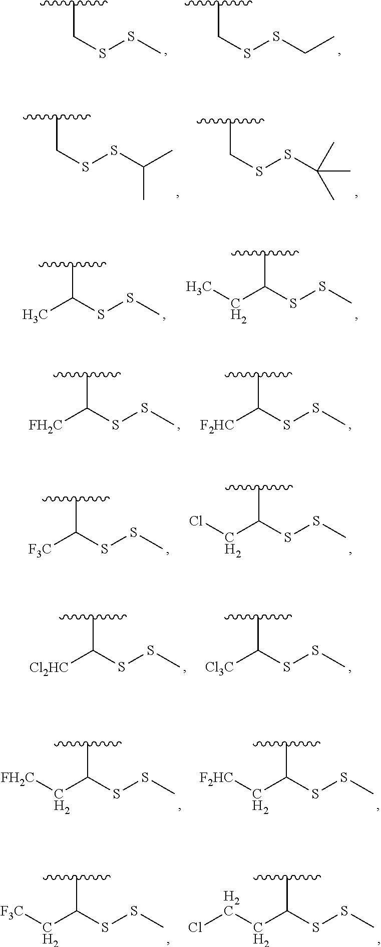

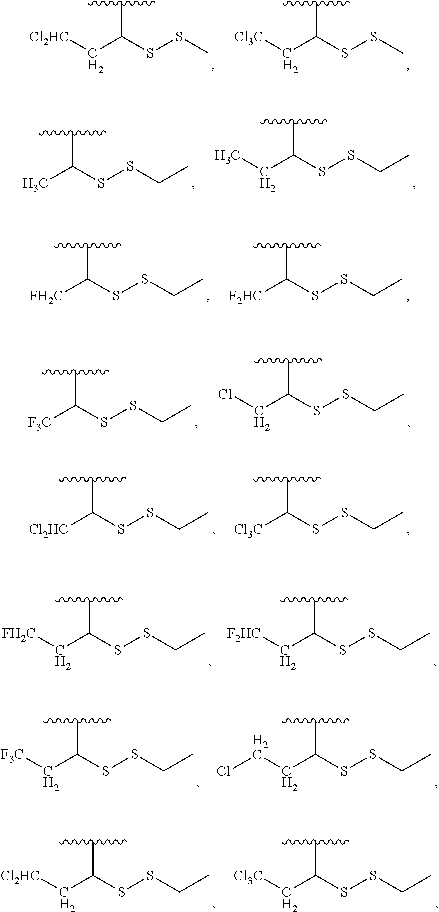

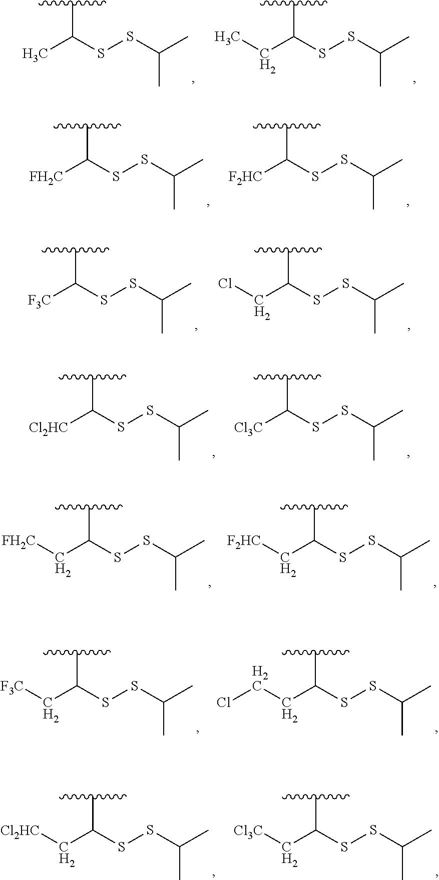

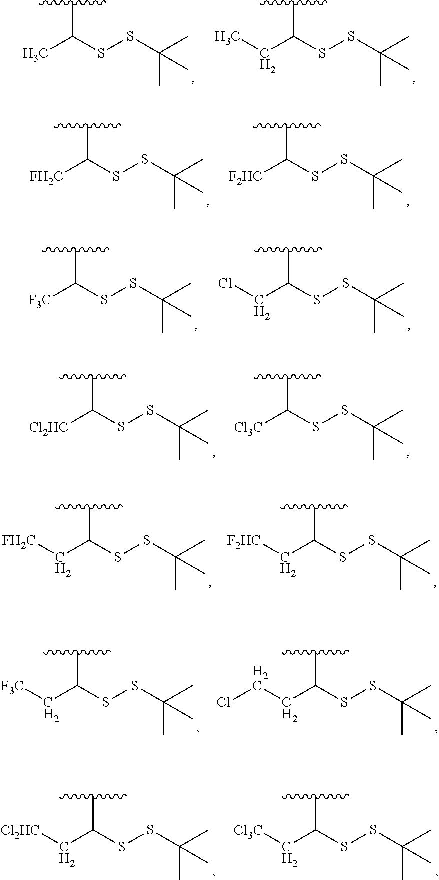

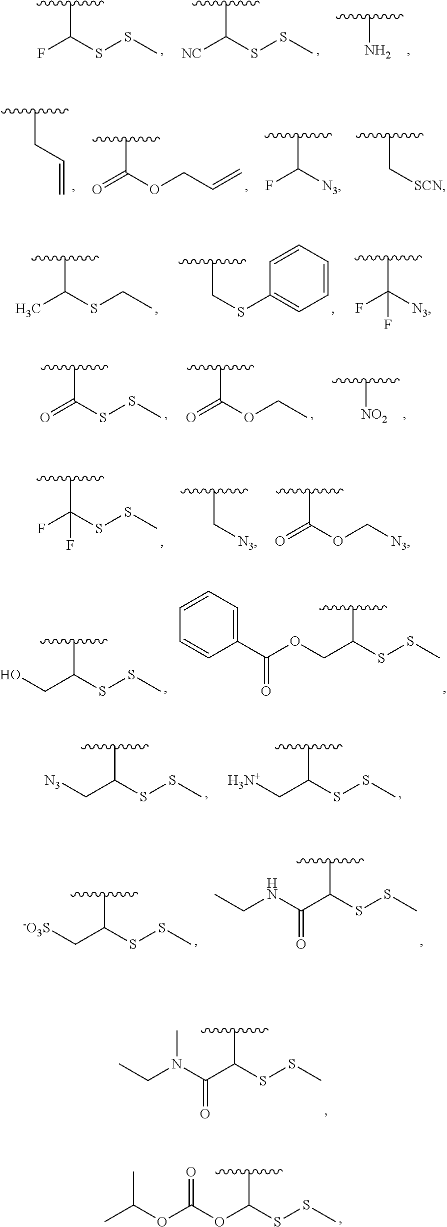

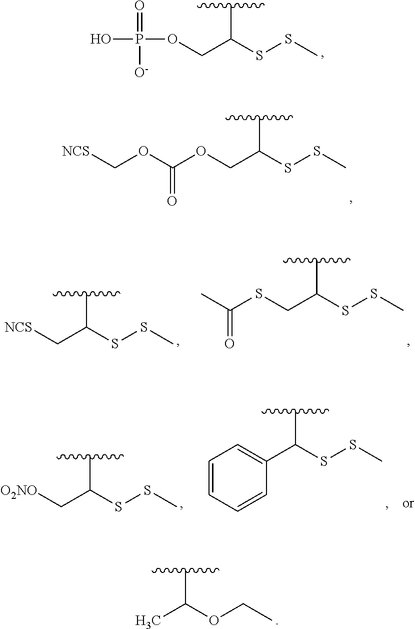

In embodiments, the polymerase-compatible cleavable moiety is:

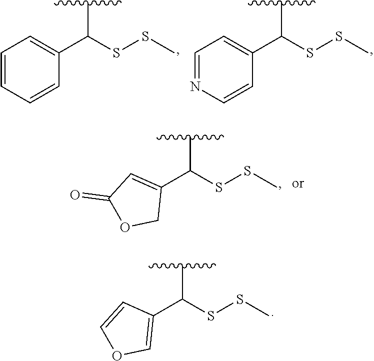

In embodiments, the reversible terminator moiety is

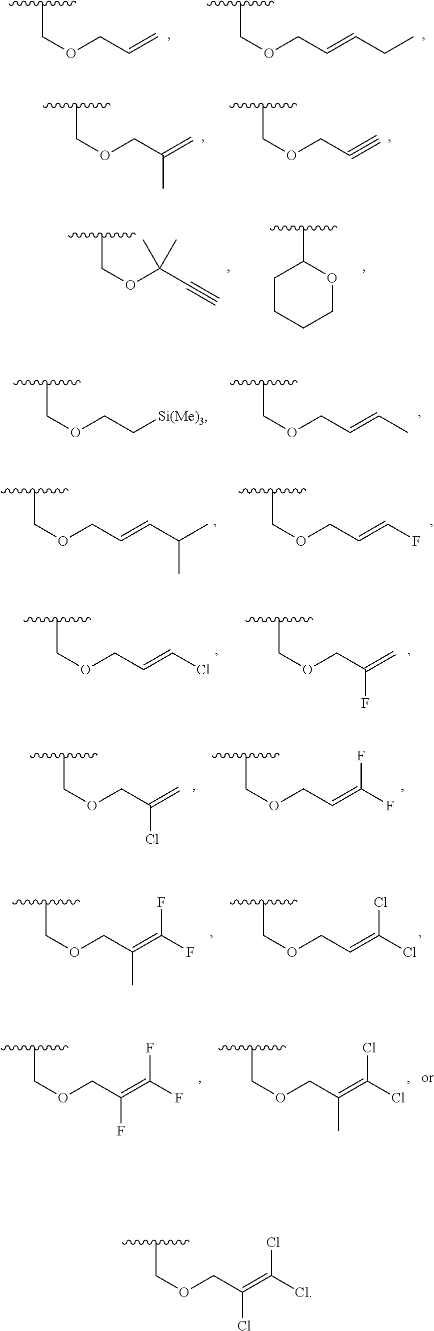

In embodiments, the reversible terminator moiety is

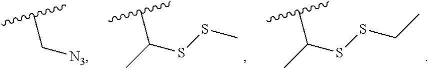

In embodiments, the reversible terminator moiety is

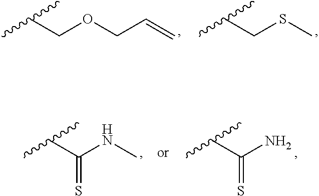

as described in U.S. Patent Publication US 2024/0150827 A1, wherein [symbol] is the point of attachment to the oxygen attached to the 3′ position of the ribose sugar of the nucleotide.

In embodiments, R⁵ is a polyphosphate moiety, triphosphate moiety, or monophosphate moiety. In embodiments, R⁵ includes one or more phosphate moieties. In embodiments, R⁵ includes one or more phosphorothioate moieties. In embodiments, R⁵ is a polyphosphate moiety. In embodiments, R⁵ is a triphosphate moiety. In embodiments, R⁵ is a monophosphate moiety. In embodiments, R⁵ is a divalent polyphosphate moiety.

In embodiments, R⁶ is hydrogen. In embodiments, R⁶ is-OH. In embodiments, R⁶ is a polymerase-compatible cleavable moiety as described herein.

In embodiments, R¹ has the formula:

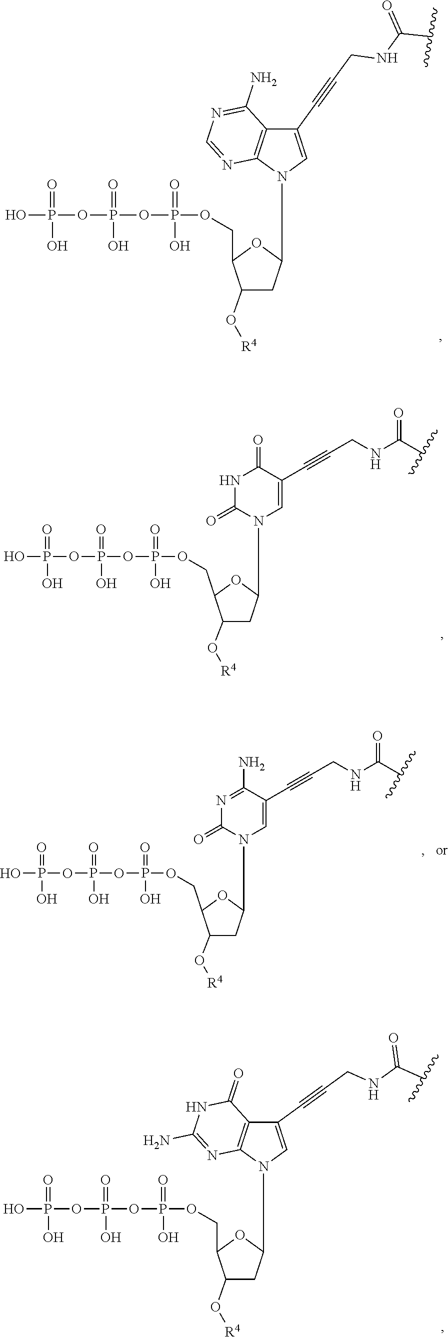

wherein R⁴ is as described herein. In embodiments, R⁴ is hydrogen. In embodiments, R⁴ is a reversible terminator moiety as described herein.

In embodiments, R¹ has the formula:

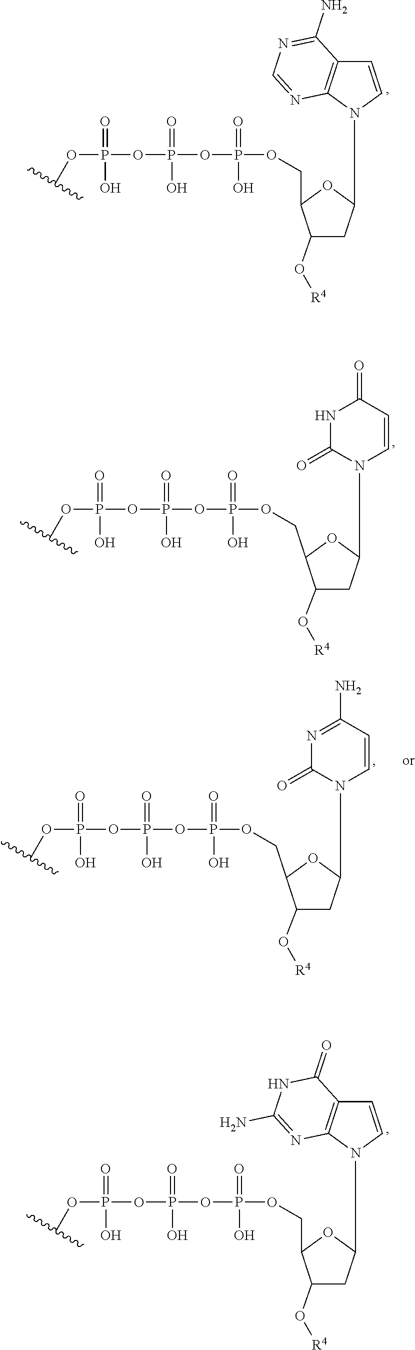

wherein R⁴ is as described herein. In embodiments, R⁴ is hydrogen. In embodiments, R⁴ is a reversible terminator moiety as described herein.

In embodiments, R¹ has the formula:

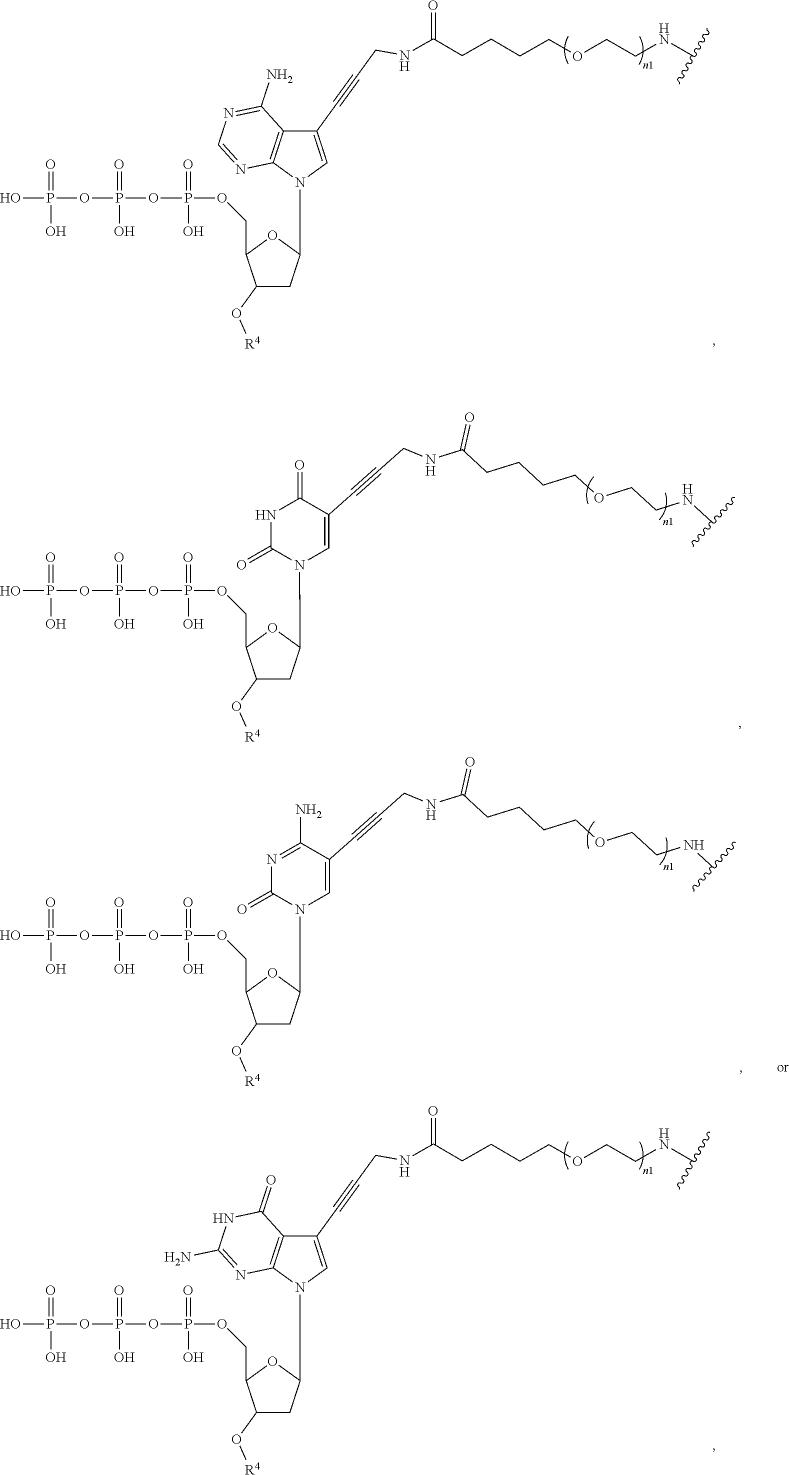

wherein n1 is an integer from 1 to 10. In embodiments, R⁴ a reversible terminator moiety as described herein. In embodiments, R⁴ is hydrogen.

In embodiments, R² is a nucleotide moiety. In embodiments, R² has the formula:

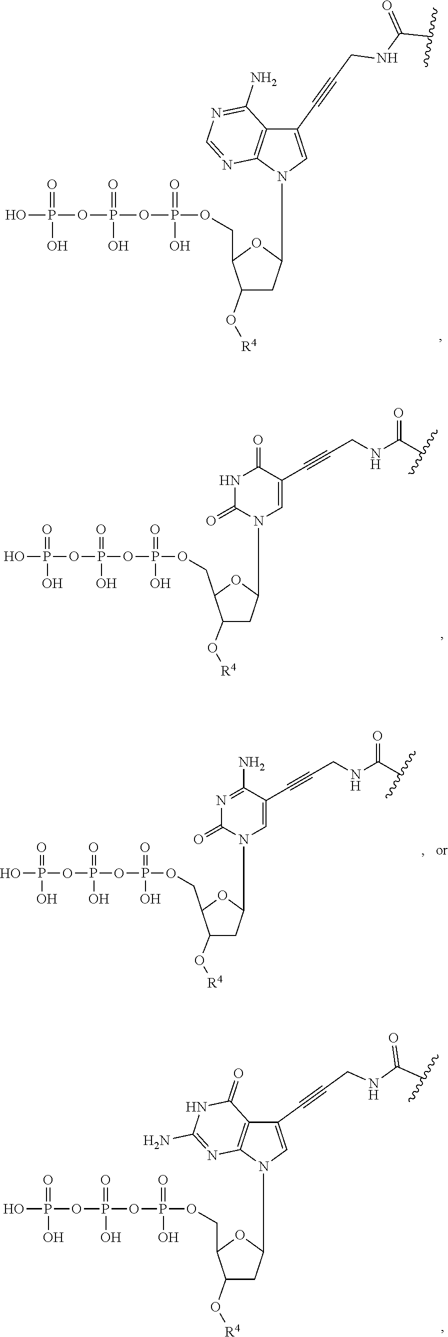

wherein R⁴ is as described herein. In embodiments, R⁴ is hydrogen. In embodiments, R⁴ is a reversible terminator moiety as described herein.

In embodiments, R² has the formula:

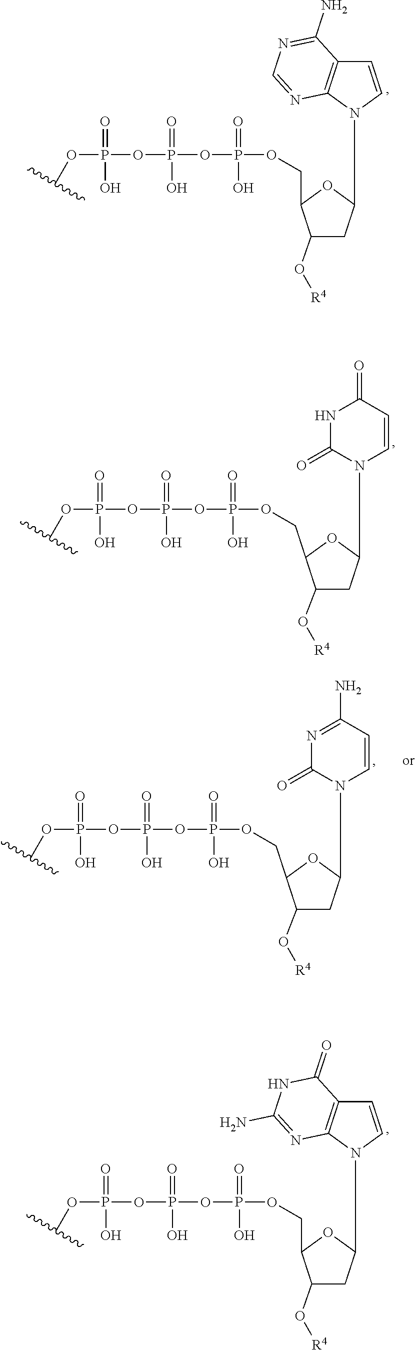

wherein R⁴ is as described herein. In embodiments, R⁴ is hydrogen. In embodiments, R⁴ is a reversible terminator moiety as described herein.

In embodiments, R² has the formula:

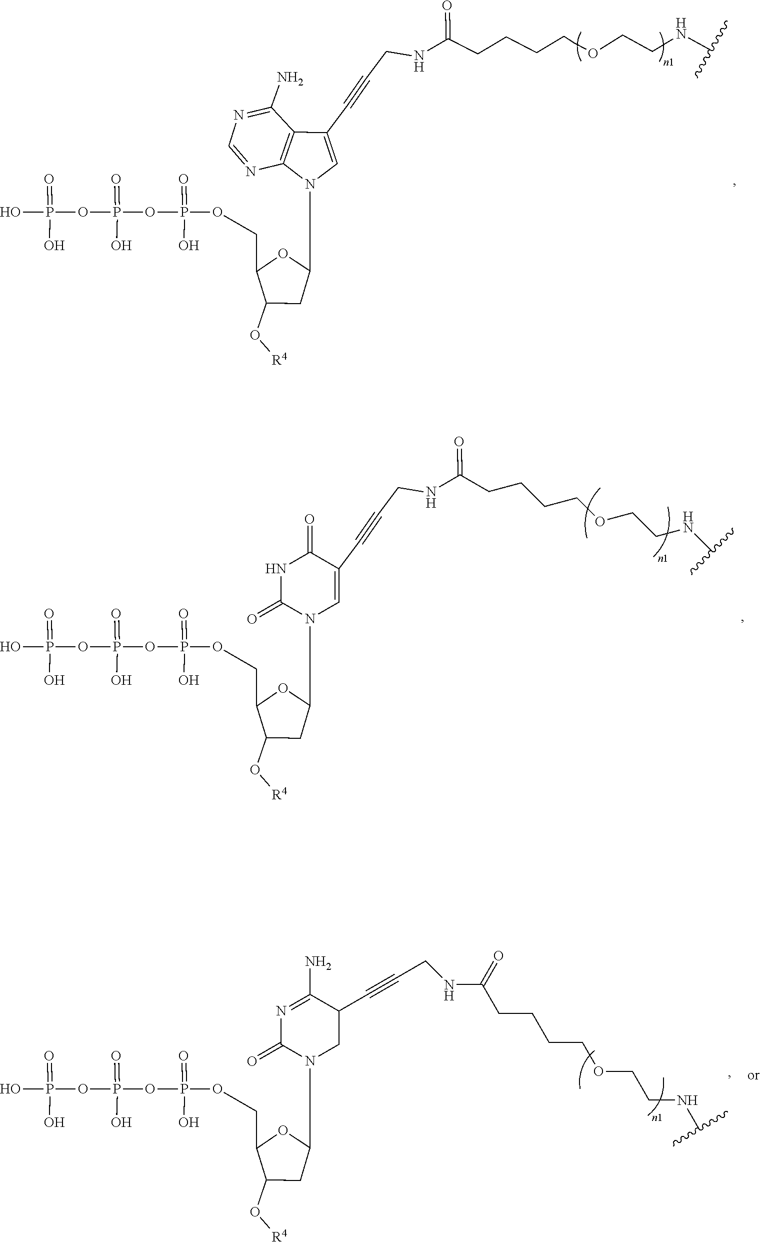

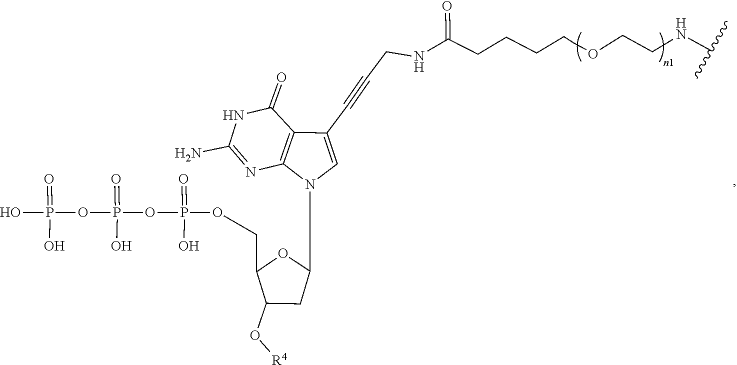

wherein n1 is an integer from 1 to 10. In embodiments, R⁴ is a reversible terminator moiety as described herein. In embodiments, R⁴ is hydrogen.

In embodiments, R² is a fluorescent moiety. In embodiments, R² is a rhodamine moiety. In embodiments, R² is a fluorescein moiety. In embodiments, R² is a triarylmethane moiety. In embodiments, R² is a cyanine moiety. In embodiments, R² is a fluorescein isothiocyanate moiety. In embodiments, R² is an indocyanine green moiety. In embodiments, R² is a coumarin moiety. In embodiments, R² is a sulforhodamine 101 moiety.

In embodiments, a fluorescent moiety is acridine dye moiety, cyanine dye moiety, fluorine dye moiety, oxazine dye moiety, phenanthridine dye moiety, or rhodamine dye moiety). In embodiments, a fluorescent moiety is a triarylmethane moiety, sulforhodamine 101 moiety, sulforhodamine B moiety, Janelia Fluor® dye moiety, naphthalimide moiety, fluorescein isothiocyanate moiety, tetramethylrhodamine-5-(and 6)-isothiocyanate moiety, cyanine moiety, Cy2® moiety, Cy3® moiety, Cy5® moiety, Cy7® moiety, 4′,6-diamidino-2-phenylindole moiety, Hoechst 33258 moiety, Hoechst 33342 moiety, Hoechst 34580 moiety, propidium-iodide moiety, or acridine orange moiety. In embodiments, a fluorescent moiety is an Indo-1 Ca²⁺ saturated moiety, Indo-1 Ca²⁺ moiety, Cascade Blue® BSA pH 7.0 moiety, Cascade Blue® moiety, LysoTracker™ Blue moiety, Alexa Fluor® 405 moiety, LysoSensor™ Blue pH 5.0 moiety, LysoSensor™ Blue moiety, DyLight™ 405 moiety, DyLight™ 350 moiety, BFP (Blue Fluorescent Protein) moiety, Alexa Fluor® 350 moiety, coumarin moiety, 7-Amino-4-methylcoumarin pH 7.0 moiety, Amino Coumarin moiety, AMCA conjugate moiety, Coumarin moiety, 7-Hydroxy-4-methylcoumarin moiety, 7-Hydroxy-4-methylcoumarin pH 9.0 moiety, 6,8-Difluoro-7-hydroxy-4-methylcoumarin pH 9.0 moiety, Hoechst 33342 moiety, Pacific Blue™ moiety, Hoechst 33258 moiety, Hoechst 33258-DNA moiety, Pacific Blue™ antibody conjugate pH 8.0 moiety, PO-PRO™-1 moiety, PO-PRO™-1-DNA moiety, POPO™-moiety, POPO™-1-DNA moiety, DAPI-DNA moiety, DAPI moiety, Marina Blue® moiety, SYTOX Blue™-DNA moiety, CFP (Cyan Fluorescent Protein) moiety, eCFP (Enhanced Cyan Fluorescent Protein) moiety, 1-Anilinonaphthalene-8-sulfonic acid (1,8-ANS) moiety, Indo-1, Ca free moiety, 1,8-ANS (1-Anilinonaphthalene-8-sulfonic acid) moiety, BO-PRO™-1-DNA moiety, BO-PRO™-1 moiety, BOBO™-1-DNA moiety, SYTO™ 45-DNA moiety, Evoglow®-Ppl moiety, Evoglow®-Bs1 moiety, Evoglow®-Bs2 moiety, Auramine O moiety, DiO moiety, LysoSensor™ Green pH 5.0 moiety, Cy2® moiety, LysoSensor™ Green moiety, Fura-2 high Ca moiety, SYTO™ 13-DNA moiety, YO-PRO™-1-DNA moiety, YOYO™-1-DNA moiety, eGFP (Enhanced Green Fluorescent Protein) moiety, LysoSensor™ Green moiety, GFP (S65T) moiety, BODIPY® FL, Sapphire moiety, BODIPY® FL conjugate moiety, MitoTracker™ Green moiety, MitoTracker™ Green, FM™, Fluorescein 0.1 M NaOH moiety, Calcein pH 9.0 moiety, Fluorescein pH 9.0 moiety, Calcein moiety, Fura-2, no Ca moiety, Fluo-4 moiety, FDA moiety, DTAF moiety, Fluorescein moiety, CFDA moiety, FITC moiety, Alexa Fluor® 488 hydrazide-water moiety, DyLight™ 488 moiety, 5-FAM pH 9.0 moiety, Alexa Fluor® 488 moiety, Rhodamine 110 moiety, Rhodamine 110 pH 7.0 moiety, Acridine Orange moiety, BCECF pH 5.5 moiety, PicoGreen® dsDNA quantitation reagent moiety, SYBR™ Green I moiety, Rhodamine Green pH 7.0 moiety, CyQUANT™ GR-DNA moiety, NeuroTrace™ 500/525, green fluorescent Nissl stain-RNA moiety, DansylCadaverine moiety, Fluoro-Emerald moiety, Nissl moiety, Fluorescein dextran pH 8.0 moiety, Rhodamine Green moiety, 5-(and-6)-Carboxy-2′, 7′-dichlorofluorescein pH 9.0 moiety, DansylCadaverine, MeOH moiety, eYFP (Enhanced Yellow Fluorescent Protein) moiety, Oregon Green® 488 moiety, Fluo-3 moiety, BCECF pH 9.0 moiety, SBFI-Na⁺ moiety, Fluo-3 Ca²⁺ moiety, Rhodamine 123 MeOH moiety, FlAsH moiety, Calcium Green-1 Ca²⁺ moiety, Magnesium Green moiety, DM-NERF pH 4.0 moiety, Calcium Green moiety, Citrine moiety, LysoSensor™ Yellow pH 9.0 moiety, TO-PRO™-1-DNA moiety, Magnesium Green Mg2'⁰ moiety, Sodium Green Na⁺ moiety, TOTO™-1-DNA moiety, Oregon Green® 514 moiety, Oregon Green® 514 antibody conjugate pH 8.0 moiety, NBD-X moiety, DM-NERF pH 7.0 moiety, NBD-X, MeOH moiety, CI-NERF pH 6.0 moiety, Alexa Fluor® 430 moiety, CI-NERF pH 2.5 moiety, Lucifer Yellow, CH moiety, LysoSensor™ Yellow pH 3.0 moiety, 6-TET, SE pH 9.0 moiety, Eosin antibody conjugate pH 8.0 moiety, Eosin moiety, 6-Carboxyrhodamine 6G pH 7.0 moiety, 6-Carboxyrhodamine 6G, hydrochloride moiety, BODIPY® R6G SE moiety, BODIPY® R6G moiety, 6-JOE moiety, Cascade Yellow moiety, mBanana moiety, Alexa Fluor® 532 moiety, Erythrosin-5-isothiocyanate pH 9.0 moiety, 6-HEX, SE pH 9.0 moiety, mOrange moiety, mHoneydew moiety, Cy3® moiety, Rhodamine B moiety, DiI moiety, 5-TAMRA-MeOH moiety, Alexa 555 moiety, DyLight™ 549 moiety, BODIPY®TMR-X, SE moiety, BODIPY® TMR-X MeOH moiety, PO-PRO™-3-DNA moiety, PO-PRO™-3 moiety, Rhodamine moiety, POPO™-3 moiety, Alexa Fluor® 546 moiety, Calcium Orange Ca²⁺ moiety, TRITC moiety, Calcium Orange moiety, Rhodaminephalloidin pH 7.0 moiety, MitoTracker™ Orange moiety, MitoTracker™ Orange MeOH moiety, Phycoerythrin moiety, Magnesium Orange moiety, R-Phycoerythrin pH 7.5 moiety, 5-TAMRA™ pH 7.0 moiety, 5-TAMRA™ moiety, Rhod-2 moiety, FM™ 1-43 moiety, Rhod-2 Ca²⁺ moiety, FM™ 1-43 lipid moiety, LOLO™-1-DNA moiety, dTomato moiety, DsRed moiety, Dapoxyl (2-aminoethyl) sulfonamide moiety, Tetramethylrhodamine dextran pH 7.0 moiety, Fluor-Ruby moiety, Resorufin moiety, Resorufin pH 9.0 moiety, mTangerine moiety, LysoTracker™ Red moiety, Lissamine rhodamine moiety, Cy 3.5 moiety, Rhodamine Red-X antibody conjugate pH 8.0 moiety, Sulforhodamine 101 moiety, JC-1 pH 8.2 moiety, JC-1 moiety, mStrawberry moiety, MitoTracker™ Red moiety, X-Rhod-1 Ca²⁺ moiety, Alexa Fluor® 568 moiety, 5-ROX™ pH 7.0 moiety, 5-ROX™ (5-Carboxy-X-rhodamine, triethylammonium salt) moiety, BO-PRO™-3-DNA moiety, BO-PRO™-3 moiety, BOBO™-3-DNA moiety, Ethidium Bromide moiety, ReAsH moiety, Calcium Crimson moiety, Calcium Crimson Ca2+ moiety, mRFP moiety, mCherry moiety, HcRed moiety, DyLight™ 594 moiety, Ethidium homodimer-1-DNA moiety, Ethidiumhomodimer moiety, Propidium Iodide moiety, SYPRO™ Ruby moiety, Propidium Iodide-DNA moiety, Alexa Fluor® 594 moiety, BODIPY® TR-X, SE moiety, BODIPY® TR-X, BODIPY® TR-X phallacidin pH 7.0 moiety, Alexa Fluor® 610 R-phycoerythrin streptavidin pH 7.2 moiety, YO-PRO™-3-DNA moiety, Di-8 ANEPPS moiety, Di-8-ANEPPS-lipid moiety, YOYO™ 3-DNA moiety, Nile Red-lipid moiety, Nile Red moiety, DyLight™ 633 moiety, mPlum moiety, TO-PRO™-3-DNA moiety, DDAO pH 9.0 moiety, Fura Red™ high Ca moiety, Allophycocyanin pH 7.5 moiety, APC (allophycocyanin) moiety, Nile Blue, EtOH moiety, TOTO™-3-DNA moiety, Cy5® moiety, BODIPY® 650/665-X, Alexa Fluor® 647 R-phycoerythrin streptavidin pH 7.2 moiety, DyLight™ 649 moiety, Alexa Fluor® 647 moiety, Fura Red™ Ca²⁺ moiety, ATTO™ 647 moiety, Fura Red™, low Ca moiety, Carboxynaphthofluorescein pH 10.0 moiety, Alexa Fluor® 660 moiety, Cy 5.5@moiety, Alexa Fluor® 680 moiety, DyLight™ 680 moiety, Alexa Fluor® 700 moiety, FM™ 4-64, 2% CHAPS moiety, or FM™ 4-64 moiety. In embodiments, the detectable moiety is a moiety of 1,1-Diethyl-4,4-carbocyanine iodide, 1,2-Diphenylacetylene, 1,4-Diphenylbutadiene, 1,4-Diphenylbutadiyne, 1,6-Diphenylhexatriene, 1,6-Diphenylhexatriene, 1-anilinonaphthalene-8-sulfonic acid, 2,7-Dichlorofluorescein, 2,5-Diphenyloxazole, 2-Di-1-ASP, 2-dodecylresorufin, 2-Methylbenzoxazole, 3,3-Diethylthiadicarbocyanine iodide, 4-Dimethylamino-4-Nitrostilbene, 5(6)-Carboxyfluorescein, 5(6)-Carboxynaphtofluorescein, 5(6)-Carboxytetramethylrhodamine B, 5-(and-6)-carboxy-2′,7′-dichlorofluorescein, 5-(and-6)-carboxy-2,7-dichlorofluorescein, 5-(N-hexadecanoyl)aminoeosin, 5-(N-hexadecanoyl)aminoeosin, 5-chloromethylfluorescein, 5-FAM, 5-ROX™, 5-TAMRA™, 5-TAMRA™, 6,8-difluoro-7-hydroxy-4-methylcoumarin, 6,8-difluoro-7-hydroxy-4-methylcoumarin, 6-carboxyrhodamine 6G, 6-HEX, 6-JOE, 6-JOE, 6-TET, 7-aminoactinomycin D, 7-Benzylamino-4-Nitrobenz-2-Oxa-1,3-Diazole, 7-Methoxycoumarin-4-Acetic Acid, 8-Benzyloxy-5,7-diphenylquinoline, 9,10-Bis(Phenylethynyl)Anthracene, 9,10-Diphenylanthracene, 9-METHYLCARBAZOLE, (CS)2Ir(μ-Cl)2Ir(CS)2, AAA, Acridine Orange, Acridine Yellow, Adams Apple Red 680, Adirondack Green 520, Alexa Fluor® 350, Alexa Fluor® 405, Alexa Fluor® 430, Alexa Fluor® 480, Alexa Fluor® 488, Alexa Fluor® 488 hydrazide, Alexa Fluor® 500, Alexa Fluor® 514, Alexa Fluor® 532, Alexa Fluor® 546, Alexa Fluor® 555, Alexa Fluor® 568, Alexa Fluor® 594, Alexa Fluor® 610, 610—R-PE, Alexa Fluor® 633, Alexa Fluor® 635, Alexa Fluor® 647, Alexa Fluor® 647-R-PE, Alexa Fluor® 660, Alexa Fluor® 680, Alexa Fluor® 680-APC, Alexa Fluor® 680-R-PE, Alexa Fluor® 700, Alexa Fluor® 750, Alexa Fluor® 790, Allophycocyanin, AmCyan1, Aminomethylcoumarin, Amplex Gold (product), Amplex Red Reagent, Amplex UltraRed, Anthracene, APC, APC-Seta-750, AsRed2, ATTO™ 390, ATTO™ 425, ATTO™ 430LS, ATTO™ 465, ATTO™ 488, ATTO™ 490LS, ATTO™ 495, ATTO™ 514, ATTO™ 520, ATTO™ 532, ATTO™ 550, ATTO™ 565, ATTO™ 590, ATTO™ 594, ATTO™ 610, ATTO™ 620, ATTO™ 633, ATTO™ 635, ATTO™ 647, ATTO™ 647N, ATTO™ 655, ATTO™ 665, ATTO™ 680, ATTO™ 700, ATTO™ 725, ATTO™ 740, ATTO™ Oxa12, ATTO™ Rho3B, ATTO™ Rho6G, ATTO™ Rho11, ATTO™ Rho12, ATTO™ Rho13, ATTO™ Rho14, ATTO™ Rho101, ATTO™ Thio12, Auramine O, Azami Green, Azami Green monomeric, B-phycoerythrin, BCECF, BCECF, Bex1, Biphenyl, Birch Yellow 580, Blue-green algae, BO-PRO™-1, BO-PRO™-3, BOBO™-1, BOBO™-3, BODIPY@630 650—X, BODIPY® 650/665-X, BODIPY® FL, BODIPY® R6G, BODIPY® TMR-X, BODIPY® TR-X, BODIPY® TR-X Ph 7.0, BODIPY® TR-X phallacidin, BODIPY® DiMe, BODIPY® Phenyl, BODIPY TMSCC, C3-Indocyanine, C3-Oxacyanine, C3-Thiacyanine Dye (EtOH), C3-Thiacyanine Dye (PrOH), C5-Indocyanine, C5-Oxacyanine, C5-Thiacyanine, C7-Indocyanine, C7-Oxacyanine, C545T, C-Phycocyanin, Calcein, Calcein red-orange, Calcium Crimson, Calcium Green-1, Calcium Orange, Calcofluor white 2MR, Carboxy SNARF-1 pH 6.0, Carboxy SNARF-1 pH 9.0, Carboxynaphthofluorescein, Cascade Blue®, Cascade Yellow, Catskill Green 540, CBQCA, CellMask™ Orange, CellTrace™ BODIPY®TR methyl ester, CellTrace™ calcein violet, CellTrace™ Far Red, CellTracker™ Blue, CellTracker™ Red CMTPX, CellTracker™ Violet BMQC, CF@405M, CF@405S, CF®488A, CF@543, CF@555, CFP, CFSE, CF™ 350, CF™ 485, Chlorophyll A, Chlorophyll B, Chromeo™ 488, Chromeo™ 494, Chromeo™ 505, Chromeo™ 546, Chromeo™ 642, Citrine, Citrine, ClOH butoxy aza-BODIPY®, ClOH C12 aza-BODIPY®, CM-H2DCFDA, Coumarin 1, Coumarin 6, Coumarin 6, Coumarin 30, Coumarin 314, Coumarin 334, Coumarin 343, Coumarine 545T, Cresyl Violet Perchlorate, CryptoLight CF1, CryptoLight CF2, CryptoLight CF3, CryptoLight CF4, CryptoLight CF5, CryptoLight CF6, Crystal Violet, Coumarin153, Cy2®, Cy3®, Cy3.5@, Cy3B®, Cy5®, Cy5.5@, Cy7®, Cyanine3 NHS ester, Cyanine5 carboxylic acid, Cyanine5 NHS ester, Cyclotella meneghiniana Kutzing, CypHer5, CypHer5 pH 9.15, CyQUANT™ GR, CyTrak Orange™, Dabcyl SE, DAF-FM™, DAMC (Weiss), dansyl cadaverine, Dansyl Glycine (Dioxane), DAPI, DAPI, DAPI, DAPI, DAPI (DMSO), DAPI (H2O), Dapoxyl (2-aminoethyl)sulfonamide, DCI, DCM, DCM, DCM (acetonitrile), DCM (MeOH), DDAO, Deep Purple, di-8-ANEPPS, DiA, Dichlorotris(1,10-phenanthroline) ruthenium(II), DiClOH C12 aza-BODIPY®, DiClOHbutoxy aza-BODIPY®, DiD, DiI, DiIC18(3), DiO, DiR, Diversa Cyan-FP, Diversa Green-FP, DM-NERF pH 4.0, DOCI, Doxorubicin, DPP pH-Probe 590-7.5, DPP pH-Probe 590-9.0, DPP pH-Probe 590-11.0, DPP pH-Probe 590-11.0, Dragon Green, DRAQ5™, DsRed, DsRed-Express, DsRed-Express2, DsRed-Express T1, dTomato, DY-350XL, DY-480, DY-480XL MegaStokes, DY-485, DY-485XL MegaStokes, DY-490, DY-490XL MegaStokes, DY-500, DY-500XL MegaStokes, DY-520, DY-520XL MegaStokes, DY-547, DY-549P1, DY-554, DY-555, DY-557, DY-590, DY-615, DY-630, DY-631, DY-633, DY-635, DY-636, DY-647, DY-649P1, DY-650, DY-651, DY-656, DY-673, DY-675, DY-676, DY-680, DY-681, DY-700, DY-701, DY-730, DY-731, DY-750, DY-751, DY-776, DY-782, Dye-28, Dye-33, Dye-45, Dye-304, Dye-1041, DyLight™ 488, DyLight™ 549, DyLight™ 633, DyLight™ 649, DyLight™ 680, E2-Crimson, E2-Orange, E2-Red/Green, EBFP, ECF, ECFP, ECL Plus, eGFP, ELF 97, Emerald, Envy Green, Eosin, Eosin Y, epicocconone, EqFP611, Erythrosin-5-isothiocyanate, Ethidium bromide, ethidium homodimer-1, Ethyl Eosin, Ethyl Nile Blue A, Ethyl-p-Dimethylaminobenzoate, Ethyl-p-Dimethylaminobenzoate, Eu2O3 nanoparticles, Eu (Soini), Eu(tta)3DEADIT, EvaGreen®, EVOblue®-30, EYFP, FAD, FITC, FITC, FlAsH (Adams), Flash Red EX, FlAsH-CCPGCC, FlAsH-CCXXCC, Fluo-3, Fluo-4, Fluo-5F, Fluorescein, Fluorescein 0.1 NaOH, Fluorescein-Dibase, fluoro-emerald, Fluorol 5G, FluoSpheres™ blue, FluoSpheres™ Crimson, FluoSpheres™ dark red, FluoSpheres™ orange, FluoSpheres™ red, FluoSpheres™ yellow-green, FM™4-64 in CTC, FM™4-64 in SDS, FM™ 1-43, FM™ 4-64, Fort Orange 600, Fura Red™, Fura Red™ Ca free, fura-2, Fura-2 Ca free, Gadodiamide, Gd-Dtpa-Bma, Gadodiamide, Gd-Dtpa-Bma, GelGreen®, GelRed®, H9-40, HcRedl, Hemo Red 720, HiLyte™ Fluor 488, HiLyte™ Fluor 555, HiLyte™ Fluor 647, HiLyte™ Fluor 680, HiLyte™ Fluor 750, HiLyte™ Plus 555, HiLyte™ Plus 647, HiLyte™ Plus 750, HmGFP, Hoechst 33258, Hoechst 33342, Hoechst-33258, Hoechst-33258, Hops Yellow 560, HPTS, HPTS, HPTS, HPTS, HPTS, indo-1, Indo-1 Ca free, Ir(Cn)2(acac), Ir(Cs)2(acac), IR-775 chloride, IR-806, Ir-OEP—CO—C1, IRDye® 650 Alkyne, IRDye® 650 Azide, IRDye® 650 Carboxylate, IRDye® 650 DBCO, IRDye® 650 Maleimide, IRDye® 650 NHS Ester, IRDye® 680LT Carboxylate, IRDye® 680LT Maleimide, IRDye® 680LT NHS Ester, IRDye® 680RD Alkyne, IRDye® 680RD Azide, IRDye® 680RD Carboxylate, IRDye® 680RD DBCO, IRDye® 680RD Maleimide, IRDye® 680RD NHS Ester, IRDye® 700 phosphoramidite, IRDye® 700DX, IRDye® 700DX, IRDye® 700DX Carboxylate, IRDye® 700DX NHS Ester, IRDye® 750 Carboxylate, IRDye® 750 Maleimide, IRDye® 750 NHS Ester, IRDye® 800 phosphoramidite, IRDye® 800CW, IRDye® 800CW Alkyne, IRDye® 800CW Azide, IRDye® 800CW Carboxylate, IRDye® 800CW DBCO, IRDye® 800CW Maleimide, IRDye® 800CW NHS Ester, IRDye® 800RS, IRDye® 800RS Carboxylate, IRDye® 800RS NHS Ester, IRDye® QC-1 Carboxylate, IRDye® QC-1 NHS Ester, Isochrysis galbana—Parke, JC-1, JC-1, JOJO™-1, Jonamac Red Evitag T2, Kaede Green, Kaede Red, kusabira orange, Lake Placid 490, LDS 751, Lissamine Rhodamine (Weiss), LOLO™-1, Lucifer Yellow CH, Lucifer Yellow CH Dilitium salt, Lumio Green, Lumio Red, Lumogen F Orange, Lumogen Red F300, Lumogen Red F300, LysoSensor™ Blue DND-192, LysoSensor™ Green DND-153, LysoSensor™ Green DND-153, LysoSensor™ Yellow/Blue DND-160 pH 3, LysoSensor™ Yellow Blue DND-160, LysoTracker™ Blue DND-22, LysoTracker™ Blue DND-22, LysoTracker™ Green DND-26, LysoTracker™ Red DND-99, LysoTracker™ Yellow HCK-123, Macoun Red Evitag T2, Macrolex® Fluorescence Red G, Macrolex® Fluorescence Yellow 10GN, Macrolex® Fluorescence Yellow 10GN, Magnesium Green, Magnesium Octaethylporphyrin, Magnesium Orange, Magnesium Phthalocyanine, Magnesium Phthalocyanine, Magnesium Tetramesitylporphyrin, Magnesium Tetraphenylporphyrin, malachite green isothiocyanate, Maple Red-Orange 620, Marina Blue®, mBanana, mBBr, mCherry, Merocyanine 540, Methyl green, Methyl green, Methyl green, Methylene Blue, Methylene Blue, mHoneyDew, MitoTracker™ Deep Red 633, MitoTracker™ Green FM™ MitoTracker™ Orange CMTMRos, MitoTracker™ Red CMXRos, monobromobimane, Monochlorobimane, Monoraphidium, mOrange, mOrange2, mPlum, mRaspberry, mRFP, mRFP1, mRFP1.2 (Wang), mStrawberry (Shaner), mTangerine (Shaner), N,N-Bis(2,4,6-trimethylphenyl)-3,4:9,10-perylenebis(dicarboximide), NADH, Naphthalene, Naphthofluorescein, NBD-X, NeuroTrace™ 500525, Nilblau perchlorate, Nile Blue, Nile Red, Nileblue A, NIR1, NIR2, NIR3, NIR4, NIR820, Octaethylporphyrin, OH butoxy aza-BODIPY®, OHC12 aza-BODIPY®, Orange Fluorescent Protein, Oregon Green® 488, Oregon Green® 488 DHPE, Oregon Green® 514, Oxazin1, Oxazin 750, Oxazine 1, Oxazine 170, P4-3, P-Quaterphenyl, P-Terphenyl, PA-GFP (post-activation), PA-GFP (pre-activation), Pacific Orange, Palladium(II) meso-tetraphenyl-tetrabenzoporphyrin, PdOEPK, PdTFPP, PerCP-Cy5.5, Perylene, Perylene bisimide pH-Probe 550-5.0, Perylene bisimide pH-Probe 550-5.5, Perylene bisimide pH-Probe 550-6.5, Perylene Green pH-Probe 720-5.5, Perylene Green Tag pH-Probe 720-6.0, Perylene Orange pH-Probe 550-2.0, Perylene Orange Tag 550, Perylene Red pH-Probe 600-5.5, Perylene diimide, Perylne Green pH-Probe 740-5.5, Phenol, Phenylalanine, pHrodo™ succinimidyl ester, Phthalocyanine, PicoGreen® dsDNA quantitation reagent, Pinacyanol-Iodide, Piroxicam, Platinum(II) tetraphenyltetrabenzoporphyrin, Plum Purple, PO-PRO™-1, PO-PRO™-3, POPO™-1, POPO™-3, POPOP, Porphin, PPO, Proflavin, PromoFluor-350, PromoFluor-405, PromoFluor-415, PromoFluor-488, PromoFluor-488LSS, PromoFluor-500LSS, PromoFluor-505, PromoFluor-510LSS, PromoFluor-514LSS, PromoFluor-520LSS, PromoFluor-532, PromoFluor-546, PromoFluor-555, PromoFluor-590, PromoFluor-610, PromoFluor-633, PromoFluor-647, PromoFluor-670, PromoFluor-680, PromoFluor-700, PromoFluor-750, PromoFluor-770, PromoFluor-780, PromoFluor-840, propidium iodide, Protoporphyrin IX, PTIR475/UF, PTIR545/UF, PtOEP, PtOEPK, PtTFPP, Pyrene, QD525, QD565, QD585, QD605, QD655, QD705, QD800, QD903, QD PbS 950, QDot™ 525, QDot™ 545, QDot™ 565, QDot™ 585, QDot™ 605, QDot™ 625, QDot™ 655, QDot™ 705, QDot™ 800, QpyMe2, QSY™ 7 QSY™ 9, QSY™ 21, QSY™ 35, quinine, Quinine Sulfate, R-phycoerythrin, ReAsH-CCPGCC, ReAsH—CCXXCC, Red Beads (Weiss), Redmond Red, Resorufin, rhod-2, Rhodamine 700 perchlorate, rhodamine, Rhodamine 6G, Rhodamine 101, rhodamine 110, Rhodamine 123, Rhodamine B, Rhodamine Green, Rhodamine pH-Probe 585-7.0, Rhodamine pH-Probe 585-7.5, Rhodamine phalloidin, Rhodamine Red-X, Rhodamine Tag pH-Probe 585-7.0, Rhodol Green, Riboflavin, Rose Bengal, Sapphire, SBFI, SBFI Zero Na, Scenedesmus sp., SensiLight PBXL-1, SensiLight PBXL-3, Seta 633-NHS, Seta-633-NHS, SeTau-380-NHS, SeTau-647-NHS, Snake-Eye Red 900, SNIR1, SNIR2, SNIR3, SNIR4, Sodium Green, Solophenyl flavine 7GFE 500, SpectrumAqua™, SpectrumBlue™, Spectrum FRed, SpectrumGold™, SpectrumGreen™, SpectrumOrange™, SpectrumRed™, Squarylium dye III, Stains All, Stilbene, Styry18 perchlorate, Sulfo-Cyanine3 carboxylic acid, Sulfo-Cyanine3 NHS ester, Sulfo-Cyanine5 carboxylic acid, Sulforhodamine 101, Sulforhodamine B, Sulforhodamine G, Suncoast Yellow, SuperGlo BFP, SuperGlo GFP, Surf Green EX, SYBR® Gold nucleic acid gel stain, SYBR® Green I, SYPRO® Ruby, SYTO™ 9, SYTO™ 11, SYTO™ 13, SYTO™ 16, SYTO™ 17, SYTO™ 45, SYTO™ 59, SYTO™ 60, SYTO™ 61, SYTO™ 62, SYTO™ 82, SYTO™ RNASelect, SYTO™ RNASelect, SYTOX Blue, SYTOX Green, SYTOX Orange, SYTOX Red, T-Sapphire, Tb (Soini), tCO, tdTomato, Terrylene, Terrylendiimide, Tetra-t-Butylazaporphine, Tetra-t-Butylnaphthalocyanine, Tetracene, Tetrakis(o-Aminophenyl)Porphyrin, Tetramesitylporphyrin, Tetramethylrhodamine, Tetraphenylporphyrin, Texas Red, Texas Red DHPE, Texas Red-X, ThiolTracker Violet, Thionin acetate, TMRE, TO-PRO™-1, TO-PRO™-3, Toluene, Topaz (Tsien1998), TOTO™-1, TOTO™-3, Tris(2,2-Bipyridyl)Ruthenium(II) chloride, Tris(4,4-diphenyl-2,2-bipyridine) ruthenium(II) chloride, Tris(4,7-diphenyl-1,10-phenanthroline) ruthenium(II) TMS, TRITC (Weiss), TRITC Dextran (Weiss), Tryptophan, Tyrosine, Vex1, Vybrant DyeCycle Green stain, Vybrant™ DyeCycle™ Orange stain, Vybrant™ DyeCycle™ Violet stain, WEGFP (post-activation), WellRED D2, WellRED D3, WellRED D4, WtGFP, WtGFP (Tsien1998), X-rhod-1, Yakima Yellow, YFP, YO-PRO™-1, YO-PRO™-3, YOYO™-1, YOYO™-1, YOYO™-1 dsDNA, YOYO™-1 ssDNA, YOYO™-3, Zinc Octaethylporphyrin, Zinc Phthalocyanine, Zinc Tetramesitylporphyrin, Zinc Tetraphenylporphyrin, ZsGreen1, or ZsYellow1. In embodiments, the fluorescent moiety is a moiety of a derivative of one of the fluorescent moieties described immediately above, wherein the derivative differs from one of the fluorescent moieties immediately above by a modification resulting from the conjugation of the fluorescent moiety to the compound described herein. Janelia Fluor® is a registered trademark of Howard Hughes Medical Institute. Cascade Blue®, SYPRO®, and Oregon Green® are registered trademarks of Life Technologies. LysoTracker™, FluoSpheres™, FM™, Fura Red™, LysoSensor®, SYBR®, TO-PRO®, TOTO™, and Marina Blue® are trademarks of Invitrogen. Pacific Blue™, PO—PRO®, POPO®, SYTOX Blue™, BO-PRO™, BOBO™, YO-PRO™ YOYO™ MitoTracker™, PicoGreen®, NeuroTrace™, Fura Red^{TM™}, CellTrace™, CellMask™ LOLO™-1, JOJO™-1, Qdot™, QSY™, CyQUANT™, DyLight® dyes, SYTO™, and SYTOX Blue™ are trademarks of Thermo Fisher. BODIPY® is a registered trademark of Molecular Probes. TAMRA™ is a trademark of Appelera. Chromeo™ is a trademark of Active Motif Chromeon GmbH. CyTRACK Orange™ and DRAQ5™ are trademarks of Biostatus Limited. EvaGreen®, GelGreen®, GelRed®, CF®, and FM™ are trademarks of Biotium. Macrolex® is a trademark of Lanxess. SpectrumFRed™, SpectrumRed™, SpectrumGold™, Spectrum Orange™ SpectrumGreen™, SpectrumAqua™, and SpectrumBlue™ Series Vysis'™ SpectrumFRed™ SpectrumRed™, SpectrumGold™, SpectrumOrange™, SpectrumGreen™, SpectrumAqua™ and SpectrumBlue™ are trademarks of Abbott Molecular Inc. HiLyte™ is a trademark of Anaspec, Inc. TRDye® is a trademark of Li-Cor Biosciences, Inc. Rox™ is a trademark of Applied Biosystems. Atto™ is a trademark of ATTO-TEC GmbH. Cy® is a registered trademark of Cytiva.

In embodiments, the fluorescent moiety has a maximum excitation wavelength between 350-400 nm, between 400-450 nm, between 450-500 nm, between 500-550 nm, between 550-600 nm, between 600-650 nm, between 650-700 nm, 700-750 nm, or between 750-800 nm. In embodiments, the fluorescent moiety has a maximum excitation wavelength of about 325 nm, 343 nm, 350 nm, 353 nm, 359 nm, 360 nm, 395 nm, 400 nm, 401 nm, 402 nm, 403 nm, 425 nm, 434 nm, 440 nm, 466 nm, 480 nm, 485 nm, 489 nm, 490 nm, 492 nm, 493 nm, 494 nm, 495 nm, 496 nm, 498 nm, 499 nm, 500 nm, 502 nm, 503 nm, 505 nm, 517 nm, 518 nm, 520 nm, 525 nm, 528 nm, 530 nm, 531 nm, 535 nm, 542 nm, 544 nm, 547 nm, 550 nm, 553 nm, 554 nm, 558 nm, 560 nm, 561 nm, 562 nm, 565 nm, 567 nm, 570 nm, 572 nm, 579 nm, 581 nm, 589 nm, 590 nm, 591 nm, 593 nm, 596 nm, 610 nm, 631 nm, 632 nm, 638 nm, 650 nm, 652 nm, 654 nm, 663 nm, 675 nm, 680 nm, 692 nm, 696 nm, 743 nm, 752 nm, 777 nm, or 782 nm.

In embodiments, the fluorescent moiety has a maximum emission wavelength between 400-450 nm, between 450-500 nm, between 500-550 nm, between 550-600 nm, between 600-650 nm, between 650-700 nm, between 700-750 nm, between 750-800 nm, or between 800-850 nm. In embodiments, the fluorescent moiety has a maximum emission of about 410 nm, 420 nm, 421 nm, 423 nm, 432 nm, 442 nm, 445 nm, 455 nm, 506 nm, 512 nm, 514 nm, 517 nm, 518 nm, 519 nm, 520 nm, 521 nm, 523 nm, 525 nm, 528 nm, 533 nm, 537 nm, 539 nm, 540 nm, 542 nm, 548 nm, 550 nm, 551 nm, 554 nm, 555 nm, 556 nm, 565 nm, 568 nm, 570 nm, 572 nm, 573 nm, 574 nm, 575 nm, 576 nm, 578 nm, 580 nm, 590 nm, 591 nm, 594 nm, 595 nm, 596 nm, 603 nm, 605 nm, 613 nm, 615 nm, 617 nm, 618 nm, 619 nm, 620 nm, 629 nm, 630 nm, 640 nm, 647 nm, 648 nm, 658 nm, 660 nm, 668 nm, 670 nm, 673 nm, 675 nm, 691 nm, 694 nm, 695 nm, 702 nm, 712 nm, 719 nm, 767 nm, 776 nm, 778 nm, 794 nm, or 804 nm. In embodiments, the fluorescent moiety has an maximum emission in the near-infrared range.

In embodiments, R³ is a bioconjugate reactive moiety. In embodiments, R³ is —N₃, avidin, streptavidin, —NH₂, —CN, —COOH,

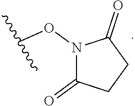 [SMILES: *ON1C(=O)CCC1=O]

In embodiments, R³ is —N₃. In embodiments, R³ is avidin. In embodiments, R³ is streptavidin. In embodiments, R³ is —NH₂. In embodiments, R³ is —CN. In embodiments, R³ is —COOH. In embodiments, R³ is

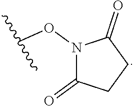 [SMILES: *ON1C(=O)CCC1=O]

In embodiments, R³ is

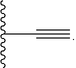 [SMILES: *C#C]

In embodiments, R³ is

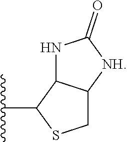 [SMILES: *C1SCC2NC(=O)NC21]

In embodiments, R³ includes
“\*MERGEFORMAT\* MERGEFORMAT —N₃, avidin, streptavidin, —NH₂, —CN, —COOH,

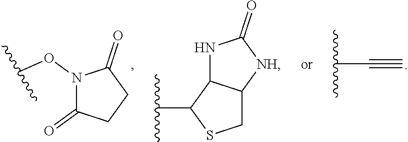

In embodiments, R³ is a bioconjugate reactive moiety. In embodiments, R³ is an activated ester group. In embodiments, R³ is an acrylamide group. In embodiments, R³ is an azide group. In embodiments, R³ is an acyl azide group. In embodiments, R³ is an acyl halide group. In embodiments, R³ is an aryl halide group. In embodiments, R³ is a silyl halide group. In embodiments, R³ is an acyl nitrile group. In embodiments, R³ is an aldehyde group. In embodiments, R³ is a ketone group. In embodiments, R³ is an alkyl sulfonate group. In embodiments, R³ is an anhydride group. In embodiments, R³ is an aziridine group. In embodiments, R³ is a boronate group. In embodiments, R³ is a carbodiimide group. In embodiments, R³ is a diazoalkane group. In embodiments, R³ is an epoxide group. In embodiments, R³ is a haloacetamide group. In embodiments, R³ is a haloplatinate group. In embodiments, R³ is a halotriazine group. In embodiments, R³ is an imido ester group. In embodiments, R³ is an isocyanate group. In embodiments, R³ is an isothiocyanate group. In embodiments, R³ is a maleimide group. In embodiments, R³ is a phosphoramidite group. In embodiments, R³ is a sulfonate ester group. In embodiments, R³ is a sulfonyl halide group. In embodiments, R³ is an alcohol group. In embodiments, R³ is a phenol group. In embodiments, R³ is a hydrazine group. In embodiments, R³ is a hydroxylamine group. In embodiments, R³ is a glycol group. In embodiments, R³ is heterocycle group. In embodiments, R³ is a thiol group. In embodiments, R³ is a carboxylic acid group. In embodiments, R³ is an amine group. In embodiments, R³ is an aniline group.

In embodiments, R³ is isothiocyanate, isocyanate, sulfonyl chloride, aldehyde, acyl azide, anhydride, azide, fluorobenzene, carbonate, N-Hydroxysuccinimide-ester (NHS-ester), imidoester, epoxide, maleimide, —COOH, —NH₂, or fluorophenylester. In embodiments, R³ is

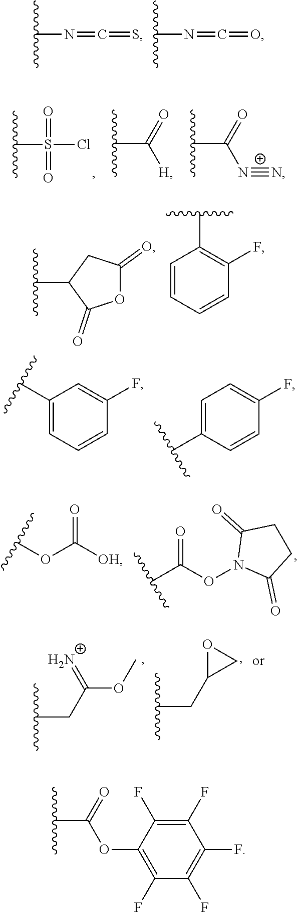

In embodiments, W¹, W², and W³ are each —O—. In embodiments, W¹, W², and W³ are each —S—. In embodiments, W¹, W², and W³ are each —NH—. In embodiments, W¹, W², and W³ are each a bond.

In embodiments, W¹ is —O—. In embodiments, W¹ is —S—. In embodiments, W¹ is —NH—. In embodiments, W¹ is —NR^{1A}—, wherein R^{1A} is hydrogen. In embodiments, W¹ is —NR^{1A}—, wherein R^{1A} is substituted alkyl. In embodiments, W¹ is —NR¹A-, wherein R^{1A} is unsubstituted alkyl.

In embodiments, W² is —O—. In embodiments, W² is —S—. In embodiments, W² is —NH—. In embodiments, W² is —NR²A-, wherein R^{2A} is hydrogen. In embodiments, W² is —NR²A-, wherein R^{2A} is substituted alkyl. In embodiments, W² is —NR²A-, wherein R^{1A} is unsubstituted alkyl.

In embodiments, W³ is —O—. In embodiments, W³ is —S—. In embodiments, W³ is —NH—. In embodiments, W³ is —NR³A-, wherein R^{3A} is hydrogen. In embodiments, W³ is —NR³—, wherein R^{3A} is substituted alkyl. In embodiments, W³ is —NR^{3A}, wherein R^{3A} is unsubstituted alkyl.

In embodiments, W³ is —NH—, and L³-R³ is

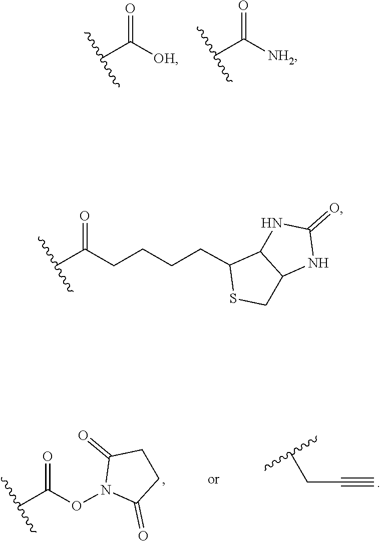

In embodiments, W³ is —NH—, and L³-R³ is

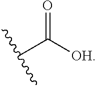 [SMILES: *C(=O)O]

In embodiments, W³ is —NH—, and L³-R³ is

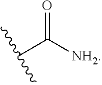 [SMILES: *C(N)=O]

In embodiments, W³ is
“\*MERGEFORMAT\* MERGEFORMAT —NH—, and L³-R³ is

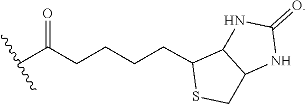 [SMILES: *C(=O)CCCCC1SCC2NC(=O)NC21]

In embodiments, W³ is —NH—, and L³-R³ is

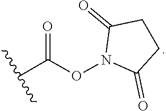 [SMILES: *C(=O)ON1C(=O)CCC1=O]

In embodiments, W³ is —NH—, and L³-R³ includes a biotin moiety. In embodiments, W³ is —NH—, and L³-R³ includes a biotin moiety, wherein the biotin moiety has the formula,

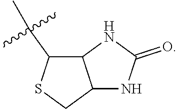 [SMILES: *C1SCC2NC(=O)NC21]

In embodiments, W³ is —NH—, and L³-R³ includes a streptavidin moiety. In embodiments, W³ is —NH—, and L³-R³ includes an avidin moiety. In embodiments, W³ is —NH—, and L³-R³ includes a streptavidin moiety and a biotin moiety. In embodiments, W³ is
“\*MERGEFORMAT\* MERGEFORMAT —NH—, and L³-R³ includes an avidin moiety and a biotin moiety. In embodiments, the avidin moiety further includes a fluorescent moiety. In embodiments, the streptavidin moiety further includes a fluorescent moiety. In embodiments, W³ is —NH—, and L³-R³ is

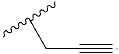 [SMILES: *CC#C]

In embodiments, R^{1A} is hydrogen or substituted or unsubstituted alkyl (e.g., C₁-C₂₀, C₁₀-C₂₀, C₁-C₈, C₁-C₆, or C₁-C₄). In embodiments, R^{1A} is hydrogen. In embodiments, R^{1A} is a substituted alkyl. In embodiments, R^{1A} is an unsubstituted alkyl. In embodiments, R^{1A} is R^{1.1A}-substituted or unsubstituted alkyl (e.g., C₁-C₂₀, C₁-C₂₀, C₁-C₈, C₁-C₆, or C₁-C₄).

R^{1.1A} is independently oxo, halogen, —CCl₃, —CBr₃, —CF₃, —CI₃, —CHCl₂, —CHBr₂, —CHF₂, —CHI₂, —CH₂Cl, —CH₂Br, —CH₂F, —CH₂I, —CN, —OH, —NH₂, —COOH, —CONH₂, —NO₂, —SH, —SO₃H,
“\*MERGEFORMAT\* MERGEFORMAT —SO₄H, —SO₂NH₂, —NHNH₂, —ONH₂, —NHC(O)NHNH₂, —NHC(O)NH₂, —NHSO₂H, —NHC(O)H,
“\*MERGEFORMAT\* MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
“\*MERGEFORMAT\* MERGEFORMAT —OCH₂C₁, —OCH₂Br, —OCH₂I, —OCH₂F, —N₃, —SF₅, unsubstituted alkyl (e.g., C₁-C₂₀, C₁₀-C₂₀,
“\*MERGEFORMAT\* MERGEFORMAT C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, R^{1A} is substituted C₁-C₂₀ alkyl. In embodiments, R^{1A} is substituted C₁₀-C₂₀ alkyl. In embodiments, R^{1A} is substituted C₁-C₈ alkyl. In embodiments, R^{1A} is substituted C₁-C₆ alkyl. In embodiments, R^{1A} is substituted C₁-C₄ alkyl. In embodiments, R^{1A} is unsubstituted C₁-C₂₀ alkyl. In embodiments, R^{1A} is unsubstituted C₁₀-C₂₀ alkyl. In embodiments, R^{1A} is unsubstituted C₁-C₈ alkyl. In embodiments, R^{1A} is unsubstituted C₁-C₆ alkyl. In embodiments, R^{1A} is unsubstituted C₁-C₄ alkyl.

In embodiments, R^{1A} is R^{1A}-substituted C₁-C₂₀ alkyl. In embodiments, R^{1A} is R^{1.A}-substituted C₁₀-C₂₀ alkyl. In embodiments, R^{1A} is R^{1.A}-substituted C₁-C₈ alkyl. In embodiments, R^{1A} is R^{11A}-substituted C₁-C₆ alkyl. In embodiments, R^{1A} is R^{1.1A}-substituted C≡C₄ alkyl.

In embodiments, R^{2A} is hydrogen or substituted or unsubstituted alkyl (e.g., C₁-C₂₀, C₁₀-C₂₀, C₁-C₈, C₁-C₆, or C₁-C₄). In embodiments, R^{2A} is hydrogen. In embodiments, R^{2A} is a substituted alkyl. In embodiments, R^{2A} is an unsubstituted alkyl. In embodiments, R^{2A} is R^{2.1A}-substituted or unsubstituted alkyl (e.g., C₁-C₂₀, C₁₀-C₂₀, C₁-C₈, C₁-C₆, or C₁-C₄).

R^{2.1A} is independently oxo, halogen, —CCl₃, —CBr₃, —CF₃, —CI₃, —CHCl₂, —CHBr₂, —CHF₂, —CHI₂, —CH₂C₁, —CH₂Br, —CH₂F, —CH₂I, —CN, —OH, —NH₂, —COOH, —CONH₂, —NO₂, —SH, —SO₃H,
“\*MERGEFORMAT\* MERGEFORMAT —SO₄H, —SO₂NH₂, —NHNH₂, —ONH₂, —NHC(O)NHNH₂, —NHC(O)NH₂, —NHSO₂H, —NHC(O)H,
“\*MERGEFORMAT\* MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
“\*MERGEFORMAT\* MERGEFORMAT —OCH₂C₁, —OCH₂Br, —OCH₂I, —OCH₂F, —N₃, —SF₅, unsubstituted alkyl (e.g., C₁-C₂₀, C₁₀-C₂₀,
“\*MERGEFORMAT\* MERGEFORMAT C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, R^{2A} is substituted C₁-C₂₀ alkyl. In embodiments, R^{2A} is substituted C₁₀-C₂₀ alkyl. In embodiments, R^{2A} is substituted C₁-C₈ alkyl. In embodiments, R^{2A} is substituted C₁-C₆ alkyl. In embodiments, R^{2A} is substituted C₁-C₄ alkyl. In embodiments, R^{2A} is unsubstituted C₁-C₂₀ alkyl. In embodiments, R^{2A} is unsubstituted C₁₀-C₂₀ alkyl. In embodiments, R^{2A} is unsubstituted C₁-C₈ alkyl. In embodiments, R^{2A} is unsubstituted C₁-C₆ alkyl. In embodiments, R^{2A} is unsubstituted C₁-C₄ alkyl.

In embodiments, R^{2A} is R^{21A}-substituted C₁-C₂₀ alkyl. In embodiments, R^{2A} is R^{21A}-substituted C₁₀-C₂₀ alkyl. In embodiments, R^{2A} is R^{21A}-substituted C₁-C₈ alkyl. In embodiments, R^{2A} is R^{2.1A}-substituted C₁-C₆ alkyl. In embodiments, R^{2A} is R^{2.1A}-substituted C₁-C₄ alkyl.

In embodiments, R^{3A} is hydrogen or substituted or unsubstituted alkyl (e.g., C₁-C₂₀, C₁₀-C₂₀, C₁-C₈, C₁-C₆, or C₁-C₄). In embodiments, R^{3A} is hydrogen. In embodiments, R^{3A} is a substituted alkyl. In embodiments, R^{3A} is an unsubstituted alkyl. In embodiments, R^{3A} is R^{3.1A}-substituted or unsubstituted alkyl (e.g., C₁-C₂₀, C₁₀-C₂₀, C₁-C₈, C₁-C₆, or C₁-C₄).

R^{3.1A} is independently oxo, halogen, —CCl₃, —CBr₃, —CF₃, —CI₃, —CHCl₂, —CHBr₂, —CHF₂, —CHI₂, —CH₂C₁, —CH₂Br, —CH₂F, —CH₂I, —CN, —OH, —NH₂, —COOH, —CONH₂, —NO₂, —SH, —SO₃H,
“\*MERGEFORMAT\* MERGEFORMAT —SO₄H, —SO₂NH₂, —NHNH₂, —ONH₂, —NHC(O)NHNH₂, —NHC(O)NH₂, —NHSO₂H, —NHC(O)H,
“\*MERGEFORMAT\* MERGEFORMAT —NHC(O)OH, —NHOH, —OCCl₃, —OCF₃, —OCBr₃, —OCI₃, —OCHCl₂, —OCHBr₂, —OCHI₂, —OCHF₂,
“\*MERGEFORMAT\* MERGEFORMAT —OCH₂Cl, —OCH₂Br, —OCH₂I, —OCH₂F, —N₃, —SF₅, unsubstituted alkyl (e.g., C₁-C₂₀, C₁₀-C₂₀,
“\*MERGEFORMAT\* MERGEFORMAT C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, R^{3A} is substituted C₁-C₂₀ alkyl. In embodiments, R^{3A} is substituted C₁₀-C₂₀ alkyl. In embodiments, R^{3A} is substituted C₁-C₈ alkyl. In embodiments, R^{3A} is substituted C₁-C₆ alkyl. In embodiments, R^{3A} is substituted C₁-C₄ alkyl. In embodiments, R^{3A} is unsubstituted C₁-C₂₀ alkyl. In embodiments, R^{3A} is unsubstituted C₁₀-C₂₀ alkyl. In embodiments, R^{3A} is unsubstituted C₁-C₈ alkyl. In embodiments, R^{3A} is unsubstituted C₁-C₆ alkyl. In embodiments, R^{3A} is unsubstituted C₁-C₄ alkyl.

In embodiments, R^{3A} is R^{3.1}A-substituted C₁-C₂₀ alkyl. In embodiments, R^{3A} is R^{3.1}A-substituted C₀-C₂₀ alkyl. In embodiments, R^{3A} is R³0.1A-substituted C₁-C₈ alkyl. In embodiments, R^{3A} is R^{3.1A}-substituted C₁-C₆ alkyl. In embodiments, R^{3A} is R^{3.1}A-substituted C₁-C₄ alkyl.

In embodiments, L¹ is a covalent linker. In embodiments, L² is a covalent linker. In embodiments, L³ is a covalent linker. In embodiments, L¹ is a bond. In embodiments, L² is a bond. In embodiments, L³ is a bond.

In embodiments, L¹ is L¹⁰¹-L¹⁰²-L¹⁰³. L¹⁰¹, L¹⁰², and L¹⁰³ are independently a bond,
“\*MERGEFORMAT\* MERGEFORMAT —C(O)O—, —NHC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L¹ is L¹⁰¹-L¹⁰²-L¹⁰³. L¹⁰¹ is a bond, —C(O)O—, —NHC(O)—, —C(O)NH—, R¹⁰¹-substituted or unsubstituted alkylene, R¹⁰¹-substituted or unsubstituted heteroalkylene,
“\*MERGEFORMAT\* MERGEFORMAT R¹⁰¹-substituted or unsubstituted cycloalkylene, R¹⁰¹-substituted or unsubstituted heterocycloalkylene, R¹⁰¹-substituted or unsubstituted arylene, or R¹⁰¹-substituted or unsubstituted heteroarylene. L¹⁰² is a bond, —C(O)O—, —NHC(O)—, —C(O)NH—, R¹⁰²-substituted or unsubstituted alkylene, R¹⁰²-substituted or unsubstituted heteroalkylene, R¹⁰²-substituted or unsubstituted cycloalkylene, R¹⁰²-substituted or unsubstituted heterocycloalkylene, R¹⁰²-substituted or unsubstituted arylene, or R¹⁰²-substituted or unsubstituted heteroarylene. L¹⁰³ is a bond, —C(O)O—, —NHC(O)—, —C(O)NH—, R¹⁰³-substituted or unsubstituted alkylene,
“\*MERGEFORMAT\* MERGEFORMAT R¹⁰³-substituted or unsubstituted heteroalkylene, R¹⁰³-substituted or unsubstituted cycloalkylene, R¹⁰³-substituted or unsubstituted heterocycloalkylene, R¹⁰³-substituted or unsubstituted arylene, or R¹⁰³-substituted or unsubstituted heteroarylene.

R¹⁰¹, R¹⁰², and R¹⁰³ are independently oxo, halogen, —CCl₃, —CBr₃, —CF₃, —CI₃, —CHCl₂, —CHBr₂, —CHF₂, —CHI₂, —CH₂C₁, —CH₂Br, —CH₂F, —CH₂I, —CN, —OH, —NH₂, —COOH, —CONH₂,
“\*MERGEFORMAT\* MERGEFORMAT —NO₂, —SH, —SO₃H, —SO₄H, —SO₂NH₂, —NHNH₂, —ONH₂, —NHC(O)NHNH₂, —NHC(O)NH₂,
“\*MERGEFORMAT\* MERGEFORMAT —NHSO₂H, —NHC(O)H, —NHC(O)OH, —NHOH, —OCCl₃, —OCF₃, —OCBr₃, —OCI₃, —OCHCl₂,
“\*MERGEFORMAT\* MERGEFORMAT —OCHBr₂, —OCHI₂, —OCHF₂, —OCH₂Cl, —OCH₂Br, —OCH₂I, —OCH₂F, —N₃, —SF₅, —NH₃⁺, —SO₃,
“\*MERGEFORMAT\* MERGEFORMAT —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L¹ is L¹⁰¹-L¹⁰²-L¹⁰³. L¹⁰¹, L¹⁰², and L¹⁰³ are independently a bond, —NH—, —S—, —O—, —C(O)—, —C(O)O—, —OC(O)—, —NHC(O)—, —C(O)NH—, —NHC(O)NH—, “\*MERGEFORMAT\* MERGEFORMAT —NHC(NH)NH—, —C(S)—, —N═N—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered). In embodiments, L¹⁰¹, L¹⁰², and L¹⁰³ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L¹⁰¹, L¹⁰², and L¹⁰³ independently include PEG. In embodiments, L¹⁰¹, L¹⁰², and L¹⁰³ independently are a cleavable linker (e.g., include one or more cleavable sites).

In embodiments, L¹ is

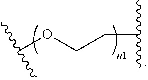

In embodiments, L¹ is

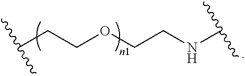

In embodiments, L¹ is

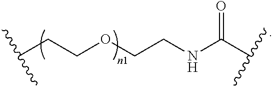

In embodiments, L¹ is

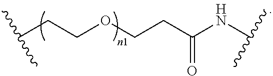

In embodiments, n1 is an integer from 1 to 10. In embodiments, n1 is 1, 2, 3, 4, 5, 6, 7, 8, 9, or 10.

In embodiments, —W₁-L₁ is

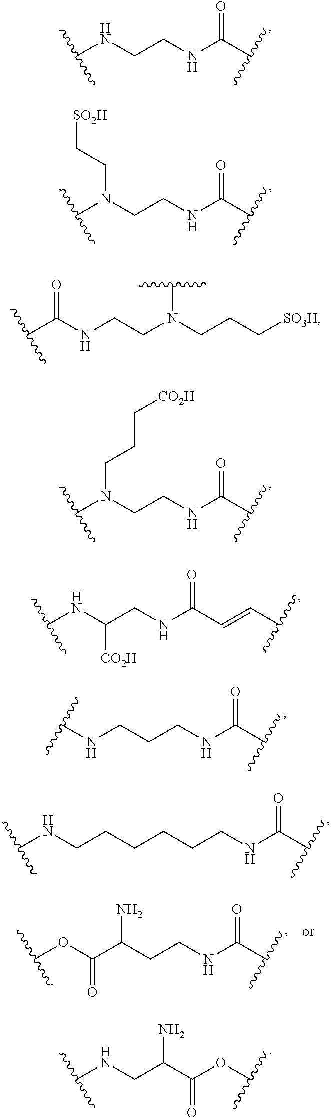

In embodiments, L² is L²⁰¹-L²⁰²-L²⁰³. L²⁰¹, L²⁰², and L²⁰³ are independently a bond,
“\*MERGEFORMAT\* MERGEFORMAT —C(O)O—, —NHC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L² is L²⁰¹-L²⁰²-L²⁰³. L²⁰¹ is a bond, —C(O)O—, —NHC(O)—, —C(O)NH—, R²⁰¹-substituted or unsubstituted alkylene, R²⁰¹-substituted or unsubstituted heteroalkylene, R²⁰¹-substituted or unsubstituted cycloalkylene, R²⁰¹-substituted or unsubstituted heterocycloalkylene, R²⁰¹-substituted or unsubstituted arylene, or R²⁰¹-substituted or unsubstituted heteroarylene. L²⁰² is a bond, —C(O)O—, —NHC(O)—, —C(O)NH—, R²⁰²-substituted or unsubstituted alkylene, R²⁰²-substituted or unsubstituted heteroalkylene, R²⁰²-substituted or unsubstituted cycloalkylene, R²⁰²-substituted or unsubstituted heterocycloalkylene, R²⁰²-substituted or unsubstituted arylene, or R²⁰²-substituted or unsubstituted heteroarylene. L²⁰³ is a bond, —C(O)O—, —NHC(O)—, —C(O)NH—, R²⁰³-substituted or unsubstituted alkylene, R²⁰³-substituted or unsubstituted heteroalkylene, R²⁰³-substituted or unsubstituted cycloalkylene, R²⁰³—substituted or unsubstituted heterocycloalkylene, R²⁰³-substituted or unsubstituted arylene, or R²⁰³-substituted or unsubstituted heteroarylene.

R²⁰¹, R²⁰², and R²⁰³ are independently oxo, halogen, —CCl₃, —CBr₃, —CF₃, —CI₃, —CHCl₂,
“\*MERGEFORMAT\* MERGEFORMAT —CHBr₂, —CHF₂, —CHI₂, —CH₂Cl, —CH₂Br, —CH₂F, —CH₂I, —CN, —OH, —NH₂, —COOH, —CONH₂,
“\*MERGEFORMAT\* MERGEFORMAT —NO₂, —SH, —SO₃H, —SO₄H, —SO₂NH₂, —NHNH₂, —ONH₂, —NHC(O)NHNH₂, —NHC(O)NH₂,
“\*MERGEFORMAT\* MERGEFORMAT —NHSO₂H, —NHC(O)H, —NHC(O)OH, —NHOH, —OCCl₃, —OCF₃, —OCBr₃, —OCI₃, —OCHCl₂,
“\*MERGEFORMAT\* MERGEFORMAT —OCHBr₂, —OCHI₂, —OCHF₂, —OCH₂C₁, —OCH₂Br, —OCH₂I, —OCH₂F, —N₃, —SF₅, —NH₃+, —SO₃,
“\*MERGEFORMAT\* MERGEFORMAT —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L² is L²⁰¹-L²⁰²-L²⁰³, and L²⁰¹-L²⁰²-L²⁰³ are independently a bond, —NH—, —S—, —O—, —C(O)—, —C(O)O—, —OC(O)—, —NHC(O)—, —C(O)NH—, —NHC(O)NH—,
“\*MERGEFORMAT\* MERGEFORMAT —NHC(NH)NH—, —C(S)—, —N═N—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered). In embodiments, L²⁰¹, L²⁰², and L²⁰³ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L²⁰¹, L²⁰², and L²⁰³ are independently include PEG.

In embodiments, L² is

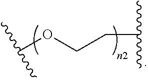

In embodiments, L² is

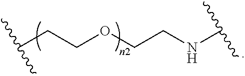

In embodiments, L² is

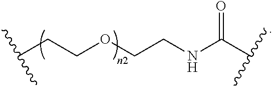

In embodiments, L² is

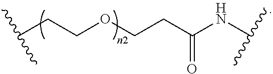

In embodiments, n2 is an integer from 1 to 10. In embodiments, n2 is 1, 2, 3, 4, 5, 6, 7, 8, 9, or 10.

In embodiments, —W²-L² is

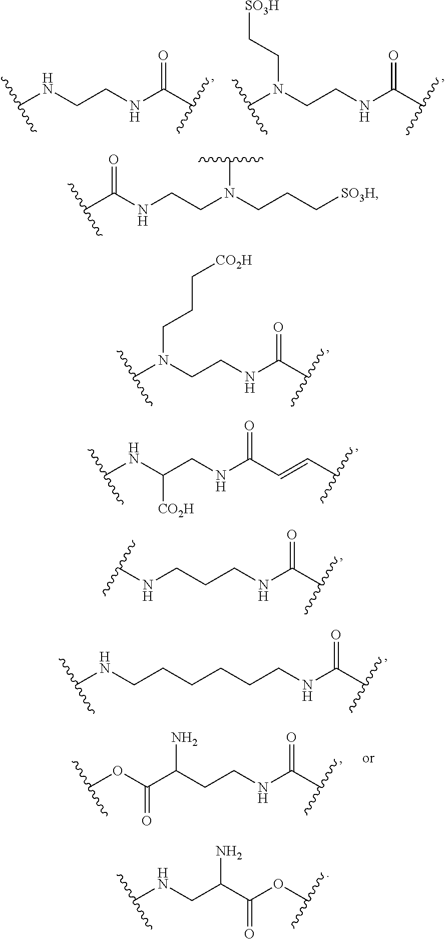

In embodiments, L³ is L³⁰¹-L³⁰²-L³⁰³. L³⁰¹, L³⁰², and L³⁰³ are independently a bond,
“\*MERGEFORMAT\* MERGEFORMAT —C(O)O—, —NHC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

In embodiments, L³ is L³⁰¹-L³⁰²-L³⁰³. L³⁰¹ is a bond, —C(O)O—, —NHC(O)—, —C(O)NH—, R³⁰¹-substituted or unsubstituted alkylene, R³⁰¹-substituted or unsubstituted heteroalkylene, R³⁰¹-substituted or unsubstituted cycloalkylene, R³⁰¹-substituted or unsubstituted heterocycloalkylene, R³⁰¹-substituted or unsubstituted arylene, or R³⁰¹-substituted or unsubstituted heteroarylene. L³⁰² is a bond, —C(O)O—, —NHC(O)—, —C(O)NH—, R³⁰²-substituted or unsubstituted alkylene, R³⁰²-substituted or unsubstituted heteroalkylene, R³⁰²-substituted or unsubstituted cycloalkylene, R³⁰²-substituted or unsubstituted heterocycloalkylene, R³⁰²-substituted or unsubstituted arylene, or R³⁰²-substituted or unsubstituted heteroarylene. L³⁰³ is a bond, —C(O)O—, —NHC(O)—, —C(O)NH—, R³⁰³-substituted or unsubstituted alkylene, R³⁰³-substituted or unsubstituted heteroalkylene, R³⁰³-substituted or unsubstituted cycloalkylene, R³⁰³-substituted or unsubstituted heterocycloalkylene, R³⁰³-substituted or unsubstituted arylene, or R³⁰³-substituted or unsubstituted heteroarylene.

R³⁰¹, R³⁰², and R³⁰³ are independently oxo, halogen, —CCl₃, —CBr₃, —CF₃, —CI₃, —CHCl₂,
“\*MERGEFORMAT\* MERGEFORMAT —CHBr₂, —CHF₂, —CHI₂, —CH₂Cl, —CH₂Br, —CH₂F, —CH₂I, —CN, —OH, —NH₂, —COOH, —CONH₂,
“\*MERGEFORMAT\* MERGEFORMAT —NO₂, —SH, —SO₃H, —SO₄H, —SO₂NH₂, —NHNH₂, —ONH₂, —NHC(O)NHNH₂, —NHC(O)NH₂,
“\*MERGEFORMAT\* MERGEFORMAT —NHSO₂H, —NHC(O)H, —NHC(O)OH, —NHOH, —OCCl₃, —OCF₃, —OCBr₃, —OCI₃, —OCHCl₂,
“\*MERGEFORMAT\* MERGEFORMAT —OCHBr₂, —OCHI₂, —OCHF₂, —OCH₂Cl, —OCH₂Br, —OCH₂I, —OCH₂F, —N₃, —SF₅, —NH₃⁺, —SO₃,
“\*MERGEFORMAT\* MERGEFORMAT —OPO₃H, —SCN, —ONO₂, unsubstituted alkyl (e.g., C₁-C₂₀, C₁₀-C₂₀, C₁-C₈, C₁-C₆, or C₁-C₄), unsubstituted heteroalkyl (e.g., 2 to 20, 8 to 20, 2 to 10, 2 to 8, 2 to 6, or 2 to 4 membered), unsubstituted cycloalkyl (e.g., C₃-C₈, C₃-C₆, or C₅-C₆), unsubstituted heterocycloalkyl (e.g., 3 to 8, 3 to 6, or 5 to 6 membered), unsubstituted aryl (e.g., C₆-C₁₀, C₁₀, or phenyl), or unsubstituted heteroaryl (e.g., 5 to 10, 5 to 9, or 5 to 6 membered).

In embodiments, L³ is L³⁰¹-L³⁰²-L³⁰³. L³⁰¹, L³⁰², and L³⁰³ are independently a bond, —NH—, —S—, —O—, —C(O)—, —C(O)O—, —OC(O)—, —NHC(O)—, —C(O)NH—, —NHC(O)NH—,
“\*MERGEFORMAT\* MERGEFORMAT —NHC(NH)NH—, —C(S)—, —N═N—, substituted or unsubstituted alkylene (e.g., C₁-C₂₀, C₁₀-C₂₀, C₁-C₈, C₁-C₆, or C₁-C₄), substituted or unsubstituted heteroalkylene (e.g., 2 to 20, 8 to 20, 2 to 10, 2 to 8, 2 to 6, or 2 to 4 membered), substituted or unsubstituted cycloalkylene (e.g., C₃-C₈, C₃-C₆, or C₅-C₆), substituted or unsubstituted heterocycloalkylene (e.g., 3 to 8, 3 to 6, or 5 to 6 membered), substituted or unsubstituted arylene (e.g., C₆-C₁₀, C₁₀, or phenylene), or substituted or unsubstituted heteroarylene (e.g., 5 to 10, 5 to 9, or 5 to 6 membered). In embodiments, L³⁰¹, L³⁰², and L³⁰³ are independently a bond, —NH—, —S—, —O—, —C(O)—, —C(O)O—, —OC(O)—, —NHC(O)—, —C(O)NH—, —NHC(O)NH—, —NHC(NH)NH—, —C(S)—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene. In embodiments, L³⁰¹, L³⁰², and L³⁰³ are independently include PEG.

In embodiments, L³ is

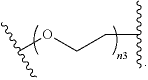

In embodiments, L³ is

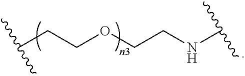

In embodiments, L³ is

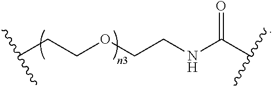

In embodiments, L³ is

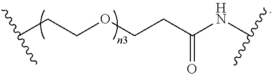

In embodiments, n3 is an integer from 1 to 10. In embodiments, n3 is 1, 2, 3, 4, 5, 6, 7, 8, 9, or 10.

In embodiments, z3 is 0. In embodiments, z3 is 1. In embodiments, z3 is 0 and R³ is
“\*MERGEFORMAT\* MERGEFORMAT —N₃, avidin, streptavidin, —NH₂, —CN,

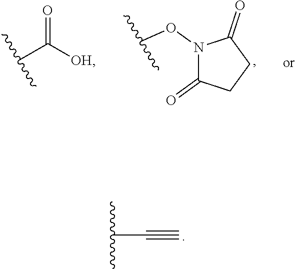

In embodiments, z3 is 0 and R³ is —N₃. In embodiments, z3 is 0 and R³ is avidin. In embodiments, z3 is 0 and R³ is streptavidin. In embodiments, z3 is 0 and R³ is —NH₂. In embodiments, z3 is 0 and R³ is —CN. In embodiments, z3 is 0 and R³ is

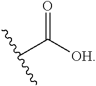 [SMILES: *C(=O)O]

In embodiments, z3 is 0 and R³ is

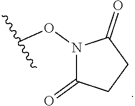 [SMILES: *ON1C(=O)CCC1=O]

In embodiments, z3 is 0 and R³

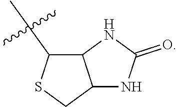 [SMILES: *C1SCC2NC(=O)NC21]

In embodiments, z3 is 0 and R³ is

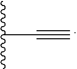 [SMILES: *C#C]

In embodiments, the compound has the formula:

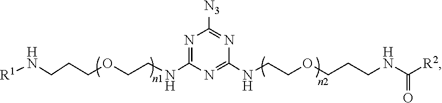

wherein n1 and n2 are independently an integer from 1 to 10. In embodiments, n1 and n2 are independently 1, 2, 3, 4, 5, 6, 7, 8, 9, or 10.

In embodiments, the compound has the formula:

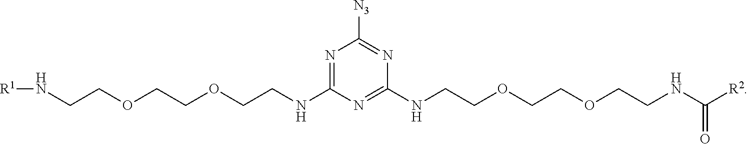

In embodiments, the compound has the formula:

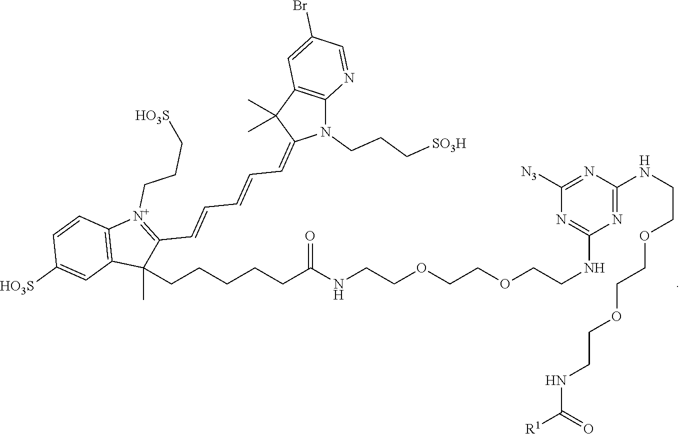

In embodiments, the compound has the formula:

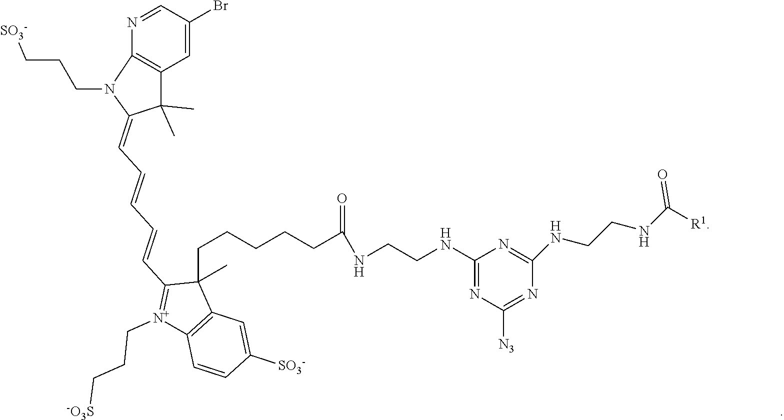

In an aspect is provided a biomolecule attached to a nucleotide construct, wherein said nucleotide construct has the formula:

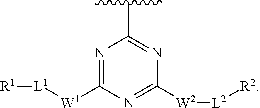

R¹ is a nucleotide moiety. R² is a nucleotide moiety or a fluorescent moiety. W¹ is —O—, —NR^{1A}—, or —S—. W² is —O—, —NR²A-, or —S—. R^{1A} and R^{2A} are independently hydrogen or substituted or unsubstituted alkyl; and L¹ and L² and are independently covalent linkers. In embodiments, the biomolecule is in a cell or tissue.

In embodiments, the biomolecule is avidin. In embodiments, the biomolecule is streptavidin. In embodiments, the biomolecule is Zymax Cy5 Streptavidin (Fisher Scientific, catalog no. 438316).

In embodiments, the biomolecule is azido-cyclodextrin, having the formula:

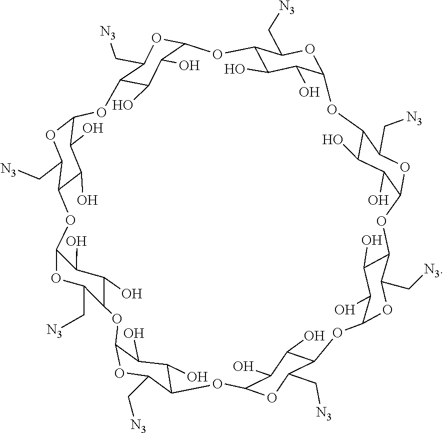 [SMILES: [N-]=[N+]=NCC1OC2OC3C(CN=[N+]=[N-])OC(OC4C(CN=[N+]=[N-])OC(OC5C(CN=[N+]=[N-])OC(OC6C(CN=[N+]=[N-])OC(OC7C(CN=[N+]=[N-])OC(OC8C(CN=[N+]=[N-])OC(OC9C(CN=[N+]=[N-])OC(OC1C(O)C2O)C(O)C9O)C(O)C8O)C(O)C7O)C(O)C6O)C(O)C5O)C(O)C4O)C(O)C3O]

In embodiments, the nucleotide construct is attached to the biomolecule via a bioconjugate linker. In embodiments, the biomolecule is further attached to a fluorescent moiety. In embodiments, the biomolecule is further attached to a plurality of fluorescent moieties.

In embodiments, the bioconjugate linker is formed via a reaction between a first bioconjugate reactive moiety and a second bioconjugate reactive moiety. In embodiments, the bioconjugate linker is the product of a reaction between the two bioconjugate reactive group (e.g., two click chemistry group). In embodiments, the first bioconjugate reactive moiety and the second bioconjugate reactive moiety are independently —N₃, avidin, streptavidin, —NH₂, —CN,
“\*MERGEFORMAT\* MERGEFORMAT —COOH,

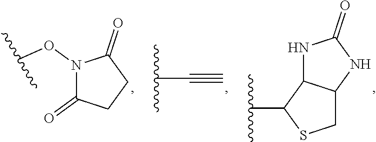

an activated ester group, an acrylamide group, an azide group, an acyl azide group, an acyl halide group, an aryl halide group, a silyl halide group, an acyl nitrile group, an aldehyde group, a ketone group, an alkyl sulfonate group, an anhydride group, an aziridine group, a boronate group, a carbodiimide group, a diazoalkane group, an epoxide group, a haloacetamide group, a haloplatinate group, a halotriazine group, an imido ester group, an isocyanate group, an isothiocyanate group, a maleimide group, a phosphoramidite group, a sulfonate ester group, a sulfonyl halide group, an alcohol group, a phenol group, a hydrazine group, a hydroxylamine group, a glycol group, heterocycle group, a thiol group, a carboxylic acid group, an amine group, or an aniline group. In embodiments, the first bioconjugate reactive moiety and the second bioconjugate reactive moiety are independently an amine moiety, aldehyde moiety, alkyne moiety, azide moiety, carboxylic acid moiety, dibenzocyclooctyne (DBCO) moiety, tetrazine moiety, epoxy moiety, isocyanate moiety, furan moiety, maleimide moiety, thiol moiety, or transcyclooctene (TCO) moiety. In embodiments, the first and the second bioconjugate reactive moieties are different. In embodiments, the first and the second bioconjugate reactive moieties are reactive with each other (e.g., an azide moiety and an DBCO moiety) and form a bioconjugate linker.

In an aspect is provided a nucleic acid polymerase complex. In embodiments, the nucleic acid polymerase complex includes a polymerase, an oligonucleotide, and a biomolecule attached to a nucleotide construct described herein, wherein the polymerase is bound to (i) a nucleotide moiety of the nucleotide construct and (ii) the oligonucleotide. In embodiments, the oligonucleotide is a primer. In embodiments, the biomolecule is avidin or streptavidin. In embodiments, the biomolecule further includes a fluorescent moiety.

For use in the methods and/or applications (e.g., sequencing applications) described herein, kits and articles of manufacture are also provided. In some embodiments, such kits comprise a carrier, package, or container that is compartmentalized to receive one or more containers such as vials, tubes, and the like, each of the container(s) comprising one of the separate elements to be used in a method described herein. Suitable containers include, for example, bottles, vials, syringes, and test tubes. The containers are formed from a variety of materials such as glass or plastic.

In an aspect is provided a kit. In embodiments, the kit includes the reagents and containers useful for performing the methods as described herein. In embodiments, the kit includes the compound as described herein. In embodiments, the kit includes a plurality of the compounds described herein. In embodiments, the kit includes a nucleotide construct as described herein. In embodiments, the kit includes a biomolecule attached to a nucleotide construct as described herein.

In embodiments, the kit includes a solid support. In embodiments, the kit includes a solid support including a cell or tissue immobilized to the surface of the solid support. In embodiments, kit includes a solid support, wherein the solid support includes a functionalized glass surface or a functionalized plastic surface (e.g., a surface including a plurality of reactive moieties).

In embodiments, the kit includes one or more oligonucleotides. The kit may also include a template nucleic acid (DNA and/or RNA), one or more primer polynucleotides, nucleoside triphosphates (including, e.g., deoxyribonucleotides, dideoxynucleotides, ribonucleotides, labeled nucleotides, and/or modified nucleotides), buffers, salts, and/or labels (e.g., fluorophores). In embodiments, the kit includes components useful for circularizing template polynucleotides using a ligation enzyme (e.g., CircLigase™ enzyme, Taq DNA Ligase, HiFi Taq DNA Ligase, T4 ligase, SplintR® ligase, or Ampligase™ DNA Ligase).

In embodiments, kits described herein include a polymerase. In embodiments, the polymerase is a DNA polymerase. In embodiments, the DNA polymerase is a thermophilic nucleic acid polymerase. In embodiments, the DNA polymerase is a modified archaeal DNA polymerase.

In embodiments, the kit includes a buffered solution. In embodiments, the kit includes an amplification solution. In embodiments, the kit includes a sequencing solution. Typically, the buffered solutions contemplated herein are made from a weak acid and its conjugate base or a weak base and its conjugate acid. For example, sodium acetate and acetic acid are buffer agents that can be used to form an acetate buffer. Other examples of buffer agents that can be used to make buffered solutions include, but are not limited to, Tris, bicine, tricine, HEPES, TES, MOPS, MOPSO and PIPES. Additionally, other buffer agents that can be used in enzyme reactions, hybridization reactions, and detection reactions are known in the art. In embodiments, the buffered solution can include Tris. With respect to the embodiments described herein, the pH of the buffered solution can be modulated to permit any of the described reactions. In some embodiments, the buffered solution can have a pH greater than pH 7.0, greater than pH 7.5, greater than pH 8.0, greater than pH 8.5, greater than pH 9.0, greater than pH 9.5, greater than pH 10, greater than pH 10.5, greater than pH 11.0, or greater than pH 11.5. In other embodiments, the buffered solution can have a pH ranging, for example, from about pH 6 to about pH 9, from about pH 8 to about pH 10, or from about pH 7 to about pH 9. In embodiments, the buffered solution can include one or more divalent cations. Examples of divalent cations can include, but are not limited to, Mg2+, Mn2+, Zn2+, and Ca2+. In embodiments, the buffered solution can contain one or more divalent cations at a concentration sufficient to permit hybridization of a nucleic acid. In embodiments, the buffered solution can contain one or more divalent cations at a concentration sufficient to permit hybridization of a nucleic acid. In embodiments, the buffered solution includes about 10 mM Tris, about 20 mM Tris, about 30 mM Tris, about 40 mM Tris, or about 50 mM Tris. In embodiments the buffered solution includes about 50 mM NaCl, about 75 mM NaCl, about 100 mM NaCl, about 125 mM NaCl, about 150 mM NaCl, about 200 mM NaCl, about 300 mM NaCl, about 400 mM NaCl, or about 500 mM NaCl. In embodiments, the buffered solution includes about 0.05 mM EDTA, about 0.1 mM EDTA, about 0.25 mM EDTA, about 0.5 mM EDTA, about 1.0 mM EDTA, about 1.5 mM EDTA or about 2.0 mM EDTA. In embodiments, the buffered solution includes about 0.01% Triton X-100, about 0.025% Triton X-100, about 0.05% Triton X-100, about 0.1% Triton X-100, or about 0.5% Triton X-100. In embodiments, the buffered solution includes 20 mM Tris pH 8.0, 100 mM NaCl, 0.1 mM EDTA, 0.025% Triton X-100. In embodiments, the buffered solution includes 20 mM Tris pH 8.0, 150 mM NaCl, 0.1 mM EDTA, 0.025% Triton X-100. In embodiments, the buffered solution includes 20 mM Tris pH 8.0, 300 mM NaCl, 0.1 mM EDTA, 0.025% Triton X-100. In embodiments, the buffered solution includes 20 mM Tris pH 8.0, 400 mM NaCl, 0.1 mM EDTA, 0.025% Triton X-100. In embodiments, the buffered solution includes 20 mM Tris pH 8.0, 500 mM NaCl, 0.1 mM EDTA, 0.025% Triton X-100. In embodiments, the buffered solution includes about 0.002% Pluronic® F-127, about 0.01% Pluronic® F-127, about 0.02% Pluronic® F-127, about 0.05% Pluronic® F-127, about 0.1% Pluronic® F-127, about 0.2% Pluronic® F-127, about 0.3% Pluronic® F-127, about 0.4% Pluronic® F-127, about 0.5% Pluronic® F-127, about 0.6% Pluronic® F-127, about 0.7% Pluronic® F-127, about 0.8% Pluronic® F-127, about 0.9% Pluronic® F-127, about 1% Pluronic® F-127, about 1.1% Pluronic® F-127, about 1.2% Pluronic® F-127, about 1.3% Pluronic® F-127, about 1.4% Pluronic® F-127, about 1.5% Pluronic® F-127, about 1.6% Pluronic® F-127, about 1.7% Pluronic® F-127, about 1.8% Pluronic® F-127, about 1.9% Pluronic® F-127, or about 2% Pluronic® F-127.

In embodiments, the kit includes one or more sequencing reaction mixtures. In embodiments, the sequencing reaction mixture includes a buffer. In embodiments, the buffer includes an acetate buffer, 3-(N-morpholino)propanesulfonic acid (MOPS) buffer, N-(2-Acetamido)-2-aminoethanesulfonic acid (ACES) buffer, phosphate-buffered saline (PBS) buffer, 4-(2-hydroxyethyl)-1-piperazineethanesulfonic acid (HEPES) buffer, N-(1,1-Dimethyl-2-hydroxyethyl)-3-amino-2-hydroxypropanesulfonic acid (AMPSO) buffer, borate buffer (e.g., borate buffered saline, sodium borate buffer, boric acid buffer), 2-Amino-2-methyl-1,3-propanediol (AMPD) buffer, N-cyclohexyl-2-hydroxyl-3-aminopropanesulfonic acid (CAPSO) buffer, 2-Amino-2-methyl-1-propanol (AMP) buffer, 4-(Cyclohexylamino)-1-butanesulfonic acid (CABS) buffer, glycine-NaOH buffer, N-Cyclohexyl-2-aminoethanesulfonic acid (CHES) buffer, tris(hydroxymethyl)aminomethane (Tris) buffer, or a N-cyclohexyl-3-aminopropanesulfonic acid (CAPS) buffer. In embodiments, the buffer is a borate buffer. In embodiments, the buffer is a CHES buffer. In embodiments, the sequencing reaction mixture includes nucleotides, wherein the nucleotides include a reversible terminating moiety and a label covalently linked to the nucleotide via a cleavable linker. In embodiments, the sequencing reaction mixture includes a buffer, DNA polymerase, detergent (e.g., Triton X), a chelator (e.g., EDTA), and/or salts (e.g., ammonium sulfate, magnesium chloride, sodium chloride, or potassium chloride).

In embodiments, the kit includes a sequencing polymerase, and one or more amplification polymerases. In embodiments, the sequencing polymerase is capable of incorporating modified nucleotides. In embodiments, the polymerase is a DNA polymerase. In embodiments, the DNA polymerase is a Pol I DNA polymerase, Pol II DNA polymerase, Pol III DNA polymerase, Pol IV DNA polymerase, Pol V DNA polymerase, Pol β DNA polymerase, Pol μ DNA polymerase, Pol λ DNA polymerase, Pol σ DNA polymerase, Pol α DNA polymerase, Pol δ DNA polymerase, Pol ε DNA polymerase, Pol η DNA polymerase, Pol ι DNA polymerase, Pol κ DNA polymerase, Pol ζ DNA polymerase, Pol γ DNA polymerase, Pol θ DNA polymerase, Pol υ DNA polymerase, or a thermophilic nucleic acid polymerase (e.g., Therminator γ, 9° N polymerase (exo-), Therminator II, Therminator III, or Therminator IX). In embodiments, the DNA polymerase is a thermophilic nucleic acid polymerase. In embodiments, the DNA polymerase is a modified archaeal DNA polymerase. In embodiments, the polymerase is a reverse transcriptase. In embodiments, the polymerase is a mutant P. abyssi polymerase (e.g., such as a mutant P. abyssi polymerase described in WO 2018/148723 or WO 2020/056044, each of which are incorporated herein by reference for all purposes). In embodiments, the kit includes a strand-displacing polymerase. In embodiments, the kit includes a strand-displacing polymerase, such as a phi29 polymerase, phi29 mutant polymerase or a thermostable phi29 mutant polymerase.

In embodiments, the kit can further include one or more biological stain(s) (e.g., any of the biological stains as described herein). For example, the kit can further include eosin and hematoxylin. In other examples, the kit can include a biological stain such as acridine orange, Bismarck brown, carmine, Coomassie blue, crystal violet, DAPI, eosin, ethidium bromide, acid fuchsine, hematoxylin, Hoechst stains, iodine, methyl green, methylene blue, neutral red, Nile blue, Nile red, osmium tetroxide, propidium iodide, rhodamine, safranin, or any combination thereof. In embodiments, the kit is designed for staining tissue samples for imaging and detecting target molecules (e.g., proteins) can be significantly expanded beyond the inclusion of fluorophores. For instance, the kit can include eosin and hematoxylin, which are classic histological stains. Eosin, a red dye, typically stains acidic components of the cell such as cytoplasmic proteins, while hematoxylin, a basic dye, binds to nucleic acids, coloring the cell nucleus blue. This combination is widely used in histopathology for detailed tissue structure visualization. Moreover, the kit can encompass stains such as acridine orange, a nucleic acid-selective fluorescent cationic dye, and Bismarck brown, which is often used for staining backgrounds in histological tissue sections. Carmine, another potential inclusion, is a natural red dye used for staining glycogen, while Coomassie blue is a popular choice for protein staining in gel electrophoresis. Crystal violet, a triarylmethane dye, can be included for staining cell walls and nuclei, and DAPI, a fluorescent stain that binds strongly to A-T rich regions in DNA, is useful in fluorescence microscopy. Ethidium bromide, a fluorescent intercalator, is also a valuable addition for its role in nucleic acid staining, especially in gel electrophoresis. Further, the kit can include acid fuchsine, used in Masson's trichrome stain; Hoechst stains, which are cell-permeable, DNA-specific blue fluorescent dyes; and iodine, commonly used in Gram staining and for staining starch in plant cells. Methyl green and methylene blue, both traditional histological stains, can be included for their affinity towards nucleic acids. Neutral red, a vital stain that accumulates in lysosomes, Nile blue and Nile red, both used for staining lipids, and osmium tetroxide, a heavy metal stain for lipid bilayers in electron microscopy, can be part of the kit. Propidium iodide, a popular red-fluorescent nuclear and chromosome counterstain, along with rhodamine, may be utilized. Safranin, commonly used in Gram staining, can be included for its ability to stain cell components like nuclei, cytoplasm, and cell walls in various colors, enhancing the contrast and detail in tissue imaging.

To facilitate visualization, biological samples can be stained using a wide variety of stains and staining techniques. In embodiments, for example, the kit may include any number of stains, including but not limited to, acridine orange, Bismarck brown, carmine, coomassie blue, cresyl violet, DAPI, eosin, ethidium bromide, acid fuchsine, haematoxylin, Hoechst stains, iodine, methyl green, methylene blue, neutral red, Nile blue, Nile red, osmium tetraoxide, propidium iodide, rhodamine, safranine and/or an immunofluorescence stain. [0294]

In an aspect is a provided a kit including a compound of formula (I):

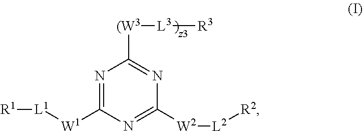

wherein R¹, R², R³, W¹, W², W³, L, L², and L³ are as described herein, including in embodiments. In embodiments, the kit includes compound A having formula:

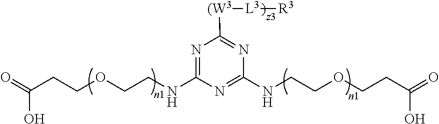

and compound B having formula:

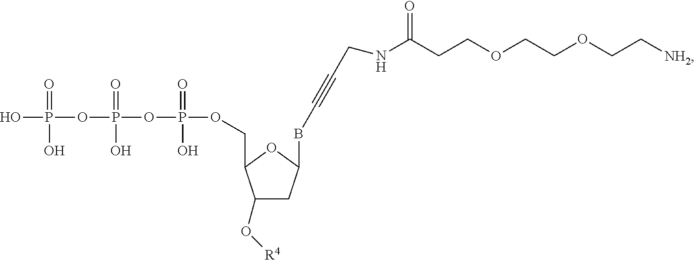

wherein n1, n2, z3, W³, L³, R³, R⁴, and B are as described herein.

### III. Methods

In an aspect is provided a method of imaging a biomolecule. In embodiments, the method includes directing an excitation beam onto a biomolecule including a detectable moiety and detecting a light emission from the detectable moiety, wherein the biomolecule is as described herein. In embodiments, the biomolecule is in or on a cell or tissue, wherein the cell or tissue is permeabilized and immobilized on a solid support or substrate described herein. In embodiments, the biomolecule is attached to a nucleotide construct as described herein. In embodiments, the biomolecule is attached to a nucleotide construct as described herein, wherein the biomolecule is avidin or streptavidin.

In embodiments, imaging a biomolecule includes directing an excitation beam onto a biomolecule. In embodiments, the excitation beam is directed from a light source, where the light source includes a laser, LED (light emitting diode), a mercury or tungsten lamp, or a super-continuous diode. In embodiments, the excitation beam has a wavelength between 200 nm to 1500 nm. In embodiments, the excitation beam has a wavelength of 405 nm, 470 nm, 488 nm, 514 nm, 520 nm, 532 nm, 561 nm, 633 nm, 639 nm, 640 nm, 800 nm, 808 nm, 912 nm, 1024 nm, or 1500 nm. In embodiments, the excitation beam has a wavelength of 405 nm, 488 nm, 532 nm, or 633 nm.

In embodiments, imaging a biomolecule includes detecting a light emission. In embodiments, detecting a light emission includes detecting light with a wavelength of 400-800 nm. In embodiments, detecting a light emission includes detecting light with a wavelength of 600 nm-900 nm. In embodiments, detecting a light emission includes detecting light with a wavelength of 1000 nm-1700 nm. In embodiments, detecting includes detecting a light emission in the near-infrared spectrum. In embodiments, detecting a light emission includes detecting light with a wavelength of 443 nm, 506 nm, 512 nm, 514 nm, 517 nm, 518 nm, 519 nm, 520 nm, 521 nm, 523 nm, 526 nm, 527 nm, 533 nm, 537 nm, 540 nm, 548 nm, 550 nm, 554 nm, 555 nm, 556 nm, 565 nm, 568 nm, 572 nm, 573 nm, 574 nm, 575 nm, 578 nm, 580 nm, 590 nm, 591 nm, 595 nm, 596 nm, 603 nm, 605 nm, 615 nm, 617 nm, 618 nm, 619 nm, 630 nm, 647 nm, 650 nm, 665 nm, 670 nm, 690 nm, 694 nm, 702 nm, 723 nm, or 775 nm. In embodiments, detecting a light emission includes detecting in an “imaging window,” wherein an imaging window refers to a range of wavelengths where tissue autofluorescence is minimal and the absorption and emission of light in tissue results in minimal light scattering (see, e.g., Pansare et al. Chem Mater. 2012 Mar. 13; 24(5): 812-827 and Wang et al. ACS Cent Sci. 2020 Aug. 26; 6(8): 1302-1316).

In an aspect is provided a method of sequencing a nucleic acid molecule. In embodiments, the method includes contacting a primer hybridized to the nucleic acid molecule with a compound or biomolecule as described herein and detecting the compound. In embodiments, the method includes sequencing the nucleic acid molecule in a cell or tissue.

In embodiments, the cell forms part of a tissue in situ. In embodiments, the cell is an isolated single cell. In embodiments, the cell is a prokaryotic cell. In embodiments, the cell is a eukaryotic cell. In embodiments, the cell is a bacterial cell, a fungal cell, a plant cell, or a mammalian cell. In embodiments, the cell is a stem cell. In embodiments, the stem cell is an embryonic stem cell, a tissue-specific stem cell, a mesenchymal stem cell, or an induced pluripotent stem cell. In embodiments, the cell is an endothelial cell, muscle cell, myocardial, smooth muscle cell, skeletal muscle cell, mesenchymal cell, epithelial cell; hematopoietic cell, such as lymphocytes, including T cell, e.g., (Th1 T cell, Th2 T cell, Th0 T cell, cytotoxic T cell); B cell, pre-B cell; monocytes; dendritic cell; neutrophils; or a macrophage. In embodiments, the cell is a stem cell, an immune cell, a cancer cell (e.g., a circulating tumor cell or cancer stem cell), a viral-host cell, or a cell that selectively binds to a desired target. In embodiments, the cell includes a T cell receptor gene sequence, a B cell receptor gene sequence, or an immunoglobulin gene sequence. In embodiments, the cell includes a Toll-like receptor (TLR) gene sequence. In embodiments, the cell includes a gene sequence corresponding to an immunoglobulin light chain polypeptide and a gene sequence corresponding to an immunoglobulin heavy chain polypeptide. In embodiments, the cell is a genetically modified cell. In embodiments, the cell is a circulating tumor cell or cancer stem cell.

In embodiments, the cell in situ is obtained from a subject (e.g., human or animal tissue). Once obtained, the cell is placed in an artificial environment in plastic or glass containers supported with specialized medium containing essential nutrients and growth factors to support proliferation.

In embodiments, the cell is obtained from a biological sample. In embodiments, the biological sample can be a biological tissue, cultured cells, or cells taken from an animal subject of interest. In embodiments, the biological sample includes material that is human origin or mouse origin. In embodiments, the biological sample is fresh, frozen, or fixed. In embodiments it can be a section or core obtained from a formalin-fixed paraffin-embedded (FFPE) tissue block. The sample can include material from a tissue section, tissue micro-array (TMA), cell pellet, core biopsy, needle biopsy, or cells obtained from a blood or serum sample. In embodiments, the biological sample is immobilized on a surface of a functionalized slide, a functionalized plate, a functionalized well, or a functionalized film. The biological sample can be contacted with two or more (e.g., 3 or more, 4 or more, 5 or more, 6 or more, 8 or more, 10 or more, 12 or more, 15 or more, 20 or more, 30 or more, 40 or more, 50 or more, 60 or more, 80 or more, or even more) antibodies, antibody fragments, or a combination thereof. The sample can be contacted with a cocktail of all the antibodies or antibody fragments, or combinations of multiple subsets of the total number of antibodies.

In embodiments, the cell is an adherent cell (e.g., epithelial cell, endothelial cell, or neural cell). Adherent cells are usually derived from tissues of organs and attach to a substrate (e.g., epithelial cells adhere to an extracellular matrix coated substrate via transmembrane adhesion protein complexes). Adherent cells typically require a substrate, e.g., tissue culture plastic, which may be coated with extracellular matrix (e.g., collagen and laminin) components to increase adhesion properties and provide other signals needed for growth and differentiation. In embodiments, the cell is a leukocyte (i.e., a white-blood cell). In embodiments, leukocyte is a granulocyte (neutrophil, eosinophil, or basophil), monocyte, or lymphocyte (T cells and B cells). In embodiments, the cell is a lymphocyte. In embodiments, the cell is a neuronal cell, an endothelial cell, epithelial cell, germ cell, plasma cell, a muscle cell, peripheral blood mononuclear cell (PBMC), a myocardial cell, or a retina cell. In embodiments, the cell is a suspension cell (e.g., a cell free-floating in the culture medium, such a lymphoblast or hepatocyte). In embodiments, the cell is a glial cell (e.g., astrocyte, radial glia), pericyte, or stem cell (e.g., a neural stem cell). In embodiments, the cell is a neuronal cell. In embodiments, the cell is an endothelial cell. In embodiments, the cell is an epithelial cell. In embodiments, the cell is a germ cell. In embodiments, the cell is a plasma cell. In embodiments, the cell is a muscle cell. In embodiments, the cell is a peripheral blood mononuclear cell (PBMC). In embodiments, the cell is a myocardial cell. In embodiments, the cell is a retina cell. In embodiments, the cell is a lymphoblast. In embodiments, the cell is a hepatocyte. In embodiments, the cell is a glial cell. In embodiments, the cell is an astrocyte. In embodiments, the cell is a radial glia. In embodiments, the cell is a pericyte. In embodiments, the cell is a stem cell. In embodiments, the cell is a neural stem cell.

In embodiments, the cell is an immune cell. In embodiments, the immune cell is a granulocyte, a mast cell, a monocyte, a neutrophil, a dendritic cell, or a natural killer (NK) cell. In embodiments, the immune cell is an adaptive cell, such as a T cell, NK cell, or a B cell. In embodiments, the cell includes a T cell receptor gene sequence, a B cell receptor gene sequence, or an immunoglobulin gene sequence. In embodiments, the immune cell is a granulocyte. In embodiments, the immune cell is a mast cell. In embodiments, the immune cell is a monocyte. In embodiments, the immune cell is a neutrophil. In embodiments, the immune cell is a dendritic cell. In embodiments, the immune cell is a natural killer (NK) cell. In embodiments, the immune cell is a T cell. In embodiments, the immune cell is a B cell. In embodiments, the cell includes a T cell receptor gene sequence. In embodiments, the cell includes a B cell receptor gene sequence. In embodiments, the cell includes an immunoglobulin gene sequence. In embodiments, the cell is a cancer cell. In embodiments, the cancer cell includes a cancer-associated gene (e.g., an oncogene associated with kinases and genes involved in DNA repair) or a cancer-associated biomarker. A “biomarker” is a substance that is associated with a particular characteristic, such as a disease or condition. A change in the levels of a biomarker may correlate with the risk or progression of a disease or with the susceptibility of the disease to a given treatment. In embodiments, the cancer is Acute Myeloid Leukemia, Adrenocortical Carcinoma, Bladder Urothelial Carcinoma, Breast Ductal Carcinoma, Breast Lobular Carcinoma, Cervical Carcinoma, Cholangiocarcinoma, Colorectal Adenocarcinoma, Esophageal Carcinoma, Gastric Adenocarcinoma, Glioblastoma Multiforme, Head and Neck Squamous Cell Carcinoma, Hepatocellular Carcinoma, Kidney Chromophobe Carcinoma, Kidney Clear Cell Carcinoma, Kidney Papillary Cell Carcinoma, Lower Grade Glioma, Lung Adenocarcinoma, Lung Squamous Cell Carcinoma, Mesothelioma, Ovarian Serous Adenocarcinoma, Pancreatic Ductal Adenocarcinoma, Paraganglioma & Pheochromocytoma, Prostate Adenocarcinoma, Sarcoma, Skin Cutaneous Melanoma, Testicular Germ Cell Cancer, Thymoma, Thyroid Papillary Carcinoma, Uterine Carcinosarcoma, Uterine Corpus Endometrioid Carcinoma, or Uveal Melanoma.

In embodiments, the cell is a neuronal cell, an endothelial cell, epithelial cell, germ cell, plasma cell, a muscle cell, peripheral blood mononuclear cell (PBMC), a myocardial cell, cancer cell, or a retina cell.

In embodiments, the tissue incudes liver tissue, kidney tissue, bone tissue, lung tissue, thymus tissue, adrenal tissue, skin tissue, bladder tissue, colon tissue, spleen tissue, or brain tissue.

In embodiments, the tissue is a tissue section. In embodiments, the tissue section includes a tissue or a cell (e.g., plurality of cells such as blood cells). In embodiments, the tissue section includes one or more cells. In embodiments, the thickness of the tissue section is about 1 μm to about 20 μm. In embodiments, the thickness of the tissue section is about 5 μm to about 12 μm. In embodiments, the thickness of the tissue section is about 8 μm to about 15 μm. In embodiments, the thickness of the tissue section is about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, or about 15 μm.

In certain embodiments, tissue sections are tumor tissue samples. Tumor samples may contain only tumor cells, or they may contain both tumor cells and non-tumor cells. In particular embodiments, a tissue section includes only non-tumor cells. In particular embodiments, the tumor is a solid tumor. In particular embodiments, the tissue section is obtained from or includes an adrenal cortical cancer, anal cancer, aplastic anemia, bile duct cancer, bladder cancer, bone cancer, bone metastasis, brain tumor, brain cancer, breast cancer, childhood cancer, cancer of unknown primary origin, Castleman disease, cervical cancer, colon/rectal cancer, endometrial cancer, esophagus cancer, Ewing family of tumors, eye cancer, gallbladder cancer, gastrointestinal carcinoid tumors, gastrointestinal stromal tumors, gestational trophoblastic disease, head or neck cancer, Kaposi sarcoma, renal cell carcinoma, laryngeal and hypopharyngeal cancer, liver cancer, non-small cell lung cancer, small cell lung cancer, lung carcinoid tumor, lymphoma of the skin, malignant mesothelioma, myelodysplasia syndrome, nasal cavity or paranasal sinus cancer, nasopharyngeal cancer, neuroblastoma, oral cavity or oropharyngeal cancer, osteosarcoma, ovarian cancer, pancreatic cancer, penile cancer, pituitary tumors, prostate cancer, retinoblastoma, rhabdomyosarcoma, salivary gland cancer, sarcoma in adult soft tissue, basal or squamous cell skin cancer, melanoma, small intestine cancer, stomach cancer, testicular cancer, throat cancer, thymus cancer, thyroid cancer, uterine sarcoma, vaginal cancer, vulvar cancer, Waldenstrom macroglobulinemia, Wilms tumor and secondary cancers caused by cancer treatment, is a tissue section obtained from a subject diagnosed with or suspected of having any of these tumors or cancers.

In embodiments, the method includes immobilizing the cell or tissue (e.g., tissue section) onto a solid support or substrate described herein, wherein the cell or tissue includes the biomolecule to be detected. In embodiments, the method includes immobilizing a plurality of cells or tissue sections onto a solid support or substrate described herein, wherein the cell or tissue includes the biomolecule to be detected. In embodiments, the method includes immobilizing 24 tissue sections (10 mm×17 mm sections). In embodiments, the method includes immobilizing 40 tissue sections (10 mm×10 mm sections). In embodiments, the method includes immobilizing 128 tissue sections (4 m×4 m sections).

In embodiments, the cell or tissue section is immobilized to a substrate. The cell or tissue may have been cultured on the surface, or the cell or tissue may have been initially cultured in suspension and then fixed to the surface. Substrates can be two- or three-dimensional and can include a planar surface (e.g., a glass slide). A substrate can include glass (e.g., controlled pore glass (CPG)), quartz, plastic (such as polystyrene (low cross-linked and high cross-linked polystyrene), polycarbonate, polypropylene and poly(methymethacrylate)), acrylic copolymer, polyamide, silicon, metal (e.g., alkanethiolate-derivatized gold), cellulose, nylon, latex, dextran, gel matrix (e.g., silica gel), polyacrolein, or composites. In embodiments, the substrate is a borosilicate glass substrate with a composition including SiO₂, Al₂O₃, B₂O₃, Li₂O, Na₂O, K₂O, MgO, CaO, SrO, BaO, ZnO, TiO₂, ZrO₂, P₂O₅, or a combination thereof (see e.g., U.S. Pat. No. 10,974,990). In embodiments, the substrate is an alkaline earth boro-aluminosilicate glass substrate. In embodiments, the substrate is a functionalized glass surface. In embodiments, the substrate is a functionalized plastic surface.

In embodiments, the solid support or substrate described herein includes one or more channels. In embodiments, the solid support or substrate includes a channel bored into solid support or substrate. In embodiments, the solid support or substrate includes a plurality of channels solid support or substrate. In embodiments, the solid support or substrate includes 2, 3, or 4 channels bored into solid support or substrate. In embodiments, the width of the channel is from about 1 to 5 mm, 5 mm to 10 mm, or 10 mm to 15 mm. In embodiments, the channel is a reaction chamber on the solid support or substrate. In embodiments, the cell or tissue is immobilized in a channel bored onto the solid support or substrate.

In embodiments, the substrate includes a polymeric coating, optionally containing bioconjugate reactive moieties capable of affixing the sample. Suitable three-dimensional substrates include, for example, spheres, microparticles, beads, membranes, slides, plates, micromachined chips, tubes (e.g., capillary tubes), microwells, microfluidic devices, channels, filters, or any other structure suitable for anchoring a sample. In embodiments, the substrate is not a flow cell. In embodiments, the substrate includes a polymer matrix material (e.g., polyacrylamide, cellulose, alginate, polyamide, cross-linked agarose, cross-linked dextran or cross-linked polyethylene glycol), which may be referred to herein as a “matrix”, “synthetic matrix”, “exogenous polymer” or “exogenous hydrogel”. In embodiments, a matrix may refer to the various components and organelles of a cell, for example, the cytoskeleton (e.g., actin and tubulin), endoplasmic reticulum, Golgi apparatus, vesicles, etc. In embodiments, the matrix is endogenous to a cell. In embodiments, the matrix is exogenous to a cell. In embodiments, the matrix includes both the intracellular and extracellular components of a cell. In embodiments, polynucleotide primers may be immobilized on a matrix including the various components and organelles of a cell. Immobilization of polynucleotide primers on a matrix of cellular components and organelles of a cell is accomplished, for example, through the interaction/reaction of complementary bioconjugate reactive moieties. In embodiments, the exogenous polymer may be a matrix or a network of extracellular components that act as a point of attachment (e.g., act as an anchor) for the cell to a substrate.

The cell or tissue may be manipulated prior to immobilizing the cell or tissue onto a solid support using known techniques in the art (see, e.g., PCT Publication WO2023076832A1). In embodiments, the method further includes cutting a sample portion from the biological sample (e.g., including cells or tissues) using a punch device such that the punch device contains the sample portion; mounting the punch device containing the sample portion onto the solid support as described herein (e.g., inverting the punch device); pushing the sample portion out of the punch device using a piston, so that all or a portion thereof of the sample portion is positioned on the solid support as described herein. In embodiments, the method further includes cutting a sample portion from the biological sample using two or more punch devices such that each punch device contains a different the sample portion; mounting each punch device containing the sample portion onto the solid support as described herein; pushing the sample portions out of the punch devices using one or more pistons so that the sample portions are positioned onto the solid support as described herein.

In embodiments, the cell is exposed to paraformaldehyde (i.e., by contacting the cell with paraformaldehyde). Any suitable permeabilization and fixation technologies can be used for making the cell available for the detection methods provided herein. In embodiments the method includes affixing single cells or tissues to a transparent substrate. Exemplary tissues include those from skin tissue, muscle tissue, bone tissue, organ tissue and the like. In embodiments, the method includes immobilizing the cell in situ to a substrate and permeabilized for delivering probes, enzymes, nucleotides and other components required in the reactions. In embodiments, the cell includes many cells from a tissue section in which the original spatial relationships of the cells are retained. In embodiments, the cell in situ is within a Formalin-Fixed Paraffin-Embedded (FFPE) sample. In embodiments, the cell is subjected to paraffin removal methods, such as methods involving incubation with a hydrocarbon solvent, such as xylene or hexane, followed by two or more washes with decreasing concentrations of an alcohol, such as ethanol. The cell may be rehydrated in a buffer, such as PBS, TBS or MOPs. In embodiments, the FFPE sample is incubated with xylene and washed using ethanol to remove the embedding wax, followed by treatment with Proteinase K to permeabilized the tissue. In embodiments, the cell is fixed with a chemical fixing agent. In embodiments, the chemical fixing agent is formaldehyde or glutaraldehyde. In embodiments, the chemical fixing agent is glyoxal or dioxolane. In embodiments, the chemical fixing agent includes one or more of ethanol, methanol, 2-propanol, acetone, and glyoxal. In embodiments, the chemical fixing agent includes formalin, Greenfix®, Greenfix® Plus, UPM, CyMol®, HOPE®, CytoSkelFix™, F-Solv®, FineFIX®, RCL2/KINFix, UMFIX, Glyo-Fixx®, Histochoice®, or PAXgene®. In embodiments, the cell is fixed within a synthetic three-dimensional matrix (e.g., polymeric material). In embodiments, the synthetic matrix includes polymeric-crosslinking material. In embodiments, the material includes polyacrylamide, poly-ethylene glycol (PEG), poly(acrylate-co-acrylic acid) (PAA), or Poly(N-isopropylacrylamide) (NIPAM). In embodiments, the cell or tissue is permeabilized and immobilized to a solid support surface. In embodiments, the cell or tissue is permeabilized and immobilized to an array (i.e., to discrete locations arranged in an array). In embodiments, the cell or tissue is immobilized to a solid support surface.

In embodiments, prior to sequencing, the method further includes (i) contacting the cell or tissue with an probe oligonucleotide including a first hybridization sequence and a second hybridization sequence and hybridizing the first hybridization sequence of the probe oligonucleotide to a first sequence of the target nucleic acid sequence and hybridizing the second hybridization sequence of the probe oligonucleotide to a second sequence of the target nucleic acid; (ii) ligating the probe oligonucleotide to form a circular oligonucleotide; (iii) amplifying the circular oligonucleotide or complement thereof to generate an amplification product; and (iv) detecting the amplification product. In embodiments, detecting the amplification product includes detecting a detectable moiety on the biomolecule described herein. In embodiments, detecting the amplification product includes detecting a detectable moiety on the biomolecule, wherein the biomolecule is attached to a nucleotide construct as described herein. In embodiments, the method further includes contacting the cell or tissue with a stain. In embodiments, the method further includes directing an excitation light to the tissue section and detecting an emission light from the detectable moiety on the biomolecule described herein and the stain. A stain is a chemical agent used to selectively color components of biological tissues or cells to enhance their visibility under a microscope. Stains typically bind to specific cellular structures or organelles, such as proteins, nucleic acids, lipids, or carbohydrates, allowing for the differentiation and identification of these structures. In embodiments, the stain is a fluorescent stain (e.g., an intrinsic stain). Intrinsic or fluorescent stains are chemical compounds that possess the inherent ability to emit fluorescence when exposed to specific wavelengths of light, thereby enabling the visualization of biological structures without the need for additional staining agents; examples include eosin, which absorbs light in the blue-green part of the spectrum (around 490-520 nm) and emits light in the green-yellow part of the spectrum (around 520-550 nm), and Hoechst stains, which bind to DNA and emit blue fluorescence around 461 nm. In embodiments, detecting includes directing an excitation light to the cell or tissue and detecting an emission light from the stain.

The probe oligonucleotide includes a first hybridization sequence and a second hybridization sequence. In embodiments, the first hybridization sequence and second hybridization sequence independently include about 5 to about 20 nucleotides. In embodiments, the first hybridization sequence and second hybridization sequence independently include about 15 to about 20 nucleotides. In embodiments, the first hybridization sequence and second hybridization sequence independently include about 20 to about 50 nucleotides.

In embodiments, the probe oligonucleotide includes an amplification primer binding sequence, a sequencing primer binding sequence, or both an amplification primer binding sequence and a sequencing primer binding sequence. In embodiments, the probe oligonucleotide includes an amplification primer binding sequence. In embodiments, the probe oligonucleotide includes a sequencing primer binding sequence. In embodiments, the probe oligonucleotide includes both an amplification primer binding sequence and a sequencing primer binding sequence. Primer binding sequences usually have a length in the range of between 3 to 36 nucleotides, also 5 to 24 nucleotides, also from 14 to 36 nucleotides. In embodiments, an amplification primer and a sequencing primer are complementary to the same primer binding sequence, or overlapping primer binding sequences. In embodiments, an amplification primer and a sequencing primer are complementary to different primer binding sequences.

In embodiments, the target sequence is about 2 to about 500 nucleotides. In embodiments, the target nucleic acid is about 5 to 50 nucleotides. In embodiments, the target sequence is about 50 to about 120 nucleotides. In embodiments, the target sequence is about 100 to about 300 nucleotides. In embodiments, the target sequence is about 50 to about 500 nucleotides.

In embodiments, the target sequence is an RNA nucleic acid sequence. In embodiments, the target sequence is a DNA nucleic acid sequence. In embodiments, the target sequence is an RNA nucleic acid sequence or DNA nucleic acid sequence from the same cell. In embodiments, the target sequence is an RNA nucleic acid sequence. In embodiments, the target sequence is messenger RNA (mRNA), transfer RNA (tRNA), micro RNA (miRNA), small interfering RNA (siRNA), small nucleolar RNA (snoRNA), small nuclear RNA (snRNA), Piwi-interacting RNA (piRNA), enhancer RNA (eRNA), or ribosomal RNA (rRNA). In embodiments, the target sequence is pre-mRNA. In embodiments, the target sequence is heterogeneous nuclear RNA (hnRNA). In embodiments, the target sequence is mRNA, tRNA (transfer RNA), rRNA (ribosomal RNA), or noncoding RNA (such as lncRNA (long noncoding RNA)). In embodiments, the target sequence are on different regions of the same RNA nucleic acid sequence.

In embodiments, the method further includes extending the probe oligonucleotide along the target sequence with a polymerase to generate an extension strand including a complement of the target sequence. In embodiments, extending the probe oligonucleotide along the target sequence occurs prior to ligating.

In embodiments, the polymerase is T4, T7, Sequenase, Taq, Klenow, Pol I DNA polymerases, SD polymerase, Bst large fragment polymerase, or a phi29 polymerase or mutant thereof. In embodiments, the strand-displacing enzyme is an SD polymerase, Bst large fragment polymerase, or a phi29 polymerase or mutant thereof. In embodiments, the strand-displacing polymerase is phi29 polymerase, phi29 mutant polymerase or a thermostable phi29 mutant polymerase. A “phi polymerase” (or “(D29 polymerase”) is a DNA polymerase from the (29 phage or from one of the related phages that, like (D29, contain a terminal protein used in the initiation of DNA replication. For example, phi29 polymerases include the B103, GA-1, PZA, Φ15, BS32, M2Y (also known as M2), Nf, G1, Cp-1, PRD1, PZE, SFS, Cp-5, Cp-7, PR4, PR5, PR722, L17, Φ21, and AV-1 DNA polymerases, as well as chimeras thereof. A phi29 mutant DNA polymerase includes one or more mutations relative to naturally-occurring wild-type phi29 DNA polymerases, for example, one or more mutations that alter interaction with and/or incorporation of nucleotide analogs, increase stability, increase read length, enhance accuracy, increase phototolerance, and/or alter another polymerase property, and can include additional alterations or modifications over the wild-type phi29 DNA polymerase, such as one or more deletions, insertions, and/or fusions of additional peptide or protein sequences. Thermostable phi29 mutant polymerases are known in the art, see for example US 2014/0322759, which is incorporated herein by reference for all purposes. For example, a thermostable phi29 mutant polymerase refers to an isolated bacteriophage phi29 DNA polymerase including at least one mutation selected from the group consisting of M8R, V51A, M97T, L123S, G197D, K209E, E221K, E239G, Q497P, K512E, E515A, and F526 (relative to wild type phi29 polymerase). In embodiments, the polymerase is a phage or bacterial RNA polymerases (RNAPs). In embodiments, the polymerase is a T7 RNA polymerase. In embodiments, the polymerase is an RNA polymerase. Useful RNA polymerases include, but are not limited to, viral RNA polymerases such as T7 RNA polymerase, T3 polymerase, SP6 polymerase, and Kll polymerase; Eukaryotic RNA polymerases such as RNA polymerase I, RNA polymerase II, RNA polymerase III, RNA polymerase IV, and RNA polymerase V; and Archaea RNA polymerase.

In embodiments, the polymerase is a reverse transcriptase or a mutant thereof. Exemplary reverse transcriptases include, but are not limited to, HIV-1 reverse transcriptase from human immunodeficiency virus type 1 (PDB 1HMV), HIV-2 reverse transcriptase from human immunodeficiency virus type 2, M-MLV reverse transcriptase from the Moloney murine leukemia virus, AMV reverse transcriptase from the avian myeloblastosis virus, and Telomerase reverse transcriptase. In embodiments, the reverse transcriptase harbors a point mutation that removes the RNase H activity. In embodiments, the reverse transcriptase includes the wild type RNase H activity.

In embodiments, ligating includes chemical ligation (e.g., enzyme-free, click-mediated ligation). Reaction conditions and protocols for chemical ligation techniques that are compatible with nucleic acid amplification methods are known in the art, for example El-Sagheer, A. H., & Brown, T. (2012). Accounts of chemical research, 45(8), 1258-1267; Manuguerra I. et al. Chem Commun (Camb). 2018; 54(36):4529-4532; and Odeh, F., et al. (2019). Molecules (Basel, Switzerland), 25(1), 3, each of which is incorporated herein by reference in their entirety.

In embodiments, ligating includes covalently binding adjacent sequences with a ligase. In embodiments, the method further includes ligating a 5′-end and a 3′-end of the probe oligonucleotide to form the circular oligonucleotide (i.e., an oligonucleotide that is a continuous strand lacking free a 5′-end and a 3′-end). In embodiments, the method includes ligating the 5′-end and the 3′-end of the probe oligonucleotide to form the circular oligonucleotide, the circular oligonucleotide includes the target nucleic acid. In embodiments, the method includes ligating the 5′-end and the 3′-end of the probe oligonucleotide to form the circular oligonucleotide, wherein the circular oligonucleotide includes oligonucleotide barcode described herein. In embodiments, the ligase is a pre-adenylated ligase. In embodiments, the ligase is a PBCV-1 DNA Ligase or mutant thereof. In embodiments, the ligase is a TS2126 RNA ligase or mutant thereof. In embodiments, ligating includes enzymatic ligation including a ligation enzyme (e.g., Circligase™ enzyme, Taq DNA Ligase, HiFi Taq DNA Ligase, T4 ligase, PBCV-1 DNA Ligase (also known as SplintR® ligase) or Ampligase® DNA Ligase, or mutant thereof). Non-limiting examples of ligases include DNA ligases such as DNA Ligase I, DNA Ligase II, DNA Ligase III, DNA Ligase IV, T4 DNA ligase, T7 DNA ligase, T3 DNA Ligase, E. coli DNA Ligase, PBCV-1 DNA Ligase (also known as SplintR® ligase) or a Taq DNA Ligase. In embodiments, the ligase enzyme includes a T4 DNA ligase, T4 RNA ligase 1, T4 RNA ligase 2, T3 DNA ligase, T7 DNA ligase, or mutant thereof. In embodiments, the enzymatic ligation is performed by a mixture of ligases. In embodiments, the ligation enzyme is selected from the group consisting of T4 DNA ligase, T4 RNA ligase 1, T4 RNA ligase 2, RtcB ligase, T3 DNA ligase, T7 DNA ligase, Taq DNA ligase, PBCV-1 DNA Ligase, a thermostable DNA ligase (e.g., 5′AppDNA/RNA ligase), an ATP dependent DNA ligase, an RNA-dependent DNA ligase (e.g., SplintR® ligase), and combinations thereof. In embodiments, enzymatic ligation includes two different ligation enzymes (e.g., SplintR® ligation and T4 DNA ligase, or SplintR® ligase and Taq DNA ligase). In embodiments, enzymatic ligation includes more than two different ligation enzymes. In embodiments, the ligase is PBCV NYs1 ligase, Blyttiomyces helicus ligase, PCBV-1 ligase, or ATCV-1 ATP-dependent DNA ligase (Z187L).

In embodiments, ligating the probe oligonucleotide generates a circular oligonucleotide. In embodiments, the circular oligonucleotide is about 10 to about 120 nucleotides, about 100 to about 1000 nucleotides, about 100 to about 300 nucleotides, about 300 to about 500 nucleotides, or about 500 to about 1000 nucleotides.

In embodiments, the circularizable oligonucleotide contains one or more functional moieties (e.g., bioconjugate reactive groups) that serve as attachment points to the cell (i.e., the internal cellular scaffold) or to the matrix in which the cell is embedded (e.g. a hydrogel). In embodiments, the bioconjugate reactive group is located at the 5′ and/or 3′ end of the oligonucleotide. In embodiments, the bioconjugate reactive group is located at an internal position of the circular oligonucleotide (e.g., the oligonucleotide contains one or more modified nucleotides, such as aminoallyl deoxyuridine 5′-triphosphate (dUTP) nucleotide(s)). In embodiments, the functional moiety can be covalently cross-linked, copolymerize with or otherwise non-covalently bound to the matrix. In embodiments, the functional moiety can react with a cross-linker. In embodiments, the functional moiety can be part of a ligand-ligand binding pair. Suitable exemplary functional moieties include an amine, acrydite, alkyne, biotin, azide, and thiol. In embodiments of crosslinking, the functional moiety is cross-linked to modified dNTP or dUTP or both. In embodiments, suitable exemplary cross-linker reactive groups include imidoester (DMP), succinimide ester (NHS), maleimide (Sulfo-SMCC), carbodiimide (DCC, EDC) and phenyl azide. Cross-linkers within the scope of the present disclosure may include a spacer moiety. In embodiments, such spacer moieties may be functionalized. In embodiments, such spacer moieties may be chemically stable. In embodiments, such spacer moieties may be of sufficient length to allow amplification of the nucleic acid bound to the matrix. In embodiments, suitable exemplary spacer moieties include polyethylene glycol, carbon spacers, photo-cleavable spacers and other spacers known to those of skill in the art and the like. In embodiments, the oligonucleotide primer contains a modified nucleotide (e.g., amino-allyl dUTP, 5-TCO-PEG4-dUTP, C₈-Alkyne-dUTP, 5-Azidomethyl-dUTP, 5-Vinyl-dUTP, or 5-Ethynyl dLTTP). For example, prior to amplification, the modified nucleotide-containing primer is attached to the cell protein matrix by using a cross-linking reagent (e.g., an amine-reactive crosslinking agent with PEG spacers, such as (PEGylated bis(sulfosuccinimidyl)suberate) (BS(PEG)₉)).

In embodiments, the method further includes amplifying a nucleic acid molecule (e.g., a nucleic acid molecule in a cell) to generate amplification products. In embodiments, the method further includes amplifying a circular oligonucleotide. In embodiments, amplifying includes contacting the flow cell assembly as described herein with one or more reagents for amplifying the target polynucleotide. Examples of reagents include but are not limited to polymerase, buffer, and nucleotides (e.g., an amplification reaction mixture). In certain embodiments the term “amplifying” refers to a method that includes a polymerase chain reaction (PCR). Conditions conducive to amplification (i.e., amplification conditions) are known and often include at least a suitable polymerase, a suitable template, a suitable primer or set of primers, suitable nucleotides (e.g., dNTPs), a suitable buffer, and application of suitable annealing, hybridization and/or extension times and temperatures. In embodiments, amplifying generates an amplicon. In embodiments, amplifying generates a rolony. In embodiments, an amplicon contains multiple, tandem copies of the circularized nucleic acid molecule of the corresponding sample nucleic acid. The number of copies can be varied by appropriate modification of the amplification reaction including, for example, varying the number of amplification cycles run, using polymerases of varying processivity in the amplification reaction and/or varying the length of time that the amplification reaction is run, as well as modification of other conditions known in the art to influence amplification yield. Generally, the number of copies of a nucleic acid in an amplicon is at least 100, 200, 500, 1000, 2000, 3000, 4000, 5000, 6000, 7000, 8000, 9000 and 10,000 copies, and can be varied depending on the application. As disclosed herein, one form of an amplicon is as a nucleic acid “ball” localized to the particle and/or well of the array. The number of copies of the nucleic acid can therefore provide a desired size of a nucleic acid “ball” or a sufficient number of copies for subsequent analysis of the amplicon, e.g., sequencing.

In embodiments, amplifying includes bridge polymerase chain reaction (bPCR) amplification, solid-phase rolling circle amplification (RCA), solid-phase exponential rolling circle amplification (eRCA), solid-phase recombinase polymerase amplification (RPA), solid-phase helicase dependent amplification (HDA), template walking amplification, or emulsion PCR on particles, or combinations of the methods. In embodiments, amplifying includes a bridge polymerase chain reaction amplification. In embodiments, amplifying includes a thermal bridge polymerase chain reaction (t-bPCR) amplification. In embodiments, amplifying includes a chemical bridge polymerase chain reaction (c-bPCR) amplification. Chemical bridge polymerase chain reactions include fluidically cycling a denaturant (e.g., formamide) and one or more additives (e.g., ethylene glycol) and maintaining the temperature within a narrow temperature range (e.g., +/−5° C.) or isothermally. In embodiments, c-bPCR does not include isothermal amplification, rather it requires minor (e.g., +/−5° C.) thermal oscillations. In contrast, thermal bridge polymerase chain reactions include thermally cycling between high temperatures (e.g., 85° C.-95° C.) and low temperatures (e.g., 60° C.-70° C.). Thermal bridge polymerase chain reactions may also include a denaturant, typically at a much lower concentration than traditional chemical bridge polymerase chain reactions. In embodiments, amplifying includes generating a double-stranded amplification product.

It will be appreciated that any of the amplification methodologies described herein or known in the art can be utilized with universal or target-specific primers to amplify the target polynucleotide. Suitable methods for amplification include, but are not limited to, the polymerase chain reaction (PCR), strand displacement amplification (SDA), transcription mediated amplification (TMA) and nucleic acid sequence-based amplification (NASBA), for example, as described in U.S. Pat. No. 8,003,354, which is incorporated herein by reference in its entirety. The above amplification methods can be employed to amplify one or more nucleic acids of interest. Additional examples of amplification processes include, but are not limited to, bridge-PCR, recombinase polymerase amplification (RPA), loop-mediated isothermal amplification (LAMP), rolling circle amplification (RCA), strand displacement amplification (SDA), rolling circle amplification (RCA) with exponential strand displacement amplification. In embodiments, amplification includes an isothermal amplification reaction. In embodiments, amplification includes bridge amplification. In general, bridge amplification uses repeated steps of annealing of primers to templates, primer extension, and separation of extended primers from templates. Because primers are attached within the core polymer, the extension products released upon separation from an initial template is also attached within the core. The 3′ end of an amplification product is then permitted to anneal to a nearby reverse primer that is also attached within the core, forming a “bridge” structure. The reverse primer is then extended to produce a further template molecule that can form another bridge. In embodiments, forward and reverse primers hybridize to primer binding sites that are specific to a particular target nucleic acid. In embodiments, forward and reverse primers hybridize to primer binding sites that have been added to, and are common among, target polynucleotides. Adding a primer binding site to target nucleic acids can be accomplished by any suitable method, examples of which include the use of random primers having common 5′ sequences and ligating adapter nucleotides that include the primer binding site. Examples of additional clonal amplification techniques include, but are not limited to, bridge PCR, solid-phase rolling circle amplification (RCA), solid-phase exponential rolling circle amplification, solid-phase recombinase polymerase amplification (RPA), solid-phase helicase dependent amplification (HDA), template walking amplification, emulsion PCR on particles (beads), or combinations of the aforementioned methods. Optionally, during clonal amplification, additional solution-phase primers can be supplemented in the microplate for enabling or accelerating amplification. In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer. In embodiments, the polymerase is T4, T7, Sequenase, Taq, Klenow, and Pol I DNA polymerases. SD polymerase, Bst large fragment polymerase, or a phi29 polymerase or mutant thereof.

In embodiments, the amplifying includes rolling circle amplification (RCA) or rolling circle transcription (RCT) (see, e.g., Lizardi et al., Nat. Genet. 19:225-232 (1998), which is incorporated herein by reference in its entirety). Several suitable rolling circle amplification methods are known in the art. For example, RCA amplifies a circular polynucleotide (e.g., DNA) by polymerase extension of an amplification primer complementary to a portion of the template polynucleotide. This process generates copies of the circular polynucleotide template such that multiple complements of the template sequence arranged end to end in tandem are generated (i.e., a concatemer) locally preserved at the site of the circle formation. In embodiments, the amplifying occurs at isothermal conditions. In embodiments, the amplifying includes hybridization chain reaction (HCR). HCR uses a pair of complementary, kinetically trapped hairpin oligomers to propagate a chain reaction of hybridization events, as described in Dirks, R. M., & Pierce, N. A. (2004) PNAS USA, 101(43), 15275-15278, which is incorporated herein by reference for all purposes. In embodiments, the amplifying includes branched rolling circle amplification (BRCA); e.g., as described in Fan T, Mao Y, Sun Q, et al. Cancer Sci. 2018; 109:2897-2906, which is incorporated herein by reference in its entirety. In embodiments, the amplifying includes hyberbranched rolling circle amplification (HRCA). Hyperbranched RCA uses a second primer complementary to the first amplification product. This allows products to be replicated by a strand-displacement mechanism, which yields drastic amplification within an isothermal reaction (Lage et al., Genome Research 13:294-307 (2003), which is incorporated herein by reference in its entirety). In embodiments, amplifying includes polymerase extension of an amplification primer.

In embodiments, the method further includes detecting the amplification products. In embodiments, detecting the amplification products includes detecting the label (e.g., the nucleic acid sequence). In embodiments, detecting the amplification products includes detecting the oligonucleotide label. In embodiments, detecting includes sequencing. In embodiments, sequencing includes extending a sequencing primer annealed to the target polynucleotide to incorporate a nucleotide containing a detectable label that indicates the identity of a nucleotide in the target polynucleotide, detecting the detectable label, and optionally repeating the extending and detecting of steps. In embodiments, sequencing includes extending a sequencing primer annealed to the target polynucleotide to incorporate a nucleotide containing a detectable label that indicates the identity of a nucleotide in the target polynucleotide, wherein the nucleotide is a nucleotide moiety of a nucleotide construct (e.g., a nucleotide construct described herein) attached to a biomolecule described herein. In embodiments, sequencing includes detecting the detectable label and repeating the extending and detecting of steps. In embodiments, the methods include sequencing one or more bases of a target nucleic acid by extending a sequencing primer hybridized to a target nucleic acid (e.g., an amplification product of a target nucleic acid). In embodiments, the sequencing includes sequencing-by-synthesis, sequencing by ligation, sequencing-by-hybridization, or pyrosequencing, and generates a sequencing read. In embodiments, generating a sequencing read includes executing a plurality of sequencing cycles, each cycle including extending the sequencing primer by incorporating a nucleotide or nucleotide analogue using a polymerase and detecting a characteristic signature indicating that the nucleotide or nucleotide analogue has been incorporated.

In embodiments, sequencing includes a plurality of sequencing cycles. In embodiments, sequencing includes 20 to 100 sequencing cycles. In embodiments, sequencing includes 50 to 100 sequencing cycles. In embodiments, sequencing includes 50 to 300 sequencing cycles. In embodiments, sequencing includes 50 to 150 sequencing cycles. In embodiments, sequencing includes at least 10, 20, 30 40, or 50 sequencing cycles. In embodiments, sequencing includes at least 10 sequencing cycles. In embodiments, sequencing includes 10 to 20 sequencing cycles. In embodiments, sequencing includes 10, 11, 12, 13, 14, or 15 sequencing cycles. In embodiments, sequencing includes (a) extending a sequencing primer by incorporating a labeled nucleotide, or labeled nucleotide analogue and (b) detecting the label to generate a signal for each incorporated nucleotide or nucleotide analogue. In embodiments, sequencing includes (a) extending a sequencing primer by incorporating a nucleotide moiety of a nucleotide construct (e.g., a nucleotide construct described herein) attached to a biomolecule described herein or a labeled nucleotide analogue thereof and (b) detecting the label to generate a signal for each incorporated nucleotide or nucleotide analogue.

In embodiments, the method includes sequencing the first and/or the second strand of a amplification product by extending a sequencing primer hybridized thereto. A variety of sequencing methodologies can be used such as sequencing-by-synthesis (SBS), pyrosequencing, sequencing by ligation (SBL), or sequencing by hybridization (SBH). Pyrosequencing detects the release of inorganic pyrophosphate (PPi) as particular nucleotides are incorporated into a nascent nucleic acid strand (Ronaghi, et al., Analytical Biochemistry 242(1), 84-9 (1996); Ronaghi, Genome Res. 11(1), 3-11 (2001); Ronaghi et al. Science 281(5375), 363 (1998); U.S. Pat. Nos. 6,210,891; 6,258,568; and. 6,274,320, each of which is incorporated herein by reference in its entirety). In pyrosequencing, released PPi can be detected by being converted to adenosine triphosphate (ATP) by ATP sulfurylase, and the level of ATP generated can be detected via light produced by luciferase. In this manner, the sequencing reaction can be monitored via a luminescence detection system. In both SBL and SBH methods, target nucleic acids, and amplicons thereof, that are present at features of an array are subjected to repeated cycles of oligonucleotide delivery and detection. SBL methods, include those described in Shendure et al. Science 309:1728-1732 (2005); U.S. Pat. Nos. 5,599,675; and 5,750,341, each of which is incorporated herein by reference in its entirety; and the SBH methodologies are as described in Bains et al., Journal of Theoretical Biology 135(3), 303-7 (1988); Drmanac et al., Nature Biotechnology 16, 54-58 (1998); Fodor et al., Science 251(4995), 767-773 (1995); and WO 1989/10977, each of which is incorporated herein by reference in its entirety.

In SBS, extension of a nucleic acid primer along a nucleic acid template is monitored to determine the sequence of nucleotides in the template. The underlying chemical process can be catalyzed by a polymerase, wherein fluorescently labeled nucleotides are added to a primer (thereby extending the primer) in a template dependent fashion such that detection of the order and type of nucleotides added to the primer can be used to determine the sequence of the template. A plurality of different nucleic acid fragments can be subjected to an SBS technique under conditions where events occurring for different templates can be distinguished due to their location in the array. In embodiments, the sequencing step includes annealing and extending a sequencing primer to incorporate a detectable label that indicates the identity of a nucleotide in the target polynucleotide, detecting the detectable label, and repeating the extending and detecting steps. In embodiments, the methods include sequencing one or more bases of a target nucleic acid by extending a sequencing primer hybridized to a target nucleic acid (e.g., an amplification product produced by the amplification methods described herein). In embodiments, the sequencing step may be accomplished by an SBS process. In embodiments, sequencing includes a sequencing by synthesis process, where individual nucleotides are identified iteratively, as they are polymerized to form a growing complementary strand. In embodiments, nucleotides added to a growing complementary strand include both a label and a reversible chain terminator that prevents further extension, such that the nucleotide may be identified by the label before removing the terminator to add and identify a further nucleotide. Such reversible chain terminators include removable 3′ blocking groups, for example as described in U.S. Pat. Nos. 10,738,072. Once such a modified nucleotide has been incorporated into the growing polynucleotide chain complementary to the region of the template being sequenced, there is no free 3′—OH group available to direct further sequence extension and therefore the polymerase cannot add further nucleotides. Once the identity of the base incorporated into the growing chain has been determined, the 3′ block may be removed to allow addition of the next successive nucleotide. By ordering the products derived using these modified nucleotides it is possible to deduce the DNA sequence of the DNA template. Non-limiting examples of suitable labels are described in U.S. Pat. Nos. 8,178,360, 5,188,934 (4,7-dichlorofluorscein dyes); U.S. Pat. No. 5,366,860 (spectrally resolvable rhodamine dyes); U.S. Pat. No. 5,847,162 (4,7-dichlororhodamine dyes); U.S. Pat. No. 4,318,846 (ether-substituted fluorescein dyes); U.S. Pat. No. 5,800,996 (energy transfer dyes); U.S. Pat. No. 5,066,580 (xanthene dyes): U.S. Pat. No. 5,688,648 (energy transfer dyes); and the like.

Sequencing includes, for example, detecting a sequence of signals. Examples of sequencing include, but are not limited to, sequencing by synthesis (SBS) processes in which reversibly terminated nucleotides carrying fluorescent dyes are incorporated into a growing strand, complementary to the target strand being sequenced. In embodiments, the nucleotides are labeled with up to four unique fluorescent dyes. In embodiments, the nucleotides are labeled with at least two unique fluorescent dyes. In embodiments, the readout is accomplished by epifluorescence imaging. A variety of sequencing chemistries are available, non-limiting examples of which are described herein.

Use of the sequencing method outlined above is a non-limiting example, as essentially any sequencing methodology which relies on successive incorporation of nucleotides into a polynucleotide chain can be used. Suitable alternative techniques include, for example, pyrosequencing methods, FISSEQ (fluorescent in situ sequencing), MPSS (massively parallel signature sequencing), or sequencing by ligation-based methods.

In embodiments, the method further includes obtaining an image of a cell or tissue. In embodiments, the imaging includes phase-contrast microscopy, bright-field microscopy, Nomarski differential-interference-contrast microscopy, dark field microscopy, electron microscopy, or cryo-electron microscopy. In embodiments, the light transmittance of the sample is measured. For example, light transmittance may be measured with a visible near-infrared optical fiber spectrometer, wherein a circular spot of light (e.g., diameter, 5 mm) is irradiated on the central part a sample and the transmitted light is collected using an optical sensor.

In embodiments, imaging a biomolecule includes an imaging modality including immunofluorescence (IF), or immunohistochemistry modality (e.g., immunostaining). In embodiments, the method includes ER staining (e.g., contacting the tissue section with a cell-permeable dye which localizes to the endoplasmic reticula), Golgi staining (e.g., contacting the tissue section with a cell-permeable dye which localizes to the Golgi), F-actin staining (e.g., contacting the tissue section with a phalloidin-conjugated dye that binds to actin filaments), lysosomal staining (e.g., contacting the tissue section with a cell-permeable dye that accumulates in the lysosome via the lysosome pH gradient), mitochondrial staining (e.g., contacting the tissue section with a cell-permeable dye which localizes to the mitochondria), nucleolar staining, or plasma membrane staining. For example, the method includes live cell imaging (e.g., obtaining images of the tissue section) prior to or during fixing, immobilizing, and permeabilizing the tissue section. Immunohistochemistry (IHC) is a powerful technique that exploits the specific binding between an antibody and antigen to detect and localize specific antigens in cells and tissue, commonly detected and examined with the light microscope. Known IHC modalities may be used, such as the protocols described in Magaki, S., Hojat, S. A., Wei, B., So, A., & Yong, W. H. (2019). Methods in molecular biology (Clifton, N.J.), 1897, 289-298, which is incorporated herein by reference. In embodiments, the additional imaging modality includes bright field microscopy, phase contrast microscopy, Nomarski differential-interference-contrast microscopy, or dark field microscopy. In embodiments, the method further includes determining the cell morphology of the tissue section (e.g., the cell boundary or cell shape) using known methods in the art. For example, to determining the cell boundary includes comparing the pixel values of an image to a single intensity threshold, which may be determined quickly using histogram-based approaches as described in Carpenter, A. et al Genome Biology 7, R100 (2006) and Arce, S., Sci Rep 3, 2266 (2013)). By “microscopic analysis” is meant the analysis of a specimen using techniques that provide for the visualization of aspects of a specimen that cannot be seen with the unaided eye, i.e., that are not within the resolution range of the normal human eye. Such techniques may include, without limitation, optical microscopy, e.g., bright field, oblique illumination, dark field, phase contrast, differential interference contrast, interference reflection, epifluorescence, confocal microscopy, CLARITY-optimized light sheet microscopy (COLM), light field microscopy, tissue expansion microscopy, etc., laser microscopy, such as, two photon microscopy, electron microscopy, and scanning probe microscopy. By “preparing a biological specimen for microscopic analysis” is generally meant rendering the specimen suitable for microscopic analysis at an unlimited depth within the specimen. In embodiments, the immobilized tissue section is imaged using “optical sectioning” techniques, such as laser scanning confocal microscopes, laser scanning 2-Photon microscopy, parallelized confocal (i.e. spinning disk), computational image deconvolution methods, and light sheet approaches. Optical sectioning microscopy methods provide information about single planes of a volume by minimizing contributions from other parts of the volume and do so without physical sectioning. The resulting “stack” of such optically sectioned images, represents a full reconstruction of the 3-dimensional features of a tissue volume. A typical confocal microscope includes a 10×/0.5 objective (dry; working distance, 2.0 mm) and/or a 20×/0.8 objective (dry; working distance, 0.55 mm), with a s z-step interval of 1 to 5 m. A typical light sheet fluorescence microscope includes an sCMOS camera, a 2×/0.5 objective lens, and zoom microscope body (magnification range of ×0.63 to ×6.3). For entire scanning of whole samples, the z-step interval is 5 or 10 m, and for image acquisition in the regions of interest, an interval in the range of 2 to 5 m may be used.

In an aspect is provided a method of making a compound of formula (I), the method including mixing compound A and compound B in a reaction vessel. Compound A has the formula:

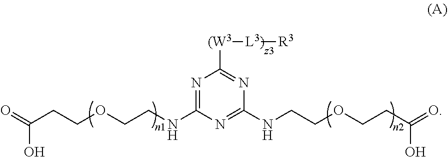

Compound B has the formula:

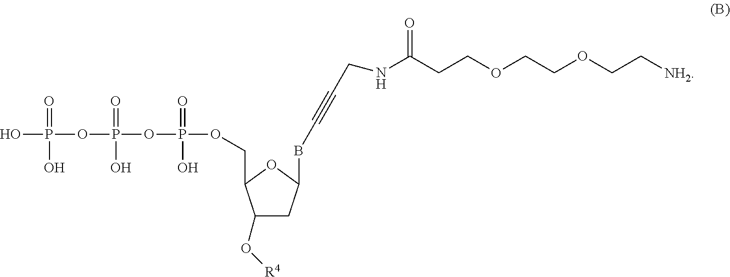

B is a nucleobase. R³ is a bioconjugate reactive moiety. R⁴ is hydrogen or a reversible terminator moiety. W³ is —O—,
“\*MERGEFORMAT\* MERGEFORMAT —NR³-A, or —S—. R^{3A} is hydrogen or substituted or unsubstituted alkyl. L³ is a covalent linker. n1 and n2 are independently an integer from 1 to 10. z3 is 0 or 1.

### EXAMPLES

### Example 1. Multivalent Nucleotide Constructs

Coverage uniformity in sequencing runs is critical for establishing reliability in sequencing results. Uniform sequencing coverage results from sequencing each base in a target region an equal number of times, which leads to an even distribution of sequencing reads across a target sequenced region. Thus, having a uniform distribution of sequencing reads across a target region enables the detection of true variants (e.g., copy number variants, single nucleotide variants, and insertions and deletions) and facilitates accurate analysis of differential gene expression between different cell types and cell populations.

Unfortunately, different commercially available next sequencing platforms suffer from coverage non-uniformity at varying degrees. Zhang et al. compared the performance metrics of NovaSeq™ 6000 and AVITI™ platforms in detecting copy number variations, single nucleotide variations, and Indels, and microsatellite instability and observed platform-specific differences in coverage uniformity (see preprint at doi.org/10.1101/2023.09.20.558622). Zhang et al. observed a higher GC dropout and lower overall uniformity from the AVITI™ data compared to the data obtained from NovaSeq™ 6000 and noted that the higher GC dropout observed in the data obtained from AVITI™ could arise from biases introduced during PCR amplification.

Furthermore, the structure of the multivalent reagent termed “Avidite” implemented in the sequencing workflow for AVITI™ is a source of variability. The Avidite reagent is composed of fluorescently labelled streptavidin and multiple biotinylated nucleotides (see, e.g., Arslan et al. Nat Biotechnol. 2024 January; 42(1):132-138). Streptavidin is a tetravalent protein that is a widely used tool in various applications owing its high binding affinity to biotin, which enables the detection and capture of a plethora of biotinylated substrates. Despite its versatility and specificity to biotinylated substrates, Xu et al. discussed the likelihood of obtaining statistical mixtures of streptavidin-biotin conjugates, rather than streptavidin-biotin conjugates with equal numbers of biotinylated substrates per streptavidin tetramer, when multiple biotinylated substrates are conjugated onto streptavidin (see Xu et al. Chem. Sci., 2020, 11, 4422-4429). Steric hindrance and the physical arrangement of the biotinylated substrate both influences the binding capacity of streptavidin and makes it difficult to precisely control the number of biotinylated substrates to add to a streptavidin tetramer. Therefore, factors contributing to the generation of statistical mixtures of multivalent streptavidin-biotin conjugates could also affect commercially available Avidite reagents and limit the consistency with which a particular multivalent streptavidin-biotin conjugate interacts with a polynucleotide template during a sequencing reaction.

Described herein are multivalent nucleotide constructs and methods of making thereof for the development of efficient sequencing reagents. FIG. 1A provides a structure of a compound described herein, which includes a central trifunctional scaffolding moiety (depicted as a triangle shape) that functions as a platform to attach a bioconjugate reactive moiety (depicted as an arc shape), a fluorescent moiety (depicted as a star shape), and a nucleotide moiety (illustrated with the pentagon bonded to a rectangle and a circle, where the rectangle depicts a nucleobase and the circle depicts a phosphate group). The bioconjugate reactive moiety, a fluorescent moiety, and a nucleotide moiety are all linked to the central trifunctional scaffold moiety via linker moieties (represented by the squiggly lines). FIG. 1B illustrates a multivalent nucleotide construct resulting from a chemical reaction of the bioconjugate reactive moiety of the compound of FIG. 1A and a second bioconjugate reactive moiety attached onto a protein (depicted as a cloud shape) via a linker moiety (represented as a squiggly line).

The multivalent nucleotide constructs described herein feature a nucleotide moiety, a fluorescent moiety, and a bioconjugate reactive moiety, all of which are scaffolded by a central ring (e.g., a triazine moiety,

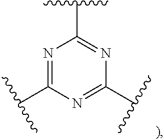 [SMILES: *c1nc(*)nc(*)n1]

which are afforded by a facile, controlled synthesis. The preparation of the multivalent nucleotide constructs described herein benefit from the inherent reactivity governed by the electronic effects of the triazine scaffold and a highly favorable bioconjugation step between the triazine scaffold bearing a bioconjugate reactive moiety (e.g., azide) and a biomolecule labelled with a second bioconjugate reactive moiety (e.g., dibenzocyclooctyne (DBCO)). The electronic effects inherent to the structure of the triazine enables the stepwise nucleophilic substitution of the moieties at R¹ and R² onto the triazine scaffold in a non-Poisson manner. Furthermore, the nitrogen atoms on the triazine scaffold adds hydrophilic character to the photostable detectable compounds described herein, which is advantageous for reactions performed in aqueous solutions and sequencing applications.

Schemes 1-3 depict protocols for deriving multivalent nucleotide constructs described herein. These protocols could be modified to develop multivalent nucleotide constructs with varying numbers and types of nucleotides and fluorescent moieties. The application of the methods described herein enable the generation of efficient and compact multivalent nucleotide constructs.

Scheme 1. Conjugation of trifunctional triazine with bioconjugate reactive moieties with modified guanine.

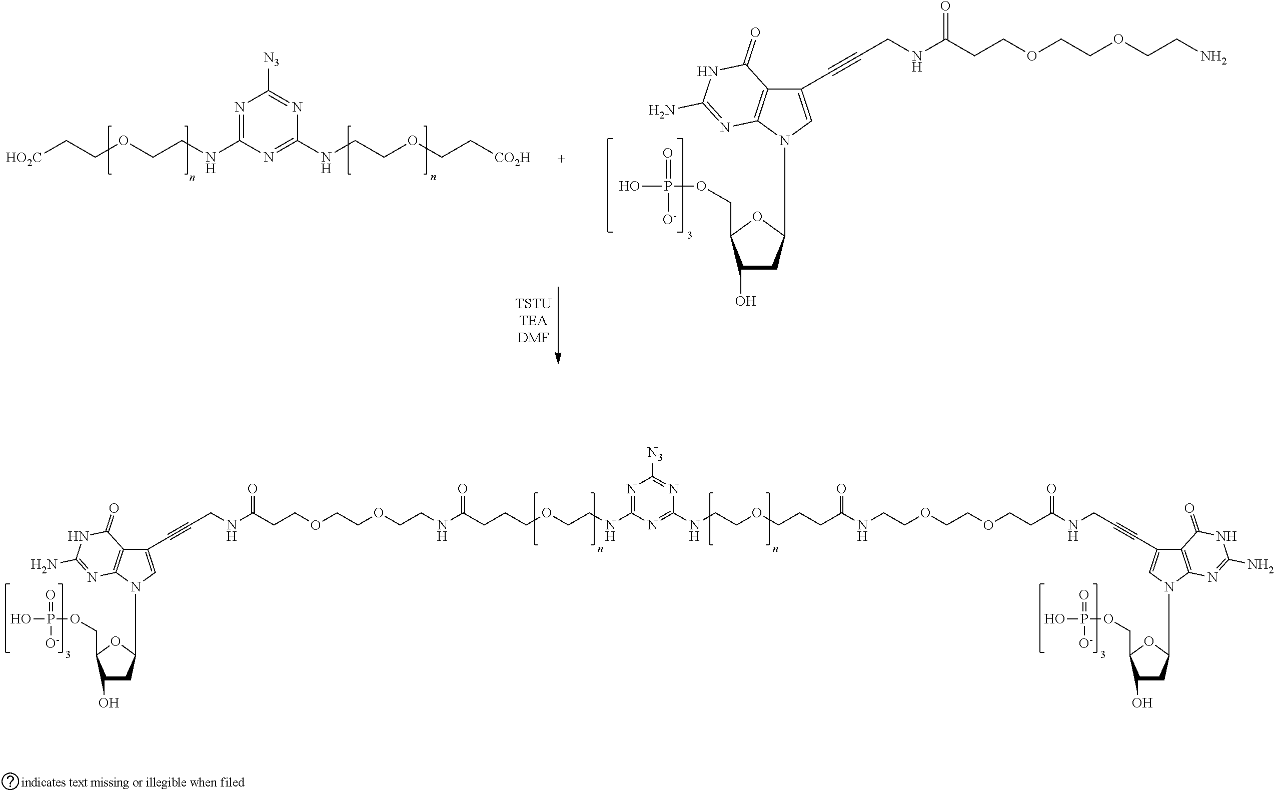

Scheme 2. Conjugation of trifunctional triazine with bioconjugate reactive moieties with modified guanine and cyanine-based fluorescent moiety.

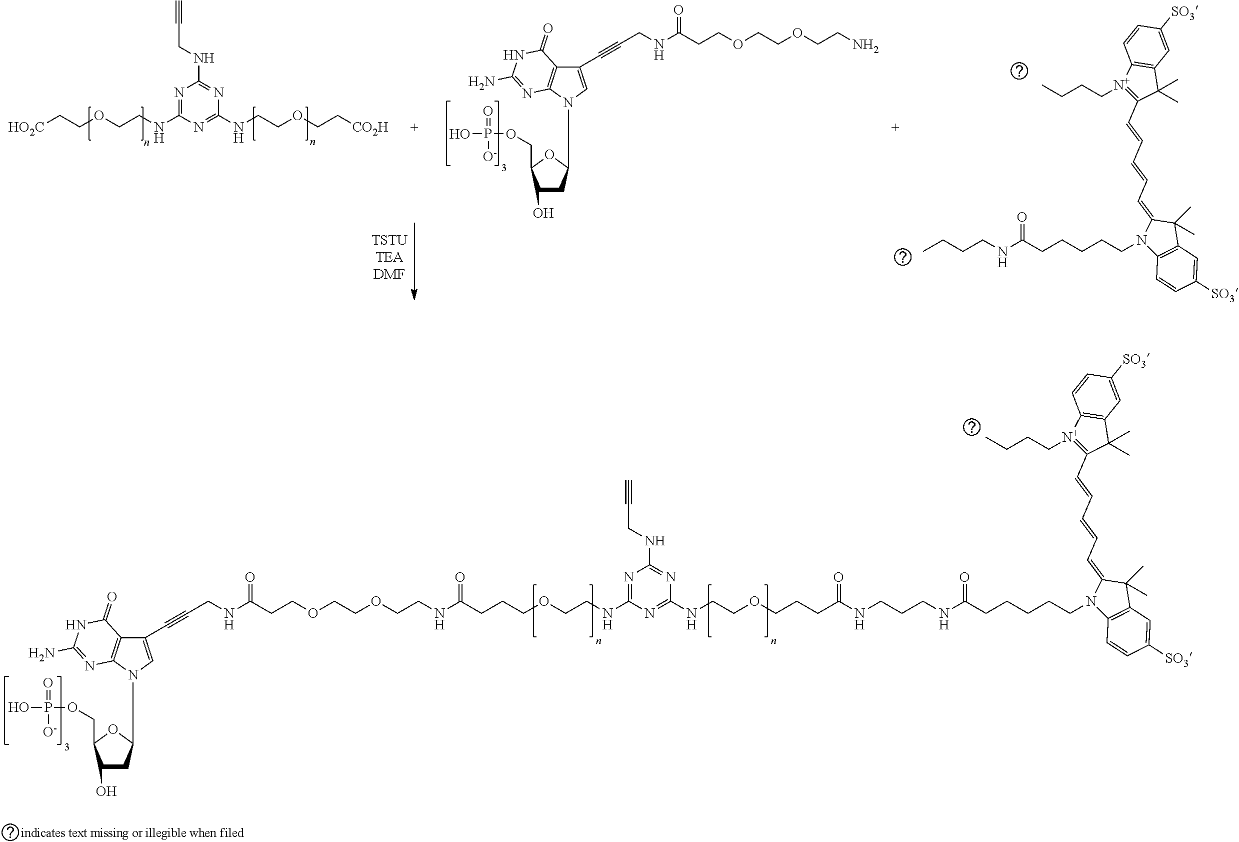

Scheme 3. Click reaction of azide functionalized construct generated in Scheme 1 with alkyne functionalized construct in Scheme 2 to generate fluorescently labelled multivalent guanine construct.

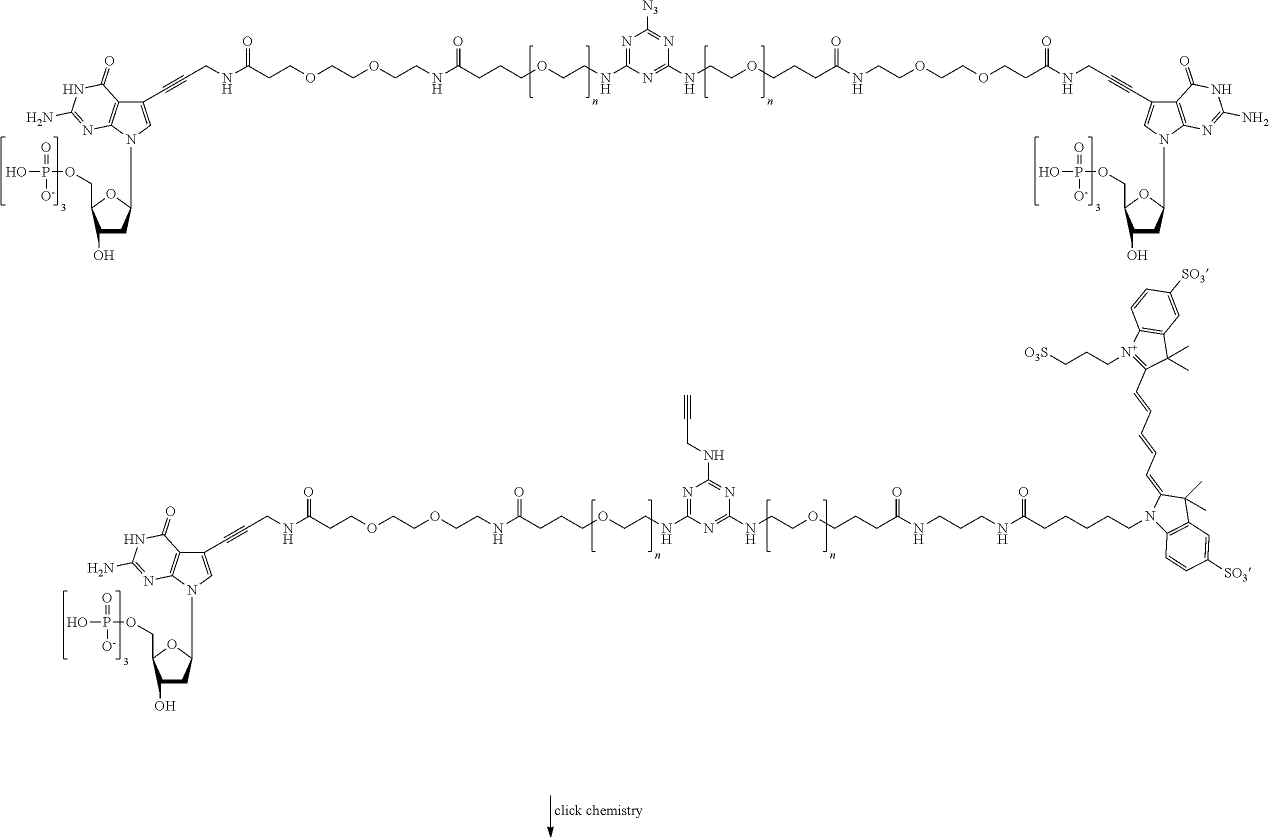

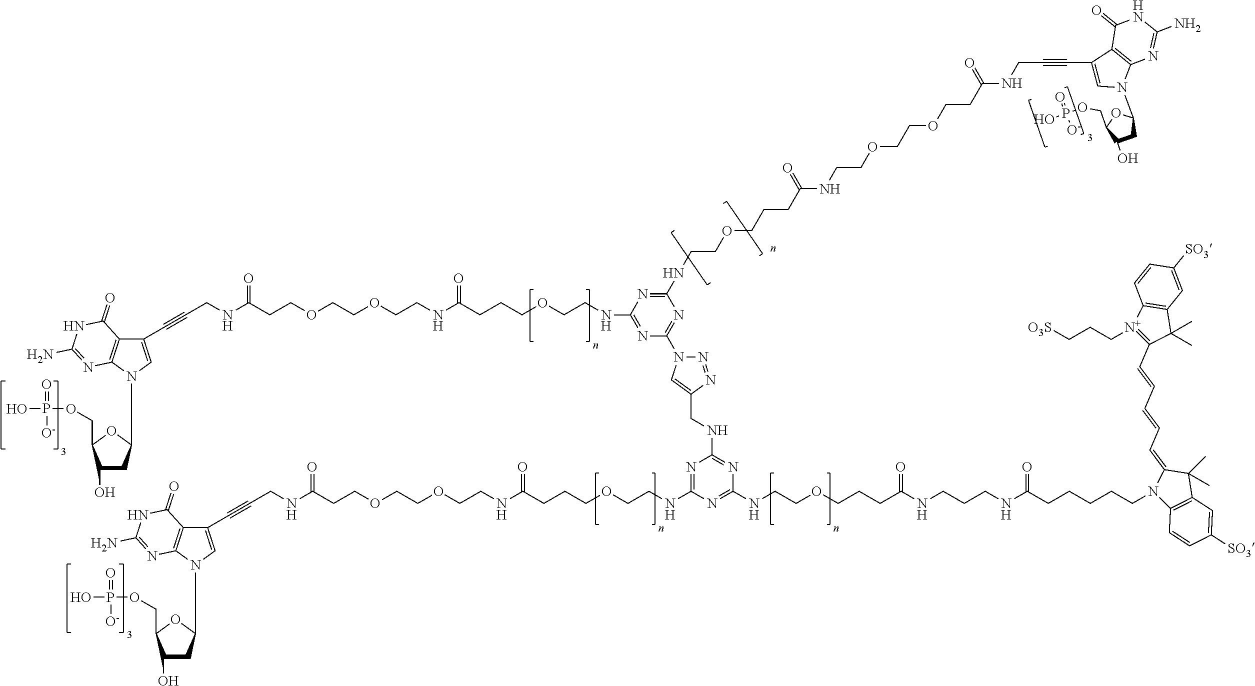

### Example 2. Spatial Transcriptomics and Proteomics

One influencing factor in the pathophysiological development of a disease stems from the aberrant gene and protein expression of disease-relevant genes and proteins along with the spatial heterogeneity in their abundance and distribution among cells and tissues. Spatial biology techniques, such as in situ sequencing, enables the scrutiny of disease-relevant biomolecules (such as lipids, carbohydrates, nucleic acids, and/or proteins) in the original context of intact tissue, which enables the evaluation of these macromolecules in relation to the tissue architecture and cellular microenvironment, both of which are governed by the intracellular and intercellular communication in situ.

Provided herein are compositions and methods for detecting target sequences in situ via the detection of a biomolecule attached to a nucleotide construct as described herein. Tissue sections may be manipulated using methods and techniques known in the art to for in situ transcriptomics workflows (see, e.g., U.S. Pat. No. 11,891,656, which is incorporated herein by reference in its entirety). For example, a tissue section including a target sequence (e.g., the mRNA transcript of the oncogene ERBB2) is detected in a tissue section adhered onto a solid support (e.g., a solid support described herein). To facilitate in situ detection of the target sequence, the tissue is contacted with a probe oligonucleotide as described herein to hybridize a first hybridization sequence and a second hybridization sequence of the probe oligonucleotide with a first sequence and a second sequence flanking the target sequence. In embodiments, 1, 2, 3, 4, 5, 6, 7, 8, 9, 10, 11, 12, 13, 14, 15, 16, 17, 18, 19, or 20 oligonucleotide probes are used to target each target sequence (e.g., mRNA transcript of interest). Following hybridization, the probe oligonucleotides are contacted with a ligase (e.g., SplintR® ligase) to ligate the ends of the probe oligonucleotides to generate a plurality of circular oligonucleotides. The resultant circular oligonucleotides are amplified using rolling circle amplification to generate amplification products and are subjected to sequencing. In embodiments, sequencing includes hybridizing a sequencing primer to the amplification product or complement thereof and extending a sequencing primer by incorporating a nucleotide moiety of a nucleotide construct attached to a biomolecule as described herein. In embodiments, the nucleotide construct and/or biomolecule includes a fluorescent moiety. In embodiments, sequencing includes detecting the fluorescent moiety for each incorporated nucleotide moiety. In embodiments, sequencing includes detecting a biomolecule attached to a nucleotide construct as described herein, wherein the biomolecule includes a fluorescent moiety. In embodiments, sequencing includes detecting a biomolecule attached to a nucleotide construct as described herein, wherein the nucleotide construct includes a fluorescent moiety. In embodiments, sequencing includes sequencing the first hybridization sequence and/or the second hybridization sequence of the probe oligonucleotide and using the sequence of the first hybridization sequence and/or the second hybridization sequence to identify the target sequence in the tissue section following bioinformatic analyses.

Other biomolecules contemplated for detection include a protein of interest (e.g., Akt). Tissue sections may be manipulated using methods and techniques known in the art to for in situ proteomics workflows (see, e.g., U.S. Pat. No. 11,891,656, which is incorporated herein by reference in its entirety). Detection of a protein of interest is performed using a protein-specific binding agent, such as an antibody with an oligonucleotide label (e.g., protein-specific antibody-oligo (Ab-O) conjugates), where the determination of the sequence of the oligonucleotide label and its association to the protein of interest is made a priori. In embodiments, the oligonucleotide label includes a target sequence as described herein. In embodiments, the tissue section is contacted with a probe oligonucleotide as described herein to hybridize a first hybridization sequence and a second hybridization sequence of the probe oligonucleotide with a first sequence and a second sequence flanking the target sequence. In embodiments, 1, 2, 3, 4, 5, 6, 7, 8, 9, 10, 11, 12, 13, 14, 15, 16, 17, 18, 19, or 20 oligonucleotide probes are used to target each target sequence (e.g., mRNA transcript of interest). Following hybridization, the probe oligonucleotides are contacted with a ligase (e.g., SplintR® ligase) to ligate the ends of the probe oligonucleotides to generate a plurality of circular oligonucleotides. The resultant circular oligonucleotides are amplified using rolling circle amplification to generate amplification products and are subjected to sequencing. In embodiments, sequencing includes hybridizing a sequencing primer to the amplification product or complement thereof and extending a sequencing primer by incorporating a nucleotide moiety of a nucleotide construct attached to a biomolecule as described herein. In embodiments, the nucleotide construct and/or biomolecule includes a fluorescent moiety. In embodiments, sequencing includes detecting the fluorescent moiety for each incorporated nucleotide moiety. In embodiments, sequencing includes detecting a biomolecule attached to a nucleotide construct as described herein, wherein the biomolecule includes a fluorescent moiety. In embodiments, sequencing includes detecting a biomolecule attached to a nucleotide construct as described herein, wherein the nucleotide construct includes a fluorescent moiety. In embodiments, sequencing includes sequencing the first hybridization sequence and/or the second hybridization sequence of the probe oligonucleotide and using the sequence of the first hybridization sequence and/or the second hybridization sequence to identify the target sequence in the tissue section following bioinformatic analyses.

What is claimed is:

1. A compound having the formula: 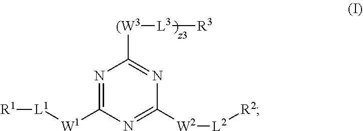
wherein
R¹ is a nucleotide moiety;
R² is a nucleotide moiety or a fluorescent moiety;
R³ is a bioconjugate reactive moiety;
W¹ is —O—, —NR^{1A}—, or —S—;
W² is —O—, —NR²A-, or —S—;
W³ is —O—, —NR³A-, or —S—;
R^{1A}, R^{2A}, and R^{3A} are independently hydrogen or substituted or unsubstituted alkyl;
L¹, L², and L³ and are independently covalent linkers; and
z3 is 0 or 1.

2. The compound of claim 1, wherein L¹ is L¹⁰¹-L¹⁰²-L¹⁰³; wherein
L¹⁰¹, L¹⁰², and L¹⁰³ are independently a bond, —C(O)O—, —NHC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

3. The compound of claim 1, wherein L¹ is 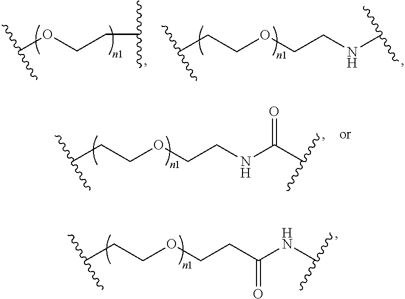
wherein n1 is an integer from 1 to 10.

4. The compound of claim 1, wherein L² is L²⁰¹-L²⁰²-L²⁰³; wherein
L²⁰¹, L²⁰², and L²⁰³ are independently a bond, —C(O)O—, —NHC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

5. The compound of claim 1, wherein L² is 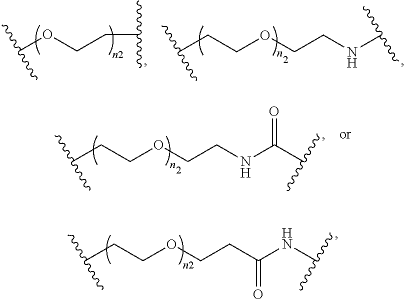
wherein n2 is an integer from 1 to 10.

6. The compound of claim 1, wherein W¹, W², and W³ are independently —NH—.

7. The compound of claim 1, wherein —W¹-L¹ and —W²-L² are independently

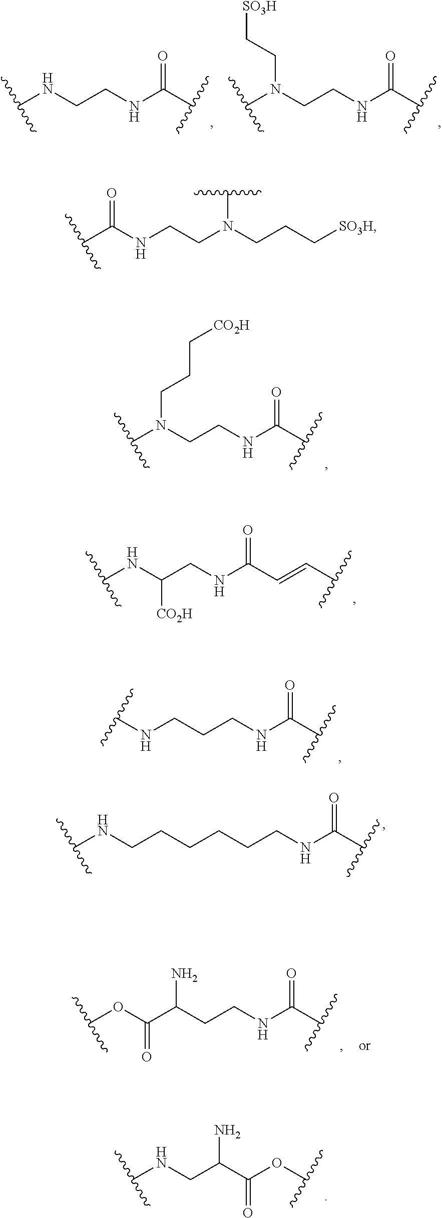

8. The compound of claim 1, wherein L³ is L³⁰¹-L³⁰²-L³⁰³; wherein L³⁰¹, L³⁰², and L³⁰³ are independently a bond, —C(O)O—, —NHC(O)—, —C(O)NH—, substituted or unsubstituted alkylene, substituted or unsubstituted heteroalkylene, substituted or unsubstituted cycloalkylene, substituted or unsubstituted heterocycloalkylene, substituted or unsubstituted arylene, or substituted or unsubstituted heteroarylene.

9. The compound of claim 1, wherein R³ is —N₃, avidin, streptavidin, —NH₂, —CN, —COOH,

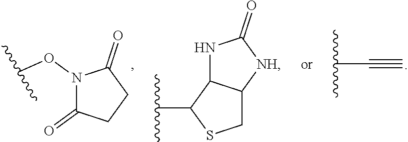

10. The compound of claim 1, wherein z3 is 0 and R³ is —N₃, avidin, streptavidin, —NH₂, —CN,

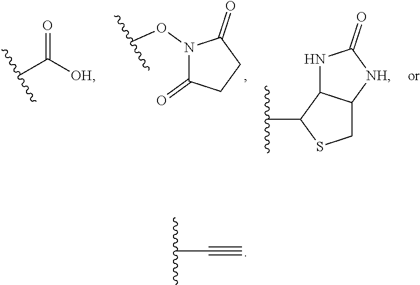

11. The compound of claim 1, wherein W³ is —NH—, and L³-R³ is

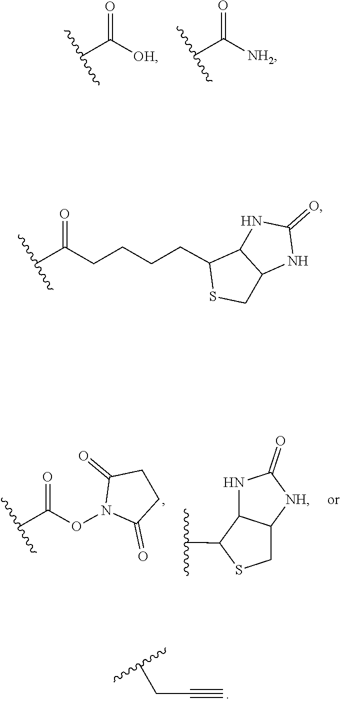

12. The compound of claim 1, wherein R¹ and R² independently have the formula: 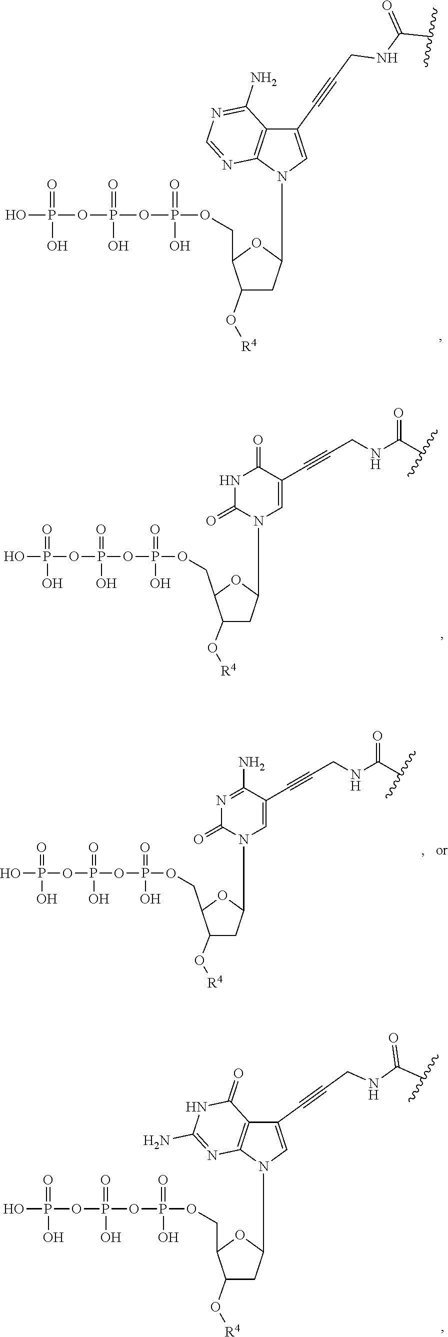
wherein R⁴ is hydrogen or a reversible terminator moiety.

13. The compound of claim 1, wherein R¹ and R² independently have the formula: 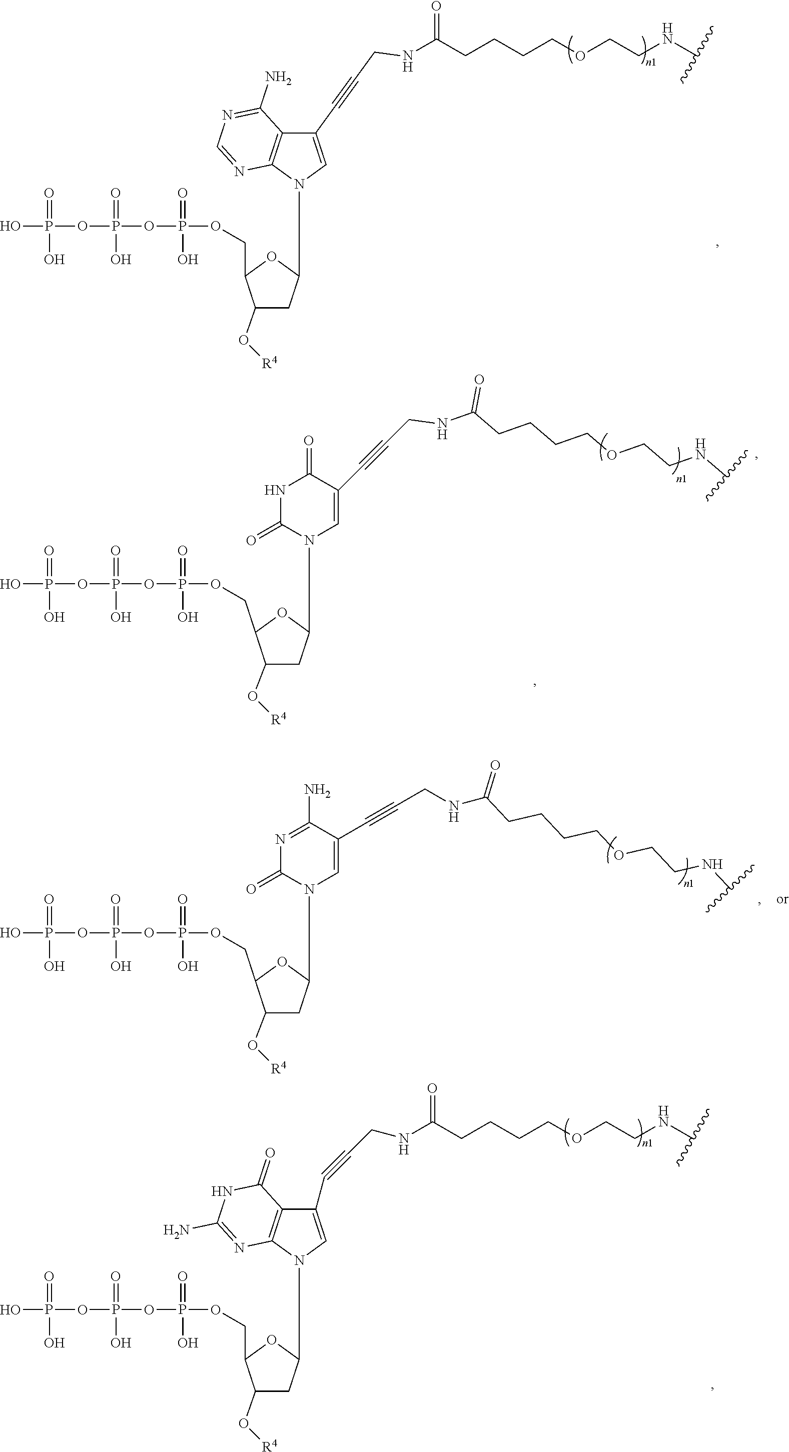
wherein
n1 is an integer from 1 to 10; and R⁴ is hydrogen or a reversible terminator moiety.

14. The compound of claim 1, having the formula: 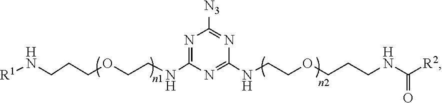
wherein n1 and n2 are independently an integer from 1 to 10.

15. The compound of claim 1, having the formula:

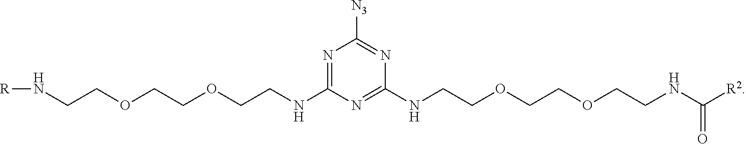

16. The compound of claim 1, having the formula:

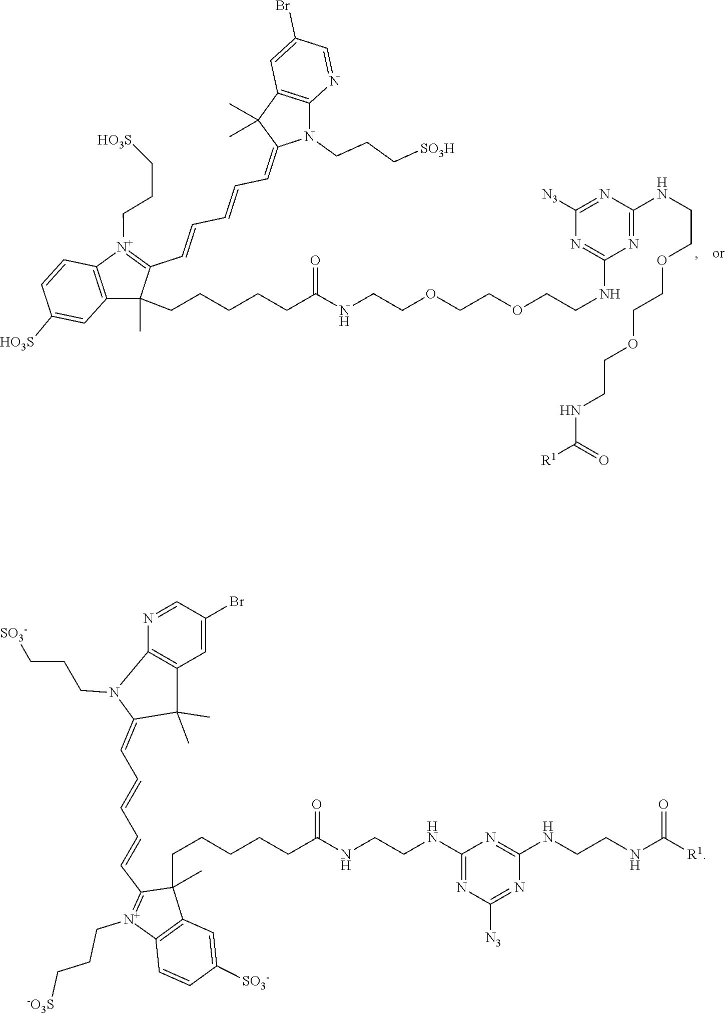

17. A biomolecule attached to a nucleotide construct, wherein said nucleotide construct has the formula: 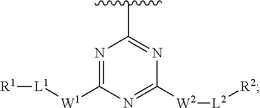
wherein
R¹ is a nucleotide moiety;
R² is a nucleotide moiety or a fluorescent moiety;
W¹ is O, NR^{1A}, or
W² is O, NR^{2A}, or S;
R^{1A} and R^{2A} are independently hydrogen or substituted or unsubstituted alkyl; and
L¹ and L² and are independently covalent linkers.

18. The biomolecule of claim 17, wherein the biomolecule is avidin or streptavidin.

19. The biomolecule of claim 17, wherein the biomolecule is further attached to a fluorescent moiety.

20. A method of imaging a target biomolecule, said method comprising directing an excitation beam onto a biomolecule comprising a detectable moiety and detecting a light emission from said detectable moiety, wherein said biomolecule is the biomolecule of claim 17.
